SEQUENCE LISTING (43 sequences)

SEQ ID NO: 1 - protein, 635 aa, Homo Sapiens
MEAPAQLLFLLLLWLPDTTGVVIGEVHENVTLHCGNISGLRGQVTWYRNNSEPVFLLSSNSSLRPAEPRFSLVDATSLHIESLSLGDEGIYTCQEILNVTQWFQVWLQVASGPYQIEVHIVATGTLPNGTLYAARGSQVDFSCNSSSRPPPVVEWWFQALNSSSESFGHNLTVNFFSLLLISPNLQGNYTCLALNQLSKRHRKVTTELLVYYPPPSAPQCWAQMASGSFMLQLTCRWDGGYPDPDFLWIEEPGGVIVGKSKLGVEMLSESQLSDGKKFKCVTSHIVGPESGASCMVQIRGPSLLSEPMKTCFTGGNVTLTCQVSGAYPPAKILWLRNLTQPEVIIQPSSRHLITQDGQNSTLTIHNCSQDLDEGYYICRADSPVGVREMEIWLSVKEPLNIGGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 615 aa, Homo Sapiens
VVIGEVHENVTLHCGNISGLRGQVTWYRNNSEPVFLLSSNSSLRPAEPRFSLVDATSLHIESLSLGDEGIYTCQEILNVTQWFQVWLQVASGPYQIEVHIVATGTLPNGTLYAARGSQVDFSCNSSSRPPPVVEWWFQALNSSSESFGHNLTVNFFSLLLISPNLQGNYTCLALNQLSKRHRKVTTELLVYYPPPSAPQCWAQMASGSFMLQLTCRWDGGYPDPDFLWIEEPGGVIVGKSKLGVEMLSESQLSDGKKFKCVTSHIVGPESGASCMVQIRGPSLLSEPMKTCFTGGNVTLTCQVSGAYPPAKILWLRNLTQPEVIIQPSSRHLITQDGQNSTLTIHNCSQDLDEGYYICRADSPVGVREMEIWLSVKEPLNIGGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 541 aa, Homo Sapiens
MAAGGSAPEPRVLVCLGALLAGWVAVGLEAVVIGEVHENVTLHCGNISGLRGQVTWYRNNSEPVFLLSSNSSLRPAEPRFSLVDATSLHIESLSLGDEGIYTCQEILNVTQWFQVWLQVASGPYQIEVHIVATGTLPNGTLYAARGSQVDFSCNSSSRPPPVVEWWFQALNSSSESFGHNLTVNFFSLLLISPNLQGNYTCLALNQLSKRHRKVTTELLVYYPPPSAPQCWAQMASGSFMLQLTCRWDGGYPDPDFLWIEEPGGVIVGKSKLGVEMLSESQLSDGKKFKCVTSHIVGPESGASCMVQIRGPSLLSEPMKTCFTGGNVTLTCQVSGAYPPAKILWLRNLTQPEVIIQPSSRHLITQDGQNSTLTIHNCSQDLDEGYYICRADSPVGVREMEIWLSVKEPLNIGGIVGTIVSLLLLGLAIISGLLLHYSPVFCWKVGNTSRGQNMDDVMVLVDSEEEEEEEEEEEEDAAVGEQEGAREGREELPKEIPKQDHIHRVTALVNGNIEQMGNGFQDLQDDSSEEQSDIVQEEDRPV

SEQ ID NO: 4 - protein, 383 aa, Homo Sapiens
VVIGEVHENVTLHCGNISGLRGQVTWYRNNSEPVFLLSSNSSLRPAEPRFSLVDATSLHIESLSLGDEGIYTCQEILNVTQWFQVWLQVASGPYQIEVHIVATGTLPNGTLYAARGSQVDFSCNSSSRPPPVVEWWFQALNSSSESFGHNLTVNFFSLLLISPNLQGNYTCLALNQLSKRHRKVTTELLVYYPPPSAPQCWAQMASGSFMLQLTCRWDGGYPDPDFLWIEEPGGVIVGKSKLGVEMLSESQLSDGKKFKCVTSHIVGPESGASCMVQIRGPSLLSEPMKTCFTGGNVTLTCQVSGAYPPAKILWLRNLTQPEVIIQPSSRHLITQDGQNSTLTIHNCSQDLDEGYYICRADSPVGVREMEIWLSVKEPLNIGG

SEQ ID NO: 5 - protein, 439 aa, Homo Sapiens
MAAGGSAPEPRVLVCLGALLAGWVAVGLEAVVIGEVHENVTLHCGNISGLRGQVTWYRNNSEPVFLLSSNSSLRPAEPRFSLVDATSLHIESLSLGDEGIYTCQEILNVTQWFQVWLQVANPPPSAPQCWAQMASGSFMLQLTCRWDGGYPDPDFLWIEEPGGVIVGKSKLGVEMLSESQLSDGKKFKCVTSHIVGPESGASCMVQIRGPSLLSEPMKTCFTGGNVTLTCQVSGAYPPAKILWLRNLTQPEVIIQPSSRHLITQDGQNSTLTIHNCSQDLDEGYYICRADSPVGVREMEIWLSVKEPLNIGGIVGTIVSLLLLGLAIISGLLLHYSPVFCWKVGNTSRGQNMDDVMVLVDSEEEEEEEEEEEEDAAVGEQEGAREREELPKEIPKQDHIHRVTALVNGNIEQMGNGFQDLQDDSSEEQSDIVQEEDRPV

SEQ ID NO: 6 - protein, 282 aa, Homo Sapiens
VVIGEVHENVTLHCGNISGLRGQVTWYRNNSEPVFLLSSNSSLRPAEPRFSLVDATSLHIESLSLGDEGIYTCQEILNVTQWFQVWLQVANPPPSAPQCWAQMASGSFMLQLTCRWDGGYPDPDFLWIEEPGGVIVGKSKLGVEMLSESQLSDGKKFKCVTSHIVGPESGASCMVQIRGPSLLSEPMKTCFTGGNVTLTCQVSGAYPPAKILWLRNLTQPEVIIQPSSRHLITQDGQNSTLTIHNCSQDLDEGYYICRADSPVGVREMEIWLSVKEPLNIGG

SEQ ID NO: 7 - protein, 429 aa, Homo Sapiens
MAGLRVLLCLGALLARQGSAGLQLLLNPSRANLSVRPNSEVLPGIHPDLEAVAIGEVHDNVTLRCGSASGSRGLVTWYRNDSEPAFLVSFNSSLPPAAPRFSLEDAGALRIEALRLEDDGNYTCQEVLNETHWFPVRLRVASGPAYVEVNISATGTLPNGTLYAARGSQVDFNCCSAAQPPPEVEWWIQTHSIPEFLGKNLSANSFTLMLMSQNLQGNYTCSATNVLSGRQRKVTTELLVYWPPPSAPQCSVEVSSESTTLELACNWDGGYPDPTFLWTEEPGGTIMGNSKLQTLSPAQLLEGKKFKCVGNHILGPESGASCVVKLSSPLLPSQPMRTCFVGGNVTLTCEVSGANPPARIQWLRNLTQPAIQPSSHYIITQQGQSSSLTIHNCSQDLDEGFYYCQAENLVGVRATNIWLSVKEPLNIGG

SEQ ID NO: 8 - protein, 641 aa, Homo Sapiens
LQLLLNPSRANLSVRPNSEVLPGIHPDLEAVAIGEVHDNVTLRCGSASGSRGLVTWYRNDSEPAFLVSFNSSLPPAAPRFSLEDAGALRIEALRLEDDGNYTCQEVLNETHWFPVRLRVASGPAYVEVNISATGTLPNGTLYAARGSQVDFNCCSAAQPPPEVEWWIQTHSIPEFLGKNLSANSFTLMLMSQNLQGNYTCSATNVLSGRQRKVTTELLVYWPPPSAPQCSVEVSSESTTLELACNWDGGYPDPTFLWTEEPGGTIMGNSKLQTLSPAQLLEGKKFKCVGNHILGPESGASCVVKLSSPLLPSQPMRTCFVGGNVTLTCEVSGANPPARIQWLRNLTQPAIQPSSHYIITQQGQSSSLTIHNCSQDLDEGFYYCQAENLVGVRATNIWLSVKEPLNIGGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 9 - protein, 233 aa, Homo Sapiens
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 10 - protein, 558 aa, Homo Sapiens
MAGLRVLLCLGALLARQGSAGLQLLLNPSRANLSVRPNSEVLPGIHPDLEAVAIGEVHDNVTLRCGSASGSRGLVTWYRNDSEPAFLVSFNSSLPPAAPRFSLEDAGALRIEALRLEDDGNYTCQEVLNETHWFPVRLRVASGPAYVEVNISATGTLPNGTLYAARGSQVDFNCCSAAQPPPEVEWWIQTHSIPEFLGKNLSANSFTLMLMSQNLQGNYTCSATNVLSGRQRKVTTELLVYWPPPSAPQCSVEVSSESTTLELACNWDGGYPDPTFLWTEEPGGTIMGNSKLQTLSPAQLLEGKKFKCVGNHILGPESGASCVVKLSSPLLPSQPMRTCFVGGNVTLTCEVSGANPPARIQWLRNLTQPAIQPSSHYIITQQGQSSSLTIHNCSQDLDEGFYYCQAENLVGVRATNIWLSVKEPLNIGGIVGTVVSLLLLGLAVVSGLTLYYSPAFWWKGGSTFRGQDMGDVMVLVDSEEEEEEEEEEEEKEDVAEEVEQETNETEELPKGISKHGHIHRVTALVNGNLDRMGNGFQEFQDDSDGQQSGIVQEDGKPV

SEQ ID NO: 11 - protein, 413 aa, Homo Sapiens
MAAGGSAPEPRVLVCLGALLAGWVAVGLEAVVIGEVHENVTLHCGNISGLRGQVTWYRNNSEPVFLLSSNSSLRPAEPRFSLVDATSLHIESLSLGDEGIYTCQEILNVTQWFQVWLQVASGPYQIEVHIVATGTLPNGTLYAARGSQVDFSCNSSSRPPPVVEWWFQALNSSSESFGHNLTVNFFSLLLISPNLQGNYTCLALNQLSKRHRKVTTELLVYYPPPSAPQCWAQMASGSFMLQLTCRWDGGYPDPDFLWIEEPGGVIVGKSKLGVEMLSESQLSDGKKFKCVTSHIVGPESGASCMVQIRGPSLLSEPMKTCFTGGNVTLTCQVSGAYPPAKILWLRNLTQPEVIIQPSSRHLITQDGQNSTLTIHNCSQDLDEGYYICRADSPVGVREMEIWLSVKEPLNIGG

SEQ ID NO: 12 - protein, 312 aa, Homo Sapiens
MAAGGSAPEPRVLVCLGALLAGWVAVGLEAVVIGEVHENVTLHCGNISGLRGQVTWYRNNSEPVFLLSSNSSLRPAEPRFSLVDATSLHIESLSLGDEGIYTCQEILNVTQWFQVWLQVANPPPSAPQCWAQMASGSFMLQLTCRWDGGYPDPDFLWIEEPGGVIVGKSKLGVEMLSESQLSDGKKFKCVTSHIVGPESGASCMVQIRGPSLLSEPMKTCFTGGNVTLTCQVSGAYPPAKILWLRNLTQPEVIIQPSSRHLITQDGQNSTLTIHNCSQDLDEGYYICRADSPVGVREMEIWLSVKEPLNIGG

SEQ ID NO: 13 - protein, 232 aa, Homo Sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 232 aa, Homo Sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 217 aa, Homo Sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 217 aa, Homo Sapiens
APNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 17 - protein, 514 aa, Homo Sapiens
VVIGEVHENVTLHCGNISGLRGQVTWYRNNSEPVFLLSSNSSLRPAEPRFSLVDATSLHIESLSLGDEGIYTCQEILNVTQWFQVWLQVANPPPSAPQCWAQMASGSFMLQLTCRWDGGYPDPDFLWIEEPGGVIVGKSKLGVEMLSESQLSDGKKFKCVTSHIVGPESGASCMVQIRGPSLLSEPMKTCFTGGNVTLTCQVSGAYPPAKILWLRNLTQPEVIIQPSSRHLITQDGQNSTLTIHNCSQDLDEGYYICRADSPVGVREMEIWLSVKEPLNIGGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - DNA, 402 bp, Homo Sapiens
atggaatggaggatctttctcttcatcctgtcaggaattgcaggtgtccactcccaggttcagctgcagcagtctggacctgagctggtgaggcctggggcttcagtgaagatgtcctgcaagccttctggatacagattcactgactacgctataagttgggtgaagcagagaactggacagggccttgagtgggttggagagatttatcctggaaatggtaatacttacttcaatgagaaattcaaggacaaggccacactgactgcagacagatcctccaacacagcctacatgcagctcagcagcctgacatctgaggactctgcggtctatttctgtgcaagaggctatgctaactacctgccctggggccaagggactctggtcactgtctctgca

SEQ ID NO: 19 - protein, 134 aa, Homo Sapiens
MEWRIFLFILSGIAGVHSQVQLQQSGPELVRPGASVKMSCKPSGYRFTDYAISWVKQRTGQGLEWVGEIYPGNGNTYFNEKFKDKATLTADRSSNTAYMQLSSLTSEDSAVYFCARGYANYLPWGQGTLVTVSA

SEQ ID NO: 20 - protein, 5 aa, Homo Sapiens
DYAIS

SEQ ID NO: 21 - protein, 17 aa, Homo Sapiens
EIYPGNGNTYFNEKFKD

SEQ ID NO: 22 - protein, 7 aa, Homo Sapiens
GYANYLP

SEQ ID NO: 23 - DNA, 381 bp, Homo Sapiens
atgaagtttccttctcaacctctgcttttactgctgtttggaatcccaggcatgatatgtgacatccagatgacacaatcttcatcctccttttctgtatctctaggagacagagtcaccattacttgtaaggcaagtgaggacatatataatcggttagcctggtatcagcagaaaccaggaaatgctcctaggctcttaatatctggtgcaaccggtttggaaactggggttccttcaagaatcagtggcagtggatctggaagggattacactctcagcattaccagtcttcagactgaagatgttggtacttattactgtcaacaatattggagtactcctcggacgttcggtggaggcaccaagttggaaatcaaa

SEQ ID NO: 24 - protein, 127 aa, Homo Sapiens
MKFPSQPLLLLLFGIPGMICDIQMTQSSSSFSVSLGDRVTITCKASEDIYNRLAWYQQKPGNAPRLLISGATGLETGVPSRISGSGSGRDYTLSITSLQTEDVGTYYCQQYWSTPRTFGGGTKLEIK

SEQ ID NO: 25 - protein, 11 aa, Homo Sapiens
KASEDIYNRLA

SEQ ID NO: 26 - protein, 7 aa, Homo Sapiens
GATGLET

SEQ ID NO: 27 - protein, 9 aa, Homo Sapiens
QQYWSTPRT

SEQ ID NO: 28 - DNA, 1647 bp, Homo Sapiens
atggccgctgggggaagcgcacctgaacctcgggtcctggtctgtctcggggctctgctggctggatgggtcgccgtcgggctggaagccgtggtcatcggggaggtccacgaaaacgtgaccctccattgcggtaatattagtgggctgaggggtcaggtcacatggtaccggaacaatagcgaaccagtgttcctgctcagctccaactctagtctgcgacctgcagagccaaggtttagtctcgtggacgccacctcactgcacatcgagtcactgagcctcggcgatgaaggaatctatacatgtcaggagattctgaatgtgactcagtggttccaggtctggctgcaggtggctagcggaccctaccagatcgaggtccatattgtggcaactgggaccctgcctaacggtacactctatgccgctaggggctcacaggtggactttagctgcaattcaagctccagaccccctccagtggtcgaatggtggttccaggccctgaactctagttcagagtcctttgggcacaacctgacagtgaatttcttttccctgctcctgatctctcccaacctgcagggtaattacacttgtctggcactcaatcagctgtctaagaggcatagaaaagtcaccacagagctcctggtgtactatccacctccaagcgcacctcagtgctgggctcagatggcatccggatctttcatgctgcagctcacttgtagatgggacggcggatatccagaccccgattttctgtggatcgaggaaccagggggtgtgattgtcggcaagtctaaactgggagtggagatgctcagtgaatcacagctgtcagatggaaagaaattcaagtgcgtcacaagccacatcgtggggcctgaaagcggtgcttcctgtatggtgcagattcgggggccatctctcctgagtgagcccatgaagacttgctttaccggcggaaacgtcacactgacttgtcaagtgagcggcgcctacccccctgctaaaatcctgtggctccggaatctgactcagcctgaggtgatcattcagccaagctcccgccacctgatcacccaggacggacagaactctaccctcacaattcataattgcagtcaggacctggatgagggctactatatctgtcgcgctgattccccagtgggagtccgagagatggaaatttggctctctgtgaaggagcccctgaacatcgggggtattgtcggcaccatcgtgagcctcctgctcctgggcctggcaatcattagcggactcctgctccactattcccctgtcttctgctggaaagtggggaacacaagcaggggtcagaatatggacgatgtgatggtcctggtggactccgaggaagaagaggaggaagaagaagaagaagaggaagatgcagcagtgggagagcaggaaggagcacgagaacgcgaggaactgcccaaggagatccctaaacaggaccacattcatcgcgtcaccgctctggtgaacggcaatatcgagcagatgggcaacggatttcaggatctgcaggacgattctagtgaggaacagagtgacattgtgcaggaggaggaccgacccgtggactataaagacgacgatgataagtaa

SEQ ID NO: 29 - DNA, 7911 bp, Homo Sapiens
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagatctgccaccatggccgctgggggaagcgcacctgaacctcgggtcctggtctgtctcggggctctgctggctggatgggtcgccgtcgggctggaagccgtggtcatcggggaggtccacgaaaacgtgaccctccattgcggtaatattagtgggctgaggggtcaggtcacatggtaccggaacaatagcgaaccagtgttcctgctcagctccaactctagtctgcgacctgcagagccaaggtttagtctcgtggacgccacctcactgcacatcgagtcactgagcctcggcgatgaaggaatctatacatgtcaggagattctgaatgtgactcagtggttccaggtctggctgcaggtggctagcggaccctaccagatcgaggtccatattgtggcaactgggaccctgcctaacggtacactctatgccgctaggggctcacaggtggactttagctgcaattcaagctccagaccccctccagtggtcgaatggtggttccaggccctgaactctagttcagagtcctttgggcacaacctgacagtgaatttcttttccctgctcctgatctctcccaacctgcagggtaattacacttgtctggcactcaatcagctgtctaagaggcatagaaaagtcaccacagagctcctggtgtactatccacctccaagcgcacctcagtgctgggctcagatggcatccggatctttcatgctgcagctcacttgtagatgggacggcggatatccagaccccgattttctgtggatcgaggaaccagggggtgtgattgtcggcaagtctaaactgggagtggagatgctcagtgaatcacagctgtcagatggaaagaaattcaagtgcgtcacaagccacatcgtggggcctgaaagcggtgcttcctgtatggtgcagattcgggggccatctctcctgagtgagcccatgaagacttgctttaccggcggaaacgtcacactgacttgtcaagtgagcggcgcctacccccctgctaaaatcctgtggctccggaatctgactcagcctgaggtgatcattcagccaagctcccgccacctgatcacccaggacggacagaactctaccctcacaattcataattgcagtcaggacctggatgagggctactatatctgtcgcgctgattccccagtgggagtccgagagatggaaatttggctctctgtgaaggagcccctgaacatcgggggtattgtcggcaccatcgtgagcctcctgctcctgggcctggcaatcattagcggactcctgctccactattcccctgtcttctgctggaaagtggggaacacaagcaggggtcagaatatggacgatgtgatggtcctggtggactccgaggaagaagaggaggaagaagaagaagaagaggaagatgcagcagtgggagagcaggaaggagcacgagaacgcgaggaactgcccaaggagatccctaaacaggaccacattcatcgcgtcaccgctctggtgaacggcaatatcgagcagatgggcaacggatttcaggatctgcaggacgattctagtgaggaacagagtgacattgtgcaggaggaggaccgacccgtggactataaagacgacgatgataagtaagttaacgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaa

SEQ ID NO: 30 - DNA, 1701 bp, Homo Sapiens
atggcagggctccgcgtgctcctctgtctcggtgctctcctcgctaggcagggttccgcagggctccagctcctcctcaatcccagccgcgccaacctgagtgtccgacctaattcagaggtgctgcccggcatccatcctgacctcgaggccgtggctattggagaagtccacgataacgtgactctgcgatgcggatccgcatctggaagtaggggactggtgacctggtacagaaacgacagtgagcccgccttcctggtgagcttcaacagctccctcccacctgcagctcctcgcttctctctggaggatgcaggtgccctccgaatcgaggccctgaggctcgaagacgatggcaactatacttgtcaggaggtgctgaatgaaacccattggtttcctgtcaggctgagagtggcttcaggaccagcatacgtggaggtcaacatcagcgctacaggcactctgcccaatggaaccctctatgcagccagggggtctcaggtggacttcaactgctgtagtgctgcacagccaccccctgaggtggaatggtggatccagacccactctattcctgagttcctgggaaagaacctctcagctaatagctttacactgatgctcatgagccagaacctgcagggaaattacacatgctcagcaactaacgtgctgagcgggcggcagcgcaaagtcaccacagagctgctcgtgtattggccaccacctagcgcacctcagtgctccgtggaggtctctagtgaaagcactaccctggagctcgcctgtaattgggacggcggataccctgatccaaccttcctgtggacagaggaaccagggggtacaatcatgggcaactccaagctgcagactctctctcccgcccagctgctcgagggcaagaagttcaagtgcgtgggtaatcatattctggggccagaatccggtgcttcttgtgtggtcaagctgtcaagccccctgctccctagccagccaatgagaacctgcttcgtcggcggaaacgtgaccctgacatgtgaggtgtccggggccaacccacccgctagaatccagtggctgcggaatctcacacagccagccattcagccctcctctcattatatcattacccagcagggccagagttcaagcctgacaatccacaactgcagccaggacctggatgagggtttttactattgtcaggcagaaaacctggtgggcgtcagagccactaatatttggctgtccgtgaaagagcctctcaatatcgggggtattgtgggcacagtggtctctctgctcctgctcggactggctgtggtcagcggactgacactctactattccccagcattctggtggaagggcggaagtacttttcggggccaggacatgggagatgtgatggtcctggtggacagcgaggaagaagaggaggaagaagaagaagaagaggaaaaagaggatgtcgcagaggaagtggagcaggaaactaacgaaaccgaggaactgccaaaggggatctccaaacacggtcatattcaccgggtcaccgctctggtgaacggcaatctcgaccgcatggggaatggtttccaggagtttcaggacgattctgacgggcagcagagtggtatcgtccaggaagatggaaagcccgtggactacaaggacgatgacgataaatga

SEQ ID NO: 31 - DNA, 7127 bp, Homo Sapiens
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattcagatctgccaccatggcagggctccgcgtgctcctctgtctcggtgctctcctcgctaggcagggttccgcagggctccagctcctcctcaatcccagccgcgccaacctgagtgtccgacctaattcagaggtgctgcccggcatccatcctgacctcgaggccgtggctattggagaagtccacgataacgtgactctgcgatgcggatccgcatctggaagtaggggactggtgacctggtacagaaacgacagtgagcccgccttcctggtgagcttcaacagctccctcccacctgcagctcctcgcttctctctggaggatgcaggtgccctccgaatcgaggccctgaggctcgaagacgatggcaactatacttgtcaggaggtgctgaatgaaacccattggtttcctgtcaggctgagagtggcttcaggaccagcatacgtggaggtcaacatcagcgctacaggcactctgcccaatggaaccctctatgcagccagggggtctcaggtggacttcaactgctgtagtgctgcacagccaccccctgaggtggaatggtggatccagacccactctattcctgagttcctgggaaagaacctctcagctaatagctttacactgatgctcatgagccagaacctgcagggaaattacacatgctcagcaactaacgtgctgagcgggcggcagcgcaaagtcaccacagagctgctcgtgtattggccaccacctagcgcacctcagtgctccgtggaggtctctagtgaaagcactaccctggagctcgcctgtaattgggacggcggataccctgatccaaccttcctgtggacagaggaaccagggggtacaatcatgggcaactccaagctgcagactctctctcccgcccagctgctcgagggcaagaagttcaagtgcgtgggtaatcatattctggggccagaatccggtgcttcttgtgtggtcaagctgtcaagccccctgctccctagccagccaatgagaacctgcttcgtcggcggaaacgtgaccctgacatgtgaggtgtccggggccaacccacccgctagaatccagtggctgcggaatctcacacagccagccattcagccctcctctcattatatcattacccagcagggccagagttcaagcctgacaatccacaactgcagccaggacctggatgagggtttttactattgtcaggcagaaaacctggtgggcgtcagagccactaatatttggctgtccgtgaaagagcctctcaatatcgggggtattgtgggcacagtggtctctctgctcctgctcggactggctgtggtcagcggactgacactctactattccccagcattctggtggaagggcggaagtacttttcggggccaggacatgggagatgtgatggtcctggtggacagcgaggaagaagaggaggaagaagaagaagaagaggaaaaagaggatgtcgcagaggaagtggagcaggaaactaacgaaaccgaggaactgccaaaggggatctccaaacacggtcatattcaccgggtcaccgctctggtgaacggcaatctcgaccgcatggggaatggtttccaggagtttcaggacgattctgacgggcagcagagtggtatcgtccaggaagatggaaagcccgtggactacaaggacgatgacgataaatgagttaacgcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 32 - protein, 548 aa, Homo Sapiens
MAAGGSAPEPRVLVCLGALLAGWVAVGLEAVVIGEVHENVTLHCGNISGLRGQVTWYRNNSEPVFLLSSNSSLRPAEPRFSLVDATSLHIESLSLGDEGIYTCQEILNVTQWFQVWLQVASGPYQIEVHIVATGTLPNGTLYAARGSQVDFSCNSSSRPPPVVEWWFQALNSSSESFGHNLTVNFFSLLLISPNLQGNYTCLALNQLSKRHRKVTTELLVYYPPPSAPQCWAQMASGSFMLQLTCRWDGGYPDPDFLWIEEPGGVIVGKSKLGVEMLSESQLSDGKKFKCVTSHIVGPESGASCMVQIRGPSLLSEPMKTCFTGGNVTLTCQVSGAYPPAKILWLRNLTQPEVIIQPSSRHLITQDGQNSTLTIHNCSQDLDEGYYICRADSPVGVREMEIWLSVKEPLNIGGIVGTIVSLLLLGLAIISGLLLHYSPVFCWKVGNTSRGQNMDDVMVLVDSEEEEEEEEEEEEDAAVGEQEGAREREELPKEIPKQDHIHRVTALVNGNIEQMGNGFQDLQDDSSEEQSDIVQEEDRPVDYKDDDDK

SEQ ID NO: 33 - protein, 566 aa, Homo Sapiens
MAGLRVLLCLGALLARQGSAGLQLLLNPSRANLSVRPNSEVLPGIHPDLEAVAIGEVHDNVTLRCGSASGSRGLVTWYRNDSEPAFLVSFNSSLPPAAPRFSLEDAGALRIEALRLEDDGNYTCQEVLNETHWFPVRLRVASGPAYVEVNISATGTLPNGTLYAARGSQVDFNCCSAAQPPPEVEWWIQTHSIPEFLGKNLSANSFTLMLMSQNLQGNYTCSATNVLSGRQRKVTTELLVYWPPPSAPQCSVEVSSESTTLELACNWDGGYPDPTFLWTEEPGGTIMGNSKLQTLSPAQLLEGKKFKCVGNHILGPESGASCVVKLSSPLLPSQPMRTCFVGGNVTLTCEVSGANPPARIQWLRNLTQPAIQPSSHYIITQQGQSSSLTIHNCSQDLDEGFYYCQAENLVGVRATNIWLSVKEPLNIGGIVGTVVSLLLLGLAVVSGLTLYYSPAFWWKGGSTFRGQDMGDVMVLVDSEEEEEEEEEEEEKEDVAEEVEQETNETEELPKGISKHGHIHRVTALVNGNLDRMGNGFQEFQDDSDGQQSGIVQEDGKPVDYKDDDDK

SEQ ID NO: 34 - DNA, 408 bp, Homo Sapiens
atgggatggagctggatctttctcttcctcttgtcaggaactgcaggtgtcctctctgaggtccagctgcaacaatctggacctgagctggtgaagcctggggcttcagtgaaaatgtcctgtaaggcttctggatacacattcactgactactacatgaagtgggtgaagcagagtcatggaaagagccttgagtggattggagatattaatcctaacaatggtggtacaacctacaaccagaagttcaagggcaaggccacattgactgtggacaaatcctccaacacagcctacatgcagttcaacagcctgacatctgaggactctgcagtctatttctgtgcaagatttcggctacgagctatggactactggggtcaaggaacctcagtcaccgtctcctca

SEQ ID NO: 35 - protein, 136 aa, Homo Sapiens
MGWSWIFLFLLSGTAGVLSEVQLQQSGPELVKPGASVKMSCKASGYTFTDYYMKWVKQSHGKSLEWIGDINPNNGGTTYNQKFKGKATLTVDKSSNTAYMQFNSLTSEDSAVYFCARFRLRAMDYWGQGTSVTVSS

SEQ ID NO: 36 - protein, 5 aa, Homo Sapiens
DYYMK

SEQ ID NO: 37 - protein, 17 aa, Homo Sapiens
DINPNNGGTTYNQKFKG

SEQ ID NO: 38 - protein, 8 aa, Homo Sapiens
FRLRAMDY

SEQ ID NO: 39 - DNA, 399 bp, Homo Sapiens
atggaatcacagactcaggtcctcatgtccctgctgttctgggtatctggtacctgtggggacattgtgatgacacagtctccatcctccctgactgtgacagcaggagagaaggtcactatgagctgcaagtccagtcagagtctgttaaacagtggagatcgaaagaactacttgacctggtaccagcagagaccagggctgcctcctaaactgttgatctactgggcatccactagggaatctggggtccctgatcgcttcacaggcagtggatctggaacagatttcactctcaccatcagcagtgtgcagtctgaagacctggcagtttatttctgtcagaatgattatatttatccgctcacgttcggtgctgggaccaagctggagctgaaa

SEQ ID NO: 40 - protein, 133 aa, Homo Sapiens
MESQTQVLMSLLFWVSGTCGDIVMTQSPSSLTVTAGEKVTMSCKSSQSLLNSGDRKNYLTWYQQRPGLPPKLLIYWASTRESGVPDRFTGSGSGTDFTLTISSVQSEDLAVYFCQNDYIYPLTFGAGTKLELK

SEQ ID NO: 41 - protein, 17 aa, Homo Sapiens
KSSQSLLNSGDRKNYLT

SEQ ID NO: 42 - protein, 7 aa, Homo Sapiens
WASTRES

SEQ ID NO: 43 - protein, 9 aa, Homo Sapiens
QNDYIYPLT

(19) United States
(12) Patent Application Publication
(10) Pub. No.: US 2020/0270343 A1
(43) Pub. Date: Aug. 27, 2020

(54) ANTI-VSIG10 ANTIBODIES AND METHODS OF USE

(71) Applicant: COMPUGEN LTD., Holon (IL)

(72) Inventors: Ofer LEVY, Shimson (IL); Gad S. COJOCARU, Ramat-HaSharon (IL); Liat DASSA, Tel Aviv (IL); Tal FRIDMAN-KFIR, Tel Aviv (IL); Ilan VAKNIN, Modiin (IL); Einav GANGULI, Baltimore, MD (US); Finav GANGULI, Raanana (IL); Drew PARDOLL, Baltimore, MD (US)

(21) Appl. No.: 16/003,356

(22) Filed: Jun. 8, 2018

Related U.S. Application Data
(60) Provisional application No. 62/516,773, filed on Jun. 8, 2017.
(60) Provisional application No. 62/551,349, filed on Aug. 29, 2017.

(51) Int. Cl. C07K 16/28; A61P 35/00; A61K 45/06; A61K 39/395

(52) U.S. Cl. CPC C07K 16/2803; A61P 35/00; A61K 45/06; A61K 39/39558; A61K 2039/505; C07K 2317/622; C07K 2317/21; C07K 2317/24; C07K 2317/565

(57) ABSTRACT

A monoclonal or polyclonal antibody or an antigen binding fragment thereof comprising an antigen binding site that binds specifically to an isolated polypeptide comprising amino acids of the soluble ectodomain of a sequence selected from the group consisting of SEQ ID NOs:3 and 5, or a fragment, thereof, or an epitope thereof; for use in treatment of cancer, wherein immune cells in the microenvironment of said cancer express said isolated polypeptide.

### BACKGROUND OF THE INVENTION

Naïve T cells must receive two independent signals from antigen-presenting cells (APC) in order to become productively activated. The first, Signal 1, is antigen-specific and occurs when T cell antigen receptors encounter the appropriate antigen-MHC complex on the APC. The fate of the immune response is determined by a second, antigen-independent signal (Signal 2) which is delivered through a T cell costimulatory molecule that engages its APC-expressed ligand. This second signal could be either stimulatory (positive costimulation) or inhibitory (negative costimulation or coinhibition). In the absence of a costimulatory signal, or in the presence of a coinhibitory signal, T-cell activation is impaired or aborted, which may lead to a state of antigen-specific unresponsiveness (known as T-cell anergy), or may result in T-cell apoptotic death.

Costimulatory molecule pairs usually consist of ligands expressed on APCs and their cognate receptors expressed on T cells. The prototype ligand/receptor pairs of costimulatory molecules are B7/CD28 and CD40/CD40L. The B7 family consists of structurally related, cell-surface protein ligands, which may provide stimulatory or inhibitory input to an immune response. Members of the B7 family are structurally related, with the extracellular domain containing at least one variable or constant immunoglobulin domain.

Both positive and negative costimulatory signals play critical roles in the regulation of cell-mediated immune responses, and molecules that mediate these signals have proven to be effective targets for immunomodulation. Based on this knowledge, several therapeutic approaches that involve targeting of costimulatory molecules have been developed, and were shown to be useful for prevention and treatment of cancer by turning on, or preventing the turning off, of immune responses in cancer patients and for prevention and treatment of autoimmune diseases and inflammatory diseases, as well as rejection of allogenic transplantation, each by turning off uncontrolled immune responses, or by induction of “off signal” by negative costimulation (or coinhibition) in subjects with these pathological conditions.

Manipulation of the signals delivered by B7 ligands has shown potential in the treatment of autoimmunity, inflammatory diseases, and transplant rejection. Therapeutic strategies include blocking of costimulation using monoclonal antibodies to the ligand or to the receptor of a costimulatory pair, or using soluble fusion proteins composed of the costimulatory receptor that may bind and block its appropriate ligand. Another approach is induction of co-inhibition using soluble fusion protein of an inhibitory ligand. These approaches rely, at least partially, on the eventual deletion of auto- or allo-reactive T cells (which are responsible for the pathogenic processes in autoimmune diseases or transplantation, respectively), presumably because in the absence of costimulation (which induces cell survival genes) T cells become highly susceptible to induction of apoptosis. Thus, novel agents that are capable of modulating costimulatory signals, without compromising the immune system's ability to defend against pathogens, are highly advantageous for treatment and prevention of such pathological conditions.

Costimulatory pathways play an important role in tumor development. Interestingly, tumors have been shown to evade immune destruction by impeding T cell activation through inhibition of co-stimulatory factors in the B7-CD28 and TNF families, as well as by attracting regulatory T cells, which inhibit anti-tumor T cell responses (see Wang (2006), “Immune Suppression by Tumor Specific CD4⁺ Regulatory T cells in Cancer”, Semin. Cancer. Biol. 16:73-79; Greenwald, et al. (2005), “The B7 Family Revisited”, Ann. Rev. Immunol. 23:515-48; Watts (2005), “TNF/TNFR Family Members in Co-stimulation of T Cell Responses”, Ann. Rev. Immunol. 23:23-68; Sadum, et al., (2007) “Immune Signatures of Murine and Human Cancers Reveal Unique Mechanisms of Tumor Escape and New Targets for Cancer Immunotherapy”, Clin. Canc. Res. 13(13): 4016-4025). Such tumor expressed co-stimulatory molecules have become attractive cancer biomarkers and may serve as tumor-associated antigens (TAAs). Furthermore, costimulatory pathways have been identified as immunologic checkpoints that attenuate T cell dependent immune responses, both at the level of initiation and effector function within tumor metastases. As engineered cancer vaccines continue to improve, it is becoming clear that such immunologic checkpoints are a major barrier to the vaccines' ability to induce therapeutic anti-tumor responses. In that regard, costimulatory molecules can serve as adjuvants for active (vaccination) and passive (antibody-mediated) cancer immunotherapy, providing strategies to thwart immune tolerance and stimulate the immune system.

Over the past decade, agonists and/or antagonists to various costimulatory proteins have been developed for treating autoimmune diseases, graft rejection, allergy and cancer. For example, CTLA4-Ig (Abatacept, Orencia®) is approved for treatment of RA, mutated CTLA4-Ig (Belatacept, Nulojix®) for prevention of acute kidney transplant rejection and by the anti-CTLA4 antibody (Ipilimumab, Yervoy®), recently approved for the treatment of melanoma. Other costimulation regulators have been approved, such as the anti-PD-1 antibodies of Merck (Keytruda®) and BMS (Opdivo®), have been approved for cancer treatments and are in testing for viral infections as well.

Accordingly, it is an object of the invention to provide VSIG10 immunomodulatory antibodies.

### BRIEF SUMMARY OF THE INVENTION

According to at least some embodiments, the invention provides an isolated antibody specifically binding to an ectodomain or soluble or secreted form of the VSIG10 protein and/or variants and/or orthologs and/or fragments, or a novel therapeutic and diagnostic compositions containing same. The isolated antibody modulates the immune system through binding to VSIG10. The term “VSIG10” is used collectively for various amino acid sequences as described herein. Optionally the isolated antibody specifically binds to a suitable epitope on any of these amino acid sequences.

By “antibody” it is meant any of monoclonal or polyclonal antibodies and antigen binding fragments and conjugates containing same, and/or alternative scaffolds.

In at least some embodiments, the immune system modulation may be used to treat cancer, even if the cancer cells do not express VSIG10. The immune system cells in the microenvironment of the cancer express VSIG10, and it is this expression that the isolated antibody modulates. Preferably, the antibody downregulates or blocks VSIG10 activity in the microenvironment of the tumor, thereby inducing immune system activity against the cancer cells.

Optionally, the ectodomain is selected from the group consisting of SEQ ID NOs:4 and 6.

Optionally, the immune infiltrating cells in the tumor microenvironment are myeloid lineage cells.

Optionally, the myeloid lineage cells are dendritic cells.

Optionally, the dendritic cells are CD1C positive dendritic cells.

Optionally, the dendritic cells are CD207 positive dendritic cells.

Optionally, the cancer cells are epithelial cells.

Optionally, the antibody or the antigen binding fragment is capable of performing an activity selected from the group consisting of: activating cytotoxic T cells (CTLs), wherein a subset of the CTLs are activated; activating NK cells, wherein a subset of the NK cells are activated; activating Th1 cells, wherein a subset of the Th1 cells are activated; decreasing or eliminating cell number and/or activity of at least one of regulatory T cells (Tregs); and increasing interferon-γ production and/or pro-inflammatory cytokine secretion; or a combination thereof.

Optionally, the antibody or the antigen binding fragment comprises a monoclonal antibody selected from the group consisting of 577-Ab and 576-Ab.

Optionally, the antibody or the antigen binding fragment comprises a monoclonal antibody binding to the same epitope as the monoclonal antibody selected from the group consisting of 577-Ab and 576-Ab.

Optionally, the antibody or the antigen binding fragment comprises a monoclonal antibody comprising a heavy chain having an amino acid sequence selected from the group consisting of SEQ ID NO:201 and SEQ ID NO:217.

Optionally, the antibody or the antigen binding fragment comprises a monoclonal antibody comprising a heavy chain having the same binding specificity as the heavy chain having an amino acid sequence selected from the group consisting of SEQ ID NO:201 and SEQ ID NO:217.

Optionally, the antibody or the antigen binding fragment comprises a monoclonal antibody comprising a light chain having an amino acid sequence selected from the group consisting of SEQ ID NO:206 and SEQ ID NO:222.

Optionally, the antibody or the antigen binding fragment comprises a monoclonal antibody comprising a light chain having the same binding specificity as the light chain having an amino acid sequence selected from the group consisting of SEQ ID NO:206 and SEQ ID NO:222.

Optionally, the antibody or the antigen binding fragment comprises any of:

a heavy chain having an amino acid sequence of SEQ ID NO: 201 and a light chain having an amino acid sequence of SEQ ID NO: 206; or

a heavy chain having an amino acid sequence of SEQ ID NO: 217 and a light chain having an amino acid sequence of SEQ ID NO: 222.

Optionally, the antibody or the antigen binding fragment comprises:

a) a heavy chain variable domain comprising a vhCDR1, vhCDR2, and vhCDR3 from an anti-VSIG10 antibody; and

b) a light chain variable domain comprising a vlCDR1, vlCDR2 and vlCDR3 from said anti-VSIG10 antibody;

wherein said anti-VSIG10 antibody is selected from the group consisting of 577-Ab and wherein said SEQ ID Nos are 202, 203, 204 for vhCDR1, vhCDR2, vhCDR3, respectively and 207, 208, 209 for vlCDR1, vlCDR2, vlCDR3 respectively; or

wherein said anti-VSIG10 antibody is selected from the group consisting of 576-Ab and wherein said SEQ ID Nos are 218, 219, 2220 for vhCDR1, vhCDR2, vhCDR3, respectively and 223, 224, 225 for vlCDR1, vlCDR2, vlCDR3 respectively.

Optionally said antigen binding domain is a scFv single chain Fv (scFv), wherein said heavy chain variable domain and said light chain variable domain are covalently attached via a scFv linker.

Optionally said anti-VSIG10 antibody is selected from the group consisting of 577-Ab and 576-Ab, and wherein said heavy chain variable domain is at least 90%, 91%, 92%, 93%, 94%, 95%, 96%, 97%, 98%, 99%, identical to a heavy chain variable domain selected from the group consisting of 577-Ab VH and 576-Ab VH, and/or wherein said light chain variable domain is at least 90%, 91%, 92%, 93%, 94%, 95%, 96%, 97%, 98%, 99%, identical to a light chain variable domain selected from the group consisting of 577-Ab VL and 576-Ab VL.

Optionally said antibody comprises an antibody that has at least 90%, 91%, 92%, 93%, 94%, 95%, 96%, 97%, 98%, 99%, identity to the heavy and light chain of an antibody selected from the group consisting of 577-Ab and 576-Ab.

Optionally the antibody or antigen binding fragment competes for binding with an antibody selected from the group consisting of 577-Ab and 576-Ab.

VSIG10 is single pass transmembrane protein from the Ig superfamily which contains 4 Ig domains. FIG. 1A shows predicted topology and domains for VSIG10. Signal peptide (SigP) was predicted using SignalP and transmembrane (TM) domain was predicted using TMHMM. Domains were predicted using Interpro. Box sizes are drawn to scale.

The full length amino acid sequence of known (wild type) VSIG10 protein (V-set and immunoglobulin domain-containing protein 10, genbank accession number: NP_061959, SEQ ID NO:3), and the amino acid sequence of VSIG10 variant (SEQ ID NO:5) are shown in FIGS. 1B and 1C, respectively.

According to at least some embodiments, there is provided an expression vector or a virus, containing at least one isolated nucleic acid sequence as described herein. According to at least some embodiments, there is provided a recombinant cell comprising an expression vector or a virus containing an isolated nucleic acid sequence as described herein, wherein the cell constitutively or inducibly expresses the polypeptide encoded by the DNA segment. According to at least some embodiments, there is provided a method of producing a VSIG10 soluble ectodomain polypeptide, or fragment or fusion protein thereof, comprising culturing the recombinant cell as described herein, under conditions whereby the cell expresses the polypeptide encoded by the DNA segment or nucleic acid and recovering said polypeptide.

According to at least some embodiments, the invention provides a use of an antibody specifically binding to VSIG10, or pharmaceutical composition comprising same, for administration as an anti-cancer vaccine, as an adjuvant for anti cancer vaccine, and/or for adoptive immunotherapy, and/or for immunotherapy of cancer as recited herein.

According to at least some embodiments, there are provided antibodies in which the antigen binding site comprises a conformational or linear epitope, and wherein the antigen binding site contains about 3-7 contiguous or non-contiguous amino acids. Optionally, the antibody is a fully human antibody, chimeric antibody, humanized or primatized antibody.

Also optionally, the antibody is selected from the group consisting of Fab, Fab′, F(ab′)2, F(ab′), F(ab), Fv or scFv fragment and minimal recognition unit.

Also optionally, the antibody is coupled to a moiety selected from a drug, a radionuclide, a fluorophore, an enzyme, a toxin, a therapeutic agent, or a chemotherapeutic agent; and wherein the detectable marker is a radioisotope, a metal chelator, an enzyme, a fluorescent compound, a bioluminescent compound or a chemiluminescent compound.

According to at least some embodiments the invention relates to protein scaffolds with specificities and affinities in a range similar to specific antibodies. According to at least some embodiments the present invention relates to an antigen-binding construct comprising a protein scaffold which is linked to one or more epitope-binding domains. Such engineered protein scaffolds are usually obtained by designing a random library with mutagenesis focused at a loop region or at an otherwise permissible surface area and by selection of variants against a given target via phage display or related techniques. According to at least some embodiments the invention relates to alternative scaffolds including, but not limited to, anticalins, DARPins, Armadillo repeat proteins, protein A, lipocalins, fibronectin domain, ankyrin consensus repeat domain, thioredoxin, chemically constrained peptides and the like. According to at least some embodiments the invention relates to alternative scaffolds that are used as therapeutic agents for treatment of cancer as recited herein, and infectious diseases, as well as for in vivo diagnostics.

Administration of the antibody or pharmaceutical composition comprising same to a subject may be described as a treatment. Optionally the treatment is combined with another moiety or therapy useful for treating cancer.

Optionally the therapy is radiation therapy, antibody therapy, chemotherapy, photodynamic therapy, adoptive T cell therapy, Treg depletion, surgery or in combination therapy with conventional drugs.

Optionally the moiety is selected from the group consisting of immunosuppressants, cytotoxic drugs, tumor vaccines, antibodies (e.g. bevacizumab, erbitux), peptides, pepti-bodies, small molecules, chemotherapeutic agents such as cytotoxic and cytostatic agents (e.g. paclitaxel, cisplatin, vinorelbine, docetaxel, gemcitabine, temozolomide, irinotecan, 5FU, carboplatin), immunological modifiers such as interferons and interleukins, immunostimulatory antibodies, growth hormones or other cytokines, folic acid, vitamins, minerals, aromatase inhibitors, RNAi, Histone Deacetylase Inhibitors, and proteasome inhibitors.

Optionally the cancer is selected from a group consisting of breast cancer, cervical cancer, ovary cancer, endometrial cancer, melanoma, bladder cancer, lung cancer, pancreatic cancer, colon cancer, prostate cancer, leukemia, acute lymphocytic leukemia, chronic lymphocytic leukemia, B-cell lymphoma, Burkitt's lymphoma, multiple myeloma, Hodgkin's lymphoma, Non-Hodgkin's lymphoma, myeloid leukemia, acute myelogenous leukemia (AML), chronic myelogenous leukemia, thyroid cancer, thyroid follicular cancer, myelodysplastic syndrome (MDS), fibrosarcomas and rhabdomyosarcomas, melanoma, uveal melanoma, teratocarcinoma, neuroblastoma, glioma, glioblastoma, benign tumor of the skin, keratoacanthomas, renal cancer, anaplastic large-cell lymphoma, esophageal squamous cells carcinoma, hepatocellular carcinoma, follicular dendritic cell carcinoma, intestinal cancer, muscle-invasive cancer, seminal vesicle tumor, epidermal carcinoma, spleen cancer, bladder cancer, head and neck cancer, stomach cancer, liver cancer, bone cancer, brain cancer, cancer of the retina, biliary cancer, small bowel cancer, salivary gland cancer, cancer of uterus, cancer of testicles, cancer of connective tissue, prostatic hypertrophy, myelodysplasia, Waldenstrom's macroglobinaemia, nasopharyngeal, neuroendocrine cancer, myelodysplastic syndrome, mesothelioma, angiosarcoma, Kaposi's sarcoma, carcinoid, oesophagogastric, fallopian tube cancer, peritoneal cancer, papillary serous mullerian cancer, malignant ascites, gastrointestinal stromal tumor (GIST), Li-Fraumeni syndrome and Von Hippel-Lindau syndrome (VHL), and wherein the cancer is non-metastatic, invasive or metastatic.

Optionally the cancer is any of melanoma, cancer of liver, renal, brain, breast, colon, lung, ovary, pancreas, prostate, stomach, endometrial cancer, multiple myeloma, Hodgkin's lymphoma, non Hodgkin's lymphoma, acute and chronic lymphoblastic leukemia and acute and chronic myeloid leukemia.

According to at least some embodiments, there is provided a method for potentiating a secondary immune response to an antigen in a patient, which method comprises administering a therapeutically effective amount of an antibody as described herein or a pharmaceutical composition comprising same.

Optionally the antigen is a cancer antigen, a viral antigen or a bacterial antigen, and the patient has received treatment with an anticancer vaccine or a viral vaccine.

According to one embodiment, detecting the presence of the polypeptide is indicative of the presence of the disease and/or its severity and/or its progress. According to another embodiment, a change in the expression and/or the level of the polypeptide compared to its expression and/or level in a healthy subject or a sample obtained therefrom is indicative of the presence of the disease and/or its severity and/or its progress. According to a further embodiment, a change in the expression and/or level of the polypeptide compared to its level and/or expression in said subject or in a sample obtained therefrom at earlier stage is indicative of the progress of the disease. According to still further embodiment, detecting the presence and/or relative change in the expression and/or level of the polypeptide is useful for selecting a treatment and/or monitoring a treatment of the disease.

According to at least some embodiments, there is provided a method, comprising obtaining a sample of cancer cells and their microenvironment from the subject; assaying said sample to detect a presence of said isolated polypeptide in an immune cell or in a cancer cell; and if said presence is detected, administering said antibody or fragment thereof, or said pharmaceutical composition, to the subject.

### BRIEF DESCRIPTION OF THE DRAWINGS

FIG. 1A shows predicted topology and domains for VSIG10;

FIG. 1B shows the full length amino acid sequence of genbank accession number: NP_061959, referred to herein as the wild type or WT VSIG10;

FIG. 1C shows the full length amino acid sequence of a variant VSIG10;

FIG. 2A-C shows VSIG10 expression in normal (A; GTEx project data), Cancer (B; TCGA primary and metastatic tumor data) and GTEx vs TCGA (C);

FIG. 3 shows that in cancer VSIG10 is expressed in epithelial cells as well as in immune cells;

FIG. 4 shows VSIG10 expression in macrophages, dendritic cells and monocytes from the Blueprint project;

FIGS. 5A and 5B shows VSIG10 expression in mouse immune cells (ref: immgen, GSE15907);

FIG. 6 shows VSIG10 expression in dendritic cells and macrophages from lung cancer tumor model (pmid: 25446897);

FIG. 7A-H shows the antibodies AB-577 and AB-576 sequences. FIG. 7A shows the heavy chain: DNA sequence (402 bp) (SEQ ID NO:200); FIG. 7B shows the heavy chain amino acids sequence (134 aa) (SEQ ID NO:201); FIG. 7C shows the light chain DNA sequence (381 bp) (SEQ ID NO:205); FIG. 7D shows the light chain amino acids sequence (127 aa) (SEQ ID NO:206); FIG. 7E shows the heavy chain: DNA sequence (408 bp) (SEQ ID NO:216); FIG. 7F shows the heavy chain amino acids sequence (136 aa) (SEQ ID NO:217); FIG. 7G shows the light chain DNA sequence (399 bp) (SEQ ID NO:221); FIG. 7H shows the light chain amino acids sequence (133 aa) (SEQ ID NO:222). The CDRs are marked in blue font and bold.

FIGS. 8A and 8B show WB analysis on HEK293 overexpressing human VSIG10 flag transfected cells and endogenous cell line expressing VSIG10 using AB-577 clonal supernatants and purified Ab (FIG. 8A) or using AB-576 clonal supernatants and purified Ab (FIG. 8B);

FIGS. 9A and 9B show the binding of AB-577 (FIG. 9A) and AB-576 (FIG. 9B) to the HEK293 cells over-expressing human VSIG10 Flag protein;

FIGS. 10A and 10B show affinity measurements using FACS application for the anti-human VSIG10 mAbs AB-577 (FIG. 10A) and AB-576 (FIG. 10B) on HEK293 cells over-expressing human VSIG10 Flag protein;

FIGS. 11A and 11B show membrane expression of human VSIG10 protein in DAN-G (left), AsPc1 (right) human cell lines transfected with human VSIG10 siRNA or non-target siRNA control, stained with AB-577 (FIG. 11A), AB-576 (FIG. 11B);

FIGS. 12A and 12B show affinity measurements using FACS application for the anti-human VSIG10 mAb AB-577 (FIG. 12A) or AB-576 (FIG. 12B) on DU-145 human cell line;

FIG. 13A shows high power microphotographs of cell blocks sections retrieved in CA and incubated with AB-577 (10 μg/ml), while FIG. 13B shows high power microphotographs of cell blocks sections retrieved in CA and incubated with AB-576 (10 g/ml);

FIG. 14A shows a microphotograph of normal colon mucosa section immunostained with AB-577, while FIG. 14B shows microphotograph of normal colon mucosa section immunostained with AB-576;

FIG. 15A shows a microphotograph of NSCLC sample section immunostained with AB-577, while FIG. 15B shows microphotograph of NSCLC sample section immunostained with AB-576;

FIG. 16 presents an illustration of the experimental system utilizing Mel-624 cells over-expressing VSIG10 and being used for activating melanoma derived T cells (TILs) with antigen specificity for either gp100 or MART 1 derived peptides;

FIGS. 17A and 17B shows that sorted, transduced Mel-624 over-express VSIG10;

FIG. 18 shows that sorted, transduced Mel-624 over-express VSIG10;

FIGS. 19A and 19B shows gMFI mean values on gated CD8+ TILs after co-culture with Mel-624 cells;

FIG. 20A-C shows IFN gamma and TNFa secreted from TILs after co-culture with Mel-624 cells;

FIGS. 21A-L show that Mel-624 over expressing VSIG10 inhibits IFN gamma secretion from TILs supernatant from TIL;

FIG. 21M shows that Mel-624 over expressing VSIG10 mediate an inhibitory effect on TILs;

FIG. 22A-I shows that Mel-624 over expressing VSIG10 inhibit TILs secretion of IFNg/TNFa and CD137 expression;

FIG. 23 is an illustration of the experimental system utilizing CHO-S-OKT3 cells over-expressing VSIG10 and being used for poly-clonal activation of primary T cells; and

FIGS. 24A and 24B shows that reduced tumor growth of the MC38 tumor model inoculated to mVSIG10 KO relative to wild-type mice with and without anti-PDL-1 treatment.

FIG. 25 shows schematic illustration of the CHO-S-IAd experimental system.

FIG. 26 shows the inhibitory effect on DO11 derived CD4 T cells, mediated by VSIG10 over expression on CHO-S-IA^{d} cells.

FIG. 27A shows IHC staining of lung cancer samples (n=110) vs. normal lung tissue (n=10) scores. Graph shows mean±SEM (P<0.01). FIG. 27B shows microphotograph of cancer and normal lung sections immunostained with AB-577 (upper panel) and anti-CD34 (lower panel).

FIG. 28 shows microphotograph of tumor and normal regions of NSCLC sample 388042C1 (upper panel) and 1224263B (lower panel) immunostained with AB-577.

FIG. 29A-D shows the expression of VSIG10 by FACS on immune cells (FIG. 29A), non-immune cells (FIG. 29B), cDCs (FIG. 29C) and myeloid DCs (FIG. 29D), presented as MFI ratio between anti-VSIG10 stained cells and isotype control.

FIG. 30: CHO-S mIAd overexpressing mouse VSIG10inhibitory effect on proliferation and cytokines secretion from mouse CD4+ T cells. Supernatant from CD4+ T cells:CHO-S mIAd co-cultures were collected and tested for cytokines by CBA kit. Response to mouse VSIG10 overexpressing cells (blue) and PDL1 (red) is compared to T cells response to control EV cells (grey) at 0.05 ug/ml OVA peptide concentration. Each dot represents the mean of quadruplicates in a single exp. n=4, p-values are plotted above each graph.

FIGS. 31A and 31B show scatter plots, demonstrating the expression of VSIG10 transcripts, that encode the VSIG10 proteins, on a virtual panel of all tissues and conditions using MED discovery engine, demonstrating differential expression of VSIG10 transcripts in several groups of cells from the immune system, mainly in leukocytes, and in various cancer conditions, such as CD10+ leukocytes from ALL and BM-CD34+ cells from AML.

FIGS. 32A and 32B show the effect of VSIG10 fusion protein (SEQ ID NO:24), and other proteins, on CD4 T cell activation, as manifested by reduced IFNγ secretion (A) and reduced expression of the activation marker CD69 (B). Each bar is the mean of duplicate cultures, the error bars indicating the standard deviation (Student t-test,*P<0.05, **p<0.01, compared with control mIgG2a.

FIGS. 33A-33E show the therapeutic effect of VSIG10-Ig (SEQ ID NO:24) treatment in the PLP139-151-induced R-EAE model in SJL mice. VSIG10-Ig (SEQ ID NO:24) was administered in a therapeutic mode from the onset of disease remission (day 19), at 100 microg/mouse i.p. 3 times per week for two weeks. Therapeutic effects of VSIG10-Ig on clinical symptoms is demonstrated as reduction in Mean Clinical Score (FIG. 33A). In addition, VSIG10-Ig treatment inhibited DTH responses to spread epitopes (PLP178-191 and MBP MBP84-104), on days 45 and 76 after R-EAE induction (FIG. 33B). Also shown is the effect of VSIG10-Ig on ex-vivo recall responses of splenocytes isolated on day 45 and 75 post disease induction (FIG. 33C) and LN cells isolated on day 45 post disease induction (FIG. 33D) as manifested by the effect of VSIG10-Ig treatment on cell proliferation and cytokine secretion (IFNg, IL-17, IL-10 and IL-4). The effect of VSIG10-Ig on cell counts in the spleen, lymph nodes and CNS as well as the different linages present within each of these tissues upon treatment with VSIG10-Ig at 100 ug/dose is shown in FIG. 33E. In this study the effect of VSIG10-Ig was studied in comparison to mIgG2a Ig control that was administered at similar dose and regimen as VSIG10-Ig.

### DETAILED DESCRIPTION OF THE INVENTION

Cancer can be considered as an inability of the patient to recognize and eliminate cancerous cells. In many instances, these transformed (e.g. cancerous) cells counteract immunosurveillance. There are natural control mechanisms that limit T-cell activation in the body to prevent unrestrained T-cell activity, which can be exploited by cancerous cells to evade or suppress the immune response. Restoring the capacity of immune effector cells-especially T cells—to recognize and eliminate cancer is the goal of immunotherapy. The field of immuno-oncology, sometimes referred to as “immunotherapy” is rapidly evolving, with several recent approvals of T cell checkpoint inhibitory antibodies such as Yervoy, Keytruda and Opdivo. These antibodies are generally referred to as “checkpoint inhibitors” because they block normally negative regulators of T cell immunity. It is generally understood that a variety of immunomodulatory signals, both costimulatory and coinhibitory, can be used to orchestrate an optimal antigen-specific immune response. Generally, these antibodies bind to checkpoint inhibitor proteins such as CTLA-4 and PD-1, which under normal circumstances prevent or suppress activation of cytotoxic T cells (CTLs). By inhibiting the checkpoint protein, for example through the use of antibodies that bind these proteins, an increased T cell response against tumors can be achieved. That is, these cancer checkpoint proteins suppress the immune response; when the proteins are blocked, for example using antibodies to the checkpoint protein, the immune system is activated, leading to immune stimulation, resulting in treatment of conditions such as cancer and infectious disease.

Based on RNA expression data, a broad expression of VSIG10 on normal and cancer epithelial cells was observed. In addition, surprisingly, VSIG10 was found to be expressed on immune cells of the myeloid lineage in both human and mice. Specifically, within immune cells, VSIG10 is most prominently expressed on sub-sets of dendritic cells (namely CD103+ cells in mice and CD1C+ in human), which are known to play a role in antigen presentation in the tumor environment. There is growing evidence for immune-checkpoints expressed on dendritic cells and other myeloid cells in the tumor environment playing an inhibitory role in the anti-tumor immune response. For example, CD103 positive dendritic cells are known to play an active role in the anti-tumor immune response and were found to be crucial for response to anti-PDL1 treatment (Immunity. 2016 Apr. 19; 44(4):924-38).

Using human and mouse in vitro experimental systems, T cell inhibitory checkpoint activity for VSIG10 was found as demonstrated by reduced cytokine secretion and activation markers expression in reductionist systems, as shown herein.

To demonstrate in vivo checkpoint activity, mice with a specific depletion of the VSIG10 gene (VSIG10 Kos) were generated. When transplanted with a syngeneic tumor model (namely MC38), VSIG10 KO exhibited reduced tumor growth in comparison to wild type littermates. Also upon treatment with a PDL1 blocking Ab, VSIG10 KO exhibited reduced tumor growth in comparison to wild type littermates.

Herein data is presented that supports VSIG10 mode of action as a T cell inhibitory ligand expressed on professional APCs (especially DCs) and epithelial cells in the tumor microenvironment.

Blocking the interaction of VSIG10 with its putative binding partner on T cells using VSIG10 specific blocking antibodies (or Abs) will therefore improve anti-cancer immunity and/or improve priming of tumor antigen-specific T cells. Therefore VSIG10 specific blocking antibodies might be used for treatment of cancer, alone or in combination with other treatment methods and/or therapeutic agents known in the art (i.e., combination therapy). Particularly, VSIG10 specific blocking antibodies might be used for treatment of cancer in combination with antibodies for other immune checkpoints, such as an anti-PD-L1 antibody, an anti-PD-L2 antibody, an anti-LAG-3 antibody, an anti-CTLA-4 antibody, an anti-TIM-3 antibody, an anti-BTLA antibody, an anti-PVRIG antibody, an anti-HVEM antibody, an anti-CEACAM1 antibody, an anti-GITR antibody, an anti-ICOS antibody, an anti-41BB antibody, an anti-OX40 antibody, an anti-KIR antibody, an anti-VISTA antibody, an anti-B7-H3 antibody, an anti-B7-H4 antibody, an anti-CD27 antibody, an anti-CD28 antibody, an anti-CD40 antibody, an anti-CD96 antibody, an anti-SIRPa antibody, an anti-CSF1R antibody, an anti-ILT2 antibody, an anti-ILT3 antibody, an anti-ILT4 antibody and an anti-ILT5 antibody, anti-CD137 antibody, anti-KIR antibody and any combination thereof. Particularly, VSIG10 specific blocking antibodies might be used for treatment of cancer in combination with antibodies blocking PD 1/PDL1 pathway. Alternatively, VSIG10 specific blocking antibodies might be used for treatment of cancer in combination with cancer vaccines, such as STING agonist formulated cancer vaccines (STINGVAX) and GVAX.

VSIG10 Proteins

The present invention provides antibodies that specifically bind to VSIG10 proteins. “Protein” in this context is used interchangeably with “polypeptide”, and includes peptides as well. The present invention provides antibodies that specifically bind to VSIG10 proteins.

Accordingly, as used herein, the term “VSIG10” or “VSIG10 protein” or “VSIG10 polypeptide” may optionally include any such protein, or variants, conjugates, or fragments thereof, including but not limited to known or wild type VSIG10, as described herein, as well as any naturally occurring splice variants, amino acid variants or isoforms, and in particular the ECD fragment of VSIG10. The term “soluble” form of VSIG10 is also used interchangeably with the terms “soluble ectodomain (ECD)” or “ectodomain” or “extracellular domain (ECD) as well as “fragments of VSIG10 polypeptides”, which may refer broadly to one or more of the following optional polypeptides:

Included within the definition of VSIG10 proteins are VSIG10 ECD fragments.

The term the “soluble ectodomain (ECD)” or “ectodomain” or “soluble” form of VSIG10 refers also to the nucleic acid sequences encoding the corresponding proteins of VSIG10 “soluble ectodomain (ECD)” or “ectodomain” or “soluble VSIG10 proteins/molecules”). Optionally, the VSIG10 ECD refers to any one of the polypeptide sequences below and/or listed in Table B below, and/or fragments or variants thereof possessing at least 80% sequence identity, more preferably at least 90% sequence identity therewith and even more preferably at least 95, 96, 97, 98 or 99% sequence identity therewith, and/or conjugates thereof, and/or polynucleotides encoding same:

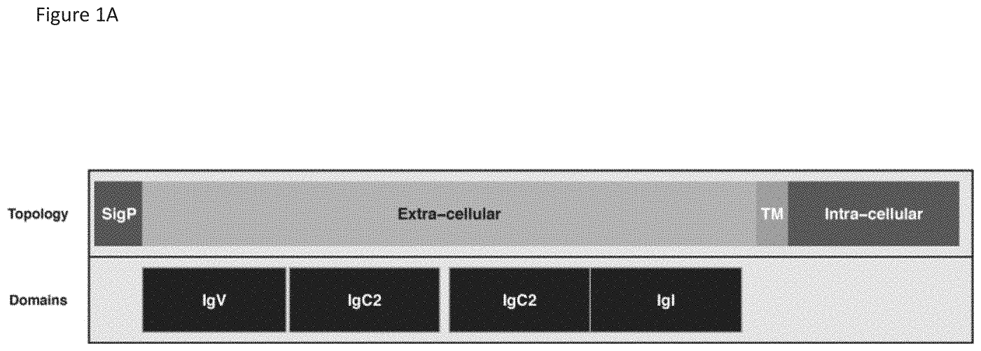

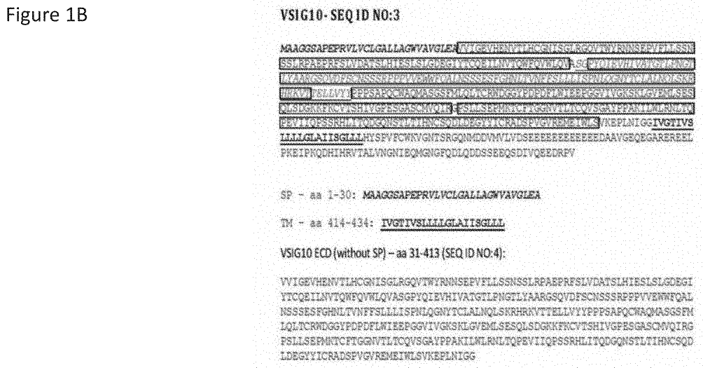

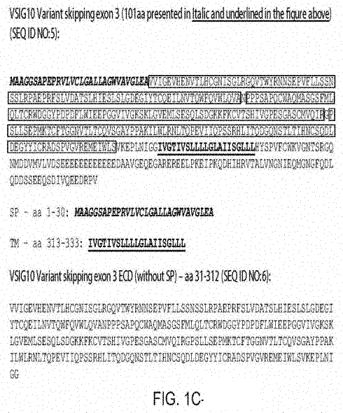

SEQ ID NO: 4, amino acid residues 31-413 (not
including signal peptide, up till transmembrane)
(FIG. 1B):
VVIGEVHENVTLHCGNISGLRGQVTWYRNNSEPVFLLSSNSSLRPAEPRF
SLVDATSLHIESLSLGDEGIYTCQEILNVTQWFQVWLQVASGPYQIEVHI
VATGTLPNGTLYAARGSQVDFSCNSSSRPPPVVEWWFQALNSSSESFGHN
LTVNFFSLLLISPNLQGNYTCLALNQLSKRHRKVTTELLVYYPPPSAPQC
WAQMASGSFMLQLTCRWDGGYPDPDFLWIEEPGGVIVGKSKLGVEMLSES
QLSDGKKFKCVTSHIVGPESGASCMVQIRGPSLLSEPMKTCFTGGNVTLT
CQVSGAYPPAKILWLRNLTQPEVIIQPSSRHLITQDGQNSTLTIHNCSQD
LDEGYYICRADSPVGVREMEIWLSVKEPLNIGG;
SEQ ID NO: 6, amino acid residues 31-312 (skipping
exon 3 variant, not including signal peptide, up
till transmembrane) (FIG. 1C):
VVIGEVHENVTLHCGNISGLRGQVTWYRNNSEPVFLLSSNSSLRPAEP
RFSLVDATSLHIESLSLGDEGIYTCQEILNVTQWFQVWLQVANPPPSAPQ
CWAQMASGSFMLQLTCRWDGGYPDPDFLWIEEPGGVIVGKSKLGVEMLSE
SQLSDGKKFKCVTSHIVGPESGASCMVQIRGPSLLSEPMKTCFTGGNVTL
TCQVSGAYPPAKILWLRNLTQPEVIIQPSSRHLITQDGQNSTLTIHNCSQ
DLDEGYYICRADSPVGVREMEIWLSVKEPLNIGG,

and variants thereof possessing at least 80% sequence identity, more preferably at least 90% sequence identity therewith and even more preferably at least 95, 96, 97, 98 or 99% sequence identity therewith. SEQ ID NOs:60-61 represent examples of the VSIG10 ECD including signal peptide.

Generally, the VSIG10 polypeptide fragments are expressed from nucleic acids that include sequences that encode a signal sequence. The signal sequence is generally cleaved from the immature polypeptide to produce the mature polypeptide lacking the signal sequence. The signal sequence of VSIG10 can be replaced by the signal sequence of another polypeptide using standard molecule biology techniques to affect the expression levels, secretion, solubility, or other property of the polypeptide. The signal peptide sequence that is used to replace the VSIG10 signal peptide sequence can be any known in the art.

Optionally, the VSIG10 ECD refers also to any one of the nucleic acid sequences encoding VSIG10 ECD polypeptides, optionally to the nucleic acid sequences set forth in SEQ ID NOs:34, 36, or fragments thereof and/or degenerative variants thereof, encoding VSIG10 ECD polypeptides set forth in SEQ ID NOs:4, 6, respectively.

Antibodies

Accordingly, the invention provides anti-VSIG10 antibodies. The antibodies of the invention are specific for the VSIG10 extracellular domain as more fully outlined herein.

As is discussed below, the term “antibody” is used generally. Antibodies that find use in the present invention can take on a number of formats as described herein, including traditional antibodies as well as antibody derivatives, fragments and mimetics, described below. In general, the term “antibody” includes any polypeptide that includes at least one antigen binding domain, as more fully described below. Antibodies may be polyclonal, monoclonal, xenogeneic, allogeneic, syngeneic, or modified forms thereof, as described herein, with monoclonal antibodies finding particular use in many embodiments. In some embodiments, antibodies of the invention bind specifically or substantially specifically to VSIG10 molecules. The terms “monoclonal antibodies” and “monoclonal antibody composition”, as used herein, refer to a population of antibody molecules that contain only one species of an antigen-binding site capable of immunoreacting with a particular epitope of an antigen, whereas the term “polyclonal antibodies” and “polyclonal antibody composition” refer to a population of antibody molecules that contain multiple species of antigen-binding sites capable of interacting with a particular antigen. A monoclonal antibody composition, typically displays a single binding affinity for a particular antigen with which it immunoreacts.

Traditional full length antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. The present invention is directed to the IgG class, which has several subclasses, including, but not limited to IgG1, IgG2, IgG3, and IgG4. Thus, “isotype” as used herein is meant any of the subclasses of immunoglobulins defined by the chemical and antigenic characteristics of their constant regions. While the exemplary antibodies herein designated “CPA” are based on IgG1 heavy constant regions, as shown in FIG. 38, the anti-VSIG10 antibodies of the invention include those using IgG2, IgG3 and IgG4 sequences, or combinations thereof. For example, as is known in the art, different IgG isotypes have different effector functions which may or may not be desirable. Accordingly, the CPA antibodies of the invention can also swap out the IgG1 constant domains for IgG2, IgG3 or IgG4 constant domains (depicted in FIG. 66), with IgG2 and IgG4 finding particular use in a number of situations, for example for ease of manufacture or when reduced effector function is desired, the latter being desired in some situations.

For the enumerated antibodies of the CHA designation, these are murine antibodies generated in hybridomas (the “H” designation), and thus in general they are humanized as is known in the art, generally in the framework regions (F1 to F4 for each of the heavy and light variable regions), and then grafted onto human IgG1, IgG2, IgG3 or IgG4 constant heavy and light domains (depicted in FIG. 66), again with IgG4 finding particular use, as is more fully described below.

The amino-terminal portion of each chain includes a variable region of about 100 to 110 or more amino acids primarily responsible for antigen recognition, generally referred to in the art and herein as the “Fv domain” or “Fv region”. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. “Variable” refers to the fact that certain segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions”.

Each VH and VL is composed of three hypervariable regions (“complementary determining regions,” “CDRs”) and four FRs, arranged from amino-terminus to carboxy-terminus in the following order: FR1-CDR1-FR2-CDR2-FR3-CDR3-FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region, although sometimes the numbering is shifted slightly as will be appreciated by those in the art; Kabat et al., SEQUENCES OF PROTEINS OF IMMUNOLOGICAL INTEREST, 5 th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below and shown in 7.

The carboxy-terminal portion of each chain defines a constant region primarily responsible for effector function. Kabat et al. collected numerous primary sequences of the variable regions of heavy chains and light chains. Based on the degree of conservation of the sequences, they classified individual primary sequences into the CDR and the framework and made a list thereof (see SEQUENCES OF IMMUNOLOGICAL INTEREST, 5 th edition, NIH publication, No. 91-3242, E. A. Kabat et al., entirely incorporated by reference).

In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains.

In the context of IgG antibodies, the IgG isotypes each have three CH regions. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat.

Accordingly, the invention provides variable heavy domains, variable light domains, heavy constant domains, light constant domains and Fc domains to be used as outlined herein. By “variable region” as used herein is meant the region of an immunoglobulin that comprises one or more Ig domains substantially encoded by any of the Vx or VX, and/or VH genes that make up the kappa, lambda, and heavy chain immunoglobulin genetic loci respectively. Accordingly, the variable heavy domain comprises vhFR1-vhCDR1-vhFR2-vhCDR2-vhFR3-vhCDR3-vhFR4, and the variable light domain comprises vlFR1-vlCDR1-vlFR2-vlCDR2-vlFR3-vlCDR3-vlFR4. By “heavy constant region” herein is meant the CH1-hinge-CH2-CH3 portion of an antibody. By “Fc” or “Fc region” or “Fc domain” as used herein is meant the polypeptide comprising the constant region of an antibody excluding the first constant region immunoglobulin domain and in some cases, part of the hinge. Thus Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cy2 and Cy3 (Cy2 and Cy3) and the lower hinge region between Cy1 (Cy1) and Cy2 (Cy2). Although the boundaries of the Fc region may vary, the human IgG heavy chain Fc region is usually defined to include residues C226 or P230 to its carboxyl-terminus, wherein the numbering is according to the EU index as in Kabat. In some embodiments, as is more fully described below, amino acid modifications are made to the Fc region, for example to alter binding to one or more FcγR receptors or to the FcRn receptor.

Thus, “Fc variant” or “variant Fc” as used herein is meant a protein comprising an amino acid modification in an Fc domain. The Fc variants of the present invention are defined according to the amino acid modifications that compose them. Thus, for example, N434S or 434S is an Fc variant with the substitution serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as M428L/N434S, and so on. For all positions discussed in the present invention that relate to antibodies, unless otherwise noted, amino acid position numbering is according to the EU index.

By “Fab” or “Fab region” as used herein is meant the polypeptide that comprises the VH, CH1, VL, and CL immunoglobulin domains. Fab may refer to this region in isolation, or this region in the context of a full length antibody, antibody fragment or Fab fusion protein. By “Fv” or “Fv fragment” or “Fv region” as used herein is meant a polypeptide that comprises the VL and VH domains of a single antibody. As will be appreciated by those in the art, these generally are made up of two chains.

Throughout the present specification, either the IMTG numbering system or the Kabat numbering system is generally used when referring to a residue in the variable domain (approximately, residues 1-107 of the light chain variable region and residues 1-113 of the heavy chain variable region) (e.g., Kabat et al., supra (1991)). EU numbering as in Kabat is generally used for constant domains and/or the Fc domains.

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning”. Specific bins are described below.

Included within the definition of “antibody” is an “antigen-binding portion” of an antibody (also used interchangeably with “antigen-binding fragment”, “antibody fragment” and “antibody derivative”). That is, for the purposes of the invention, an antibody of the invention has a minimum functional requirement that it bind to a VSIG10 antigen. As will be appreciated by those in the art, there are a large number of antigen fragments and derivatives that retain the ability to bind an antigen and yet have alternative structures, including, but not limited to, (i) the Fab fragment consisting of VL, VH, CL and CH1 domains, (ii) the Fd fragment consisting of the VH and CH1 domains, (iii) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883, entirely incorporated by reference), (iv) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448, all entirely incorporated by reference), (v) “domain antibodies” or “dAb” (sometimes referred to as an “immunoglobulin single variable domain”, including single antibody variable domains from other species such as rodent (for example, as disclosed in WO 00/29004), nurse shark and Camelid V-HH dAbs, (vi) SMIPs (small molecule immunopharmaceuticals), camelbodies, nanobodies and IgNAR.

Still further, an antibody or antigen-binding portion thereof (antigen-binding fragment, antibody fragment, antibody portion) may be part of a larger immunoadhesion molecules (sometimes also referred to as “fusion proteins”), formed by covalent or noncovalent association of the antibody or antibody portion with one or more other proteins or peptides. Examples of immunoadhesion molecules include use of the streptavidin core region to make a tetrameric scFv molecule and use of a cysteine residue, a marker peptide and a C-terminal polyhistidine tag to make bivalent and biotinylated scFv molecules. Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

In general, the anti-VSIG10 antibodies of the invention are recombinant. “Recombinant” as used herein, refers broadly with reference to a product, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “recombinant antibody”, as used herein, includes all antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and V_{L} regions of the recombinant antibodies are sequences that, while derived from and related to human germline V_{H} and V_{L} sequences, may not naturally exist within the human antibody germline repertoire in vivo.

A. Optional Antibody Engineering

The antibodies of the invention can be modified, or engineered, to alter the amino acid sequences by amino acid substitutions.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

As discussed herein, amino acid substitutions can be made to alter the affinity of the CDRs for the VSIG10 protein (including both increasing and decreasing binding, as is more fully outlined below), as well as to alter additional functional properties of the antibodies. For example, the antibodies may be engineered to include modifications within the Fc region, typically to alter one or more functional properties of the antibody, such as serum half-life, complement fixation, Fc receptor binding, and/or antigen-dependent cellular cytotoxicity. Furthermore, an antibody according to at least some embodiments of the invention may be chemically modified (e.g., one or more chemical moieties can be attached to the antibody) or be modified to alter its glycosylation, again to alter one or more functional properties of the antibody. Such embodiments are described further below. The numbering of residues in the Fc region is that of the EU index of Kabat.

In one embodiment, the hinge region of C_{H1} is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In some embodiments, amino acid substitutions can be made in the Fc region, in general for altering binding to FcγR receptors. By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII-1 (CD16), and FcγRIII-2 (CD16-2), as well as any undiscovered mouse FcγRs or FcγR isoforms or allotypes.

There are a number of useful Fc substitutions that can be made to alter binding to one or more of the FcγR receptors. Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell. Similarly, decreased binding to FcγRIIb (an inhibitory receptor) can be beneficial as well in some circumstances. Amino acid substitutions that find use in the present invention include those listed in U.S. Ser. Nos. 11/124,620 (particularly FIG. 41) and U.S. Pat. No. 6,737,056, both of which are expressly incorporated herein by reference in their entirety and specifically for the variants disclosed therein. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 299T and 297N.

In addition, the antibodies of the invention are modified to increase its biological half-life. Various approaches are possible. For example, one or more of the following mutations can be introduced: T252L, T254S, T256F, as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Additional mutations to increase serum half life are disclosed in U.S. Pat. Nos. 8,883,973, 6,737,056 and 7,371,826, and include 428L, 434A, 434S, and 428L/434S.

In yet other embodiments, the Fc region is altered by replacing at least one amino acid residue with a different amino acid residue to alter the effector functions of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region is modified to increase the ability of the antibody to mediate antibody dependent cellular cytotoxicity (ADCC) and/or to increase the affinity of the antibody for an Fcγ receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgG1 for FcγRI, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 are shown to improve binding to FcγRIII. Additionally, the following combination mutants are shown to improve FcγRIII binding: T256A/S298A, S298A/E333A, S298A/K224A and S298A/E333A/K334A. Furthermore, mutations such as M252Y/S254T/T256E or M428L/N434S improve binding to FcRn and increase antibody circulation half-life (see Chan C A and Carter P J (2010) Nature Rev Immunol 10:301-316).

In still another embodiment, the antibody can be modified to abrogate in vivo Fab arm exchange. Specifically, this process involves the exchange of IgG4 half-molecules (one heavy chain plus one light chain) between other IgG4 antibodies that effectively results in bispecific antibodies which are functionally monovalent. Mutations to the hinge region and constant domains of the heavy chain can abrogate this exchange (see Aalberse, R C, Schuurman J., 2002, Immunology 105:9-19).

In still another embodiment, the glycosylation of an antibody is modified. For example, an aglycosylated antibody can be made (i.e., the antibody lacks glycosylation). Glycosylation can be altered to, for example, increase the affinity of the antibody for antigen or reduce effector function such as ADCC. Such carbohydrate modifications can be accomplished by, for example, altering one or more sites of glycosylation within the antibody sequence, for example N297. For example, one or more amino acid substitutions can be made that result in elimination of one or more variable region framework glycosylation sites to thereby eliminate glycosylation at that site.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies.

Such carbohydrate modifications can be accomplished by, for example, expressing the antibody in a host cell with altered glycosylation machinery. Cells with altered glycosylation machinery have been described in the art and can be used as host cells in which to express recombinant antibodies according to at least some embodiments of the invention to thereby produce an antibody with altered glycosylation. For example, the cell lines Ms704, Ms705, and Ms709 lack the fucosyltransferase gene, FUT8 (α (1,6) fucosyltransferase), such that antibodies expressed in the Ms704, Ms705, and Ms709 cell lines lack fucose on their carbohydrates. The Ms704, Ms705, and Ms709 FUT8 cell lines are created by the targeted disruption of the FUT8 gene in CHO/DG44 cells using two replacement vectors (see U.S. Patent Publication No. 20040110704 by Yamane et al. and Yamane-Ohnuki et al. (2004) Biotechnol Bioeng 87:614-22). As another example, EP 1,176,195 by Hanai et al. describes a cell line with a functionally disrupted FUT8 gene, which encodes a fucosyl transferase, such that antibodies expressed in such a cell line exhibit hypofucosylation by reducing or eliminating the a 1,6 bond-related enzyme. Hanai et al. also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases (e.g., β(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180). Alternatively, the fucose residues of the antibody may be cleaved off using a fucosidase enzyme. For example, the fucosidase α-L-fucosidase removes fucosyl residues from antibodies (Tarentino, A. L. et al. (1975) Biochem. 14:5516-23).

Another modification of the antibodies herein that is contemplated by the invention is pegylation or the addition of other water soluble moieties, typically polymers, e.g., in order to enhance half-life. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies according to at least some embodiments of the invention. See for example, EP 0 154 316 by Nishimura et al. and EP 0 401 384 by Ishikawa et al.

In addition to substitutions made to alter binding affinity to FcγRs and/or FcRn and/or increase in vivo serum half life, additional antibody modifications can be made, as described in further detail below.

In some cases, affinity maturation is done. Amino acid modifications in the CDRs are sometimes referred to as “affinity maturation”. An “affinity matured” antibody is one having one or more alteration(s) in one or more CDRs which results in an improvement in the affinity of the antibody for antigen, compared to a parent antibody which does not possess those alteration(s). In some cases, although rare, it may be desirable to decrease the affinity of an antibody to its antigen, but this is generally not preferred.

In some embodiments, one or more amino acid modifications are made in one or more of the CDRs of the VISG1 antibodies of the invention. In general, only 1 or 2 or 3-amino acids are substituted in any single CDR, and generally no more than from 1, 2, 3. 4, 5, 6, 7, 8 9 or 10 changes are made within a set of CDRs. However, it should be appreciated that any combination of no substitutions, 1, 2 or 3 substitutions in any CDR can be independently and optionally combined with any other substitution.

Affinity maturation can be done to increase the binding affinity of the antibody for the VSIG10 antigen by at least about 10% to 50-100-150% or more, or from 1 to 5 fold as compared to the “parent” antibody. Preferred affinity matured antibodies will have nanomolar or even picomolar affinities for the VSIG10 antigen. Affinity matured antibodies are produced by known procedures. See, for example, Marks et al., 1992, Biotechnology 10:779-783 that describes affinity maturation by variable heavy chain (VH) and variable light chain (VL) domain shuffling. Random mutagenesis of CDR and/or framework residues is described in: Barbas, et al. 1994, Proc. Nat. Acad. Sci, USA 91:3809-3813; Shier et al., 1995, Gene 169:147-155; Yelton et al., 1995, J. Immunol. 155:1994-2004; Jackson et al., 1995, J. Immunol. 154(7):3310-9; and Hawkins et al, 1992, J. Mol. Biol. 226:889-896, for example.

Alternatively, amino acid modifications can be made in one or more of the CDRs of the antibodies of the invention that are “silent”, e.g. that do not significantly alter the affinity of the antibody for the antigen. These can be made for a number of reasons, including optimizing expression (as can be done for the nucleic acids encoding the antibodies of the invention).

Thus, included within the definition of the CDRs and antibodies of the invention are variant CDRs and antibodies; that is, the antibodies of the invention can include amino acid modifications in one or more of the CDRs of the enumerated antibodies of the invention. In addition, as outlined below, amino acid modifications can also independently and optionally be made in any region outside the CDRs, including framework and constant regions.

In certain embodiments, an antibody of the invention comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on preferred anti-VSIG10 antibodies isolated and produced using methods herein, or conservative modifications thereof, and wherein the antibodies retain the desired functional properties of the anti-VSIG10 antibodies according to at least some embodiments of the invention, respectively.

In various embodiments, the anti-VSIG10 antibody can be, for example, human antibodies, humanized antibodies or chimeric antibodies.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody according to at least some embodiments of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody according to at least some embodiments of the invention can be replaced with other amino acid residues from the same side chain family and the altered antibody can be tested for retained function (i.e., the functions set forth in (c) through (j) above) using the functional assays described herein.

VSIG10 Antibodies

The present invention provides anti-VSIG10 antibodies. (For convenience, “anti-VSIG10 antibodies” and “VSIG10 antibodies” are used interchangeably). The anti-VSIG10 antibodies of the invention specifically bind to human VSIG10, and preferably the ECD of human VISG10, as depicted in FIG. 1.

According to at least some embodiments of the invention, VSIG10 antibody, antigen-binding fragment or conjugate thereof optionally and preferably mediates at least one of the following effects:

(i) increases in immune response, (ii) increases in activation of αβ and/or γδ T cells, (iii) increases in cytotoxic T cell activity, (iv) increases in NK and/or NKT cell activity, (v) alleviation of αβ and/or γδ T-cell suppression, (vi) increases in pro-inflammatory cytokine secretion, (vii) increases in IL-2 secretion; (viii) increases in interferon-γ production, (ix) increases in Th1 response, (x) decreases in Th2 response, (xi) decreases or eliminates cell number and/or activity of at least one of regulatory T cells (Tregs), myeloid derived suppressor cells (MDSCs), iMCs, mesenchymal stromal cells, TIE2-expressing monocytes, (xii) reduces regulatory cell activity, and/or the activity of one or more of myeloid derived suppressor cells (MDSCs), iMCs, mesenchymal stromal cells, TIE2-expressing monocytes, (xiii) decreases or eliminates M2 macrophages, (xiv) reduces M2 macrophage pro-tumorigenic activity, (xv) decreases or eliminates N2 neutrophils, (xvi) reduces N2 neutrophils pro-tumorigenic activity, (xvii) reduces inhibition of T cell activation, (xviii) reduces inhibition of CTL activation, (xix) reduces inhibition of NK and/or NKT cell activation, (xx) reverses αβ and/or γδ T cell exhaustion, (xxi) increases αβ and/or γδ T cell response, (xxii) increases activity of cytotoxic cells, (xxiii) stimulates antigen-specific memory responses, (xxiv) elicits apoptosis or lysis of cancer cells, (xxv) stimulates cytotoxic or cytostatic effect on cancer cells, (xxvi) induces direct killing of cancer cells, (xxvii) increases Th17 activity (xxviii) increases priming of tumor antigen specific T cells, (xxix) increases priming of tumor antigen specific CD4+ T cells. and/or (xxx) increases priming of tumor antigen specific CD8+ T cells.

According to at least some embodiments, the invention further provides the use of VSIG10 antibody, antigen-binding fragment or conjugate thereof, or a composition comprising same for treatment of cancer or infectious disease, wherein said antibody or antigen-binding fragment is an immunostimulatory antibody which mediates any combination of at least one of the following immunostimulatory effects on immunity:

(i) increases in immune response, (ii) increases in activation of αβ and/or γδ T cells, (iii) increases in cytotoxic T cell activity, (iv) increases in NK and/or NKT cell activity, (v) alleviation of αβ and/or γδ T-cell suppression, (vi) increases in pro-inflammatory cytokine secretion, (vii) increases in IL-2 secretion; (viii) increases in interferon-γ production, (ix) increases in Th1 response, (x) decreases in Th2 response, (xi) decreases or eliminates cell number and/or activity of at least one of regulatory T cells (Tregs), myeloid derived suppressor cells (MDSCs), iMCs, mesenchymal stromal cells, TIE2-expressing monocytes, (xii) reduces regulatory cell activity, and/or the activity of one or more of myeloid derived suppressor cells (MDSCs), iMCs, mesenchymal stromal cells, TIE2-expressing monocytes, (xiii) decreases or eliminates M2 macrophages, (xiv) reduces M2 macrophage pro-tumorigenic activity, (xv) decreases or eliminates N2 neutrophils, (xvi) reduces N2 neutrophils pro-tumorigenic activity, (xvii) reduces inhibition of T cell activation, (xviii) reduces inhibition of CTL activation, (xix) reduces inhibition of NK and/or NKT cell activation, (xx) reverses αβ and/or γδ T cell exhaustion, (xxi) increases αβ and/or γδ T cell response, (xxii) increases activity of cytotoxic cells, (xxiii) stimulates antigen-specific memory responses, (xxiv) elicits apoptosis or lysis of cancer cells, (xxv) stimulates cytotoxic or cytostatic effect on cancer cells, (xxvi) induces direct killing of cancer cells, (xxvii) increases Th17 activity (xxviii) increases priming of tumor antigen specific T cells, (xxix) increases priming of tumor antigen specific CD4+ T cells. and/or (xxx) increases priming of tumor antigen specific CD8+ T cells.

According to at least some embodiments, the invention further provides the use of VSIG10 antibody, antigen-binding fragment or conjugate thereof, or a composition comprising same for treatment of cancer or infectious disease, wherein assessment of treatment can be done using assays that evaluate one or more of the following:

(i) increases in immune response, (ii) increases in activation of αβ and/or γδ T cells, (iii) increases in cytotoxic T cell activity, (iv) increases in NK and/or NKT cell activity, (v) alleviation of αβ and/or γδ T-cell suppression, (vi) increases in pro-inflammatory cytokine secretion, (vii) increases in IL-2 secretion; (viii) increases in interferon-γ production, (ix) increases in Th1 response, (x) decreases in Th2 response, (xi) decreases or eliminates cell number and/or activity of at least one of regulatory T cells (Tregs), myeloid derived suppressor cells (MDSCs), iMCs, mesenchymal stromal cells, TIE2-expressing monocytes, (xii) reduces regulatory cell activity, and/or the activity of one or more of myeloid derived suppressor cells (MDSCs), iMCs, mesenchymal stromal cells, TIE2-expressing monocytes, (xiii) decreases or eliminates M2 macrophages, (xiv) reduces M2 macrophage pro-tumorigenic activity, (xv) decreases or eliminates N2 neutrophils, (xvi) reduces N2 neutrophils pro-tumorigenic activity, (xvii) reduces inhibition of T cell activation, (xviii) reduces inhibition of CTL activation, (xix) reduces inhibition of NK and/or NKT cell activation, (xx) reverses αβ and/or γδ T cell exhaustion, (xxi) increases αβ and/or γδ T cell response, (xxii) increases activity of cytotoxic cells, (xxiii) stimulates antigen-specific memory responses, (xxiv) elicits apoptosis or lysis of cancer cells, (xxv) stimulates cytotoxic or cytostatic effect on cancer cells, (xxvi) induces direct killing of cancer cells, (xxvii) increases Th17 activity, (xxviii) increases priming of tumor antigen specific T cells, (xxix) increases priming of tumor antigen specific CD4+ T cells. and/or (xxx) increases priming of tumor antigen specific CD8+ T cells.

Specific binding for VSIG10 or a VSIG10 epitope can be exhibited, for example, by an antibody having a KD of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the VSIG10 antigen or epitope.

Also, specific binding for a particular antigen or an epitope can be exhibited, for example, by an antibody having a KA or Ka for a VSIG10 antigen or epitope of at least 20-, 50-, 100-, 500-, 1000-, 5,000-, 10,000- or more times greater for the epitope relative to a control, where KA or Ka refers to an association rate of a particular antibody-antigen interaction.

In some embodiments, the anti-VSIG10 antibodies of the invention bind to human VSIG10 with a K_{D} of 100 nM or less, 50 nM or less, 10 nM or less, or 1 nM or less (that is, higher binding affinity), or 1 pM or less, wherein K_{D} is determined by known methods, e.g. surface plasmon resonance (SPR, e.g. Biacore assays), ELISA, KINEXA, and most typically SPR at 250 or 370° C.

B. Specific Anti-VSIG10 Antibodies

The invention provides antigen binding domains, including full length antibodies, which contain a number of specific, enumerated sets of 6 CDRs.

The invention further provides variable heavy and light domains as well as full length heavy and light chains.

As discussed herein, the invention further provides variants of the above components, including variants in the CDRs, as outlined above. In addition, variable heavy chains can be 80%, 90%, 95%, 98% or 99% identical to the “VH” sequences herein, and/or contain from 1, 2, 3, 4, 5, 6, 7, 8, 9, 10 amino acid changes, or more, when Fc variants are used. Variable light chains are provided that can be 80%, 90%, 95%, 98% or 99% identical to the “VL” sequences herein, and/or contain from 1, 2, 3, 4, 5, 6, 7, 8, 9, 10 amino acid changes, or more, when Fc variants are used. Similarly, heavy and light chains are provided that are 80%, 90%, 95%, 98% or 99% identical to the “HC” and “LC” sequences herein, and/or contain from 1, 2, 3, 4, 5, 6, 7, 8, 9, 10 amino acid changes, or more, when Fc variants are used.

Furthermore, the present invention provides a number of CHA antibodies, which are murine antibodies generated from hybridomas. As is well known the art, the six CDRs are useful when put into either human framework variable heavy and variable light regions or when the variable heavy and light domains are humanized.

In addition, the framework regions of the variable heavy and variable light chains can be humanized as is known in the art (with occasional variants generated in the CDRs as needed), and thus humanized variants of the VH and VL chains can be generated. Furthermore, the humanized variable heavy and light domains can then be fused with human constant regions, such as the constant regions from IgG1, IgG2, IgG3 and IgG4.

In particular, as is known in the art, murine VH and VL chains can be humanized as is known in the art, for example, using the IgBLAST program of the NCBI website, as outlined in Ye et al. Nucleic Acids Res. 41:W34-W40 (2013), herein incorporated by reference in its entirety for the humanization methods. IgBLAST takes a murine VH and/or VL sequence and compares it to a library of known human germline sequences. As shown herein, for the humanized sequences generated herein, the databases used were IMGT human VH genes (F+ORF, 273 germline sequences) and IMGT human VL kappa genes (F+ORF, 74 germline sequences).

In some embodiments, the anti-VSIG10 antibodies of the present invention include anti-VSIG10 antibodies wherein the V_{H} and V_{L} sequences of different anti-VSIG10 antibodies can be “mixed and matched” to create other anti-VSIG10 antibodies. VSIG10 binding of such “mixed and matched” antibodies can be tested using the binding assays described above. e.g., ELISAs). In some embodiments, when V_{H} and V_{L} chains are mixed and matched, a V_{H} sequence from a particular V_{H}/V_{L} pairing is replaced with a structurally similar V_{H} sequence. Likewise, in some embodiments, a V_{L} sequence from a particular V_{H}/V_{L} pairing is replaced with a structurally similar V_{L} sequence. For example, the V_{H} and V_{L} sequences of homologous antibodies are particularly amenable for mixing and matching.

Accordingly, the antibodies of the invention comprise CDR amino acid sequences selected from the group consisting of (a) sequences as listed herein; (b) sequences that differ from those CDR amino acid sequences specified in (a) by 1, 2, 3, 4, 5, 6, 7, 8, 9, 10 or more amino acid substitutions; (c) amino acid sequences having 90% or greater, 95% or greater, 98% or greater, or 99% or greater sequence identity to the sequences specified in (a) or (b); (d) a polypeptide having an amino acid sequence encoded by a polynucleotide having a nucleic acid sequence encoding the amino acids as listed herein.

Additionally included in the definition of VSIG10 antibodies are antibodies that share identity to the VSIG10 antibodies enumerated herein. That is, in certain embodiments, an anti-VSIG10 antibody according to the invention comprises heavy and light chain variable regions comprising amino acid sequences that are homologous to isolated anti-VSIG10 amino acid sequences of preferred anti-VSIG10 immune molecules, respectively, wherein the antibodies retain the desired functional properties of the parent anti-VSIG10 antibodies. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available commercially), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J Mol. Biol. 215:403-10. BLAST protein searches can be performed with the XBLAST program, score=50, wordlength=3 to obtain amino acid sequences homologous to the antibody molecules according to at least some embodiments of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

In general, the percentage identity for comparison between VSIG10 antibodies is at least 75%, at least 80%, at least 90%, with at least about 95, 96, 97, 98 or 99% percent identity being preferred. The percentage identity may be along the whole amino acid sequence, for example the entire heavy or light chain or along a portion of the chains. For example, included within the definition of the anti-VSIG10 antibodies of the invention are those that share identity along the entire variable region (for example, where the identity is 95 or 98% identical along the variable regions), or along the entire constant region, or along just the Fc domain.

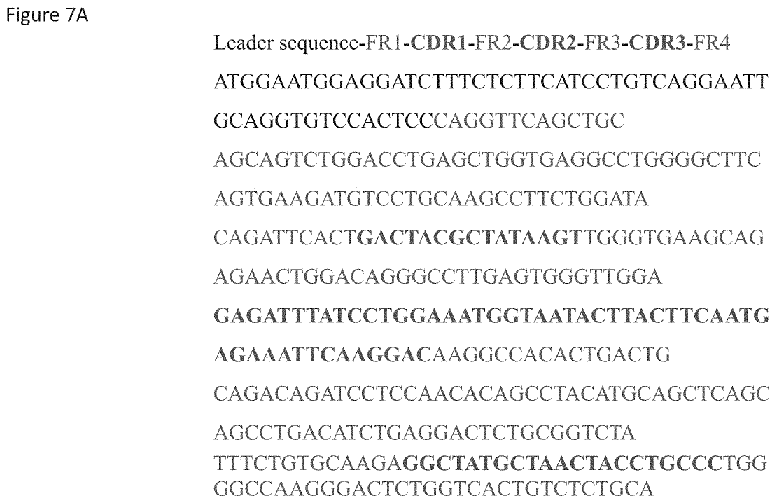

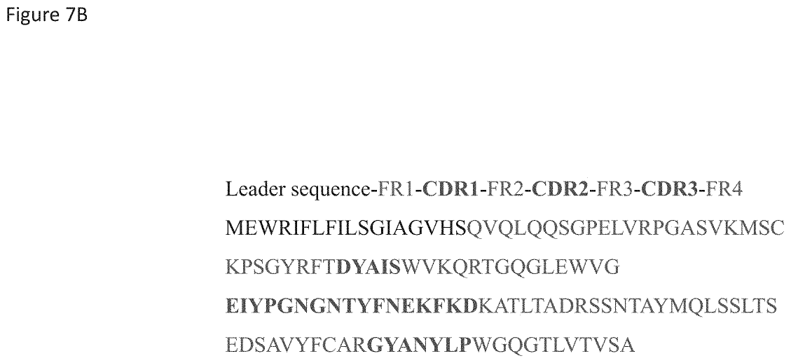

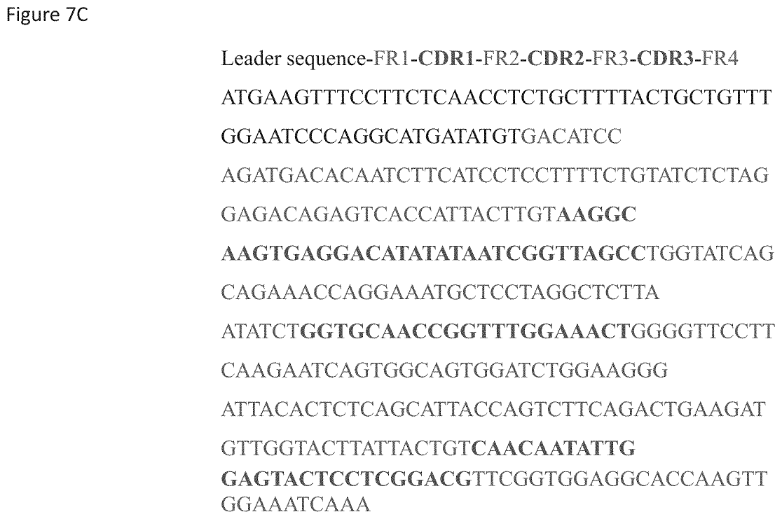

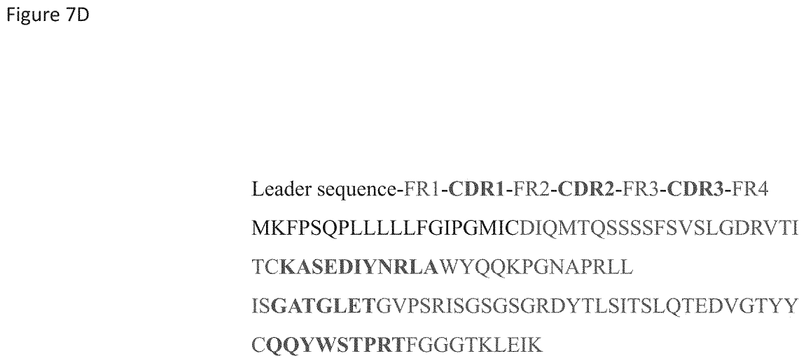

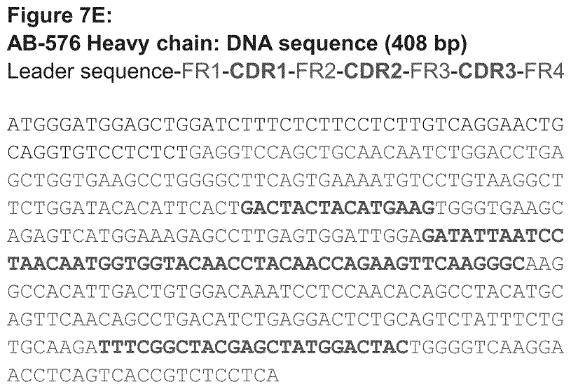

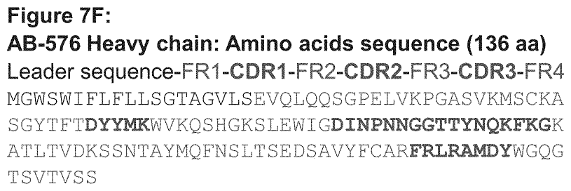

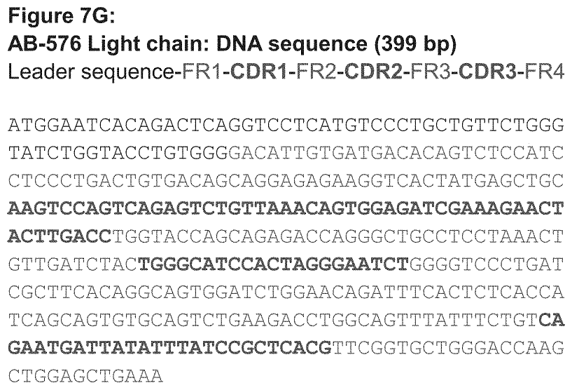

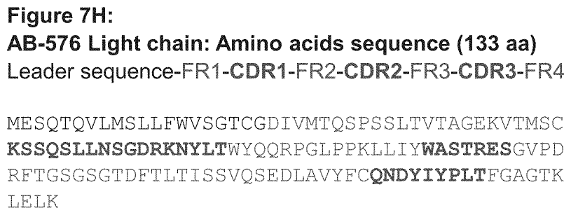

In addition, also included are sequences that may have the identical CDRs but changes in the variable domain (or entire heavy or light chain). For example, VSIG10 antibodies include those with CDRs identical to those shown in FIG. 7 but whose identity along the variable region can be lower, for example 95 or 98% percent identical.

C. VSIG10 Antibodies that Compete for Binding with Enumerated Antibodies

The present invention provides not only the enumerated antibodies but additional antibodies that compete with the enumerated antibodies to specifically bind to the VSIG10 molecule.

Additional antibodies that compete with the enumerated antibodies are generated, as is known in the art and generally outlined below. Competitive binding studies can be done as is known in the art, generally using SPR/Biacore® binding assays, as well as ELISA and cell-based assays.

Generation of Additional Antibodies

Additional antibodies to human VSIG10 can be done as is well known in the art, using well known methods such as those outlined in the examples. Thus, additional anti-VSIG10 antibodies can be generated by traditional methods such as immunizing mice (sometimes using DNA immunization, for example, such as is used by Aldevron), followed by screening against human VSIG10 protein and hybridoma generation, with antibody purification and recovery. Additionally or alternatively, anti-VSIG10 antibodies may be generated through phage display as is known in the art.

Monoclonal antibodies (mAbs) of the present invention can be produced by a variety of techniques, including conventional monoclonal antibody methodology e.g., the standard somatic cell hybridization technique of Kohler and Milstein (1975) Nature 256:495. Although somatic cell hybridization procedures are preferred, in principle, other techniques for producing monoclonal antibody can be employed e.g., viral or oncogenic transformation of B lymphocytes.

A preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

According to at least some embodiments of the invention, the antibodies are human monoclonal antibodies. Such human monoclonal antibodies directed against VSIG10 can be generated using transgenic or transchromosomic mice carrying parts of the human immune system rather than the mouse system. These transgenic and transchromosomic mice include mice referred to herein as the HuMAb Mouse® and KM Mouse®, respectively, and are collectively referred to herein as “human Ig mice.” The HuMAb Mouse™. (Medarex. Inc.) contains human immunoglobulin gene miniloci that encode unrearranged human heavy (.mu. and .gamma.) and .kappa. light chain immunoglobulin sequences, together with targeted mutations that inactivate the endogenous.mu. and .kappa. chain loci (see e.g., Lonberg, et al. (1994) Nature 368(6474): 856-859). Accordingly, the mice exhibit reduced expression of mouse IgM or .kappa., and in response to immunization, the introduced human heavy and light chain transgenes undergo class switching and somatic mutation to generate high affinity human IgGkappa. monoclonal (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13: 65-93, and Harding, F. and Lonberg, N. (1995) Ann. N.Y. Acad. Sci. 764:536-546). The preparation and use of the HuMab Mouse®, and the genomic modifications carried by such mice, is further described in Taylor, L. et al. (1992) Nucleic Acids Research 20:6287-6295; Chen, J. et al. (1993) International Immunology 5:647-656; Tuaillon et al. (1993) Proc. Natl. Acad. Sci. USA 90:3720-3724; Choi et al. (1993) Nature Genetics 4:117-123; Chen, J. et al. (1993) EMBO J. 12: 821-830; Tuaillon et al. (1994) J. Immunol. 152:2912-2920; Taylor, L. et al. (1994) International Immunology 6:579-591; and Fishwild, D. et al. (1996) Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al.

In another embodiment, human antibodies according to at least some embodiments of the invention can be raised using a mouse that carries human immunoglobulin sequences on transgenes and transchomosomes, such as a mouse that carries a human heavy chain transgene and a human light chain transchromosome. Such mice, referred to herein as “KM Mice™.”, are described in detail in PCT Publication WO 02/43478 to Ishida et al.

Still further, alternative transgenic animal systems expressing human immunoglobulin genes are available in the art and can be used to raise anti-VSIG10 antibodies according to at least some embodiments of the invention. For example, an alternative transgenic system referred to as the Xenomouse (Abgenix, Inc.) can be used; such mice are described in, for example, U.S. Pat. Nos. 5,939,598; 6,075,181; 6,114,598; 6, 150,584 and 6,162,963 to Kucherlapati et al.

Moreover, alternative transchromosomic animal systems expressing human immunoglobulin genes are available in the art and can be used to raise anti-VSIG10 antibodies according to at least some embodiments of the invention. For example, mice carrying both a human heavy chain transchromosome and a human light chain transchromosome, referred to as “TC mice” can be used; such mice are described in Tomizuka et al. (2000) Proc. Natl. Acad Sci. USA 97:722-727. Furthermore, cows carrying human heavy and light chain transchromosomes have been described in the art (Kuroiwa et al. (2002) Nature Biotechnology 20:889-894) and can be used to raise anti-VSIG10 antibodies according to at least some embodiments of the invention.

Human monoclonal antibodies according to at least some embodiments of the invention can also be prepared using phage display methods for screening libraries of human immunoglobulin genes. Such phage display methods for isolating human antibodies are established in the art. See for example: U.S. Pat. Nos. 5,223,409; 5,403,484; and U.S. Pat. No. 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Human monoclonal antibodies according to at least some embodiments of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

Immunization of Human IG Mice

When human Ig mice are used to raise human antibodies according to at least some embodiments of the invention, such mice can be immunized with a purified or enriched preparation of VSIG10 antigen and/or recombinant VSIG10, or VSIG10 fusion protein, as described by Lonberg, N. et al. (1994) Nature 368(6474): 856-859; Fishwild, D. et al. (1996) Nature Biotechnology 14: 845-851; and PCT Publication WO 98/24884 and WO 01/14424. Preferably, the mice will be 6-16 weeks of age upon the first infusion. For example, a purified or recombinant preparation (5-50 .mu.g) of VSIG10 antigen can be used to immunize the human Ig mice intraperitoneally.

Prior experience with various antigens by others has shown that the transgenic mice respond when initially immunized intraperitoneally (IP) with antigen in complete Freund's adjuvant, followed by every other week IP immunizations (up to a total of 6) with antigen in incomplete Freund's adjuvant. However, adjuvants other than Freund's are also found to be effective. In addition, whole cells in the absence of adjuvant are found to be highly immunogenic. The immune response can be monitored over the course of the immunization protocol with plasma samples being obtained by retroorbital bleeds. The plasma can be screened by ELISA (as described below), and mice with sufficient titers of anti-VSIG10 human immunoglobulin can be used for fusions. Mice can be boosted intravenously with antigen 3 days before sacrifice and removal of the spleen. It is expected that 2-3 fusions for each immunization may need to be performed. Between 6 and 24 mice are typically immunized for each antigen. Usually both HCo7 and HCo12 strains are used. In addition, both HCo7 and HCo12 transgene can be bred together into a single mouse having two different human heavy chain transgenes (HCo7/HCo 12). Alternatively or additionally, the KM Mouse® strain can be used.

Generation of Hybridomas Producing Human Monoclonal Antibodies

To generate hybridomas producing human monoclonal antibodies according to at least some embodiments of the invention, splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to one-sixth the number of P3X63-Ag8.653 nonsecreting mouse myeloma cells (ATCC, CRL 1580) with 50% PEG. Cells are plated at approximately 2×10-5 in flat bottom microtiter plate, followed by a two week incubation in selective medium containing 20% fetal Clone Serum, 18% “653” conditioned media, 5% origen (IGEN), 4 mM L-glutamine, 1 mM sodium pyruvate, 5 mM HEPES, 0.055 mM 2-mercaptoethanol, 50 units/ml penicillin, 50 mg/ml streptomycin, 50 mg/ml gentamycin and 1×HAT (Sigma; the HAT is added 24 hours after the fusion). After approximately two weeks, cells can be cultured in medium in which the HAT is replaced with HT. Individual wells can then be screened by ELISA for human monoclonal IgM and IgG antibodies. Once extensive hybridoma growth occurs, medium can be observed usually after 10-14 days. The antibody secreting hybridomas can be replated, screened again, and if still positive for human IgG, the monoclonal antibodies can be subcloned at least twice by limiting dilution. The stable subclones can then be cultured in vitro to generate small amounts of antibody in tissue culture medium for characterization.

To purify human monoclonal antibodies, selected hybridomas can be grown in two-liter spinner-flasks for monoclonal antibody purification. Supernatants can be filtered and concentrated before affinity chromatography with protein A-Sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD280 using 1.43 extinction coefficient. The monoclonal antibodies can be aliquoted and stored at −80 degrees C.

Generation of Transfectomas Producing Monoclonal Antibodies

Antibodies according to at least some embodiments according to at least some embodiments of the invention also can be produced in a host cell transfectoma using, for example, a combination of recombinant DNA techniques and gene transfection methods as is well known in the art (e.g., Morrison, S. (1985) Science 229:1202).

For example, to express the antibodies, or antibody fragments thereof, DNAs encoding partial or full-length light and heavy chains, can be obtained by standard molecular biology techniques (e.g., PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the VH segment is operatively linked to the CH segments within the vector and the VK segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors according to at least some embodiments of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or .beta.-globin promoter. Still further, regulatory elements composed of sequences from different sources, such as the SR alpha. promoter system, which contains sequences from the SV40 early promoter and the long terminal repeat of human T cell leukemia virus type 1 (Takebe, Y. et al. (1988) Mol. Cell. Biol. 8:466-472).

In addition to the antibody chain genes and regulatory sequences, the recombinant expression vectors according to at least some embodiments of the invention may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vectors encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies according to at least some embodiments of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Preferred mammalian host cells for expressing the recombinant antibodies according to at least some embodiments of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. In particular, for use with NSO myeloma cells, another preferred expression system is the GS gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Characterization of Antibody Binding to Antigen

Antibodies according to at least some embodiments of the invention can be tested for binding to VSIG10 by, for example, standard ELISA. Briefly, microtiter plates are coated with purified VSIG10 at 0.25 .mu.g/ml in PBS, and then blocked with 5% bovine serum albumin in PBS. Dilutions of antibody (e.g., dilutions of plasma from -immunized mice) are added to each well and incubated for 1-2 hours at 37 degrees C. The plates are washed with PBS/Tween and then incubated with secondary reagent (e.g., for human antibodies, a goat-anti-human IgG Fc-specific polyclonal reagent) conjugated to alkaline phosphatase for 1 hour at 37 degrees C. After washing, the plates are developed with pNPP substrate (1 mg/ml), and analyzed at OD of 405-650. Preferably, mice which develop the highest titers will be used for fusions.

An ELISA assay as described above can also be used to screen for hybridomas that show positive reactivity with VSIG10 immunogen. Hybridomas that bind with high avidity to VSIG10 are subcloned and further characterized. One clone from each hybridoma, which retains the reactivity of the parent cells (by ELISA), can be chosen for making a 5-10 vial cell bank stored at −140 degrees C., and for antibody purification.

To purify anti-VSIG10 antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD280 using 1.43 extinction coefficient. The monoclonal antibodies can be aliquoted and stored at −80 degrees C.

To determine if the selected anti-VSIG10 monoclonal antibodies bind to unique epitopes, each antibody can be biotinylated using commercially available reagents (Pierce, Rockford, Ill.). Competition studies using unlabeled monoclonal antibodies and biotinylated monoclonal antibodies can be performed using VSIG10 coated-ELISA plates as described above. Biotinylated mAb binding can be detected with a strep-avidin-alkaline phosphatase probe.

To determine the isotype of purified antibodies, isotype ELISAs can be performed using reagents specific for antibodies of a particular isotype. For example, to determine the isotype of a human monoclonal antibody, wells of microtiter plates can be coated with 1 .mu.g/ml of anti-human immunoglobulin overnight at 4 degrees C. After blocking with 1% BSA, the plates are reacted with 1 mug/ml or less of test monoclonal antibodies or purified isotype controls, at ambient temperature for one to two hours. The wells can then be reacted with either human IgG1 or human IgM-specific alkaline phosphatase-conjugated probes. Plates are developed and analyzed as described above.

Anti-VSIG10 human IgGs can be further tested for reactivity with VSIG10 antigen, respectively, by Western blotting. Briefly, VSIG10antigen can be prepared and subjected to sodium dodecyl sulfate polyacrylamide gel electrophoresis. After electrophoresis, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

Alternative Scaffolds

According to at least some embodiments the invention relates to protein scaffolds with specificities and affinities in a range similar to specific antibodies. According to at least some embodiments the present invention relates to an antigen-binding construct comprising a protein scaffold which is linked to one or more epitope-binding domains. Such engineered protein scaffolds are usually obtained by designing a random library with mutagenesis focused at a loop region or at an otherwise permissible surface area and by selection of variants against a given target via phage display or related techniques. According to at least some embodiments the invention relates to alternative scaffolds including, but not limited to, anticalins, DARPins, Armadillo repeat proteins, protein A, lipocalins, fibronectin domain, ankyrin consensus repeat domain, thioredoxin, chemically constrained peptides and the like. According to at least some embodiments the invention relates to alternative scaffolds that are used as therapeutic agents for treatment of cancer, infectious diseases as well as for in vivo diagnostics.

According to at least some embodiments the invention further provides a pharmaceutical composition comprising an antigen binding construct as described herein a pharmaceutically acceptable carrier.

The term ‘Protein Scaffold’ as used herein includes but is not limited to an immunoglobulin (Ig) scaffold, for example an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions. Such protein scaffolds may comprise antigen-binding sites in addition to the one or more constant regions, for example where the protein scaffold comprises a full IgG. Such protein scaffolds will be capable of being linked to other protein domains, for example protein domains which have antigen-binding sites, for example epitope-binding domains or ScFv domains.

A “domain” is a folded protein structure which has tertiary structure independent of the rest of the protein. Generally, domains are responsible for discrete functional properties of proteins and in many cases may be added, removed or transferred to other proteins without loss of function of the remainder of the protein and/or of the domain. A “single antibody variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain.

The phrase “immunoglobulin single variable domain” refers to an antibody variable domain (VH, V HH, V L) that specifically binds an antigen or epitope independently of a different V region or domain. An immunoglobulin single variable domain can be present in a format (e.g., homo- or hetero-multimer) with other, different variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is the same as an “immunoglobulin single variable domain” which is capable of binding to an antigen as the term is used herein. An immunoglobulin single variable domain may be a human antibody variable domain, but also includes single antibody variable domains from other species such as rodent (for example, as disclosed in WO 00/29004), nurse shark and Camelid V HH dAbs. Camelid V HH are immunoglobulin single variable domain polypeptides that are derived from species including camel, llama, alpaca, dromedary, and guanaco, which produce heavy chain antibodies naturally devoid of light chains. Such V HH domains may be humanised according to standard techniques available in the art, and such domains are still considered to be “domain antibodies” according to the invention. As used herein “VH includes camelid V HH domains. NARV are another type of immunoglobulin single variable domain which were identified in cartilaginous fish including the nurse shark. These domains are also known as Novel Antigen Receptor variable region (commonly abbreviated to V(NAR) or NARV). For further details see Mol. Immunol. 44, 656-665 (2006) and US20050043519A.

The term “epitope-binding domain” refers to a domain that specifically binds an antigen or epitope independently of a different V region or domain, this may be a domain antibody (dAb), for example a human, camelid or shark immunoglobulin single variable domain or it may be a domain which is a derivative of a scaffold selected from the group consisting of CTLA-4 (Evibody); lipocalin; Protein A derived molecules such as Z-domain of Protein A (Affibody, SpA), A-domain (Avimer/Maxibody); Heat shock proteins such as GroEI and GroES; transferrin (trans-body); ankyrin repeat protein (DARPin); peptide aptamer; C-type lectin domain (Tetranectin); human &#947;-crystallin and human ubiquitin (affilins); PDZ domains; scorpion toxinkunitz type domains of human protease inhibitors; Armadillo repeat proteins, thioredoxin, and fibronectin (adnectin); which has been subjected to protein engineering in order to obtain binding to a ligand other than the natural ligand.

Loops corresponding to CDRs of antibodies can be substituted with heterologous sequence to confer different binding properties i.e. Evibodies. For further details see Journal of Immunological Methods 248 (1-2), 31-45 (2001) Lipocalins are a family of extracellular proteins which transport small hydrophobic molecules such as steroids, bilins, retinoids and lipids. They have a rigid secondary structure with a numer of loops at the open end of the conical structure which can be engineered to bind to different target antigens. Anticalins are between 160-180 amino acids in size, and are derived from lipocalins. For further details see Biochim Biophys Acta 1482: 337-350 (2000), U.S. Pat. No. 7,250,297B1 and US20070224633. An affibody is a scaffold derived from Protein A of Staphylococcus aureus which can be engineered to bind to antigen. The domain consists of a three-helical bundle of approximately 58 amino acids. Libraries have been generated by randomisation of surface residues. For further details see Protein Eng. Des. SeI. 17, 455-462 (2004) and EP1641818A1 Avimers are multidomain proteins derived from the A-domain scaffold family. The native domains of approximately 35 amino acids adopt a defined disulphide bonded structure. Diversity is generated by shuffling of the natural variation exhibited by the family of A-domains. For further details see Nature Biotechnology 23(12), 1556-1561 (2005) and Expert Opinion on Investigational Drugs 16(6), 909-917 (June 2007) A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem 274, 24066-24073 (1999).

Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two alpha helices;-beta turn. They can be engineered to bind different target antigens by randomising residues in the first alpha-helix and a beta-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1.

Fibronectin is a scaffold which can be engineered to bind to antigen. Adnectins consists of a backbone of the natural amino acid sequence of the 10th domain of the 15 repeating units of human fibronectin type III (FN3). Three loops at one end of the beta;-sandwich can be engineered to enable an Adnectin to specifically recognize a therapeutic target of interest. For further details see Protein Eng. Des. SeI. 18, 435-444 (2005), US200801 39791, WO2005056764 and U.S. Pat. No. 6,818,418B1.

Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5. 783-797 (2005).

Microbodies are derived from naturally occurring microproteins of 25-50 amino acids in length which contain 3-4 cysteine bridges—examples of microproteins include KalataBI and conotoxin and knottins. The microproteins have a loop which can be engineered to include upto 25 amino acids without affecting the overall fold of the microprotein. For further details of engineered knottin domains, see WO2008098796.

Other epitope binding domains include proteins which have been used as a scaffold to engineer different target antigen binding properties include human &#947; beta-crystallin and human ubiquitin (affilins), kunitz type domains of human protease inhibitors, PDZ-domains of the Ras-binding protein AF-6, scorpion toxins (charybdotoxin), C-type lectin domain (tetranectins) are reviewed in Chapter 7—Non-Antibody Scaffolds from Handbook of Therapeutic Antibodies (2007, edited by Stefan Dubel) and Protein Science 15:14-27 (2006). Epitope binding domains of the present invention could be derived from any of these alternative protein domains.

Conjugates or Immunoconjugates

In another aspect, the present invention features antibody-drug conjugates (ADCs), used for example for treatment of cancer, consisting of an antibody (or antibody fragment such as a single-chain variable fragment [scFv]) linked to a payload drug (often cytotoxic). The antibody causes the ADC to bind to the target cancer cells. Often the ADC is then internalized by the cell and the drug is released into the cell. Because of the targeting, the side effects are lower and give a wider therapeutic window. Hydrophilic linkers (e.g., PEG4Mal) help prevent the drug being pumped out of resistant cancer cells through MDR (multiple drug resistance) transporters. ADCs based on cleavable linkers are thought to have a less favorable therapeutic window, but targets (tumor cell surface antigens) that do not get internalized efficiently seem more suitable for cleavable linkers.

In another aspect, the present invention features immunoconjugates comprising an anti-VSIG10 antibody, or a fragment thereof, conjugated to a therapeutic moiety, such as a cytotoxin, a drug (e.g., an immune modulator) or a radiotoxin. Such conjugates are referred to herein as “immunoconjugates”. Immunoconjugates that include one or more cytotoxins are referred to as “immunotoxins.” A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Other preferred examples of therapeutic cytotoxins that can be conjugated to an antibody according to at least some embodiments of the invention include duocarmycins, calicheamicins, maytansines and auristatins, and derivatives thereof. An example of a calicheamicin antibody conjugate is commercially available (Mylotarg™; Wyeth).

Cytotoxins can be conjugated to antibodies according to at least some embodiments of the invention using linker technology available in the art. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D).

For further discussion of types of cytotoxins, linkers and methods for conjugating therapeutic agents to antibodies, see also Saito, G. et al. (2003) Adv. Drug Deliv. Rev. 55:199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3:1089-1091; Senter, P. D. and Springer, C. J. (2001) Adv. Drug Deliv. Rev. 53:247-264.

Antibodies of the present invention also can be conjugated to a radioactive isotope to generate cytotoxic radiopharmaceuticals, also referred to as radioimmunoconjugates. Examples of radioactive isotopes that can be conjugated to antibodies for use diagnostically or therapeutically include, but are not limited to, iodine 131, indium 111, yttrium 90 and lutetium 177. Methods for preparing radioimmunconjugates are established in the art. Examples of radioimmunoconjugates are commercially available, including Zevalin (IDEC Pharmaceuticals) and Bexxar. (Corixa Pharmaceuticals), and similar methods can be used to prepare radioimmunoconjugates using the antibodies according to at least some embodiments of the invention.

The antibody conjugates according to at least some embodiments of the invention can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor or interferon-.gamma.; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Bispecific Molecules

In another aspect, the present invention features bispecific molecules comprising an anti-VSIG10 antibody, or a fragment thereof, according to at least some embodiments of the invention. An antibody according to at least some embodiments of the invention, or antigen-binding portions thereof, can be derivatized or linked to another functional molecule, e.g., another peptide or protein (e.g., another antibody or ligand for a receptor) to generate a bispecific molecule that binds to at least two different binding sites or target molecules. The antibody according to at least some embodiments of the invention may in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule according to at least some embodiments of the invention, an antibody can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, the present invention includes bispecific molecules comprising at least one first binding specificity for VSIG10 and a second binding specificity for a second target epitope. According to at least some embodiments of the invention, the second target epitope is an Fc receptor, e.g., human Fc gamma RI (CD64) or a human Fc alpha receptor (CD89). Therefore, the invention includes bispecific molecules capable of binding both to Fc gamma. R, Fc alpha R or Fc epsilon R expressing effector cells (e.g., monocytes, macrophages or polymorphonuclear cells (PMNs)), and to target cells expressing VSIG10, respectively. These bispecific molecules target VSIG10 expressing cells to effector cell and trigger Fc receptor-mediated effector cell activities, such as phagocytosis of an VSIG10 expressing cells, antibody dependent cell-mediated cytotoxicity (ADCC), cytokine release, or generation of superoxide anion.

According to at least some embodiments of the invention in which the bispecific molecule is multispecific, the molecule can further include a third binding specificity, in addition to an anti-Fc binding specificity and an anti-6f binding specificity. In one embodiment, the third binding specificity is an anti-enhancement factor (EF) portion, e.g., a molecule which binds to a surface protein involved in cytotoxic activity and thereby increases the immune response against the target cell.

The “anti-enhancement factor portion” can be an antibody, functional antibody fragment or a ligand that binds to a given molecule, e.g., an antigen or a receptor, and thereby results in an enhancement of the effect of the binding determinants for the Fc receptor or target cell antigen. The “anti-enhancement factor portion” can bind an Fc receptor or a target cell antigen. Alternatively, the anti-enhancement factor portion can bind to an entity that is different from the entity to which the first and second binding specificities bind. For example, the anti-enhancement factor portion can bind a cytotoxic T-cell (e.g., via CD2, CD3, CD8, CD28, CD4, CD40, ICAM-1 or other immune cell that results in an increased immune response against the target cell).

According to at least some embodiments of the invention, the bispecific molecules comprise as a binding specificity at least one antibody, or an antibody fragment thereof, including, e.g., an Fab, Fab′, F(ab′).sub.2, Fv, or a single chain Fv. The antibody may also be a light chain or heavy chain dimer, or any minimal fragment thereof such as a Fv or a single chain construct as described in Ladner et al. U.S. Pat. No. 4,946,778, the contents of which is expressly incorporated by reference.

In one embodiment, the binding specificity for an Fcγ receptor is provided by a monoclonal antibody, the binding of which is not blocked by human immunoglobulin G (IgG). As used herein, the term “IgG receptor” refers to any of the eight.gamma.-chain genes located on chromosome 1. These genes encode a total of twelve transmembrane or soluble receptor isoforms which are grouped into three Fc.gamma. receptor classes: Fc gamma R1 (CD64), Fc gamma RII(CD32), and Fc gamma.RIII (CD16). In one preferred embodiment, the Fc gamma. receptor a human high affinity Fc.gamma RI. The human Fc gammaRI is a 72 kDa molecule, which shows high affinity for monomeric IgG (10 8-10-9 M.-1).

The production and characterization of certain preferred anti-Fc gamma. monoclonal antibodies are described by Fanger et al. in PCT Publication WO 88/00052 and in U.S. Pat. No. 4,954,617, the teachings of which are fully incorporated by reference herein. These antibodies bind to an epitope of Fc.gamma.R1, FcyRII or FcyRIII at a site which is distinct from the Fc.gamma. binding site of the receptor and, thus, their binding is not blocked substantially by physiological levels of IgG. Specific anti-Fc.gamma.RI antibodies useful in this invention are mAb 22, mAb 32, mAb 44, mAb 62 and mAb 197. The hybridoma producing mAb 32 is available from the American Type Culture Collection, ATCC Accession No. HB9469. In other embodiments, the anti-Fcy receptor antibody is a humanized form of monoclonal antibody 22 (H22). The production and characterization of the H22 antibody is described in Graziano, R. F. et al. (1995) J. Immunol. 155 (10): 4996-5002 and PCT Publication WO 94/10332. The H22 antibody producing cell line is deposited at the American Type Culture Collection under the designation HAO22CLI and has the accession no. CRL 11177.

In still other preferred embodiments, the binding specificity for an Fc receptor is provided by an antibody that binds to a human IgA receptor, e.g., an Fc-alpha receptor (Fc alpha.RI(CD89)), the binding of which is preferably not blocked by human immunoglobulin A (IgA). The term “IgA receptor” is intended to include the gene product of one alpha.-gene (Fc alpha.RI) located on chromosome 19. This gene is known to encode several alternatively spliced transmembrane isoforms of 55 to 10 kDa.

Fc.alpha.RI (CD89) is constitutively expressed on monocytes/macrophages, eosinophilic and neutrophilic granulocytes, but not on non-effector cell populations. Fc alpha RI has medium affinity (Approximately 5X10-7 M-1) for both IgA1 and IgA2, which is increased upon exposure to cytokines such as G-CSF or GM-CSF (Morton, H. C. et al. (1996) Critical Reviews in Immunology 16:423-440). Four FcaRI-specific monoclonal antibodies, identified as A3, A59, A62 and A77, which bind Fc.alpha.RI outside the IgA ligand binding domain, have been described (Monteiro, R. C. et al. (1992) J. Immunol. 148:1764).

Fc. alpha. RI and Fc gamma. RI are preferred trigger receptors for use in the bispecific molecules according to at least some embodiments of the invention because they are (1) expressed primarily on immune effector cells, e.g., monocytes, PMNs, macrophages and dendritic cells; (2) expressed at high levels (e.g., 5,000-100,000 per cell); (3) mediators of cytotoxic activities (e.g., ADCC, phagocytosis); (4) mediate enhanced antigen presentation of antigens, including self-antigens, targeted to them.

While human monoclonal antibodies are preferred, other antibodies which can be employed in the bispecific molecules according to at least some embodiments of the invention are murine, chimeric and humanized monoclonal antibodies.

The bispecific molecules of the present invention can be prepared by conjugating the constituent binding specificities, e.g., the anti-FcR and anti-VSIG10 binding specificities, using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyld-ithio)propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Preferred conjugating agents are SATA and sulfo-SMCC, both available from Pierce Chemical Co. (Rockford, Ill.).

When the binding specificities are antibodies, they can be conjugated via sulfhydryl bonding of the C-terminus hinge regions of the two heavy chains. In a particularly preferred embodiment, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific molecule is a mAbXmAb, mAbXFab, FabXF(ab′)2 or ligandXFab fusion protein. A bispecific molecule according to at least some embodiments of the invention can be a single chain molecule comprising one single chain antibody and a binding determinant, or a single chain bispecific molecule comprising two binding determinants. Bispecific molecules may comprise at least two single chain molecules. Methods for preparing bispecific molecules are described for example in U.S. Pat. Nos. 5,260,203; 5,455,030; 4,881,175; 5,132,405; 5,091,513; 5,476,786; 5,013,653; 5,258,498; and 5,482,858.

Binding of the bispecific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. For example, the FcR-antibody complexes can be detected using e.g., an enzyme-linked antibody or antibody fragment which recognizes and specifically binds to the antibody-FcR complexes. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma. counter or a scintillation counter or by autoradiography.

Nucleic Acid Compositions

Nucleic acid compositions encoding the anti-VSIG10 antibodies of the invention are also provided, as well as expression vectors containing the nucleic acids and host cells transformed with the nucleic acid and/or expression vector compositions. As will be appreciated by those in the art, the protein sequences depicted herein can be encoded by any number of possible nucleic acid sequences, due to the degeneracy of the genetic code.

The nucleic acid compositions that encode the VSIG10 antibodies will depend on the format of the antibody. For traditional, tetrameric antibodies containing two heavy chains and two light chains are encoded by two different nucleic acids, one encoding the heavy chain and one encoding the light chain. These can be put into a single expression vector or two expression vectors, as is known in the art, transformed into host cells, where they are expressed to form the antibodies of the invention. In some embodiments, for example when scFv constructs are used, a single nucleic acid encoding the variable heavy chain-linker-variable light chain is generally used, which can be inserted into an expression vector for transformation into host cells. The nucleic acids can be put into expression vectors that contain the appropriate transcriptional and translational control sequences, including, but not limited to, signal and secretion sequences, regulatory sequences, promoters, origins of replication, selection genes, etc.

Preferred mammalian host cells for expressing the recombinant antibodies according to at least some embodiments of the invention include Chinese Hamster Ovary (CHO cells), PER.C6, HEK293 and others as is known in the art.

The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art.

To create a scFv gene, the V_{H}- and V_{L}-encoding DNA fragments are operatively linked to another fragment encoding a flexible linker, e.g., encoding the amino acid sequence (Gly4-Ser)3, such that the V_{H} and V_{L} sequences can be expressed as a contiguous single-chain protein, with the V_{L} and V_{H} regions joined by the flexible linker (see e.g., Bird et al. (1988) Science 242:423-426; Huston et al. (1988) Proc. Natl. Acad. Sci. USA 85:5879-5883; McCafferty et al., (1990) Nature 348:552-554).

### II. Formulations of Anti-VSIG10 Antibodies

The therapeutic compositions used in the practice of the foregoing methods can be formulated into pharmaceutical compositions comprising a carrier suitable for the desired delivery method. Suitable carriers include any material that when combined with the therapeutic composition retains the anti-tumor function of the therapeutic composition and is generally non-reactive with the patient's immune system. Examples include, but are not limited to, any of a number of standard pharmaceutical carriers such as sterile phosphate buffered saline solutions, bacteriostatic water, and the like (see, generally, Remington's Pharmaceutical Sciences 16^{th} Edition, A. Osal., Ed., 1980). Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN™, PLURONICS™ or polyethylene glycol (PEG).

In a preferred embodiment, the pharmaceutical composition that comprises the antibodies of the invention may be in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine. The formulations to be used for in vivo administration are preferrably sterile. This is readily accomplished by filtration through sterile filtration membranes or other methods.

Administration of the pharmaceutical composition comprising antibodies of the present invention, preferably in the form of a sterile aqueous solution, may be done in a variety of ways, including, but not limited to subcutaneously and intravenously. Subcutaneous administration may be preferable in some circumstances because the patient may self-administer the pharmaceutical composition. Many protein therapeutics are not sufficiently potent to allow for formulation of a therapeutically effective dose in the maximum acceptable volume for subcutaneous administration. This problem may be addressed in part by the use of protein formulations comprising arginine-HCl, histidine, and polysorbate (see WO 04091658). Fc polypeptides of the present invention may be more amenable to subcutaneous administration due to, for example, increased potency, improved serum half-life, or enhanced solubility.

As is known in the art, protein therapeutics are often delivered by IV infusion or bolus. The antibodies of the present invention may also be delivered using such methods. For example, administration may venious be by intravenous infusion with 0.9% sodium chloride as an infusion vehicle.

In addition, any of a number of delivery systems are known in the art and may be used to administer the Fc variants of the present invention. Examples include, but are not limited to, encapsulation in liposomes, microparticles, microspheres (eg. PLA/PGA microspheres), and the like. Alternatively, an implant of a porous, non-porous, or gelatinous material, including membranes or fibers, may be used. Sustained release systems may comprise a polymeric material or matrix such as polyesters, hydrogels, poly(vinylalcohol), polylactides, copolymers of L-glutamic acid and ethyl-L-gutamate, ethylene-vinyl acetate, lactic acid-glycolic acid copolymers such as the LUPRON DEPOT®, and poly-D-(−)-3-hydroxyburyric acid. The antibodies disclosed herein may also be formulated as immunoliposomes. A liposome is a small vesicle comprising various types of lipids, phospholipids and/or surfactant that is useful for delivery of a therapeutic agent to a mammal. Liposomes containing the antibody are prepared by methods known in the art, such as described in Epstein et al., 1985, Proc Natl Acad Sci USA, 82:3688; Hwang et al., 1980, Proc Natl Acad Sci USA, 77:4030; U.S. Pat. Nos. 4,485,045; 4,544,545; and PCT WO 97/38731. Liposomes with enhanced circulation time are disclosed in U.S. Pat. No. 5,013,556. The components of the liposome are commonly arranged in a bilayer formation, similar to the lipid arrangement of biological membranes. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. A chemotherapeutic agent or other therapeutically active agent is optionally contained within the liposome (Gabizon et al., 1989, J National Cancer Inst 81:1484).

The antibodies may also be entrapped in microcapsules prepared by methods including but not limited to coacervation techniques, interfacial polymerization (for example using hydroxymethylcellulose or gelatin-microcapsules, or poly-(methylmethacylate) microcapsules), colloidal drug delivery systems (for example, liposomes, albumin microspheres, microemulsions, nano-particles and nanocapsules), and macroemulsions. Such techniques are disclosed in Remington's Pharmaceutical Sciences 16th edition, Osol, A. Ed., 1980. Sustained-release preparations may be prepared. Suitable examples of sustained-release preparations include semipermeable matrices of solid hydrophobic polymer, which matrices are in the form of shaped articles, e.g. films, or microcapsules. Examples of sustained-release matrices include polyesters, hydrogels (for example poly(2-hydroxyethyl-methacrylate), or poly(vinylalcohol)), polylactides (U.S. Pat. No. 3,773,919), copolymers of L-glutamic acid and gamma ethyl-L-glutamate, non-degradable ethylene-vinyl acetate, degradable lactic acid-glycolic acid copolymers such as the LUPRON DEPOT® (which are injectable microspheres composed of lactic acid-glycolic acid copolymer and leuprolide acetate), poly-D-(−)-3-hydroxybutyric acid, and ProLease® (commercially available from Alkermes), which is a microsphere-based delivery system composed of the desired bioactive molecule incorporated into a matrix of poly-DL-lactide-co-glycolide (PLG).

The dosing amounts and frequencies of administration are, in a preferred embodiment, selected to be therapeutically or prophylactically effective. As is known in the art, adjustments for protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

The concentration of the antibody in the formulation may vary from about 0.1 to 100 weight %. In a preferred embodiment, the concentration of the Fc variant is in the range of 0.003 to 1.0 molar. In order to treat a patient, a therapeutically effective dose of the Fc variant of the present invention may be administered. By “therapeutically effective dose” herein is meant a dose that produces the effects for which it is administered. The exact dose will depend on the purpose of the treatment, and will be ascertainable by one skilled in the art using known techniques. Dosages may range from 0.0001 to 100 mg/kg of body weight or greater, for example 0.1, 1, 10, or 50 mg/kg of body weight, with 1 to 10 mg/kg being preferred.

### III. Methods of Using Anti-VSIG10 Antibodies

Once made, the anti-VSIG10 antibodies of the invention find use in a number of different applications.

A. Therapeutic Uses

The anti-VSIG10 antibodies of the invention find use in treating patients, such as human subjects, generally with a condition associated with VSIG10. The term “treatment” as used herein, refers to both therapeutic treatment and prophylactic or preventative measures, which in this example relates to treatment of cancer; however, also as described below, uses of antibodies and pharmaceutical compositions are also provided for treatment of infectious disease, sepsis, and/or for inhibiting an undesirable immune activation that follows gene therapy. Those in need of treatment include those already with cancer as well as those in which the cancer is to be prevented. Hence, the mammal to be treated herein may have been diagnosed as having the cancer or may be predisposed or susceptible to the cancer. As used herein the term “treating” refers to preventing, delaying the onset of, curing, reversing, attenuating, alleviating, minimizing, suppressing, halting the deleterious effects or stabilizing of discernible symptoms of the above-described cancerous diseases, disorders or conditions. It also includes managing the cancer as described above. By “manage” it is meant reducing the severity of the disease, reducing the frequency of episodes of the disease, reducing the duration of such episodes, reducing the severity of such episodes, slowing/reducing cancer cell growth or proliferation, slowing progression of at least one symptom, amelioration of at least one measurable physical parameter and the like. For example, immunostimulatory anti-VSIG10 immune molecules should promote T cell or NK or cytokine immunity against target cells, e.g., cancer, infected or pathogen cells and thereby treat cancer or infectious diseases by depleting the cells involved in the disease condition.

The VSIG10 antibodies of the invention are provided in therapeutically effective dosages. A “therapeutically effective dosage” of an anti-VSIG10 immune molecule according to at least some embodiments of the present invention preferably results in a decrease in severity of disease symptoms, an increase in frequency and duration of disease symptom-free periods, an increase in lifespan, disease remission, or a prevention or reduction of impairment or disability due to the disease affliction. For example, for the treatment of VSIG10 positive tumors, a “therapeutically effective dosage” preferably inhibits cell growth or tumor growth by at least about 20%, more preferably by at least about 40%, even more preferably by at least about 60%, and still more preferably by at least about 80% relative to untreated subjects. The ability of a compound to inhibit tumor growth can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, or otherwise ameliorate symptoms in a subject.

One of ordinary skill in the art would be able to determine a therapeutically effective amount based on such factors as the subject's size, the severity of the subject's symptoms, and the particular composition or route of administration selected.

1. Cancer Treatment

The VSIG10 antibodies of the invention find particular use in the treatment of cancer. In general, the antibodies of the invention are immunomodulatory, in that rather than directly attack cancerous cells, the anti-VSIG10 antibodies of the invention stimulate the immune system, generally by inhibiting the action of VSIG10. Thus, unlike tumor-targeted therapies, which are aimed at inhibiting molecular pathways that are crucial for tumor growth and development, and/or depleting tumor cells, cancer immunotherapy is aimed to stimulate the patient's own immune system to eliminate cancer cells, providing long-lived tumor destruction. Various approaches can be used in cancer immunotherapy, among them are therapeutic cancer vaccines to induce tumor-specific T cell responses, and immunostimulatory antibodies (i.e. antagonists of inhibitory receptors=immune checkpoints) to remove immunosuppressive pathways.

Clinical responses with targeted therapy or conventional anti-cancer therapies tend to be transient as cancer cells develop resistance, and tumor recurrence takes place. However, the clinical use of cancer immunotherapy in the past few years has shown that this type of therapy can have durable clinical responses, showing dramatic impact on long term survival. However, although responses are long term, only a small number of patients respond (as opposed to conventional or targeted therapy, where a large number of patients respond, but responses are transient).

By the time a tumor is detected clinically, it has already evaded the immune-defense system by acquiring immunoresistant and immunosuppressive properties and creating an immunosuppressive tumor microenvironment through various mechanisms and a variety of immune cells.

Accordingly, the anti-VSIG10 antibodies of the invention are useful in treating cancer. Due to the nature of an immuno-oncology mechanism of action, VSIG10 does not necessarily need to be overexpressed on or correlated with a particular cancer type; that is, the goal is to have the anti-VSIG10 antibodies de-suppress T cell and NK cell activation, such that the immune system will go after the cancers.

“Cancer,” as used herein, refers broadly to any neoplastic disease (whether invasive or metastatic) characterized by abnormal and uncontrolled cell division causing malignant growth or tumor (e.g., unregulated cell growth.) The term “cancer” or “cancerous” as used herein should be understood to encompass any neoplastic disease (whether invasive, non-invasive or metastatic) which is characterized by abnormal and uncontrolled cell division causing malignant growth or tumor, non-limiting examples of which are described herein. This includes any physiological condition in mammals that is typically characterized by unregulated cell growth. Examples of cancer are exemplified in the working examples and also are described within the specification.

Non-limiting examples of cancer that can be treated using anti-VSIG10 antibodies include, but are not limited to, carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; multiple myeloma and post-transplant lymphoproliferative disorder (PTLD).

Other cancers amenable for treatment by the present invention include, but are not limited to, carcinoma, lymphoma, blastoma, sarcoma, and leukemia or lymphoid malignancies. More particular examples of such cancers include colorectal, bladder, ovarian, melanoma, squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. Preferably, the cancer is selected from the group consisting of colorectal cancer, breast cancer, rectal cancer, non-small cell lung cancer, non-Hodgkin's lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma. In an exemplary embodiment the cancer is an early or advanced (including metastatic) bladder, ovarian or melanoma. In another embodiment the cancer is colorectal cancer. The cancerous conditions amenable for treatment of the invention include cancers that express or do not express VSIG10 and further include non-metastatic or non-invasive as well as invasive or metastatic cancers wherein VSIG10 expression by immune, stromal or diseased cells suppress antitumor responses and anti-invasive immune responses. The method of the present invention is particularly suitable for the treatment of vascularized tumors.

“Cancer therapy” herein refers to any method which prevents or treats cancer or ameliorates one or more of the symptoms of cancer. Typically such therapies will comprises administration of immunostimulatory anti-VSIG10 antibodies (including antigen-binding fragments) either alone or in combination with chemotherapy or radiotherapy or other biologics and for enhancing the activity thereof, i.e., in individuals wherein expression of VSIG10 suppresses antitumor responses and the efficacy of chemotherapy or radiotherapy or biologic efficacy.

2. Combination Therapies in Cancer

As is known in the art, combination therapies comprising a therapeutic antibody targeting an immunotherapy target and an additional therapeutic agent, specific for the disease condition, are showing great promise. For example, in the area of immunotherapy, there are a number of promising combination therapies using a chemotherapeutic agent (either a small molecule drug or an anti-tumor antibody) with immuno-oncology antibodies like anti-PD-1, and as such, the anti-VSIG10 antibodies outlined herein can be substituted in the same way. Any chemotherapeutic agent exhibiting anticancer activity can be used according to the present invention; various non-limiting examples are described in the specification.

The underlying scientific rationale for the dramatic increased efficacy of combination therapy claims that immune checkpoint blockade as a monotherapy will induce tumor regressions only when there is pre-existing strong anti-tumor immune response to be ‘unleashed’ when the pathway is blocked. However, in most patients and tumor types the endogenous anti-tumor immune responses are weak, and thus the induction of anti-tumor immunity is required for the immune checkpoint blockade to be effective. According to at least some embodiments of the present invention, VSIG10-specific antibodies, antibody fragments, conjugates and compositions comprising same, are used for treatment of all types of cancer in cancer immunotherapy in combination therapy.

The terms “in combination with” and “co-administration” are not limited to the administration of said prophylactic or therapeutic agents at exactly the same time. Instead, it is meant that the anti-VSIG10 antibody and the other agent or agents are administered in a sequence and within a time interval such that they may act together to provide a benefit that is increased versus treatment with only either anti-VSIG10 antibody of the present invention or the other agent or agents. It is preferred that the anti-VSIG10 antibody and the other agent or agents act additively, and especially preferred that they act synergistically. Such molecules are suitably present in combination in amounts that are effective for the purpose intended. The skilled medical practitioner can determine empirically, or by considering the pharmacokinetics and modes of action of the agents, the appropriate dose or doses of each therapeutic agent, as well as the appropriate timings and methods of administration.

Accordingly, the antibodies of the present invention may be administered concomitantly with one or more other therapeutic regimens or agents. The additional therapeutic regimes or agents may be used to improve the efficacy or safety of the anti-VSIG10 antibody. Also, the additional therapeutic regimes or agents may be used to treat the same disease or a comorbidity rather than to alter the action of the VSIG10 antibody. For example, a VSIG10 antibody of the present invention may be administered to the patient along with chemotherapy, radiation therapy, or both chemotherapy and radiation therapy.

The VSIG10 antibodies of the present invention may be administered in combination with one or more other prophylactic or therapeutic agents, including but not limited to cytotoxic agents, chemotherapeutic agents, cytokines, growth inhibitory agents, anti-hormonal agents, kinase inhibitors, anti-angiogenic agents, cardioprotectants, immunostimulatory agents, immunosuppressive agents, agents that promote proliferation of hematological cells, angiogenesis inhibitors, protein tyrosine kinase (PTK) inhibitors, or other therapeutic agents.

According to at least some embodiments, the anti VSIG10 immune molecules could be used in combination with any of the known in the art standard of care cancer treatment (as can be found, for example, in http://www.cancer.gov/cancertopics).

For example, the combination therapy can include an anti VSIG10 antibody combined with at least one other therapeutic or immune modulatory agent, other compounds or immunotherapies, or immunostimulatory strategy as described herein. including, but not limited to, tumor vaccines, adoptive T cell therapy, Treg depletion, antibodies (e.g. bevacizumab, Erbitux), peptides, pepti-bodies, small molecules, chemotherapeutic agents such as cytotoxic and cytostatic agents (e.g. paclitaxel, cisplatin, vinorelbine, docetaxel, gemcitabine, temozolomide, irinotecan, 5FU, carboplatin), immunological modifiers such as interferons and interleukins, immunostimulatory antibodies, growth hormones or other cytokines, folic acid, vitamins, minerals, aromatase inhibitors, RNAi, Histone Deacetylase Inhibitors, proteasome inhibitors, doxorubicin (Adriamycin), cisplatin bleomycin sulfate, carmustine, chlorambucil, and cyclophosphamide hydroxyurea which, by themselves, are only effective at levels which are toxic or subtoxic to a patient. Cisplatin is intravenously administered as a 100 mg/dose once every four weeks and Adriamycin is intravenously administered as a 60-75 mg/ml dose once every 21 days.

According to at least some embodiments of the present invention, therapeutic agents that can be used in combination with anti-VSIG10 antibodies are other potentiating agents that enhance anti-tumor responses, e.g. other anti-immune checkpoint antibodies or other potentiating agents that are primarily geared to increase endogenous anti-tumor responses, such as Radiotherapy, Cryotherapy, Conventional/classical chemotherapy potentiating anti-tumor immune responses, Targeted therapy potentiating anti-tumor immune responses, Anti-angiogenic therapy, Therapeutic agents targeting immunosuppressive cells such as Tregs and MDSCs, Immunostimulatory antibodies, Cytokine therapy, Therapeutic cancer vaccines, Adoptive cell transfer.

In some embodiments, anti-VSIG10 antibodies are used in combination with Bisphosphonates, especially amino-bisphosphonates (ABP), which have shown to have anti-cancer activity. Some of the activities associated with ABPs are on human γδT cells that straddle the interface of innate and adaptive immunity and have potent anti-tumour activity.

Targeted therapies can also stimulate tumor-specific immune response by inducing the immunogenic death of tumor cells or by engaging immune effector mechanisms (Galluzzi et al, 2012, Nature Reviews—Drug discovery, Volume 11, pages 215-233).

According to at least some embodiments of the invention, targeted therapies used as agents for combination with anti VSIG10 immune molecules for treatment of cancer are as described herein.

In some embodiments, anti-VSIG10 antibodies are used in combination with therapeutic agents targeting regulatory immunosuppressive cells such as regulatory T cells (Tregs) and myeloid derived suppressor cells (MDSCs). A number of commonly used chemotherapeutics exert non-specific targeting of Tregs and reduce the number or the immunosuppressive capacity of Tregs or MDSCs (Facciabene A. et al 2012 Cancer Res; 72(9) 2162-71; Byrne WL. et al 2011, Cancer Res. 71:691520; Gabrilovich D I. and Nagaraj S, Nature Reviews 2009 Volume 9, pages 162-174). In this regard, metronomic therapy with some chemotherapy drugs results in immunostimulatory rather than immunosuppressive effects, via modulation of regulatory cells. Thus, according to at least some embodiments of the present invention, anti-VSIG10 immune molecule for cancer immunotherapy is used in combination with drugs selected from but not limited to cyclophosphamide, gemcitabine, mitoxantrone, fludarabine, fludarabine, docetaxel, paclitaxel, thalidomide and thalidomide derivatives.

In some embodiments, anti-VSIG10 antibodies are used in combination with novel Treg-specific targeting agents including: 1) depleting or killing antibodies that directly target Tregs through recognition of Treg cell surface receptors such as anti-CD25 mAbs daclizumab, basiliximab or 2) ligand-directed toxins such as denileukin diftitox (Ontak)—a fusion protein of human IL-2 and diphtheria toxin, or LMB-2—a fusion between an scFv against CD25 and Pseudomonas exotoxin and 3) antibodies targeting Treg cell surface receptors such as CTLA4, PD-1, OX40 and GITR or 4) antibodies, small molecules or fusion proteins targeting other NK receptors such as previously identified.

In some embodiments, anti-VSIG10 antibodies are used in combination with any of the options described below for disrupting Treg induction and/or function, including TLR (toll like receptors) agonists; agents that interfere with the adenosinergic pathway, such as ectonucleotidase inhibitors, or inhibitors of the A2A adenosine receptor; TGF-β inhibitors, such as fresolimumab, lerdelimumab, metelimumab, trabedersen, LY2157299, LY210976; blockade of Tregs recruitment to tumor tissues including chemokine receptor inhibitors, such as the CCR4/CCL2/CCL22 pathway.

In some embodiments, anti-VSIG10 antibodies are used in combination with any of the options described below for inhibiting the immunosuppressive tumor microenvironment, including inhibitors of cytokines and enzymes which exert immunosuppressive activities, such as IDO (indoleamine-2,3-dioxygenase) inhibitors; inhibitors of anti-inflammatory cytokines which promote an immunosuppressive microenvironment, such as IL-10, L-35, L-4 and IL-13; Bevacizumab® which reduces Tregs and favors the differentiation of DCs.

In some embodiments, anti-VSIG10 antibodies are used in combination with any of the options described below for targeting MDSCs (myeloid-derived suppressive cells), including promoting their differentiation into mature myeloid cells that do not have suppressive functions by Vitamin D3, or Vitamin A metabolites, such as retinoic acid, all-trans retinoic acid (ATRA); inhibition of MDSCs suppressive activity by COX2 inhibitors, phosphodiesterase 5 inhibitors like sildenafil, ROS inhibitors such as nitroaspirin.

In some embodiments, anti-VSIG10 antibodies are used in combination with immunostimulatory antibodies or other agents which potentiate anti-tumor immune responses (Pardoll J Exp Med. 2012; 209(2): 201-209). Immunostimulatory antibodies promote anti-tumor immunity by directly modulating immune functions, i.e. blocking other inhibitory targets or enhancing immunostimulatory proteins. According to at least some embodiments of the present invention, anti—VSIG10 immune molecules for cancer immunotherapy is used in combination with antagonistic antibodies targeting additional immune checkpoints including anti-CTLA4 mAbs, such as ipilimumab, tremelimumab; anti-PD-1 such as nivolumab BMS-936558/MDX-110⁶/ONO-4538, AMP224, CT-011, MK-3475, anti-PDL-1 antagonists such as BMS-936559/MDX-1105, MEDI4736, RG-7446/MPDL3280A; Anti-LAG-3 such as IMP-321), anti-TIM-3, anti-BTLA, anti-B7-H4, anti-B7-H3, Anti-VISTA; Agonistic antibodies targeting immunostimulatory proteins, including anti-CD40 mAbs such as CP-870,893, lucatumumab, dacetuzumab; anti-CD137 mAbs such as BMS-663513 urelumab, PF-05082566; anti-OX40 mAbs, such as anti-OX40; anti-GITR mAbs such as TRX518; anti-CD27 mAbs, such as CDX-1127; and anti-ICOS mAbs.

In some embodiments, anti-VSIG10 antibodies are used in combination with cytokines. A number of cytokines are in preclinical or clinical development as agents potentiating anti-tumor immune responses for cancer immunotherapy, including among others: IL-2, IL-7, IL-12, IL-15, IL-17, IL-18 and IL-21, IL-23, IL-27, GM-CSF, IFNα (interferon α), IFNβ, and IFNγ. However, therapeutic efficacy is often hampered by severe side effects and poor pharmacokinetic properties. Thus, in addition to systemic administration of cytokines, a variety of strategies can be employed for the delivery of therapeutic cytokines and their localization to the tumor site, in order to improve their pharmacokinetics, as well as their efficacy and/or toxicity, including antibody-cytokine fusion molecules (immunocytokines), chemical conjugation to polyethylene glycol (PEGylation), transgenic expression of cytokines in autologous whole tumor cells, incorporation of cytokine genes into DNA vaccines, recombinant viral vectors to deliver cytokine genes, etc. In the case of immunocytokines, fusion of cytokines to tumor-specific antibodies or antibody fragments allows for targeted delivery and therefore improved efficacy and pharmacokinetics, and reduced side effects.

In some embodiments, anti-VSIG10 antibodies are used in combination with cancer vaccines. Therapeutic cancer vaccines allow for improved priming of T cells and improved antigen presentation, and can be used as therapeutic agents for potentiating anti-tumor immune responses (Mellman I. et al., 2011, Nature, 480:22-29; Schlom J, 2012, J Natl Cancer Inst; 104:599-613).

Several types of therapeutic cancer vaccines are in preclinical and clinical development. These include for example:

1) Whole tumor cell vaccines, in which cancer cells removed during surgery are treated to enhance their immunogenicity, and injected into the patient to induce immune responses against antigens in the tumor cells. The tumor cell vaccine can be autologous, i.e. a patient's own tumor, or allogeneic which typically contain two or three established and characterized human tumor cell lines of a given tumor type, such as the GVAX vaccine platforms.

2) Tumor antigen vaccines, in which a tumor antigen (or a combination of a few tumor antigens), usually proteins or peptides, are administered to boost the immune system (possibly with an adjuvant and/or with immune modulators or attractants of dendritic cells such as GM-CSF). The tumor antigens may be specific for a certain type of cancer, but they are not made for a specific patient.

3) Vector-based tumor antigen vaccines and DNA vaccines can be used as a way to provide a steady supply of antigens to stimulate an anti-tumor immune response.

Vectors encoding for tumor antigens are injected into the patient (possibly with proinflammatory or other attractants such as GM-CSF), taken up by cells in vivo to make the specific antigens, which would then provoke the desired immune response. Vectors may be used to deliver more than one tumor antigen at a time, to increase the immune response. In addition, recombinant virus, bacteria or yeast vectors should trigger their own immune responses, which may also enhance the overall immune response.

4) Oncolytic virus vaccines, such as OncoVex/T-VEC, which involves the intratumoral injection of replication-conditional herpes simplex virus which preferentially infects cancer cells. The virus, which is also engineered to express GM-CSF, is able to replicate inside a cancer cell causing its lysis, releasing new viruses and an array of tumor antigens, and secreting GM-CSF in the process. Thus, such oncolytic virus vaccines enhance DCs function in the tumor microenvironment to stimulate anti-tumor immune responses.

5) Dendritic cell vaccines (Palucka and Banchereau, 2102, Nat. Rev. Cancer, 12(4):265-277): Dendritic cells (DCs) phagocytose tumor cells and present tumor antigens to tumor specific T cells. In this approach, DCs are isolated from the cancer patient and primed for presenting tumor-specific T cells. To this end several methods can be used: DCs are loaded with tumor cells or lysates; DCs are loaded with fusion proteins or peptides of tumor antigens; coupling of tumor antigens to DC-targeting mAbs. The DCs are treated in the presence of a stimulating factor (such as GM-CSF), activated and matured ex vivo, and then re-infused back into the patient in order provoke an immune response to the cancer cells. Dendritic cells can also be primed in vivo by injection of patients with irradiated whole tumor cells engineered to secrete stimulating cytokines (such as GM-CSF). Similar approaches can be carried out with monocytes. Sipuleucel-T (Provenge), a therapeutic cancer vaccine which has been approved for treatment of advanced prostate cancer, is an example of a dendritic cell vaccine.

In some embodiments, anti-VSIG10 antibodies are used in combination with adoptive T cell therapy or adoptive cell transfer (ACT), which involves the ex vivo identification and expansion of autologous naturally occurring tumor specific T cells, which are then adoptively transferred back into the cancer patient (Restifo et al, 2013, Cancer Immunol. Immunother. 62(4):727-36 (2013) Epub Dec. 4 2012). Cells that are infused back into a patient after ex vivo expansion can traffic to the tumor and mediate its destruction. Prior to this adoptive transfer, hosts can be immunodepleted by irradiation and/or chemotherapy. The combination of lymphodepletion, adoptive cell transfer, and a T cell growth factor (such as IL-2), can lead to prolonged tumor eradication in tumor patients. A more novel approach involves the ex vivo genetic modification of normal peripheral blood T cells to confer specificity for tumor-associated antigens. For example, clones of TCRs of T cells with particularly good anti-tumor responses can be inserted into viral expression vectors and used to infect autologous T cells from the patient to be treated. Another option is the use of chimeric antigen receptors (CARs) which are essentially a chimeric immunoglobulin-TCR molecule, also known as a T-body. CARs have antibody-like specificities and recognize MHC-nonrestricted structures on the surface of target cells (the extracellular target-binding module), grafted onto the TCR intracellular domains capable of activating T cells (Restifo et al Cancer Immunol. Immunother. 62(4):727-36 (2013) Epub Dec. 4, 2012; and Shi et al, Nature 493:111-115 2013.

The VSIG10 antibodies and the one or more other therapeutic agents can be administered in either order or simultaneously. The composition can be linked to the agent (as an immunocomplex) or can be administered separately from the agent. In the latter case (separate administration), the composition can be administered before, after or concurrently with the agent or can be co-administered with other known therapies, e.g., an anti-cancer therapy, e.g., radiation.

Co-administration of the humanized anti-VSIG10 immune molecules, according to at least some embodiments of the present invention with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells which would render them unreactive with the antibody. In other embodiments, the subject can be additionally treated with an agent that modulates, e.g., enhances or inhibits, the expression or activity of Fcy or Fcy receptors by, for example, treating the subject with a cytokine.

Target-specific effector cells, e.g., effector cells linked to compositions (e.g., human antibodies, multispecific and bispecific molecules) according to at least some embodiments of the present invention can also be used as therapeutic agents. Effector cells for targeting can be human leukocytes such as macrophages, neutrophils or monocytes. Other cells include eosinophils, natural killer cells and other IgG- or IgA-receptor bearing cells. If desired, effector cells can be obtained from the subject to be treated. The target-specific effector cells can be administered as a suspension of cells in a physiologically acceptable solution. The number of cells administered can be in the order of 10⁻⁸ to 10⁻⁹ but will vary depending on the therapeutic purpose. In general, the amount will be sufficient to obtain localization at the target cell, e.g., a tumor cell expressing VSIG10 proteins, and to effect cell killing e.g., by, e.g., phagocytosis. Routes of administration can also vary.

Therapy with target-specific effector cells can be performed in conjunction with other techniques for removal of targeted cells. For example, anti-tumor therapy using the compositions (e.g., human antibodies, multispecific and bispecific molecules) according to at least some embodiments of the present invention and/or effector cells armed with these compositions can be used in conjunction with chemotherapy. Additionally, combination immunotherapy may be used to direct two distinct cytotoxic effector populations toward tumor cell rejection. For example, anti-VSIG10 immune molecules linked to anti-Fc-γ RI or anti-CD3 may be used in conjunction with IgG- or IgA-receptor specific binding agents.

Bispecific and multispecific molecules according to at least some embodiments of the present invention can also be used to modulate FcγR or FcγR levels on effector cells, such as by capping and elimination of receptors on the cell surface. Mixtures of anti-Fc receptors can also be used for this purpose.

The therapeutic compositions (e.g., human antibodies, alternative scaffolds multispecific and bispecific molecules and immunoconjugates) according to at least some embodiments of the present invention which have complement binding sites, such as portions from IgG1, -2, or -3 or IgM which bind complement, can also be used in the presence of complement. In one embodiment, ex vivo treatment of a population of cells comprising target cells with a binding agent according to at least some embodiments of the present invention and appropriate effector cells can be supplemented by the addition of complement or serum containing complement. Phagocytosis of target cells coated with a binding agent according to at least some embodiments of the present invention can be improved by binding of complement proteins. In another embodiment target cells coated with the compositions (e.g., human antibodies, multispecific and bispecific molecules) according to at least some embodiments of the present invention can also be lysed by complement. In yet another embodiment, the compositions according to at least some embodiments of the present invention do not activate complement.

The therapeutic compositions (e.g., human antibodies, alternative scaffolds multispecific and bispecific molecules and immunoconjugates) according to at least some embodiments of the present invention can also be administered together with complement. Thus, according to at least some embodiments of the present invention there are compositions, comprising human antibodies, multispecific or bispecific molecules and serum or complement. These compositions are advantageous in that the complement is located in close proximity to the human antibodies, multispecific or bispecific molecules. Alternatively, the human antibodies, multispecific or bispecific molecules according to at least some embodiments of the present invention and the complement or serum can be administered separately.

The anti-VSIG10 immune molecules, according to at least some embodiments of the present invention, can be used as neutralizing antibodies. A neutralizing antibody (Nabs), is an antibody that is capable of binding and neutralizing or inhibiting a specific antigen thereby inhibiting its biological effect. NAbs will partially or completely abrogate the biological action of an agent by either blocking an important surface molecule needed for its activity or by interfering with the binding of the agent to its receptor on a target cell.

According to an additional aspect of the present invention the therapeutic agents can be used to prevent pathologic inhibition of T cell activity, such as that directed against cancer cells.

Thus, according to an additional aspect of the present invention there is provided a method of treating cancer as recited herein, and/or for promoting immune stimulation by administering to a subject in need thereof an effective amount of any one of the therapeutic agents and/or a pharmaceutical composition comprising any of the therapeutic agents and further comprising a pharmaceutically acceptable diluent or carrier.

According to at least some embodiments, immune cells, preferably T cells, can be contacted in vivo or ex vivo with the therapeutic agents to modulate immune responses.

The T cells contacted with the therapeutic agents can be any cell which expresses the T cell receptor, including α/β and γ/δ T cell receptors. T-cells include all cells which express CD3, including T-cell subsets which also express CD4 and CDS. T-cells include both naive and memory cells and effector cells such as CD8+ cytotoxic T lymphocytes (CTL). T-cells also include cells such as Th1, Tc1, Th2, Tc2, Th3, Th9, Th17, Th22, Treg, follicular helper cells (T_{FH}) and Tr1 cells. T-cells also include NKT-cells iNKT, α/β NKT and γ/δ NKT cells, and similar unique classes of the T-cell lineage.

VSIG10 blocking antibodies can also be used in combination with bispecific antibodies that target Fcα or Fcγ receptor-expressing effectors cells to tumor cells (see, e.g., U.S. Pat. Nos. 5,922,845 and 5,837,243). Bispecific antibodies can be used to target two separate antigens. For example anti-Fc receptor/anti-tumor antigen (e.g., Her-2/neu) bispecific antibodies have been used to target macrophages to sites of tumor. This targeting may more effectively activate tumor specific responses. The T cell arm of these responses would be augmented by the use of VSIG10 blockade. Alternatively, antigen may be delivered directly to DCs by the use of bispecific antibodies which bind to tumor antigen and a dendritic cell specific cell surface marker.

Tumors evade host immune surveillance by a large variety of mechanisms. Many of these mechanisms may be overcome by the inactivation of proteins which are expressed by the tumors and which are immunosuppressive. These include among others TGF-β (Kehrl, J. et al. (1986) J. Exp. Med. 163: 1037-1050), IL-10 (Howard, M. & O'Garra, A. (1992) Immunology Today 13: 198-200), and Fas ligand (Hahne, M. et al. (1996) Science 274: 1363-1365). Antibodies to each of these entities may be used in combination with anti-VSIG10 to counteract the effects of the immunosuppressive agent and favor tumor immune responses by the host.

Other antibodies which may be used to activate host immune responsiveness can be used in combination with anti-VSIG10. These include molecules on the surface of dendritic cells which activate DC function and antigen presentation. Anti-CD40 antibodies are able to substitute effectively for T cell helper activity (Ridge, J. et al. (1998) Nature 393: 474-478) and can be used in conjunction with VSIG10 antibodies (Ito, N. et al. (2000) Immunobiology 201 (5) 527-40). Activating antibodies to T cell costimulatory molecules such as OX-40 (Weinberg, A. et al. (2000) Immunol 164: 2160-2169), 4-1BB (Melero, I. et al. (1997) Nature Medicine 3: 682-685 (1997), and ICOS (Hutloff, A. et al. (1999) Nature 397: 262-266) as well as antibodies which block the activity of negative costimulatory molecules such as CTLA-4 (e.g., U.S. Pat. No. 5,811,097, implimumab) or BTLA (Watanabe, N. et al. (2003) Nat Immunol 4:670-9), B7-H4 (Sica, G L et al. (2003) Immunity 18:849-61) PD-1 (may also provide for increased levels of T cell activation. Bone marrow transplantation is currently being used to treat a variety of tumors of hematopoietic origin. While graft versus host disease is a consequence of this treatment, therapeutic benefit may be obtained from graft vs. tumor responses. VSIG10 blockade can be used to increase the effectiveness of the donor engrafted tumor specific T cells.

There are also several experimental treatment protocols that involve ex vivo activation and expansion of antigen specific T cells and adoptive transfer of these cells into recipients in order to antigen-specific T cells against tumor (Greenberg, R. & Riddell, S. (1999) Science 285: 546-51). These methods may also be used to activate T cell responses to infectious agents such as CMV. Ex vivo activation in the presence of anti-VSIG10 immune molecules may be expected to increase the frequency and activity of the adoptively transferred T cells.

Optionally, antibodies to VSIG10 can be combined with an immunogenic agent, such as cancerous cells, purified tumor antigens (including recombinant proteins, peptides, and carbohydrate molecules), cells, and cells transfected with genes encoding immune stimulating cytokines (He et al (2004) J. Immunol. 173:4919-28). Non-limiting examples of tumor vaccines that can be used include peptides of MUC1 for treatment of colon cancer, peptides of MUC-1/CEA/TRICOM for the treatment of ovary cancer, or tumor cells transfected to express the cytokine GM-CSF (discussed further below).

In humans, some tumors have been shown to be immunogenic such as RCC. It is anticipated that by raising the threshold of T cell activation by VSIG10 blockade, we may expect to activate tumor responses in the host.

VSIG10 blockade is likely to be most effective when combined with a vaccination protocol. Many experimental strategies for vaccination against tumors have been devised (see Rosenberg, S., 2000, Development of Cancer Vaccines, ASCO Educational Book Spring: 60-62; Logothetis, C., 2000, ASCO Educational Book Spring: 300-302; Khayat, D. 2000, ASCO Educational Book Spring: 414-428; Foon, K. 2000, ASCO Educational Book Spring: 730-738; see also Restifo, N. and Sznol, M., Cancer Vaccines, Ch. 61, pp. 3023-3043 in DeVita, V. et al. (eds.), 1997, Cancer: Principles and Practice of Oncology. Fifth Edition). In one of these strategies, a vaccine is prepared using autologous or allogeneic tumor cells. These cellular vaccines have been shown to be most effective when the tumor cells are transduced to express GM-CSF. GM-CSF has been shown to be a potent activator of antigen presentation for tumor vaccination (Dranoff et al. (1993) Proc. Natl. Acad. Sci U.S.A. 90: 3539-43).

The study of gene expression and large scale gene expression patterns in various tumors has led to the definition of so-called tumor specific antigens (Rosenberg, S A (1999) Immunity 10: 281-7). In many cases, these tumor specific antigens are differentiation antigens expressed in the tumors and in the cell from which the tumor arose, for example melanocyte antigens gp100, MAGE antigens, and Trp-2. More importantly, many of these antigens can be shown to be the targets of tumor specific T cells found in the host. VSIG10 blockade may be used in conjunction with a collection of recombinant proteins and/or peptides expressed in a tumor in order to generate an immune response to these proteins. These proteins are normally viewed by the immune system as self-antigens and are therefore tolerant to them. The tumor antigen may also include the protein telomerase, which is required for the synthesis of telomeres of chromosomes and which is expressed in more than 85% of human cancers and in only a limited number of somatic tissues (Kim, N et al. (1994) Science 266: 2011-2013). (These somatic tissues may be protected from immune attack by various means). Tumor antigen may also be “neo-antigens” expressed in cancer cells because of somatic mutations that alter protein sequence or create fusion proteins between two unrelated sequences (i.e. bcr-ab1 in the Philadelphia chromosome), or idiotype from B cell tumors.

Other tumor vaccines may include the proteins from viruses implicated in human cancers such a Human Papilloma Viruses (HPV), Hepatitis Viruses (HBV and HCV) and Kaposi's Herpes Sarcoma Virus (KHSV). Another form of tumor specific antigen which may be used in conjunction with VSIG10 blockade is purified heat shock proteins (HSP) isolated from the tumor tissue itself. These heat shock proteins contain fragments of proteins from the tumor cells and these HSPs are highly efficient at delivery to antigen presenting cells for eliciting tumor immunity (Suot, R & Srivastava, P (1995) Science 269:1585-1588; Tamura, Y. et al. (1997) Science 278:117-120).

Dendritic cells (DC) are potent antigen presenting cells that can be used to prime antigen-specific responses. DC's can be produced ex vivo and loaded with various protein and peptide antigens as well as tumor cell extracts (Nestle, F. et al. (1998) Nature Medicine 4: 328-332). DCs may also be transduced by genetic means to express these tumor antigens as well. DCs have also been fused directly to tumor cells for the purposes of immunization (Kugler, A. et al. (2000) Nature Medicine 6:332-336). As a method of vaccination, DC immunization may be effectively combined with VSIG10 blockade to activate more potent anti-tumor responses.

Use of the therapeutic agents according to at least some embodiments of the invention as adjuvant for cancer vaccination:

Immunization against tumor-associated antigens (TAAs) is a promising approach for cancer therapy and prevention, but it faces several challenges and limitations, such as tolerance mechanisms associated with self-antigens expressed by the tumor cells. Costimulatory molecules such as B7.1 (CD80) and B7.2 (CD86) have improved the efficacy of gene-based and cell-based vaccines in animal models and are under investigation as adjuvant in clinical trials. This adjuvant activity can be achieved either by enhancing the costimulatory signal or by blocking inhibitory signal that is transmitted by negative costimulators expressed by tumor cells (Neighbors et al., 2008 J Immunother.; 31(7):644-55).

According to at least some embodiments of the invention, any one of polyclonal or monoclonal antibody and/or antigen-binding fragments and/or conjugates containing same, and/or alternative scaffolds, specific to any one of VSIG10 proteins, can be used as adjuvant for cancer vaccination. According to at least some embodiments, the invention provides methods for improving immunization against TAAs, comprising administering to a patient an effective amount of any one of polyclonal or monoclonal antibody and/or antigen-binding fragments and/or conjugates containing same, and/or alternative scaffolds, specific to any one of VSIG10 proteins.

In some embodiments the invention provides the use of VSIG10 antibodies to perform one or more of the following in a subject in need thereof: (a) upregulating pro-inflammatory cytokines; (b) increasing T-cell proliferation and/or expansion; (c) increasing interferon-γ or TNF-α production by T-cells; (d) increasing IL-2 secretion; (e) stimulating antibody responses; (f) inhibiting cancer cell growth; (g) promoting antigenic specific T cell immunity; (h) promoting CD4⁺ and/or CD8⁺ T cell activation; (i) alleviating T-cell suppression; (j) promoting NK cell activity; (k) promoting apoptosis or lysis of cancer cells; and/or (l) cytotoxic or cytostatic effect on cancer cells.

In other embodiments the invention provides the use of an immunostimulatory antibody, antigen-binding fragment or conjugate thereof according to at least some embodiments of the invention (optionally in a pharmaceutical composition) to antagonize at least one immune inhibitory effect of the VSIG10.

Such an antibody, antigen-binding fragment or conjugate thereof optionally and preferably mediates at least one of the following effects:

(i) increases in immune response, (ii) increases in activation of αβ and/or γδ T cells, (iii) increases in cytotoxic T cell activity, (iv) increases in NK and/or NKT cell activity, (v) alleviation of αβ and/or γδ T-cell suppression, (vi) increases in pro-inflammatory cytokine secretion, (vii) increases in IL-2 secretion; (viii) increases in interferon-γ production, (ix) increases in Th1 response, (x) decreases in Th2 response, (xi) decreases or eliminates cell number and/or activity of at least one of regulatory T cells (Tregs).

3. Assessment of Treatment

Generally the anti-VSIG10 antibodies of the invention are administered to patients with cancer, and efficacy is assessed, in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays. For example, evaluation of changes in immune status (e.g. presence of ICOS+ CD4+ T cells following ipi treatment) along with “old fashioned” measurements such as tumor burden, size, invasiveness, LN involvement, metastasis, etc. can be done. Thus, any or all of the following can be evaluated: the inhibitory effects of VSIG10 on CD4⁺ T cell activation or proliferation, CD8⁺ T (CTL) cell activation or proliferation, CD8⁺ T cell-mediated cytotoxic activity and/or CTL mediated cell depletion, NK cell activity and NK mediated cell depletion, the potentiating effects of VSIG10 on Treg cell differentiation and proliferation and Treg- or myeloid derived suppressor cell (MDSC)-mediated immunosuppression or immune tolerance, and/or the effects of VSIG10 on proinflammatory cytokine production by immune cells, e.g., IL-2, IFN-γ or TNF-α production by T or other immune cells.

In some embodiments, assessment of treatment is done by evaluating immune cell proliferation, using for example, CFSE dilution method, Ki67 intracellular staining of immune effector cells, and 3H-Thymidine incorporation method,

In some embodiments, assessment of treatment is done by evaluating the increase in gene expression or increased protein levels of activation-associated markers, including one or more of: CD25, CD69, CD137, ICOS, PD1, GITR, OX40, and cell degranulation measured by surface expression of CD107A.

In general, gene expression assays are done as is known in the art. See for example Goodkind et al., Computers and Chem. Eng. 29(3):589 (2005), Han et al., Bioinform. Biol. Insights 11/15/15 9(Suppl. 1):29-46, Campo et al., Nod. Pathol. 2013 January; 26 suppl. 1:S97-S110,

the gene expression measurement techniques of which are expressly incorporated by reference herein.

In general, protein expression measurements are also similarly done as is known in the art.

In some embodiments, assessment of treatment is done by assessing cytotoxic activity measured by target cell viability detection via estimating numerous cell parameters such as enzyme activity (including protease activity), cell membrane permeability, cell adherence, ATP production, co-enzyme production, and nucleotide uptake activity. Specific examples of these assays include, but are not limited to, Trypan Blue or PI staining, ⁵¹Cr or ³⁵S release method, LDH activity, MTT and/or WST assays, Calcein-AM assay, Luminescent based assay, and others.

In some embodiments, assessment of treatment is done by assessing T cell activity measured by cytokine production, measure either intracellularly in culture supernatant using cytokines including, but not limited to, IFNg, TNFa, GM-CSF, IL2, IL6, IL4, IL5, IL10, IL13 using well known techniques.

Accordingly, assessment of treatment can be done using assays that evaluate one or more of the following: (i) increases in immune response, (ii) increases in activation of αβ and/or γδ T cells, (iii) increases in cytotoxic T cell activity, (iv) increases in NK and/or NKT cell activity, (v) alleviation of αβ and/or γδ T-cell suppression, (vi) increases in pro-inflammatory cytokine secretion, (vii) increases in IL-2 secretion; (viii) increases in interferon-γ production, (ix) increases in Th1 response, (x) decreases in Th2 response, (xi) decreases or eliminates cell number and/or activity of at least one of regulatory T cells (Tregs.

Assays to Measure Efficacy

In one embodiment, the signaling pathway assay measures increases or decreases in immune response as measured for an example by phosphorylation or de-phosphorylation of different factors, or by measuring other post translational modifications. An increase in activity indicates immunostimulatory activity. Appropriate increases in activity are outlined below.

In one embodiment, the signaling pathway assay measures increases or decreases in activation of αβ and/or γδ T cells as measured for an example by cytokine secretion or by proliferation or by changes in expression of activation markers like for an example CD137, CD107a, PD1, etc. An increase in activity indicates immunostimulatory activity. Appropriate increases in activity are outlined below.

In one embodiment, the signaling pathway assay measures increases or decreases in cytotoxic T cell activity as measured for an example by direct killing of target cells like for an example cancer cells or by cytokine secretion or by proliferation or by changes in expression of activation markers like for an example CD137, CD107a, PD1, etc. An increase in activity indicates immunostimulatory activity. Appropriate increases in activity are outlined below.

In one embodiment, the signaling pathway assay measures increases or decreases in NK and/or NKT cell activity as measured for an example by direct killing of target cells like for an example cancer cells or by cytokine secretion or by changes in expression of activation markers like for an example CD107a, etc. An increase in activity indicates immunostimulatory activity. Appropriate increases in activity are outlined below.

In one embodiment, the signaling pathway assay measures increases or decreases in αβ and/or γδ T-cell suppression, as measured for an example by cytokine secretion or by proliferation or by changes in expression of activation markers like for an example CD137, CD107a, PD1, etc. An increase in activity indicates immunostimulatory activity. Appropriate increases in activity are outlined below.

In one embodiment, the signaling pathway assay measures increases or decreases in pro-inflammatory cytokine secretion as measured for example by ELISA or by Luminex or by Multiplex bead based methods or by intracellular staining and FACS analysis or by Alispot etc. An increase in activity indicates immunostimulatory activity. Appropriate increases in activity are outlined below.

In one embodiment, the signaling pathway assay measures increases or decreases in IL-2 secretion as measured for example by ELISA or by Luminex or by Multiplex bead based methods or by intracellular staining and FACS analysis or by Alispot etc. An increase in activity indicates immunostimulatory activity. Appropriate increases in activity are outlined below.

In one embodiment, the signaling pathway assay measures increases or decreases in interferon-γ production as measured for example by ELISA or by Luminex or by Multiplex bead based methods or by intracellular staining and FACS analysis or by Alispot etc. An increase in activity indicates immunostimulatory activity. Appropriate increases in activity are outlined below.

In one embodiment, the signaling pathway assay measures increases or decreases in Th1 response as measured for an example by cytokine secretion or by changes in expression of activation markers. An increase in activity indicates immunostimulatory activity. Appropriate increases in activity are outlined below.

In one embodiment, the signaling pathway assay measures increases or decreases in Th2 response as measured for an example by cytokine secretion or by changes in expression of activation markers. An increase in activity indicates immunostimulatory activity. Appropriate increases in activity are outlined below.

In one embodiment, the signaling pathway assay measures increases or decreases cell number and/or activity of at least one of regulatory T cells (Tregs), as measured for example by flow cytometry or by IHC. A decrease in response indicates immunostimulatory activity. Appropriate decreases are the same as for increases, outlined below.

In one embodiment, the signaling pathway assay measures increases or decreases in M2 macrophages cell numbers, as measured for example by flow cytometry or by IHC. A decrease in response indicates immunostimulatory activity. Appropriate decreases are the same as for increases, outlined below.

In one embodiment, the signaling pathway assay measures increases or decreases in M2 macrophage pro-tumorigenic activity, as measured for an example by cytokine secretion or by changes in expression of activation markers. A decrease in response indicates immunostimulatory activity. Appropriate decreases are the same as for increases, outlined below.

In one embodiment, the signaling pathway assay measures increases or decreases in N2 neutrophils increase, as measured for example by flow cytometry or by IHC. A decrease in response indicates immunostimulatory activity. Appropriate decreases are the same as for increases, outlined below.

In one embodiment, the signaling pathway assay measures increases or decreases in N2 neutrophils pro-tumorigenic activity, as measured for an example by cytokine secretion or by changes in expression of activation markers. A decrease in response indicates immunostimulatory activity. Appropriate decreases are the same as for increases, outlined below.

In one embodiment, the signaling pathway assay measures increases or decreases in inhibition of T cell activation, as measured for an example by cytokine secretion or by proliferation or by changes in expression of activation markers like for an example CD137, CD107a, PD1, etc. An increase in activity indicates immunostimulatory activity. Appropriate increases in activity are outlined below.

In one embodiment, the signaling pathway assay measures increases or decreases in inhibition of CTL activation as measured for an example by direct killing of target cells like for an example cancer cells or by cytokine secretion or by proliferation or by changes in expression of activation markers like for an example CD137, CD107a, PD1, etc. An increase in activity indicates immunostimulatory activity. Appropriate increases in activity are outlined below.

In one embodiment, the signaling pathway assay measures increases or decreases in αβ and/or γδ T cell exhaustion as measured for an example by changes in expression of activation markers. A decrease in response indicates immunostimulatory activity. Appropriate decreases are the same as for increases, outlined below.

In one embodiment, the signaling pathway assay measures increases or decreases αβ and/or γδ T cell response as measured for an example by cytokine secretion or by proliferation or by changes in expression of activation markers like for an example CD137, CD107a, PD1, etc. An increase in activity indicates immunostimulatory activity. Appropriate increases in activity are outlined below.

In one embodiment, the signaling pathway assay measures increases or decreases in stimulation of antigen-specific memory responses as measured for an example by cytokine secretion or by proliferation or by changes in expression of activation markers like for an example CD45RA, CCR7 etc. An increase in activity indicates immunostimulatory activity. Appropriate increases in activity are outlined below. .

In one embodiment, the signaling pathway assay measures increases or decreases in apoptosis or lysis of cancer cells as measured for an example by cytotoxicity assays such as for an example MTT, Cr release, Calcine AM, or by flow cytometry based assays like for an example CFSE dilution or propidium iodide staining etc. An increase in activity indicates immunostimulatory activity. Appropriate increases in activity are outlined below.

In one embodiment, the signaling pathway assay measures increases or decreases in stimulation of cytotoxic or cytostatic effect on cancer cells. as measured for an example by cytotoxicity assays such as for an example MTT, Cr release, Calcine AM, or by flow cytometry based assays like for an example CFSE dilution or propidium iodide staining etc. An increase in activity indicates immunostimulatory activity. Appropriate increases in activity are outlined below.

In one embodiment, the signaling pathway assay measures increases or decreases direct killing of cancer cells as measured for an example by cytotoxicity assays such as for an example MTT, Cr release, Calcine AM, or by flow cytometry based assays like for an example CFSE dilution or propidium iodide staining etc. An increase in activity indicates immunostimulatory activity. Appropriate increases in activity are outlined below.

In one embodiment, the signaling pathway assay measures increases or decreases Th17 activity as measured for an example by cytokine secretion or by proliferation or by changes in expression of activation markers. An increase in activity indicates immunostimulatory activity. Appropriate increases in activity are outlined below.

In one embodiment, the signaling pathway assay measures increases or decreases in induction of complement dependent cytotoxicity and/or antibody dependent cell-mediated cytotoxicity, as measured for an example by cytotoxicity assays such as for an example MTT, Cr release, Calcine AM, or by flow cytometry based assays like for an example CFSE dilution or propidium iodide staining etc. An increase in activity indicates immunostimulatory activity. Appropriate increases in activity are outlined below.

In one embodiment, T cell activation is measured for an example by direct killing of target cells like for an example cancer cells or by cytokine secretion or by proliferation or by changes in expression of activation markers like for an example CD137, CD107a, PD1, etc. For T-cells, increases in proliferation, cell surface markers of activation (e.g. CD25, CD69, CD137, PD1), cytotoxicity (ability to kill target cells), and cytokine production (e.g. IL-2, IL-4, IL-6, IFN-g, TNF-a, IL-10, IL-17A) would be indicative of immune modulation that would be consistent with enhanced killing of cancer cells.

In one embodiment, NK cell activation is measured for example by direct killing of target cells like for an example cancer cells or by cytokine secretion or by changes in expression of activation markers like for an example CD107a, etc. For NK cells, increases in proliferation, cytotoxicity (ability to kill target cells and increases CD107a, granzyme, and perforin expression), cytokine production (e.g. IFN-g and TNF), and cell surface receptor expression (e.g. CD25) would be indicative of immune modulation that would be consistent with enhanced killing of cancer cells.

In one embodiment, γδ T cell activation is measured for example by cytokine secretion or by proliferation or by changes in expression of activation markers.

In one embodiment, Th1 cell activation is measured for example by cytokine secretion or by changes in expression of activation markers.

Appropriate increases in activity or response (or decreases, as appropriate as outlined above), are increases of 10%, 20%, 30%, 40%, 50%, 60%, 70%, 80%, 90%, 95% or 98 to 99% percent over the signal in either a reference sample or in control samples, for example test samples that do not contain an anti-VSIG10 antibody of the invention.

Similarly, increases of at least one-, two-, three-, four- or five-fold as compared to reference or control samples show efficacy.

4. Treatment of Pathogen Infections

According to at least some embodiments, anti-VSIG10 antibodies may optionally be used for treating infectious disease, for the same reasons that cancer can be treated: chronic infections are often characterized by varying degrees of functional impairment of virus-specific T-cell responses, and this defect is a principal reason for the inability of the host to eliminate the persisting pathogen. Although functional effector T cells are initially generated during the early stages of infection, they gradually lose function during the course of the chronic infection as a result of persistent exposure to foreign antigen, giving rise to T cell exhaustion. Exhausted T cells express high levels of multiple co-inhibitory receptors such as CTLA-4, PD-1, and LAG3 (Crawford et al., Curr Opin Immunol. 2009; 21:179-186; Kaufmann et al., J Immunol 2009; 182:5891-5897, Sharpe et al., Nat Immunol 2007; 8:239-245). PD-1 overexpression by exhausted T cells was observed clinically in patients suffering from chronic viral infections including HIV, HCV and HBV (Crawford et al., Curr Opin Immunol 2009; 21:179-186; Kaufmann et al., J Immunol 2009; 182:5891-5897, Sharpe et al., Nat Immunol 2007; 8:239-245). There has been some investigation into this pathway in additional pathogens, including other viruses, bacteria, and parasites (Hofmeyer et al., J Biomed Biotechnol. Vol 2011, Art. ID 451694, Bhadra et al., Proc Natl. Acad Sci. 2011; 108(22):9196-201). For example, the PD-1 pathway was shown to be involved in controlling bacterial infection using a sepsis model induced by the standard cecal ligation and puncture method. The absence of PD-1 in knockout mice protected from sepsis-induced death in this model (Huang et al., PNAS 2009: 106; 6303-6308).

T cell exhaustion can be reversed by blocking co-inhibitory pathways such as PD-1 or CTLA-4 (Rivas et al., J Immunol. 2009; 183:4284-91; Golden-Mason et al., J Virol. 2009; 83:9122-30; Hofmeyer et al., J Biomed Biotechnol. Vol 2011, Art. ID 451694), thus allowing restoration of anti-viral immune function. The therapeutic potential of co-inhibition blockade for treating viral infection was extensively studied by blocking the PD-1/PD-L1 pathway, which was shown to be efficacious in several animal models of infection including acute and chronic simian immunodeficiency virus (SIV) infection in rhesus macaques (Valu et al., Nature 2009; 458:206-210) and in mouse models of chronic viral infection, such as lymphocytic choriomeningitis virus (LCMV) (Barber et al., Nature. 2006; 439:682-7), and Theiler's murine encephalomyelitis virus (TMEV) model in SJL/J mice (Duncan and Miller PLoS One. 2011; 6:e18548). In these models PD-1/PD-L1 blockade improved anti-viral responses and promoted clearance of the persisting viruses. In addition, PD-1/PD-L1 blockade increased the humoral immunity manifested as elevated production of specific anti-virus antibodies in the plasma, which in combination with the improved cellular responses leads to decrease in plasma viral loads and increased survival.

As used herein the term “infectious disorder and/or disease” and/or “infection”, used interchangeably, includes any disorder, disease and/or condition caused by presence and/or growth of pathogenic biological agent in an individual host organism. As used herein the term “infection” comprises the disorder, disease and/or condition as above, exhibiting clinically evident illness (i.e., characteristic medical signs and/or symptoms of disease) and/or which is asymptomatic for much or all of it course. As used herein the term “infection” also comprises disorder, disease and/or condition caused by persistence of foreign antigen that lead to exhaustion T cell phenotype characterized by impaired functionality which is manifested as reduced proliferation and cytokine production. As used herein the term “infectious disorder and/or disease” and/or “infection”, further includes any of the below listed infectious disorders, diseases and/or conditions, caused by a bacterial infection, viral infection, fungal infection and/or parasite infection.

Anti-VSIG10 antibodies can be administered alone or in combination with one or more additional therapeutic agents used for treatment of bacterial infections, viral infection, fungal infections, optionally as described herein.

That is, an infected subject is administered an anti-VSIG10 antibody that antagonizes at least one VSIG10 mediated effect on immunity, e.g., its inhibitory effect on cytotoxic T cells or NK activity and/or its inhibitory effect on the production of proinflammatory cytokines, or inhibits the stimulatory effect of VSIG10 on TRegs thereby prompting the depletion or killing of the infected cells or the pathogen, and potentially resulting in disease remission based on enhanced killing of the pathogen or infected cells by the subject's immune cells.

5. Treatment of Sepsis

According to at least some embodiments, anti-VSIG10 antibodies be used for treating sepsis. As used herein, the term “sepsis” or “sepsis related condition” encompasses Sepsis, Severe sepsis, Septic shock, Systemic inflammatory response syndrome (SIRS), Bacteremia, Septicemia, Toxemia, Septic syndrome.

Upregulation of inhibitory proteins has lately emerged as one of the critical mechanisms underlying the immunosuppression in sepsis. The PD-1/PDL-1 pathway, for example, appears to be a determining factor of the outcome of sepsis, regulating the delicate balance between effectiveness and damage by the antimicrobial immune response. During sepsis in an experimental model, peritoneal macrophages and blood monocytes markedly increased PD-1 levels, which was associated with the development of cellular dysfunction (Huang et al 2009 PNAS 106:6303-6308). Similarly, in patients with septic shock the expression of PD-1 on peripheral T cells and of PDL-1 on monocytes was dramatically upregulated (Zhang et al 2011 Crit. Care 15:R70). Recent animal studies have shown that blockade of the PD-1/PDL-1 pathway by anti-PD1 or anti-PDL1 antibodies improved survival in sepsis (Brahmamdam et al 2010 J. Leukoc. Biol. 88:233-240; Zhang et al 2010 Critical Care 14:R220; Chang et al 2013 Critical Care 17:R85). Similarly, blockade of CTLA-4 with anti-CTLA4 antibodies improved survival in sepsis (Inoue et al 2011 Shock 36:38-44; Chang et al 2013 Critical Care 17:R85). Taken together, these findings suggest that blockade of inhibitory proteins, including negative costimulatory molecules, is a potential therapeutic approach to prevent the detrimental effects of sepsis (Goyert and Silver, J Leuk. Biol., 88(2): 225-226, 2010).

According to some embodiments, the invention provides treatment of sepsis using anti-VSIG10 antibodies, either alone or in combination with known therapeutic agent effective for treating sepsis, such as those therapies that block the cytokine storm in the initial hyperinflammatory phase of sepsis, and/or with therapies that have immunostimulatory effect in order to overcome the sepsis-induced immunosuppression phase.

Combination with standard of care treatments for sepsis, as recommended by the “International Guidelines for Management of Severe Sepsis and Septic Shock” (Dellinger et al 2013 Intensive Care Med 39:165-228), some of which are described below.

Broad spectrum antibiotics having activity against all likely pathogens (bacterial and/or fungal—treatment starts when sepsis is diagnosed, but specific pathogen is not identified)—example Cefotaxime (Claforan®), Ticarcillin and clavulanate (Timentin®), Piperacillin and tazobactam (Zosyn®), Imipenem and cilastatin (Primaxin®), Meropenem (Merrem®), Clindamycin (Cleocin), Metronidazole (Flagyl®), Ceftriaxone (Rocephin®), Ciprofloxacin (Cipro®), Cefepime (Maxipime®), Levofloxacin (Levaquin®), Vancomycin or any combination of the listed drugs.

Vasopressors: example Norepinephrine, Dopamine, Epinephrine, vasopressin

Steroids: example: Hydrocortisone, Dexamethasone, or Fludrocortisone, intravenous or otherwise

Inotropic therapy: example Dobutamine for sepsis patients with myocardial dysfunction

Recombinant human activated protein C (rhAPC), such as drotrecogin alfa (activated) (DrotAA).

β-Blockers Additionally Reduce Local and Systemic Inflammation.

Metabolic interventions such as pyruvate, succinate or high dose insulin substitutions.

Combination with novel potential therapies for sepsis:

Selective inhibitors of sPLA2-IIA (such as LY315920NA/S-5920). Rationale: The Group IIA secretory phospholipase A2 (sPLA2-IIA), released during inflammation, is increased in severe sepsis, and plasma levels are inversely related to survival.

Phospholipid emulsion (such as GR270773). Rationale: Preclinical and ex vivo studies show that lipoproteins bind and neutralize endotoxin, and experimental animal studies demonstrate protection from septic death when lipoproteins are administered. Endotoxin neutralization correlates with the amount of phospholipid in the lipoprotein particles.

anti-TNF-α antibody: Rationale: Tumor necrosis factor-α (TNF-α) induces many of the pathophysiological signs and symptoms observed in sepsis

anti-CD14 antibody (such as IC14). Rationale: Upstream recognition molecules, like CD14, play key roles in the pathogenesis. Bacterial cell wall components bind to CD14 and co-receptors on myeloid cells, resulting in cellular activation and production of proinflammatory mediators. An anti-CD14 monoclonal antibody (IC14) has been shown to decrease lipopolysaccharide-induced responses in animal and human models of endotoxemia.

Inhibitors of Toll-like receptors (TLRs) and their downstream signaling pathways. Rationale: Infecting microbes display highly conserved macromolecules (e.g., lipopolysaccharides, peptidoglycans) on their surface. When these macromolecules are recognized by pattern-recognition receptors (called Toll-like receptors [TLRs]) on the surface of immune cells, the host's immune response is initiated. This may contribute to the excess systemic inflammatory response that characterizes sepsis. Inhibition of several TLRs is being evaluated as a potential therapy for sepsis, in particular TLR4, the receptor for Gram-negative bacteria outer membrane lipopolysaccharide or endotoxin. Various drugs targeting TLR4 expression and pathway have a therapeutic potential in sepsis (Wittebole et al 2010 Mediators of Inflammation Vol 10 Article ID 568396). Among these are antibodies targeting TLR4, soluble TLR4, Statins (such as Rosuvastatin®, Simvastatin®), Ketamine, nicotinic analogues, eritoran (E5564), resatorvid (TAK242). In addition, antagonists of other TLRs such as chloroquine, inhibition of TLR-2 with a neutralizing antibody (anti-TLR-2).

Lansoprazole Through its Action on SOCS1 (Suppressor of Cytokine Secretion)

Talactoferrin or Recombinant Human Lactoferrin. Rationale: Lactoferrin is a glycoprotein with anti-infective and anti-inflammatory properties found in secretions and immune cells. Talactoferrin alfa, a recombinant form of human lactoferrin, has similar properties and plays an important role in maintaining the gastrointestinal mucosal barrier integrity. Talactoferrin showed efficacy in animal models of sepsis, and in clinical trials in patients with severe sepsis (Guntupalli et al Crit Care Med. 2013; 41(3):706-716).

Milk fat globule EGF factor VIII (MFG-E8)—a bridging molecule between apoptotic cells and phagocytes, which promotes phagocytosis of apoptotic cells.

Agonists of the ‘cholinergic anti-inflammatory pathway’, such as nicotine and analogues. Rationale: Stimulating the vagus nerve reduces the production of cytokines, or immune system mediators, and blocks inflammation. This nerve “circuitry”, called the “inflammatory reflex”, is carried out through the specific action of acetylcholine, released from the nerve endings, on the α7 subunit of the nicotinic acetylcholine receptor (α7nAChR) expressed on macrophages, a mechanism termed ‘the cholinergic anti-inflammatory pathway’. Activation of this pathway via vagus nerve stimulation or pharmacologic α7 agonists prevents tissue injury in multiple models of systemic inflammation, shock, and sepsis (Matsuda et al 2012 J Nippon Med Sch. 79:4-18; Huston 2012 Surg. Infect. 13:187-193).

Therapeutic agents targeting the inflammasome pathways. Rationale: The inflammasome pathways greatly contribute to the inflammatory response in sepsis, and critical elements are responsible for driving the transition from localized inflammation to deleterious hyperinflammatory host response (Cinel and Opal 2009 Crit. Care Med. 37:291-304; Matsuda et al 2012 J Nippon Med Sch. 79:4-18).

Stem cell therapy. Rationale: Mesenchymal stem cells (MSCs) exhibit multiple beneficial properties through their capacity to home to injured tissue, activate resident stem cells, secrete paracrine signals to limit systemic and local inflammatory response, beneficially modulate immune cells, promote tissue healing by decreasing apoptosis in threatened tissues and stimulating neoangiogenesis, and exhibit direct antimicrobial activity. These effects are associated with reduced organ dysfunction and improved survival in sepsis animal models, which have provided evidence that MSCs may be useful therapeutic adjuncts (Wannemuehler et al 2012 J. Surg. Res. 173:113-26).

Combination of anti-VSIG10 antibody with other immunomodulatory agents, such as immunostimulatory antibodies, cytokine therapy, immunomodulatory drugs. Such agents bring about increased immune responsiveness, especially in situations in which immune defenses (whether innate and/or adaptive) have been degraded, such as in sepsis-induced hypoinflammatory and immunosuppressive condition. Reversal of sepsis-induced immunoparalysis by therapeutic agents that augments host immunity may reduce the incidence of secondary infections and improve outcome in patients who have documented immune suppression (Hotchkiss et al 2013 Lancet Infect. Dis. 13:260-268; Payen et al 2013 Crit Care. 17:118).

Immunostimulatory antibodies promote immune responses by directly modulating immune functions, i.e. blocking other inhibitory proteins or by enhancing costimulatory proteins. Experimental models of sepsis have shown that immunostimulation by antibody blockade of inhibitory proteins, such as PD-1, PDL-1 or CTLA-4 improved survival in sepsis (Brahmamdam et al 2010 J. Leukoc. Biol. 88:233-240; Zhang et al 2010 Critical Care 14:R220; Chang et al 2013 Critical Care 17:R85; Inoue et al 2011 Shock 36:38-44), pointing to such immunostimulatory agents as potential therapies for preventing the detrimental effects of sepsis-induced immunosuppression (Goyert and Silver J Leuk. Biol. 88(2):225-226, 2010). Immunostimulatory antibodies include: 1) Antagonistic antibodies targeting inhibitory immune checkpoints include anti-CTLA4 mAbs (such as ipilimumab, tremelimumab), Anti-PD-1 (such as nivolumab BMS-936558/MDX-1106/ONO-4538, AMP224, CT-011, lambrozilumab MK-3475), Anti-PDL-1 antagonists (such as BMS-936559/MDX-1105, MEDI4736, RG-7446/MPDL3280A); Anti-LAG-3 such as IMP-321), Anti-TIM-3, Anti-BTLA, Anti-B7-H4, Anti-B7-H3, anti-VISTA. 2) Agonistic antibodies enhancing immunostimulatory proteins include Anti-CD40 mAbs (such as CP-870,893, lucatumumab, dacetuzumab), Anti-CD137 mAbs (such as BMS-663513 urelumab, PF-05082566), Anti-OX40 mAbs (such as Anti-OX40), Anti-GITR mAbs (such as TRX518), Anti-CD27 mAbs (such as CDX-1127), and Anti-ICOS mAbs.

Cytokines which directly stimulate immune effector cells and enhance immune responses can be used in combination with anti-GEN antibody for sepsis therapy: IL-2, IL-7, IL-12, IL-15, IL-17, IL-18 and IL-21, IL-23, IL-27, GM-CSF, IFNα (interferon α), IFNβ, IFNγ. Rationale: Cytokine-based therapies embody a direct attempt to stimulate the patient's own immune system. Experimental models of sepsis have shown administration of cytokines, such as IL-7 and IL-15, promote T cell viability and result in improved survival in sepsis (Unsinger et al 2010 J. Immunol. 184:3768-3779; Inoue et al 2010 J. Immunol. 184:1401-1409). Interferon-γ (IFNγ) reverses sepsis-induced immunoparalysis of monocytes in vitro. An in vivo study showed that IFNγ partially reverses immunoparalysis in vivo in humans. IFNγ and granulocyte-macrophage colony-stimulating factor (GM-CSF) restore immune competence of ex vivo stimulated leukocytes of patients with sepsis (Mouktaroudi et al Crit Care. 2010; 14: P17; Leentjens et al Am J Respir Crit Care Med Vol 186, pp 838-845, 2012).

Immunomodulatory drugs such as thymosin al. Rationale: Thymosin α 1 (Tα1) is a naturally occurring thymic peptide which acts as an endogenous regulator of both the innate and adaptive immune systems. It is used worldwide for treating diseases associated with immune dysfunction including viral infections such as hepatitis B and C, certain cancers, and for vaccine enhancement. Notably, recent development in immunomodulatory research has indicated the beneficial effect of Ta1 treatment in septic patients (Wu et al. Critical Care 2013, 17:R8).

In the above-described sepsis therapies preferably a subject with sepsis or at risk of developing sepsis because of a virulent infection, e.g., one resistant to antibiotics or other drugs, will be administered an immunostimulatory anti-VSIG10 antibody or antigen-binding fragment according to the invention, which antibody antagonizes at least one VSIG10 mediated effect on immunity, e.g., its inhibitory effect on cytotoxic T cells or NK activity and/or its inhibitory effect on the production of proinflammatory cytokines, or inhibits the stimulatory effect of VSIG10 on TRegs thereby promoting the depletion or killing of the infected cells or the pathogen, and potentially resulting in disease remission based on enhanced killing of the pathogen or infected cells by the subject's endogenous immune cells. Because sepsis may rapidly result in organ failure, in this embodiment it may be beneficial to administer anti-VSIG10 antibody fragments such as Fabs rather than intact antibodies as they may reach the site of sepsis and infection quicker than intact antibodies. In such treatment regimens antibody half-life may be of lesser concern due to the sometimes rapid morbidity of this disease.

B. Diagnostic Uses

The anti-VSIG10 antibodies provided also find use in the in vitro or in vivo diagnosis, including imaging, of tumors that over-express VSIG10. It should be noted, however, that as discussed herein, VSIG10, as an immuno-oncology target protein, is not necessarily overexpressed on cancer cells rather within the immune infiltrates in the cancer. In some instances it is; rather, the mechanism of action, activation of immune cells such as T cells and NK cells, that results in cancer diagnosis. Accordingly, anti-VSIG10 antibodies can be used to diagnose cancer.

Generally, diagnosis can be done in several ways. In one embodiment, a tissue from a patient, such as a biopsy sample, is contacted with a VSIG10 antibody, generally labeled, such that the antibody binds to the endogenous VSIG10. The level of signal is compared to that of normal non-cancerous tissue either from the same patient or a reference sample, to determine the presence or absence of cancer. The biopsy sample can be from a solid tumor, a blood sample (for lymphomas and leukemias such as ALL, T cell lymphoma, etc).

In general, in this embodiment, the anti-VSIG10 is labeled, for example with a fluorophore or other optical label, that is detected using a fluorometer or other optical detection system as is well known in the art. In an alternate embodiment, a secondary labeled antibody is contacted with the sample, for example using an anti-human IgG antibody from a different mammal (mouse, rat, rabbit, goat, etc.) to form a sandwich assay as is known in the art. Alternatively, the anti-VSIG10 mAb could be directly labeled (i.e. biotin) and detection can be done by a secondary Ab directed to the labeling agent in the art.

Once over-expression of VSIG10 is seen, treatment can proceed with the administration of an anti-VSIG10 antibody according to the invention as outlined herein.

In other embodiments, in vivo diagnosis is done. Generally, in this embodiment, the anti-VSIG10 antibody (including antibody fragments) is injected into the patient and imaging is done. In this embodiment, for example, the antibody is generally labeled with an optical label or an MRI label, such as a gadolinium chelate, radioactive labeling of mAb (including fragments).

In some embodiments, the antibodies described herein are used for both diagnosis and treatment, or for diagnosis alone. When anti-VSIG10 antibodies are used for both diagnosis and treatment, some embodiments rely on two different anti-VSIG10 antibodies to two different epitopes, such that the diagnostic antibody does not compete for binding with the therapeutic antibody, although in some cases the same antibody can be used for both. For example, this can be done using antibodies that are in different bins, e.g. that bind to different epitopes on VSIG10, such as outlined herein. Thus included in the invention are compositions comprising a diagnostic antibody and a therapeutic antibody, and in some embodiments, the diagnostic antibody is labeled as described herein. In addition, the composition of therapeutic and diagnostic antibodies can also be co-administered with other drugs as outlined herein.

As will be appreciated by those in the art, for ex vivo or in vitro assays, murine antibodies can be used.

In many embodiments, a diagnostic antibody is labeled. By “labeled” herein is meant that the antibodies disclosed herein have one or more elements, isotopes, or chemical compounds attached to enable the detection in a screen or diagnostic procedure. In general, labels fall into several classes: a) immune labels, which may be an epitope incorporated as a fusion partner that is recognized by an antibody, b) isotopic labels, which may be radioactive or heavy isotopes, c) small molecule labels, which may include fluorescent and colorimetric dyes, or molecules such as biotin that enable other labeling methods, and d) labels such as particles (including bubbles for ultrasound labeling) or paramagnetic labels that allow body imagining. Labels may be incorporated into the antibodies at any position and may be incorporated in vitro or in vivo during protein expression, as is known in the art.

Diagnosis can be done either in vivo, by administration of a diagnostic antibody that allows whole body imaging as described below, or in vitro, on samples removed from a patient. “Sample” in this context includes any number of things, including, but not limited to, bodily fluids (including, but not limited to, blood, urine, serum, lymph, saliva, anal and vaginal secretions, perspiration and semen), as well as tissue samples such as result from biopsies of relevant tissues.

In some embodiments, in vivo imaging is done, including but not limited to ultrasound, CT scans, X-rays, MRI and PET scans, as well as optical techniques, such as those using optical labels for tumors near the surface of the body.

In vivo imaging of diseases associated with VSIG10 may be performed by any suitable technique. For example, 99Tc-labeling or labeling with another .beta.-ray emitting isotope may be used to label anti-VSIG10 antibodies. Variations on this technique may include the use of magnetic resonance imaging (MRI) to improve imaging over gamma camera techniques.

In one embodiment, the present invention provides an in vivo imaging method wherein an anti-VSIG10 antibody is conjugated to a detection-promoting agent, the conjugated antibody is administered to a host, such as by injection into the bloodstream, and the presence and location of the labeled antibody in the host is assayed. Through this technique and any other diagnostic method provided herein, the present invention provides a method for screening for the presence of disease-related cells in a human patient or a biological sample taken from a human patient.

For diagnostic imaging, radioisotopes may be bound to an anti-VSIG10 antibody either directly, or indirectly by using an intermediary functional group. Useful intermediary functional groups include chelators, such as ethylenediaminetetraacetic acid and diethylenetriaminepentaacetic acid (see for instance U.S. Pat. No. 5,057,313), in such diagnostic assays involving radioisotope-conjugated anti-VSIG10 antibodies, the dosage of conjugated anti-VSIG10 antibody delivered to the patient typically is maintained at as low a level as possible through the choice of isotope for the best combination of minimum half-life, minimum retention in the body, and minimum quantity of isotope, which will permit detection and accurate measurement.

In addition to radioisotopes and radio-opaque agents, diagnostic methods may be performed using anti-VSIG10 antibodies that are conjugated to dyes (such as with the biotin-streptavidin complex), contrast agents, fluorescent compounds or molecules and enhancing agents (e.g. paramagnetic ions) for magnetic resonance imaging (MRI) (see, e.g., U.S. Pat. No. 6,331,175, which describes MRI techniques and the preparation of antibodies conjugated to a MRI enhancing agent). Such diagnostic/detection agents may be selected from agents for use in magnetic resonance imaging, and fluorescent compounds.

In order to load an anti-VSIG10 antibody with radioactive metals or paramagnetic ions, it may be necessary to react it with a reagent having a long tail to which are attached a multiplicity of chelating groups for binding the ions. Such a tail may be a polymer such as a polylysine, polysaccharide, or other derivatized or derivatizable chain having pendant groups to which can be bound chelating groups such as, e.g., porphyrins, polyamines, crown ethers, bisthiosemicarbazones, polyoximes, and like groups known to be useful for this purpose.

Chelates may be coupled to anti-VSIG10 antibodies using standard chemistries. A chelate is normally linked to an anti-VSIG10 antibody by a group that enables formation of a bond to the molecule with minimal loss of immunoreactivity and minimal aggregation and/or internal cross-linking.

Examples of potentially useful metal-chelate combinations include 2-benzyl-DTPA and its monomethyl and cyclohexyl analogs, used with diagnostic isotopes in the general energy range of 60 to 4,000 keV, such as ¹²⁵I, ¹²³I, ¹²⁴I, ⁶²CU, ⁶⁴Cu, ¹⁸F, ¹¹¹In, ⁶⁷Ga, ⁹⁹Tc, ⁹⁴Tc, ¹¹C, ¹³N, ⁵O, and ⁷⁶Br, for radio-imaging.

Labels include a radionuclide, a radiological contrast agent, a paramagnetic ion, a metal, a fluorescent label, a chemiluminescent label, an ultrasound contrast agent and a photoactive agent. Such diagnostic agents are well known and any such known diagnostic agent may be used. Non-limiting examples of diagnostic agents may include a radionuclide such as 110In, 111In, 177Lu, 18F, 52Fe, 62Cu, 64Cu, 67Cu, 67Ga, 68Ga, 86Y, 90Y, 89Zr, 94mTc, 94Tc, 99mTc, 120I, 123I, 124I, 125I, 131I, 154-158Gd, 32P, 11C, 13N, 150, 186Re, 188Re, 51Mn, 52mMn, 55Co, 72As, 75Br, 76Br, 82mRb, 83Sr, or other .gamma.-, .beta.-, or positron-emitters.

Paramagnetic ions of use may include chromium (III), manganese (II), iron (III), iron (II), cobalt (II), nickel (III), copper (III), neodymium (III), samarium (III), ytterbium (III), gadolinium (III), vanadium (II), terbium (III), dysprosium (III), holmium (III) or erbium (III), Metal contrast agents may include lanthanum (III), gold (III), lead (II) or bismuth (III).

Ultrasound contrast agents may comprise liposomes, such as gas filled liposomes. Radiopaque diagnostic agents may be selected from compounds, barium compounds, gallium compounds, and thallium compounds.

These and similar chelates, when complexed with non-radioactive metals, such as manganese, iron, and gadolinium may be useful for MRI diagnostic methods in connection with anti-VSIG10 antibodies. Macrocyclic chelates such as NOTA, DOTA, and TETA are of use with a variety of metals and radiometals, most particularly with radionuclides of gallium, yttrium, and copper, respectively. Such metal-chelate complexes may be made very stable by tailoring the ring size to the metal of interest. Other ring-type chelates such as macrocyclic polyethers, which are of interest for stably binding nuclides, such as 223Ra may also be suitable in diagnostic methods.

Thus, the present invention provides diagnostic anti-VSIG10 antibody conjugates, wherein the anti-VSIG10 antibody conjugate is conjugated to a contrast agent (such as for magnetic resonance imaging, computed tomography, or ultrasound contrast-enhancing agent) or a radionuclide that may be, for example, a .gamma.-, .beta.-, .alpha.-, Auger electron-, or positron-emitting isotope.

Anti-VSIG10 antibodies may also be useful in, for example, detecting expression of an antigen of interest in specific cells, tissues, or serum. For diagnostic applications, the antibody typically will be labeled with a detectable moiety for in vitro assays. As will be appreciated by those in the art, there are a wide variety of suitable labels for use in in vitro testing. Suitable dyes for use in this aspect of the invention include, but are not limited to, fluorescent lanthanide complexes, including those of Europium and Terbium, fluorescein, rhodamine, tetramethylrhodamine, eosin, erythrosin, coumarin, methyl-coumarins, quantum dots (also referred to as “nanocrystals”; see U.S. Ser. No. 09/315,584, hereby incorporated by reference), pyrene, Malacite green, stilbene, Lucifer Yellow, Cascade Blue™, Texas Red, Cy dyes (Cy3, Cy5, etc.), alexa dyes (including Alexa, phycoerythin, bodipy, and others described in the 6th Edition of the Molecular Probes Handbook by Richard P. Haugland, hereby expressly incorporated by reference.

Stained tissues may then be assessed for radioactivity counting as an indicator of the amount of VSIG10-associated peptides in the tumor. The images obtained by the use of such techniques may be used to assess biodistribution of VSIG10 in a patient, mammal, or tissue, for example in the context of using VSIG10 as a biomarker for the presence of invasive cancer cells.

### EXAMPLES

### Example 1: Expression Analysis of VSIG10 Proteins

Methods Used for Expression Analysis:

A transcriptome reference was obtained from Omicsoft which was based on Gencode gene model (http://www.arrayserver.com/wiki/index.php?title=Omicsoft_GenCode_Gene_Model). All RNA sequencing reads were aligned to the transcriptome sequences first. This alignment allowed for non-unique mapping because isoforms share many exons. Each read was then assigned genomic coordinates and exon junctions based on the transcriptome matching. The remaining unmapped reads were aligned directly to the genome by considering one or more exon junctions. Finally, read counts were normalized as described by Bo et al. (Bioinformatics 2010, 26 (4): 493-500) and converted to gene expression values as described by Trapnell et al (Nat Biotechnol. 2010 May; 28(5):511-5).

Genotype-Tissue Expression (GTEx) data (http://www.nature.com/ng/journal/v45/n6/full/ng.2653. html; http://www.gtexportal.org/home/) and cancer tissues data from The Cancer Genome Atlas (TCGA) (http://cancergenome.nih.gov/) were obtained from Omicsoft (http://www.omicsoft.com/oncoland-service).

The correlation analysis was conducted per tumor type and only correlations where both genes were expressed above 0.5 RPKM with at least 100 samples in the same tumor type, were considered. These gene expression signatures were tested for enrichment of interacting proteins, pathways and disease genes. Enrichment p-values were calculated for each tumor type and the mean−log(p-value) was used to rank the scoring gene sets.

Methods: Genes correlation: FPKM values were transformed to log 2 (FPKM+0.1). Samples with value that fulfills log 2 (FPKM+0.1)<log 2(0.1) for at least one of the genes, were omitted. Pearson Correlation Coefficient (PCC) and the Least Squared Estimators for the regression line were computed for the 2 lists (one list per gene). PCCs with lower value than 0.6 were omitted as well as PCCs that failed to show significant value when testing the linear correlation between the expression levels of the 2 genes.

Gene Enrichment analysis: Pathway, interaction and disease data were obtained from Reactome (http://www.reactome.org) and KEGG Pathways (http://www.genome.jp/kegg). To identify pathways and processes that were enriched within a given gene list, a hyper-geometric-based enrichment analysis was implemented. The hyper-geometric p-value was calculated using the R program (http://www.R-project.org) with the following command: phyper(x−1, m, n−m, k and lower.tail=FALSE), where x is the number of genes from the gene list that are members of the pathway, m is the number of genes in the pathway, n is the total number of unique genes in all pathways, and k is the number of genes from the list that were present in at least one pathway. The resulting p-value is indicative of the likelihood of enriching for a specific pathway by chance given the size of the gene list. The same analytical procedure was applied to gene interactions where all genes interacting with a given gene were treated as a pathway; or genes associated with a disease where all associated genes were treated as a pathway.

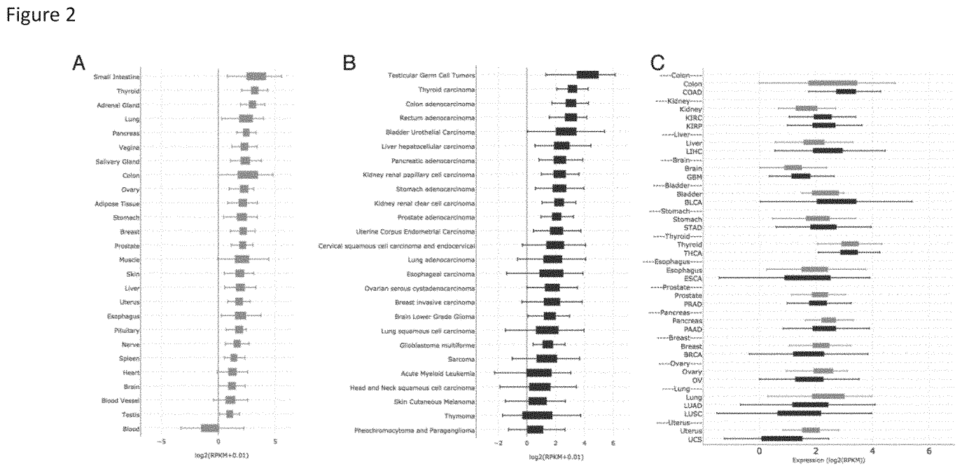

It was shown that VSIG10 is expressed in both normal and cancer tissues. FIG. 2 shows VSIG10 expression in normal (A; GTEx project data), Cancer (B; TCGA primary and metastatic tumor data) and GTEx vs TCGA (C). In both normal and cancer, it was shown to be highly expressed in solid tissues and shows low blood expression. In matched normal vs cancer, it was shown to be enriched in kidney, liver and bladder cancers compared to normal (FIG. 2C).

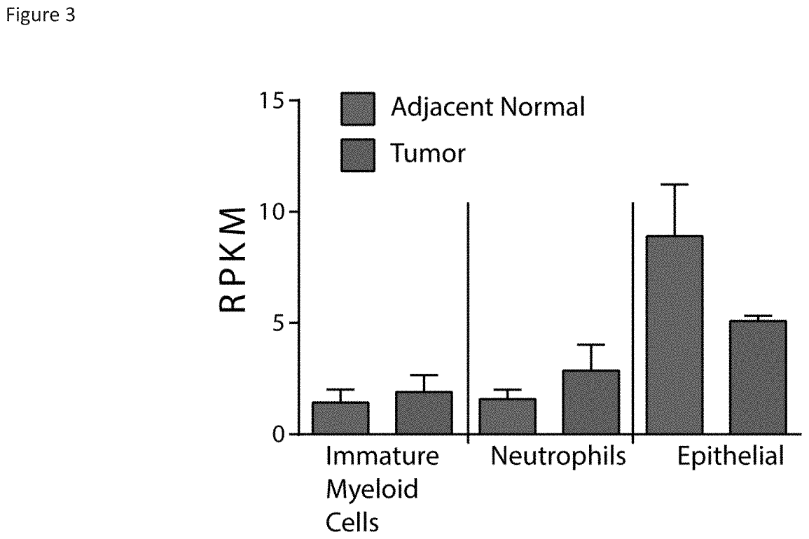

FIG. 3 shows that in cancer VSIG10 is expressed in epithelial cells as well as in immune cells. Specifically, FIG. 3 shows that VSIG10 Expression in epithelial, neutrophil and immature myeloid cells sorted from non-small cell lung cancers and adjacent normal tissues (pmid: 26940867).

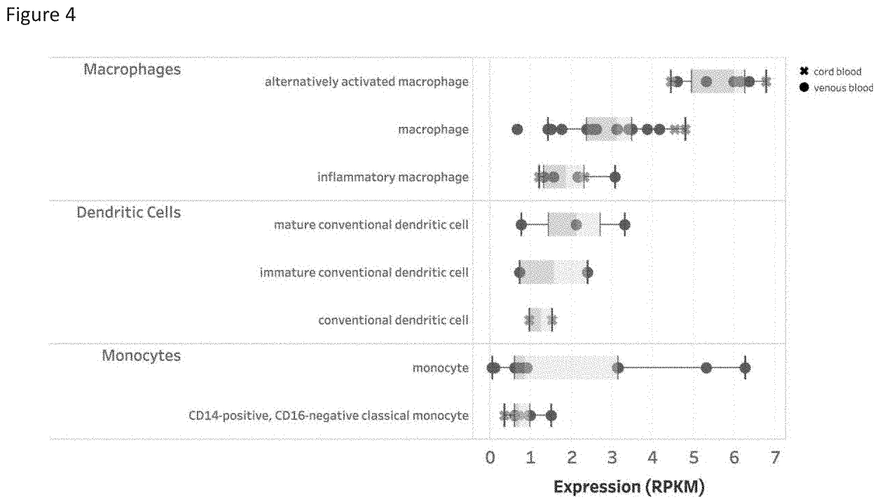

FIG. 4 shows VSIG10 expression in macrophages, dendritic cells and monocytes from the Blueprint project. FIG. 4 further demonstrates that in sorted normal primary cells, VSIG10 is expressed in human immune cells such as macrophages, dendritic cells and monocytes.

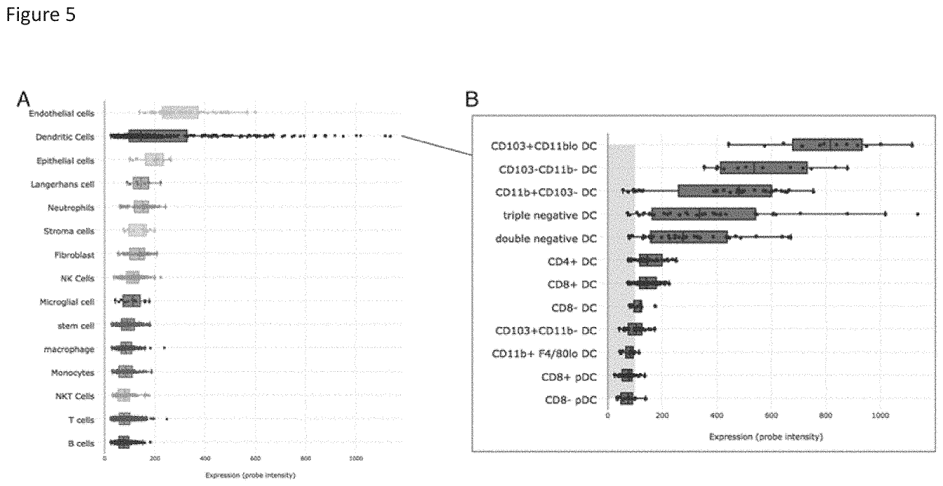

FIG. 5 shows VSIG10 expression in mouse immune cells (ref: immgen, GSE15907). Specifically, FIG. 5 demonstrates that in mice, VSIG10 is highly expressed in dendritic cells sorted from tissues and within dendritic cell subpopulations it is highest expressed in CD103+ dendritic cells.

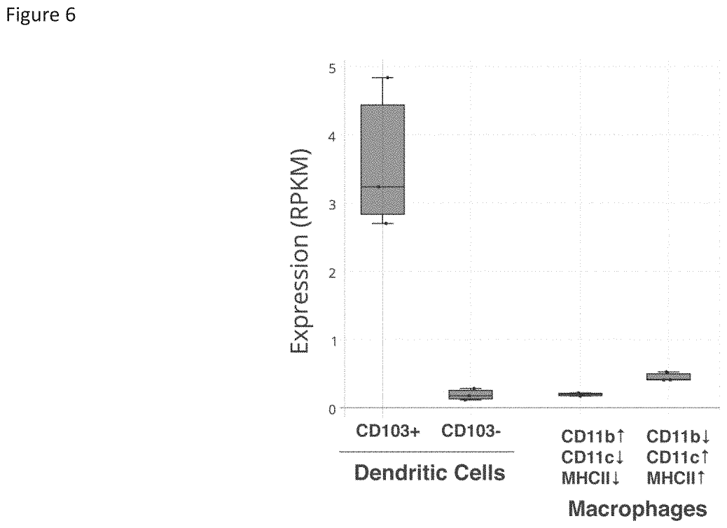

Another study in mice where DC103+ cells were sorted from the tumor showed that VSIG10 is highly expressed in the CD103+ dendritic cell population in the tumor microenvironment (FIG. 6). FIG. 6 shows VSIG10 expression in dendritic cells and macrophages from lung cancer tumor model (pmid: 25446897).

VSIG10 was shown to be expressed in both epithelial and professional antigen presenting cells. However, experimental in vivo data presented in Example 4 below suggests that the cancer relevant immune-modulatory effect of VSIG10 is mainly contributed from the immune cells. In this study, tumor growth was assessed in mice with genomic deletion of the VSIG10 gene. In this model, in which tumor growth inhibition was observed upon VSIG10 gene depletion, VSIG10 is not expressed on the host immune system but only expressed on the engrafted cancer cells. Therefore, the effect observed comes from the lack of VSIG10 is the mouse immune cells. This data supports a critical role for VSIG10 expressed on immune cells for a functional and effective immune response which is likely to occur via antigen presentation, suggesting an effect as both single agent and in combination therapy. The expression of VSIG10 on subsets of dendritic cells (which are known to play a role in T cell priming) suggests a potential role of VSIG10 in this process and therefor favors combination therapy with cancer vaccines.

### Example 2: Generation and Characterization of Custom Abs Against VSIG10: AB-577 and AB-576

This Example relates to raising monoclonal antibodies specific to VSIG10 human protein.

1. Generation of Mouse Monoclonal Antibodies Against Human VSIG10 Protein

Mouse monoclonal antibody was raised at Genscript (USA) using MonoExpress™ Custom Monoclonal Antibody Service Package.

Antibodies against human VSIG10 protein were raised by immunizing BalbC mice with recombinant VSIG10 protein comprised of the extra cellular domain fused to human IgG1 (SEQ ID NO: 1). The stages included the immunization, cell fusion and screening, subcloning and Abs production and purification.

1.2 Materials & Methods

Reagents used in this study:

Stable pool of HEK293 cells over expressing human VSIG10 flag protein (Human VSIG10 flag amino acid sequence SEQ ID NO: 214; Human VSIG10 flag nucleic acid sequence SEQ ID NO: 210; pMSCV plasmid with Human VSIG10 flag sequence SEQ ID NO: 211)

Stable pool of HEK293 cells over expressing mouse VSIG10 flag protein (Mouse VSIG10 flag amino acid sequence SEQ ID NO: 215; Mouse VSIG10 flag nucleic acid sequence SEQ ID NO: 212; pCDNA3.1 plasmid with mouse VSIG10 flag sequence SEQ ID NO: 213)

Stable pool of HEK293 cells transduced with the empty pMSCV vector

Stable pool of HEK293 cells transfected with the empty pCDNA3.1+ vector

VSIG10-ECD-Fc (H:H) recombinant Fc fusion protein-Human ECD of VSIG10 fused to the Fc domain of Human IgG1 (SEQ ID NO: 1)

Mouse IgG1, kappa (Biolegend cat.400166)

ON-TARGETplus Human VSIG10 siRNA—SMARTpool, Dharmacon, Cat#L-020362-02

ON TARGET plus non targeting siRNA, Dharmacon, Cat#D-001810-01-05

Lipofectamine® RNAiMAX Transfection Reagent, ThermoFisher cat. 13778150

Goat anti mouse PE (Jackson cat. 115-116-146)

goat anti mouse-HRP (Jackson cat#115-035-062)

Fixable viability stain 450 (BD Horizon cat #562247)

FACS buffer: 0.5% BSA+0.05% Sodium azide+2 mM EDTA in PBS

Methods:

1.2.1 Anti Human VSIG10 mAbs Generation:

Monoclonal antibodies generation at Genscript including the following stages:

Animal immunization: 5 Balb/c mice were immunized with VSIG10 protein.

The immunization protocol included primary immunization and additional three boosts. The immune response was tested by ELISA using the immunized sera 7 days after each boost. The Immune sera were taken after the final boosting and diluted sera were tested by WB (Genscript and Compugen) and by FACS (Compugen).

Cell Fusion and Screening

Cell fusion and clone plating: one round of cell fusion was performed by electro-fusion. All fused cells from each cell fusion were plated into ten to fifteen 96-well plates.

Primary binder screening: Screen the conditioned medium by ELISA with VSIG10 Fc fusion protein.

Confirmatory screening: the positive supernatants from the primary binder screening was reconfirmed by the VSIG10 protein and by negative control protein, then preform WB test.

Clone expansion and frozen: Clones were expanded into 24-well plates, 2 ml of supernatants (conditioned media, pre clonal sups) for each clone were selected, and cells were froze down.

Subcloning, Expansion and Cryopreservation

Sub-cloning: sub-cloned by limiting dilution to ensure that the sub-clones are derived from a single parental cell. The clones will be carried for a maximum of 3 generations

Subcloning screening: screened by ELISA and WB.

Monoclone cryopreservation: Two stable sub-clonal cell lines of each parental clone were cryopreserved based on the result of ELISA.

Isotyping assay was preformed to all the subcloning-supernatant (clonal sups).

Antibody Production

Antibody production was carried out in roller bottles with serum free medium and low endotoxin.

Antibodies will be purified by Protein A affinity column chromatography. The purified antibodies were dialyzed against PBS buffer for storage.

5. Hybridoma sequencing—sequencing for the variable domain and leader sequence.

1.2.2 Analysis of the Clonal Sups and Purified Antibodies AB-577 and AB-576 in Western Blot (WB) Application

Whole cell extracts of HEK293 transuded cells over expressing the human VSIG10 flag protein or whole cell extracts of HEK293 cells transuded with an empty vector were analyzed by WB using the clonal sup of AB-577 and AB-577 purified mAb or the clonal sup of AB-576 and AB-576 purified mAb (Genscript) described above. The sups were tested as well as the purified Ab at a final concentration of 10 ug/ml in 5% TTBS/BSA.

Staining was followed by a secondary antibody goat anti mouse-HRP (Jackson cat#115-035-062) diluted 1:10,000 in 5% milk/TBST.

1.2.3. Protein Expression Analysis by Flow Cytometry (FACS)

The cell surface expression of VSIG10 protein was analyzed by FACS. Human cell lines were stained with VioBlue reagent diluted 1:1000 in PBS. Cells were incubated 10 min at R.T. and then washed once with PBS. To detect the human VSIG10 protein, cells were stained with a custom monoclonal anti-human VSIG10 mAbs AB-577 (Genscript) diluted to a concentration of 10 ug/ml, or with a custom monoclonal anti-human VSIG10 mAbs AB-576 (Genscript) diluted to a concentration of 10 ug/ml, or mIgG1 kappa Isotype control at the same concentration.

1.2.4 VSIG10 Knock Down

Knock down of endogenous human VSIG10 was carried out by transient transfection of siRNA. Transfection of 120 pmol VSIG10 siRNA pool or scrambled siRNA performed by Lipofectamine® RNAiMAX Transfection Reagent, as listed above in materials & methods and according to the manufacture procedure. 48-72 hours post transfection, cells were collected for further analysis by qRT-PCR, FACS and WB.

1.2.5 Analysis of AB-577 and AB-576 mAbs in Immunohistochemistry (IHC) Application

Paraffin blocks of HEK293 overexpressing VSIG10 (OX cells); HEK293 cells transfected with empty vector (EV cells), DU-145 cells expressing endogenous VSIG10 was prepared. FFPE NSCLC and normal colon tissues were also stained.

IHC calibration assay was performed at Smart Assay (Israel).

Standard five micrometer sections of this blocks were used in series of IHC experiments. In each experiment sections were subjected to heat induced epitope retrieval (HIER) procedure using three different buffers: citric buffer (pH 6.0)—CB, citraconic anhydride buffer—CA and TE (pH 8.0), one set of sections was processed without retrieval.

AB-577 or AB-576 antibody was applied to four types of sections (three heat retrieved and one non-retrieved). Bound primary antibodies were revealed using horseradish peroxidase based detection system.

Results

AB-577 and AB-576 were identified as mouse IgG1, kappa. They were sequenced (sequences are depicted in FIG. 7 and below) and further characterized in WB, FACS and IHC.

SEQ ID NO:201 depicts the 577Ab variable heavy chain amino acids sequence and in SEQ ID NO:206 depicts the 577Ab variable light chain amino acids sequence. The corresponding nucleic acid sequences are depicted in SEQ ID Nos 200 and 205, respectively. The heavy chain CDRs are depicted in SEQ ID Nos 202, 203 and 204 for HC-CDR1, HC-CDR2 and HC-CDR3, respectively:

SEQ ID NO: 202 577Ab HC-CDR1:
DYAIS
SEQ ID NO: 203 577Ab HC-CDR2:
EIYPGNGNTYFNEKFKD
SEQ ID NO: 204 577Ab HC-CDR3:
GYANYLP

The light chain CDRs are depicted in SEQ ID Nos: 207, 208 and 209 for LC-CDR1, LC-CDR2 and LC-CDR3, respectively:

SEQ ID NO: 207 577Ab LC-CDR1:
KASEDIYNRLA
SEQ ID NO: 208 577Ab LC-CDR2:
GATGLET
SEQ ID NO: 209 577Ab LC-CDR3:
QQYWSTPRT

SEQ ID NO:217 depicts the 576-Ab variable heavy chain amino acids sequence and in SEQ ID NO:222 depicts the 576-Ab variable light chain amino acids sequence. The corresponding nucleic acid sequences are depicted in SEQ ID Nos 216 and 221, respectively. The heavy chain CDRs are depicted in SEQ ID Nos 218, 219 and 220 for HC-CDR1, HC-CDR2 and HC-CDR3, respectively:

SEQ ID NO: 218 576Ab HC-CDR1:
DYYMK
SEQ ID NO: 219 576Ab HC-CDR2:
DINPNNGGTTYNQKFKG
SEQ ID NO: 220 576Ab HC-CDR3:
FRLRAMDY

The light chain CDRs are depicted in SEQ ID Nos: 223, 224 and 225 for LC-CDR1, LC-CDR2 and LC-CDR3, respectively:

SEQ ID NO: 223 576Ab LC-CDR1:
KSSQSLLNSGDRKNYLT
SEQ ID NO: 224 576Ab LC-CDR2:
WASTRES
SEQ ID NO: 225 576Ab LC-CDR3:
QNDYIYPLT

1.3.1 Analysis of Hybridoma Supernatant (Clonal Sup) and Purified AB-577 or AB-576 Anti Human VSIG10 Antibodies by WB

The performance of the AB-577 or AB-576 clonal supernatants and purified antibodies against human VSIG10 in WB application were tested using HEK293 cell over-expressing human VSIG10 Flag. HEK293 cells transduced with an empty vector (EV) were used as a negative control.

The purified antibodies were screened also on DAN-G, DU-145, LOVO and ZR-75 cell lines endogenously expressing VSIG10 protein.

FIG. 7 shows the antibody AB-577 and AB576 sequences. FIG. 7A shows the heavy chain: DNA sequence (402 bp) (SEQ ID NO:200). FIG. 7B shows the heavy chain: Amino acids sequence (134 aa) (SEQ ID NO:201). FIG. 7C shows the light chain: DNA sequence (381 bp) (SEQ ID NO:205). FIG. 7D shows the light chain: Amino acids sequence (127 aa) (SEQ ID NO:206). FIG. 7E shows the heavy chain: DNA sequence (408 bp) (SEQ ID NO:216); FIG. 7F shows the heavy chain amino acids sequence (136 aa) (SEQ ID NO:217) FIG. 7G shows the light chain DNA sequence (399 bp) (SEQ ID NO:221); FIG. 7H shows the light chain amino acids sequence (133 aa) (SEQ ID NO:222). The CDRs are marked in blue font and bold.

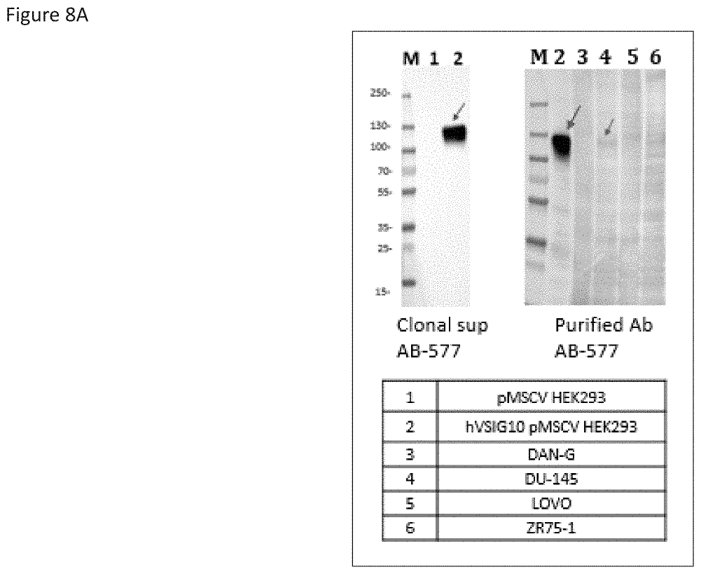

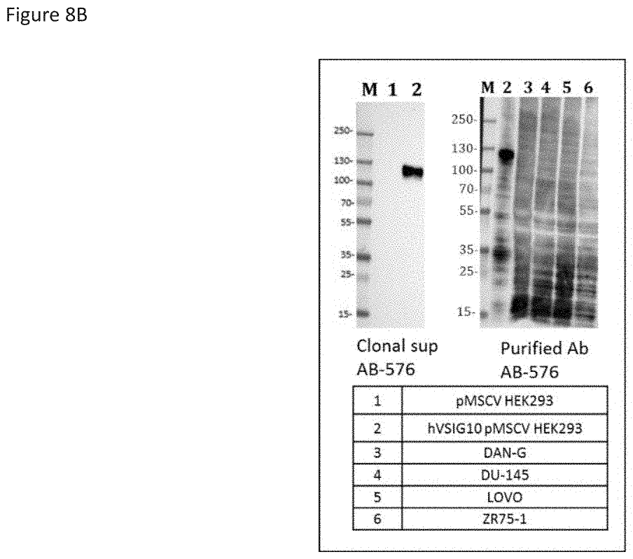

FIGS. 8A and B show WB analysis on HEK293 overexpressing human VSIG10 flag transfected cells and endogenous cell line expressing VSIG10 using AB-577 clonal supernatants and purified Ab (FIG. 8A) or using AB-576 clonal supernatants and purified Ab (FIG. 8B). HEK293 transduced with an empty vector (lane 1), whole cells extract of HEK293 cells expressing the human VSIG10 (lane 2), DAN-G (lane 3), DU-145 (lane 4), LOVO (lane 5) or ZR-75 (lane 6) were analyzed using the AB-577 or AB576 clonal supernatants (left) and purified antibodies (right). Detection was carried out using Goat Anti mouse-HRP.

FIG. 8A shows a band corresponding to size of ˜120 kDa (Calculated Mw is 60 kDa) with AB-577 clonal supernatant (left) and purified mAb (right) in the HEK293 human VSIG10 flag extract (20 ug) as oppose to the negative control cells extracts (HEK293 EV cells). Band was also observed in DU-145 cells extract (40 ug) at a lower level of intensity.

FIG. 8B shows a band corresponding to size of ˜120 kDa (Calculated Mw is 60 kDa) with AB-576 clonal supernatant (left) and purified mAb (right) in the HEK293 human VSIG10 flag extract (20 ug) as oppose to the negative control cells extracts (HEK293 EV cells).

The performance of the purified mAbs against human VSIG10, in FACS application were tested using HEK293 cells over-expressing human VSIG10 Flag. HEK293 cells transfected with an empty vector were used as a negative control.

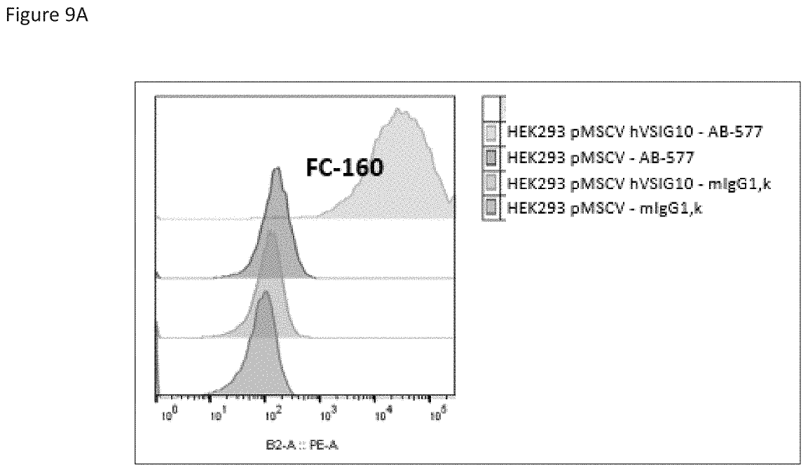

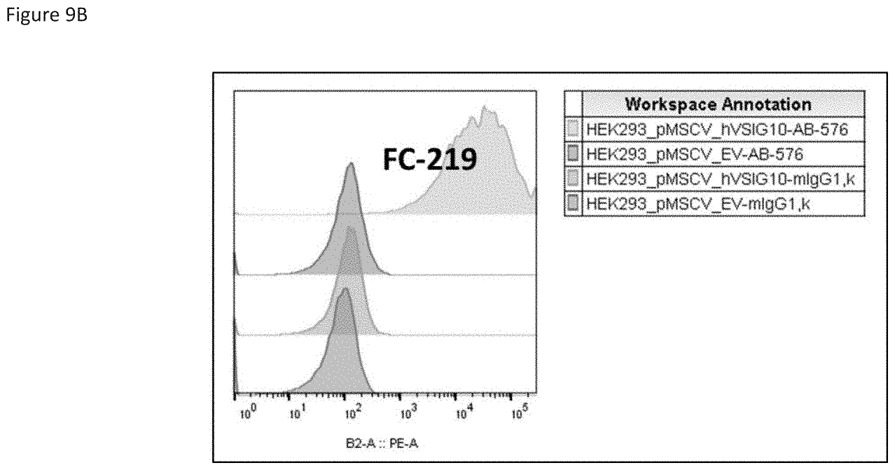

As shown in FIG. 9, the binding of AB-577 (FIG. 9A) and AB-576 (FIG. 9B) to the HEK293 cells over-expressing human VSIG10 Flag protein (purple) is significantly higher than the binding of AB-577 and AB-576 to the empty vector cells (green) (160 fold and 219 fold change MFI ratio, respectively).

In particular, FIG. 9 shows FACS analysis using anti human VSIG10 mAb AB-577 (FIG. 9A) and AB-576 (FIG. 9B) on HEK293 cells over-expressing human VSIG10 Flag protein. HEK293 cells over-expressing the human VSIG10 Flag (purple) or HEK293 transfected with empty vector (green) were analyzed by FACS using AB-577 or AB576. Detection was carried out using Goat anti mouse PE secondary Ab.

The mAb binding was evaluated on HEK293 cells over expressing human VSIG10 as well. The affinity of AB-577 and of AB-576 was determined by FACS titration on HEK293 cells transfected to over express VSIG10 as compared to HEK293 cells transfected with empty vector.

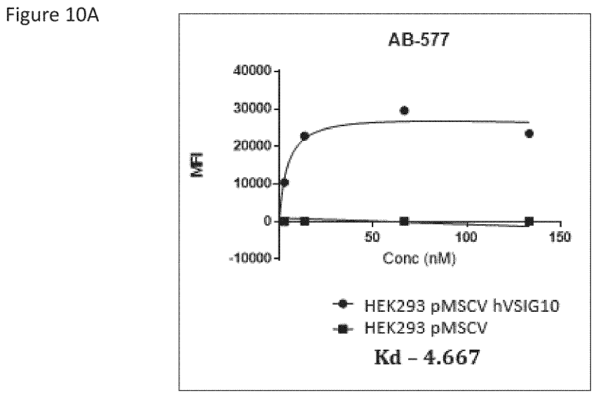

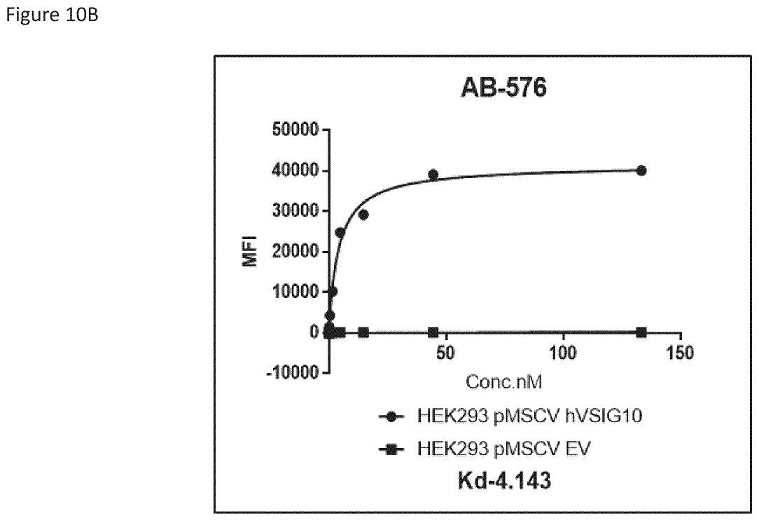

FIG. 10 shows affinity measurements using FACS application for the anti-human VSIG10 mAbs AB-577 (FIG. 10A) and AB-576 (FIG. 10B) on HEK293 cells over-expressing human VSIG10 Flag protein. HEK293 cells over-expressing the human VSIG10 Flag (dots) or HEK293 transfected with empty vector (square) were analyzed by FACS using AB-577 (FIG. 10A) in 4 concentrations or by AB-576 (FIG. 10B) in 8 concentrations. Detection was carried out using Goat Anti mouse-PE secondary Ab.

As shown in FIG. 10A, the binding AB-577 to HEK293 cells over-expressing human VSIG10 flag protein (dots) compared to the EV cells (squares) was performed and the affinity was determined based on the Kd values calculated from the binding curve. The Kd value for AB-577 is 4.667 (nM).

As shown in FIG. 10B. the binding AB-576 to HEK293 cells over-expressing human VSIG10 flag protein (dots) compared to the EV cells (squares) was performed and the affinity was determined based on the Kd values calculated from the binding curve. The Kd value for AB-576 is 4.143 (nM).

To confirm endogenous expression of VSIG10 protein in DAN-G and AsPc1 human cell lines, human VSIG10 siRNA pool was used for knock down as described in Material & Methods. 72 hours post siRNA transfection, cells were harvested for further analysis by qRT-PCR and FACS.

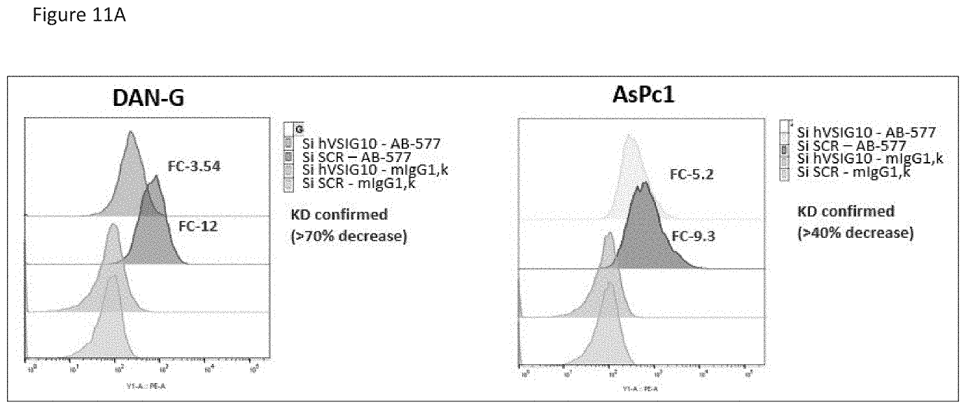

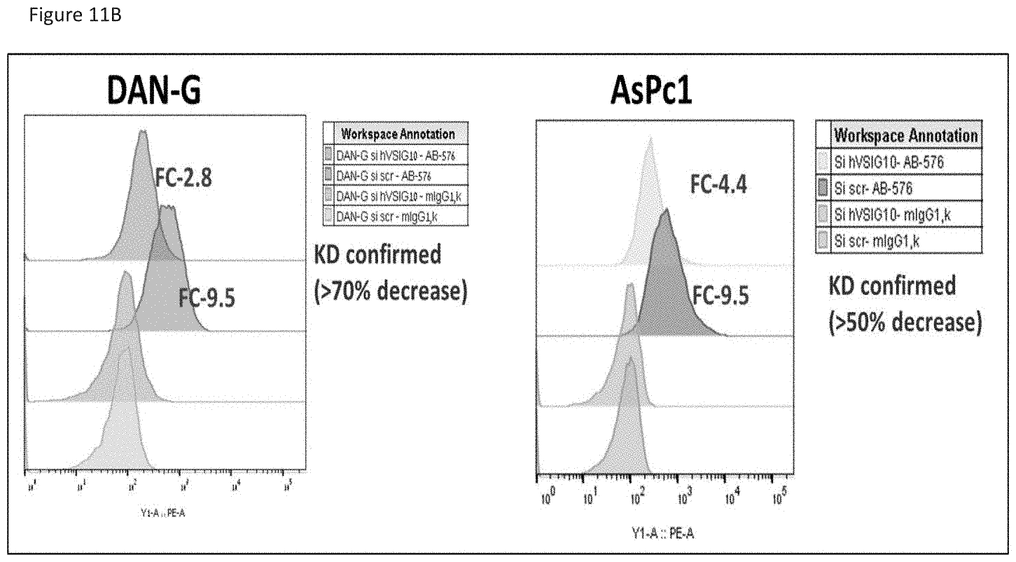

FIG. 11 shows membrane expression of human VSIG10 protein in DAN-G (left), AsPc1 (right) human cell lines transfected with human VSIG10 siRNA or non-target siRNA control. DAN-G and AsPc1 cells transfected with Human VSIG10 siRNA were stained with AB-577 (FIG. 11A), AB-576 (FIG. 11B) or with mIgG1,K isotype. Cells transfected with Scrambled siRNA were stained with AB-577 (FIG. 11A), AB-576 (FIG. 11B) or isotype control.

As shown in FIG. 11A membrane expressions of human VSIG10 protein using FACS analysis is reduced in cells transfected with VSIG10 siRNA. The MFI ratio of anti VSIG10 versus isotype control) in DAN-G cell line (left) is decreased from 12 fold to 3.54 fold using AB-577, and in AsPc1 cell line (right) from 9.3 to 5.2 fold.

As shown in FIG. 11B membrane expressions of human VSIG10 protein using FACS analysis is reduced in cells transfected with VSIG10 siRNA. The MFI ratio of anti VSIG10 versus isotype control) in DAN-G cell line (left) is decreased from 9.5 fold to 2.8 fold using AB-576, and in AsPc1 cell line (right) from 9.5 to 4.4 fold.

Endogenous expression of VSIG10 was also confirmed by siRNA knock down using AB-577 or AB-576 in DU-145 and ZR-75 human cell line (data not shown). These results confirmed both Ab specificity in the cells tested.

The mAb binding was evaluated on DU-145 human cell line endogenously expressing VSIG10. The affinity of AB-577 or of AB-576 was determined by FACS titration on DU-145 human cell line compared to isotype control antibody (mIgG1, kappa).

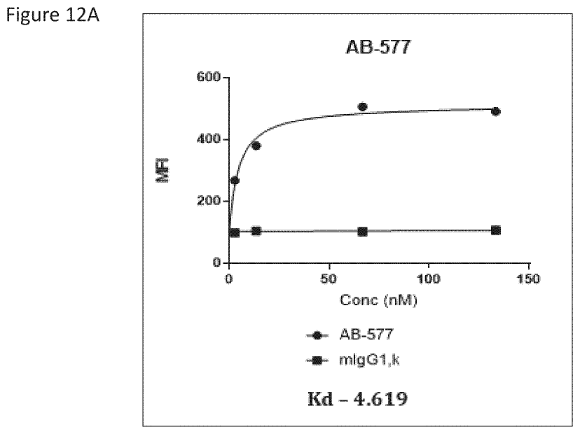

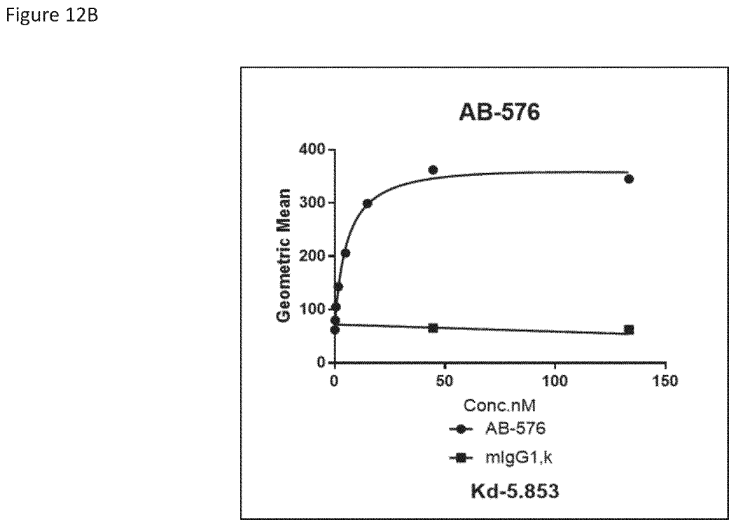

FIG. 12A shows affinity measurements using FACS application for the anti-human VSIG10 mAb AB-577 on DU-145 human cell line. DU-145 human cell line stained with AB-577 (dots) or stained with mIgG1, kappa (square) were analyzed by FACS in 4 concentrations. Detection was carried out using Goat Anti mouse-PE secondary Ab.

As shown in FIG. 12A, binding of AB-577 to DU-145 human cell line endogenously expressing VSIG10 protein (dots) compared to the isotype control (squares) was performed and the affinity was determined based on the Kd values calculated from the binding curve. The Kd value for AB-577 is 4.619(nM).

As shown in FIG. 12B, binding of AB-576 to ASPC-1 human cell line endogenously expressing VSIG10 protein (dots) compared to the isotype control (squares) was performed and the affinity was determined based on the Kd values calculated from the binding curve. The Kd value for AB-576 is 5.853 (nM). FIG. 12B shows affinity measurements using FACS application for the anti-human VSIG10 mAb AB-576 on ASPC-1 human cell line. ASPC-1 human cell line stained with AB-576 (dots) or stained with mIgG1, kappa (square) were analyzed by FACS in 8 concentrations. Detection was carried out using Goat Anti mouse-PE secondary Ab.

The performance of AB-577 and AB-576 against human VSIG10 were tested in IHC application at Smart Assay (Israel) on paraffin blocks of HEK293 cell over-expressing human VSIG10 Flag, DU-145 cells expressing endogenous VSIG10 and HEK293 cells transduced with an empty vector that were used as a negative control.

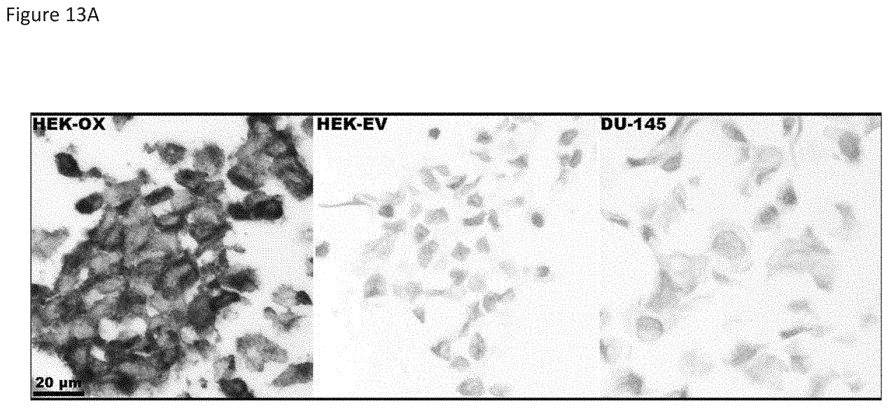

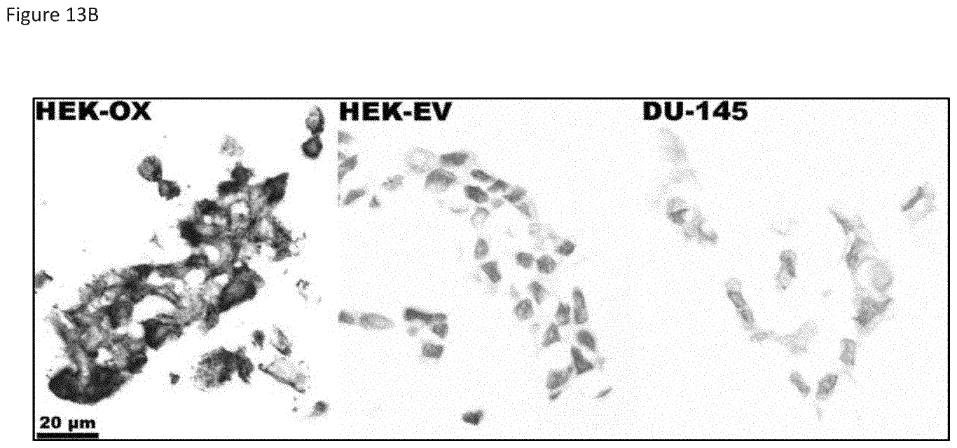

AB-577 or AB-576 were titrated on HEK293 OX cells, EV cells and DU-145 cells, as described in Material and Methods. Selected conditions of 10 ug/ml and two antigen retrieval methods (CA, CB) were used for further evaluation on human tissues. FIG. 13 represents the selected conditions. FIG. 13A shows high power microphotographs of cell blocks sections retrieved in CA and incubated with AB-577 (10 μg/ml). FIG. 13B shows high power microphotographs of cell blocks sections retrieved in CA and incubated with AB-576 (10 μg/ml).

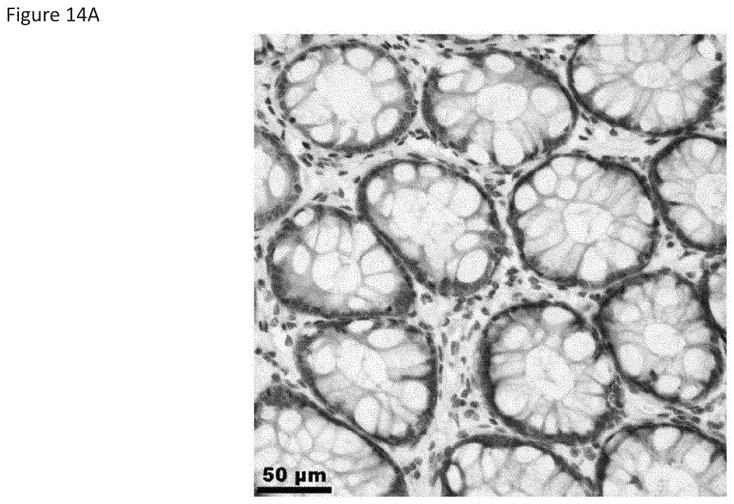

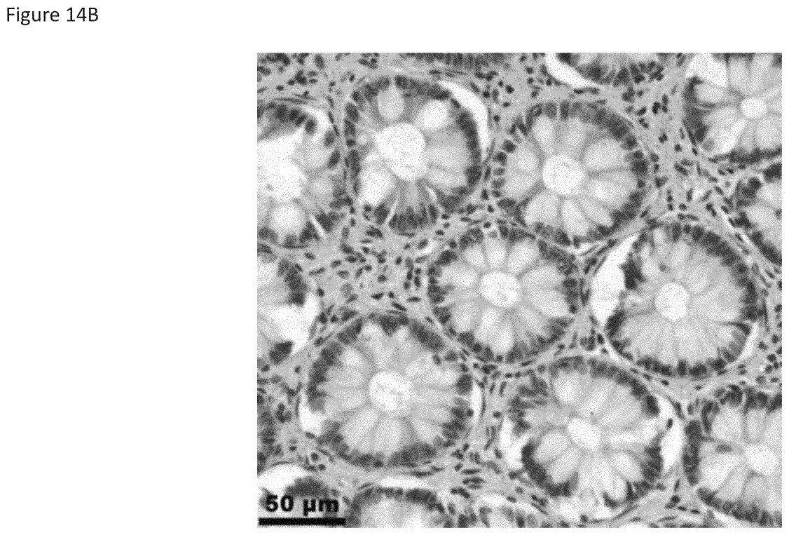

AB-577 was applied at the above condition on normal human colon and of non-small cell lung carcinoma (NSCLC) sample. Sections of the model cell block of HEK cells overexpressing VSIG10 (HEK-OX) were used as positive control. Three types of sections were mounted onto the same slides. No immunostaining was found in CB retrieved sections. Immunostaining in CA retrieved colon section is confined to epithelial cells, FIG. 14A. FIG. 14A shows a microphotograph of normal colon mucosa section immunostained with AB-577.

AB-576 was applied at the above condition on normal human colon and of non-small cell lung carcinoma (NSCLC) sample. Sections of the model cell block of HEK cells overexpressing VSIG10 (HEK-OX) were used as positive control. Three types of sections were mounted onto the same slides. No immunostaining was found in CB retrieved sections. Immunostaining in CA retrieved colon section is in Muscular layer of the colon shows “patchy” immunostaining of smooth muscle cells and immunostaining of Schwann (but not ganglion) cells of myenteric plexus, FIG. 14B. FIG. 14B shows microphotograph of normal colon mucosa section immunostained with AB-576.

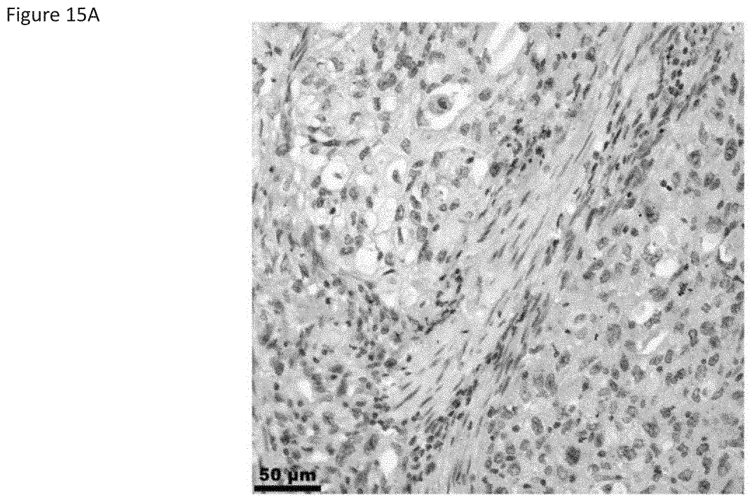

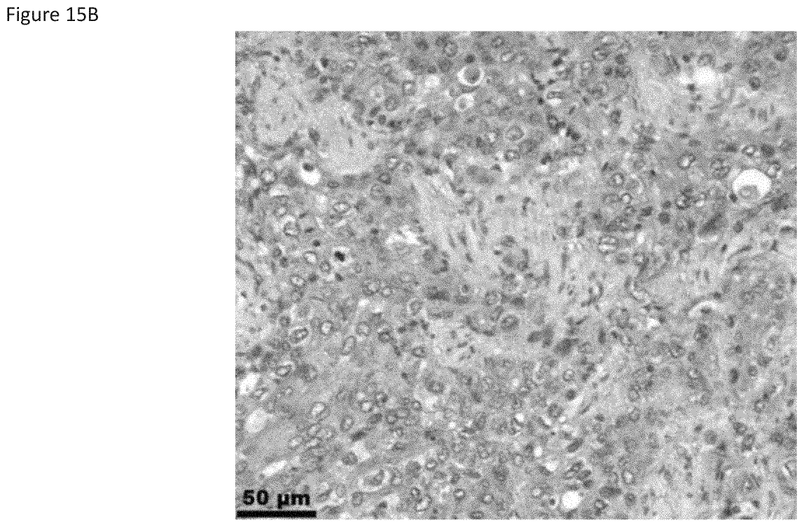

NSCLC sample shows very weak cytoplasmic staining in cancer cells with no staining in other cells types, FIG. 15A. FIG. 15A shows a microphotograph of NSCLC sample section immunostained with AB-577. Cancer cells show very weak cytoplasmic immunostaining. No staining is seen in stromal cells.

Cancer cells show cytoplasmic immunostaining. Little or no staining in stromal cells, FIG. 15B. FIG. 15B shows microphotograph of NSCLC sample section immunostained with AB-576 Cancer cells show cytoplasmic immunostaining. Little or no staining in stromal cells.

### Example 3: VSIG10 Immunohistochemistry Expression Study (Smart Assay)

The aim of the study was to identify the optimized antibody and assay conditions, for VSIG10 custom antibodies and to assess VSIG10 expression, using immunohistochemistry (IHC) in formalin-fixed, paraffin-embedded (FFPE) human tissues sections.

The IHC calibration study was performed at Smart Assay (Israel). The study employed screening of custom anti VSIG10 monoclonal antibodies at five concentrations on positive and negative control human cell lines and human samples from colon cancer, colon normal and Non-Small Cell Lung cancer (NSCLC) tissues, using FFPE sections (4 μm) of the cells and tissues blocks treated with three antigen retrieval methods.

Selected Abs were used to analyze VSIG10 expression in several TMAs. These studies are on-going. In this report, AB-577 was employed on lung TMA as well as on lung adenocarcinoma full face sections.

VSIG10—IHC Study on Tissue Microarrays (TMAs)

One section of the lung cancer tissue array with normal lung tissue (Biomax, cat.BC041115d) was immunostained with anti human VSIG10 mAb AB-577 according to the previously established protocol at the calibration stage. Parallel sections was incubated with isotype control antibody as the negative control and with anti CD-34 rabbit monoclonal antibody (clone EP373Y, abcam, cat.ab81289) as the positive control to demonstrate “stainability” of the tissue cores. Sections of normal human colon, NSCLC and cell block comprising HEK293 cells overexpressing VSIG10 samples used for the protocol establishment were processed in parallel with the tissue arrays sections as the positive control. For each core the relative number of stained cancer cells with AB-577 and intensity of immunostaining were assessed. Intensity of the staining is presented in a semi-quantitative fashion: 0—no staining, 1—weak staining, 2—moderate staining, 3—strong staining. The IHC score (H-Score) was generated by multiplying the intensity of immunostaining by the approximate percentage of correspondingly stained cells. The cut off is 10% (i.e. if less than 10% of cells are stained the sample is regarded as negative—score=0).

Results

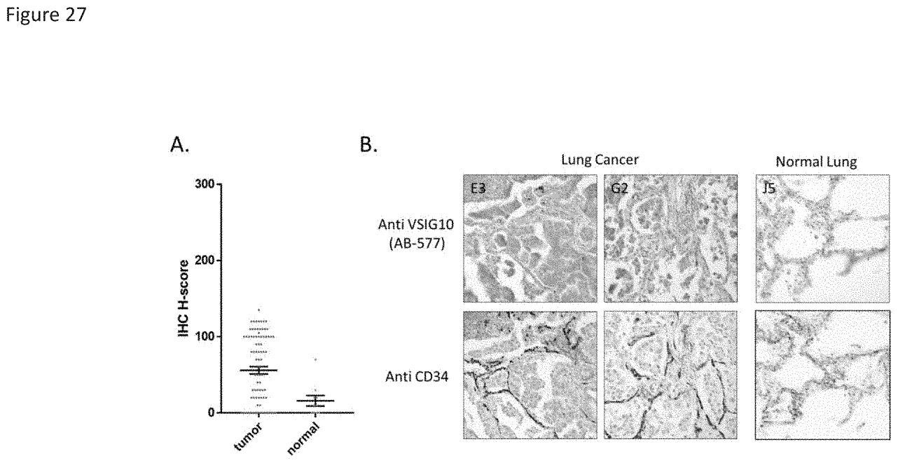

AB-577 anti-human VSIG10 mAb was tested in IHC at Smart Assay (Israel) on lung FFPE TMA (Biomax, cat.BC041115d) containing 110 cancer and 10 normal cores. As shown in FIG. 27, VSIG10 staining with AB-577 is significantly higher in the tumor cores, compared with the normal cores. The staining pattern observed shows weak to moderate cytoplasmic immunostaining of cancer cells and strong immunostaining of endothelial cells within stromal septa surrounding nests of cancer cells. In the normal sections, weak or no immunostaining was observed. Most of the immunostained cells could be identified as endothelial cells while majority of pneumocytes (alveolar epithelial cells) appeared to be non-stained. The TMA was stained for CD34 to assess cores quality across the samples. As shown in FIG. 27B (lower panel) comparable staining is observed. FIG. 27A shows IHC staining of lung cancer samples (n=110) vs. normal lung tissue (n=10) scores. Graph shows mean±SEM (P<0.01). FIG. 27B shows microphotograph of cancer and normal lung sections immunostained with AB-577 (upper panel) and anti-CD34 (lower panel).

VSIG10—IHC Study on Full Face Sections of NSCLC Tissue

Ten sections of full face lung cancer tissues (Asterand: 1173336B, 388042C1, 1180033B, 1197680B, 1201822B, 1071274B, 1195121B, 1189482B, 1092793B, and 1224263B) were immunostained with anti human VSIG10 mAb AB-577 according to the previously established protocol at the calibration stage. Parallel sections were incubated with isotype control antibody as the negative control.

For each section the tumor region was analyzed as well as two non-tumor adjacent regions (alveolar and bronchial). The staining of the cells with AB-577 and intensity of immunostaining were assessed.

Results

AB-577 anti human VSIG10 mAb was tested in IHC application at Smart Assay (Israel) on lung FFPE samples as described above. Analysis was performed separately on the tumor region, normal alveolar region, and normal bronchial region. As shown in Table 1, VSIG10 expression is positive in 7 out of 10 cancer tissues, and only in one case of normal lung tissue (1092793B). There is positive staining in 4 samples in the alveolar macrophages. Due to the lack of specific macrophages marker this staining could not be determined as VSIG10 specific but could be due to the cells origin. In 3 cases staining is observed in the endothelial cells, mostly in the large vessels. The combined data suggest higher expression in tumor region compared with normal region.

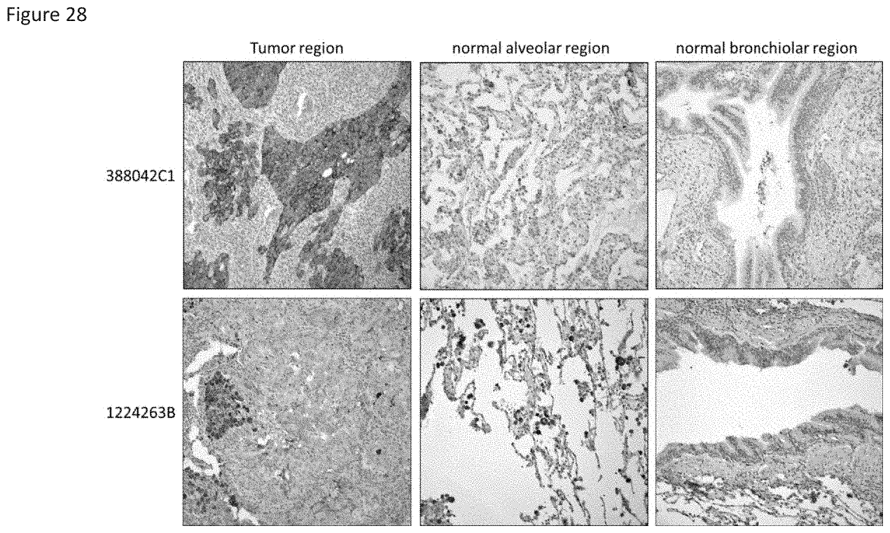

FIG. 28 present two cases of full face section staining. In the upper panel 388042C1 section, showing prominent staining of most of cancer cells and no staining in normal lung tissue (both alveolar and respiratory epithelia). Few stained cells in the normal regions are macrophages. In the lower panel 1224263B section, showing moderate staining of most of cancer cells and no staining in normal lung tissue. In the alveolar region stained cells are alveolar macrophages, and in the bronchial region respiratory epithelium staining is very weak to none, few stained cells in the lumen are alveolar macrophages.

FIG. 28 shows microphotograph of tumor and normal regions of NSCLC sample 388042C1 (upper panel) and 1224263B (lower panel) immunostained with AB-577.

TABLE 1
Summary of anti human VSIG10 mAb Ab-577 IHC staining in
cancer and non-tumor adjacent regions of 10 lung adenocarcinoma sections.

| | | | Alveolar | Respiratory | | |
|---|---|---|---|---|---|---|
| Sample | Ca cells | Pneumocytes | macrophages | epithelium | Endothelial cells | Notes |
| 1173336B | − | − | non-specific | − | − | granular cytoplasmic staining in single |
| | | | | | | connective tissue cells (mast |
| | | | | | | cells?); staining of alveolar macrophages |
| | | | | | | apparently non-specific: |
| | | | | | | pprominent staining of these cells in |
| | | | | | | “isotrype control” section |
| 388042C1 | + to +++ | − | occasional | − | occasional in | granular cytoplasmic staining in single |
| | | | | | larger vessels, | connective tissue cells (mast cells?) |
| | | | | | not in capillaries | |
| 1180033B | − to + | NP | NP | NP | − | |
| 1197680B | − | NP | − | NP | − | |
| 1201822B | − to + | − | + | NP | + in larger | part of the section lost |
| | | | | | vessels, not in | |
| | | | | | capillaries | |
| 1071274B | − to + | − | − | − | − | staining very weak |
| 1195121B | ± | − | + | − | − | very weak staining in few foci of Ca cells |
| 1189482B | − | NP | NP | NP | − | |
| 1092793B | + to ++ | − to + | + | + to ++ | + in larger vessels | weak to moderate staining in multiple |
| | | | | | and in some | cell types: stromal cells, smooth muscle |
| | | | | | capillaries | cells; chondrocytes; glandular epithelium |
| 1224263B | − to ++ | − | + | ± | + in larger | very weak if any staining in resp, |
| | | | | | vessels, not in | |
| | | | | | capillaries | |

### Summary

In this study, monoclonal Ab for IHC application was calibrated, to assess CGEN VSIG10 expression in tissues.

Few Abs were identified as potential Abs that can be further use in IHC studies. AB-577 was further processed to evaluate VSIG10 expression pattern in NSCLC tissues.

Preliminary data generated on one TMA consist of 110 cancer individuals indicates differential expression in the NSCLC samples as compare to the staining observed in the normal lung tissue samples.

### Example 4: Effect of VSIG10 on TILs Specific Response to Human Melanoma

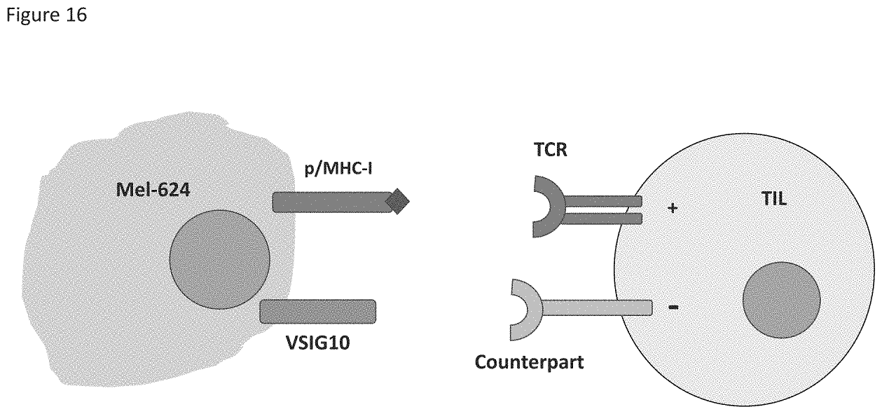

Mel-624 is HLA-A2 positive human melanoma cell line, which express MART-1 and gp-100 antigens. We have co-cultured TILs with cognate mock or VSIG10-transduced MEL-624 melanoma cells, to assess an effect of VISIG10-transduced on TILs activity. (FIG. 16). An inhibitory effect of VSIG10 protein in TILs specific response to melanoma cells, demonstrated below, could supports the hypothesis that VSIG10 is an immune modulator of tumor-specific T cells.

FIG. 16 presents an illustration of the experimental system utilizing Mel-624 cells over-expressing VSIG10 and being used for activating melanoma derived T cells (TILs) with antigen specificity for either gp100 or MART 1 derived peptides.

A differential magnitude of T cell activation is expected between VSIG10 over expressing and mock transduced melanoma cell lines.

TIL:Mel-624 assay was established as a tool to screen protentional immune-modulatory ligands. The aim of the study described herein was to evaluate the potency of VSIG10 to modulate function of human melanoma-derived TILs co-cultured with melanoma target cells with forced expression of VSIG10.

Materials and Methods

Materials

Reagents

Iscove's Modified Dulbecco's Medium-IMDM (Biological Industries, 01-058-1A).

Dulbecco's Modified Eagle's Medium-DMEM (Biological Industries, 01-055-1A)

RPMI 1640 (Biological Industries, 01-100-1A)

Human Serum (Sigma, H3667)

Fetal Bovine Serum-FBS (Biological Industries, 04-127-1A)

Glutamax (Life technologies, 35050-038)

Na-Pyruvate (Biological Industries, 03-042-1B)

Penicillin-Streptomycin Solution (Biological Industries, 03-031-1B)

MEM Non-Essential Amino Acids Solution (Biological Industries, 01-340-1B)

HEPES (Biological Industries 03-025-1B)

Cell dissociation buffer (Life technologies, 13151-014)

Recombinant human IL2 (Biolegend, 589106)

Trypan blue, 0.4% diluted (Biological Industries, 03-102-1B)

PBS (Biological Industries, 02-023-1A)

Viability dye (BD horizon, 562247)

Human Trustain FcX (Biolegend),

APC/Cy7-anti human CD8a (Biolegend, 300926; 309804)

PE-anti human CD137 (Biolegend, 309804)

PE-anti HLA-A2 (Biolegend, 3443306)

APC-anti PDL1 (Biolegend, 329708)

CBA Human Th1, Th2, Th17 cytokine kit (BD Biosciences, 560484)

Mouse anti humanVSIG10-Ab577 10 ug/mL (Genscript)

mIgG1 10 ug/mL (Biolegend, 400166)

PE AffiniPure F(ab′)₂ Fragment Goat Anti-Mouse IgG (H+L) 1:100(Jackson ImmunoResearch, 115-116-146)

Equipment

Incubator 37° C., 5% CO2 (Binder)

Centrifuge (Eppendorf, 5810R)

Countess II cell automated counter (life technologies)

Plate shaker DTS4

Macsquant analyzer (Milteneyi)

Cells

Tumor-infiltrating lymphocyte (TIL) micro-cultures were initiated and expanded from tumor specimens taken from resected metastases of melanoma patients (as described in Uzana et al, JI 2012). Briefly, TILs were cultured for 14 days in complete medium and IL-2. After initiation period cells stained with FITC-conjugated HLA-A*0201/MART-126-35 dextramer (or gp100209-217) and anti-human CD8. CD8+ lymphocytes, positively stained by the dextramer (CD8+/dextramer+ cells), were sorted by a BD FACSAria (BD Biosciences) and directly cloned at one or two cells/well in 96-well plates in the presence of ortho-anti-CD3, rhIL-2 (6000 IU/ml), and 4 Gy-irradiated allogeneic PBMCs as feeder cells. After 5 days IL-2 was added and renewed every 2 days until day 14, then the clones were assayed for IFNγ secretion in a peptide-specific manner following their co-incubation with MART-126-35-pulsed T2 cells using ELISA reagents. The MART-126-35-reactive clones were further expanded in a second-round exposure to ortho-anti-CD3 (30 ng/ml) and (6000 IU/ml) rhIL-2 in the presence of 50-fold excess of irradiated feeder cells. This study was conducted in Haddassa medical institute. The TILs, which were sent to Compugen LTD under service agreement, kept in liquid nitrogen (10-20×10⁶/vial) and thawed one day before co-culture with target cells. Tumor-infiltrating lymphocytes (TILs) from resected metastases of three melanoma patients were used:

TIL-209-HLA-A2-gp100 specific (CL-309)

TIL-463-F4-HLA-A2-gp100 specific (CL-311/2)

TIL-463-F5-HLA-A2-gp100 specific (CL-313/4)

TIL-412-HLA-A2-Mart1 specific (CL-315)

Human melanoma cells Mel-624 (CL-218) overexpressing VSIG10 or PDL1 or empty vector transduced cells (mock) as control (transduction described in methods section).

Methods

Cell Culture

TILs were cultured in IMDM supplemented with 10% human serum, 1% Glutamax, 1% Na-Pyruvate, 1% MEM Non-Essential Amino Acids Solution, 1% Penicillin-Streptomycin Solution, 300 IU/ml of rhIL2. Cells were seeded in T75 standing flask (suspended cells) 24 hr prior to co-culture in 37° C., 5% CO2 incubator.

Mel-624 transduced cells were cultured in DMEM supplemented with 10% FBS, 10 mM HEPES, 1% Glutamax and 1% Penicillin-Streptomycin Solution. cells were thawed 5 days prior to co-culture in T75 flask (adherent cells) and cultured for 30 days in which co-cultures with TILs were done.

Gene Over-Expression in Mel-624 Cells

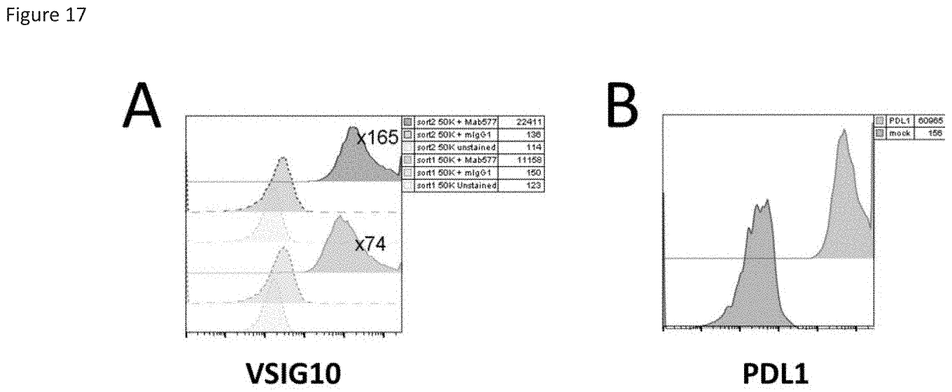

To asses VSIG10 effects on TILs as a ligand on target cells we overexpressed VSIG10 gene in Mel-624. Briefly, full length cloning of human VSIG10 construct containing flag tag in internal domain was performed at Sirion Biotech by gene synthesis for optimized sequence and cloned into lentiviral expression plasmid pcLV-CMV-MCS-IRES-Puro to produce Lentivirus particles (virions). Mel-624 cells were transduced using lentivirus particles (HIV-based, VSVG pseudotyped) with Hexadimethrine bromide (polybrene catalog number H9268). A no gene construct (pcLV-CMV-MCS-IRES-Puro) was used as negative control (designated Mel-624/mock). Human PDL1 transduced target cells were established in parallel to Mel-624/VISIG10 cells and were used as a positive control in the assay (designated Mel-624/PDL1). Puromycinresistant cells were used to generate stable pool, banked, thawed 3-5 days prior to assay and cultured (full medium as described above supplemented with 0.5 ug/mL Puromycin (InvivoGen, ant-pr)) for no more than 30 days. Since low expression levels of VSIG10 were shown 30×10\hat{ }6 transduced cells from stable pool were stained with anti VSIG10 antibody followed by sorting out of high-expressing cell population using FACS sorter (FIG. 17). This process was repeated on 2 independent population of cells (from stable pool batch). This cell population was banked separately and designated Mel-624-hVSIG10 high1 (sorting protocol 17-1933) and Mel-624-hVSIG10 high2 (sorting protocol 17-1931). Over expression validation is presented in FIG. 17.

FIG. 17 shows that sorted, transduced Mel-624 over-express VSIG10. 5e4 cells were stained with viability dye, washed and stained with anti-VSIG10 (Mab577) followed by a secondary antibody goat anti mouse-PE or anti PDL1-APC. Dead cells were subtracted from the analysis. A sort1/high1 over expressing cells (light blue) are lower in overexpression compared to sort2/high2 over expressing cells (dark blue). B. PDL1 is over expressed in 390-fold higher than the control.

Co-Culture

TILs are thawed 24 hr before as described above, washed twice with wash media (RPMI, 10% FBS, 1% Pen-Sterp) to dispose of residual IL-2 and seeded in 96-well tissue culture plate 5e4 cells per well. Mel-624 over expressing VSIG10 or PDL1 or mock cells were harvested with cell dissociation buffer, washed twice with wash media and seeded in co-culture plate (pre-seeded with TILs) 5e4 cells per well. Effector to target ratio—1:1. Final co-culture volume 200 uL. 96-well plate was incubated overnight (˜18 hr) in 37° C., 5% CO2 incubator. Seeding in plate edges (row A/H; column 1/12) was refrained. Each test was done in quadruplets. A total of 4 experiments were done per each TIL. Due to HLA-A2 discrepancy in 2 experiments the control used was parental cells Mel-624.

HLA-A2 and Target Validation

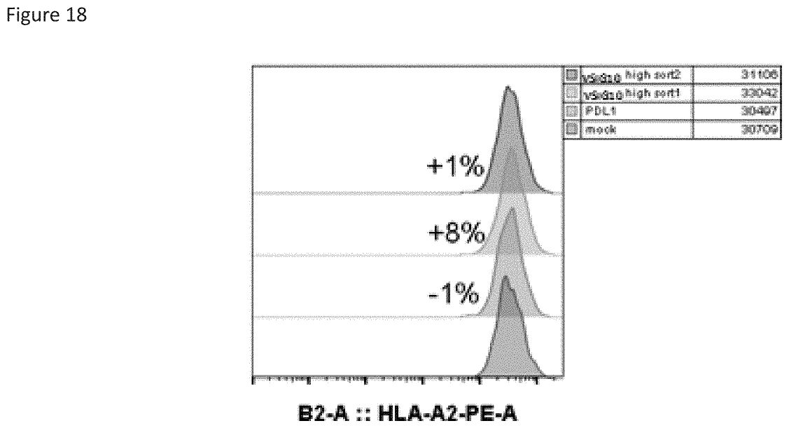

Mel-624 cells used for co-culture were stained for viability dye, washed and stained with PE-anti-HLA-A2 or APC-anti-human PDL1 or anti VSIG10 for 30 min in 4° C. Cells were run In MACSquant and expression was analyzed by FlowJo software. <30% discrepancy in HLA-A2 levels between the tested Mel-624 cells VSIG10 or PDL1 and the control were admissible (FIG. 18). This validation was done at each experiment repeat on the same cells that were harvested and seeded for co-culture. Representative data as presented in FIG. 18.

FIG. 18 shows that sorted, transduced Mel-624 over-express VSIG10. 5e4 cells were stained with viability dye, washed and stained with PE-anti-HLA-A2. Dead cells were subtracted from the analysis. All over expressing populations express comparable HLA-A2 levels.

Assessment of TILs Functional capacity with staining for activation marker is shown.

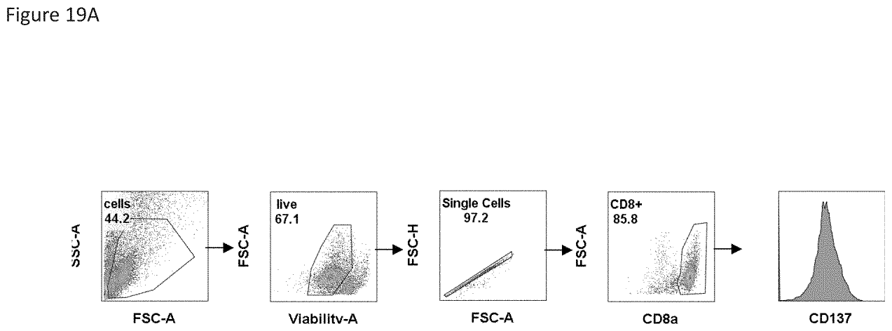

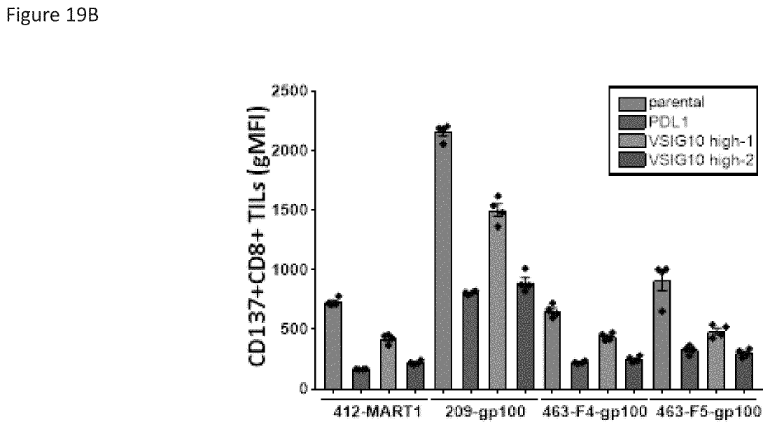

T cell activity was assessed based on detection of IFNγ in co-culture supernatants or by measuring changes in activation marker CD137 surface expression. After 18 hr co-culture plate were centrifuged. Cell pellets were stained with viability dye, washed and stained with antibody mix anti human-CD8a-APC/Cy7, anti-human-CD137-PE (Biolegend, 300926; 309804) and human Trustain. After 30 min incubation at 4° C. samples were washed and run in MACSquant (Milteney). Representative data is shown in FIG. 19.

FIG. 19 shows gMFI mean values on gated CD8+ TILs after co-culture with Mel-624 cells. Cell pellet from co-culture were stained with viability dye, washed and stained with APC/Cy7-anti-human-CD8a, PE-anti-human-CD137 and Trustain. A. gating strategy B. representative data.

Cytometric Bead Array-CBA

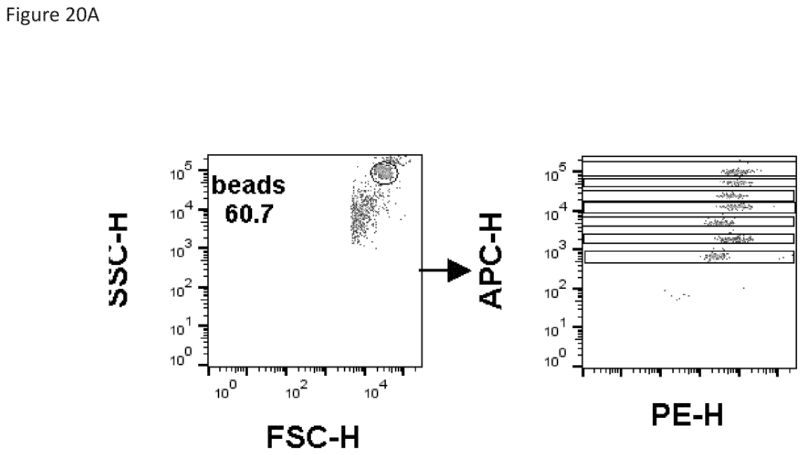

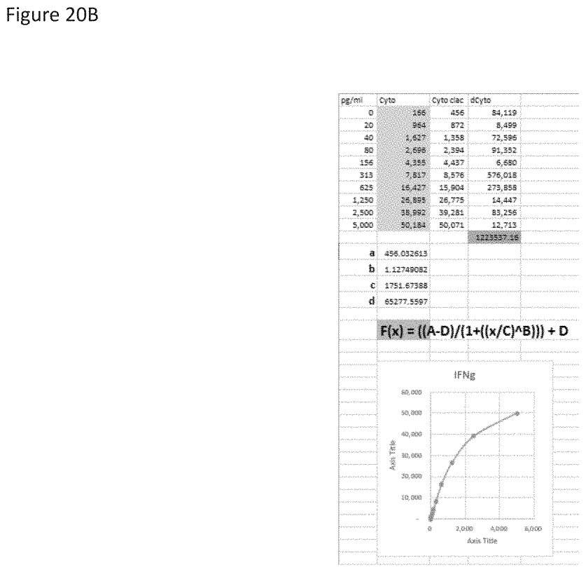

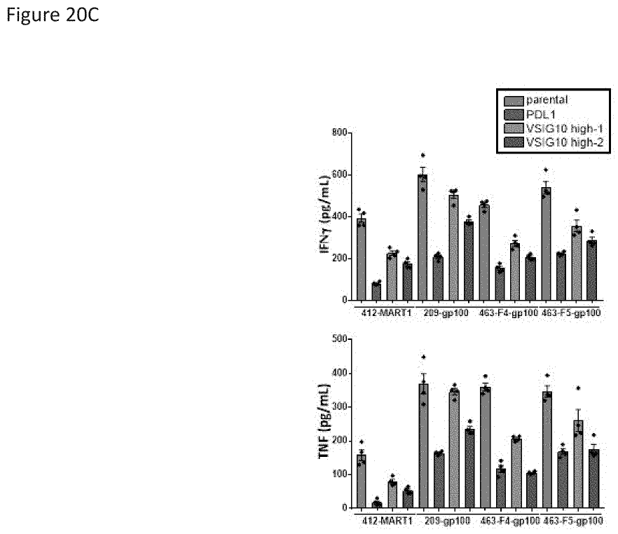

Co-culture supernatants were collected after overnight co-culture and tested for cytokines by CBA. In brief, capture antibody bead mix was prepared by adding 2 uL/sample per each cytokine capture bead and 12.5 uL mix was added to 96 well polypropylene plate. 50 uL supernatant was then added followed by 12.5 uL detection reagent and spin down. Plates were incubated in plate shaker for 3 hr, 1500 rpm, washed and read in MACSquant. Representative data is shown in FIG. 20.

FIG. 20 shows IFNg and TNFa secreted from TILs after co-culture with Mel-624 cells. Co-culture supernatant is analyzed by CBA kit. gMFI of PE-H channel is plotted per each cytokine and by creating a standard curve the amount of secreted cytokines is calculated. A. gating strategy. B. representative standard curve for IFN gamma. C. representative data secreted.

Data Analysis

All FACS files were analyzed by FlowJo software.

Two tailed paired parametric T-test was calculated for 4 repeats per TIL or by comparing 4 TILs to each other. P<0.05 was referred to as statistically significant

Results

VSIG10 Mediates an Immune-Modulatory Effect on TILs

4 co-culture experiment were conducted, in each 4 TILs were used (total n=4 per TILF).

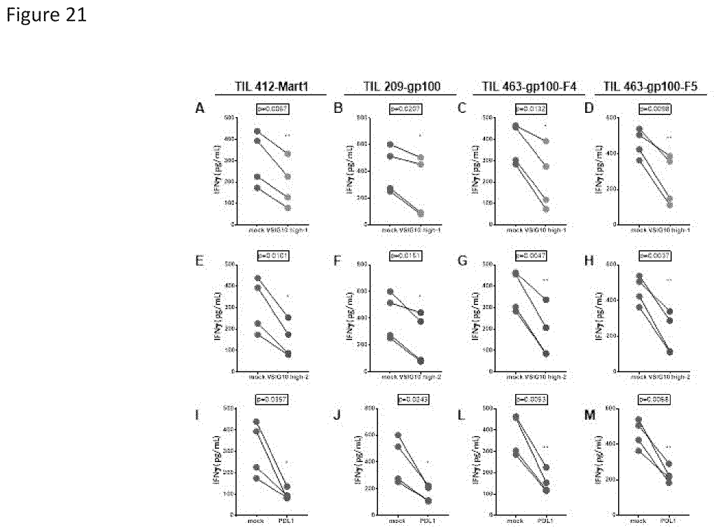

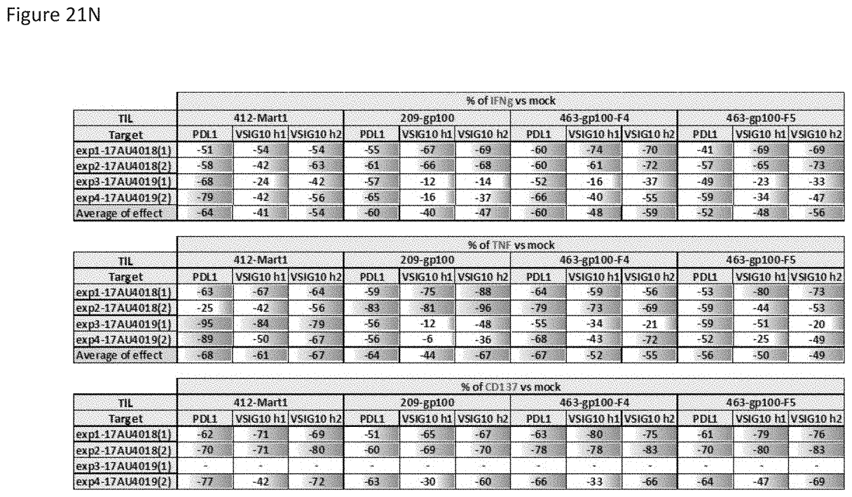

FIG. 21 shows that Mel-624 over expressing VSIG10 inhibits IFN gamma secretion from TILs supernatant from TIL. Mel co-cultures were collected and tested for cytokines by CBA kit. IFN gamma secretion upon TILs co-culture with Mel-624 over expressing VSIG10 or PDL1 or Mel-624 mock is plotted. response to over expressing cells VSIG10 high1 (light blue A-D), VSIG10 high2 (dark blue E-H) and PDL1 (red I-M) is compared to TILs response to control Mel-624-mock (grey). Each dot represents the mean of quadruplets in a single experiment. n=4 per TIL, p-values are plotted above each graph.

FIG. 21N shows that Mel-624 over expressing VSIG10 mediate an inhibitory effect on TILs. The mean of quadruplets for each over-expressing cell was compared to the mean of the control. The percentage of effect is indicated for IFN gamma, TNF and CD137 readouts. CD137 expression in exp3 was not obtained due to technical issue.

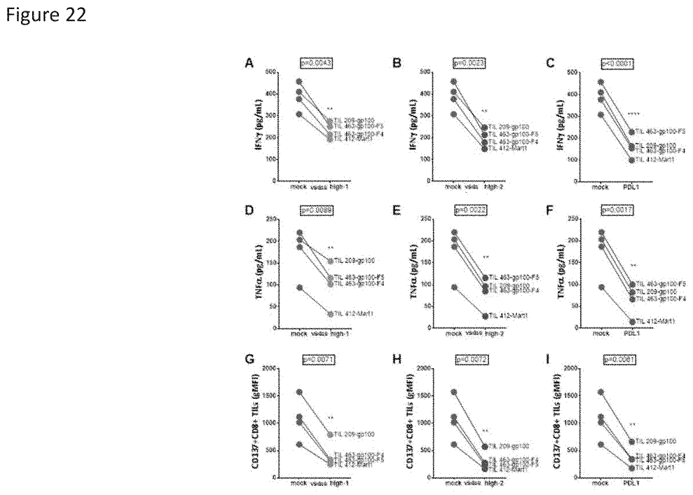

FIG. 22 shows that Mel-624 over expressing VSIG10 inhibit TILs secretion of IFNg/TNFa and CD137 expression. Supernatant from TIL:Mel co-cultures were collected and tested for cytokines by CBA kit. Cell pellet was stained for CD137 and CD8. IFN gamma (A-C), TNFa (D-F) secretion and CD137 expression (G-I) are plotted. Response to over expressing cells VSIG10 high1 (light blue A,D,G), VSIG10 high2 (dark blue B,E,H) and PDL1 (red C,F,I) is compared to TILs response to control Mel-624-mock (grey). Each dot represents 4 experiments per same TIL. p-values are plotted above each graph.

IFN gamma levels secreted from TILs in co-culture with melanoma cells over expressing VSIG10 was decreased in 4/4 TILs tested and compared to the control (FIG. 21 A-H, FIG. 22A-B). Accordingly, TNFa secretion was decreased and CD137 was downregulated (FIGS. 22 D-E and G-H, respectively). The magnitude of the inhibitory effect varies across experiments (FIG. 21N) however these inhibitory effects were found statistically significant and assay was validated with positive control PDL1 (FIG. 21 I-M). Without wishing to be limited by a single hypothesis, was presumed that Mel-624/VSIG10-high2 could have a more pronounced suppressive effect on TILs than of Mel-624/VSIG10-high1due to higher expression level of VISIG10 in ‘high2’ cells, the observed trend has not reached statistical significant. The effects described above were also compared between the different TILs and were found statistically significant.

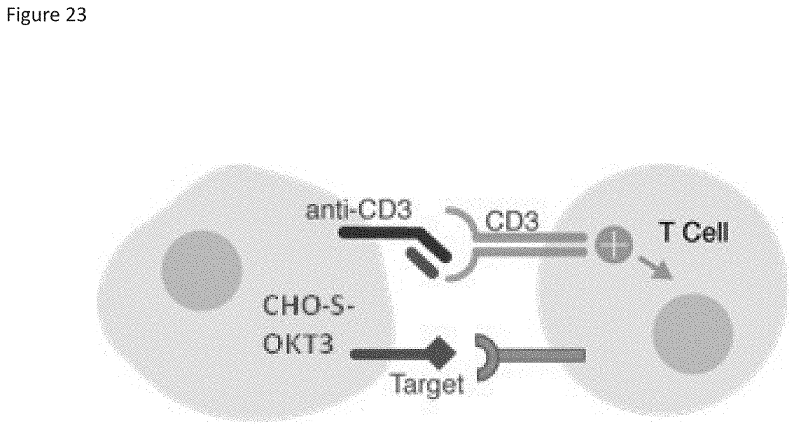

Preliminary data in an additional experimental system points-out for a T cell inhibitory effect for VSIG10 (data not shown). In this experimental system, schematically illustrated in FIG. 23, VSIG10 is co-expressed on CHO-S cells with a membrane bound form of anti-CD3 (clone OKT3). CHO-S OKT3 cells ectopically expressing (by viral transduction) VSIG10 or empty vector were co-incubated with primary T cells isolated from peripheral blood of healthy donors. T cell activation was measured after 5 days of co-incubation.

It was demonstrated herein that VSIG10 over expression on Mel-624 induces an inhibitory effect on TILs. This effect is manifested by a decrease of IFN gamma and TNFa secretion and downregulation of the activation marker CD137. These effects are shown in 4 independent experiments each testing 4 different TILs, three of which are gp100 specific and one is MART-1 specific. Assay was validated with a relevant positive control, PDL1. The sum of effects seen across 4 repeats induced by VSIG10 meets assay criteria by which a >30% assay window is required. These results provide in-vitro functional validation for VSIG10 that support the hypothesis that VSIG10 is an inhibitory immune-modulator.

FIG. 23 is an illustration of the experimental system utilizing CHO-S-OKT3 cells over-expressing VSIG10 and being used for poly-clonal activation of primary T cells.

In a first set of experiments, transient expression of VSIG10 resulted in marked inhibition of T cell activation as demonstrated by reduced cytokine secretion and reduced expression of activation markers. Additional experiments with stable transduction of VSIG10 exhibited T cell inhibition in some of the tested donors but didn't exhibit inhibition in others. In some of the experiments, anti-VSIG10 Ab#577 was added and caused a partial restoration of the VSIG10-mediated T cell inhibition.

These results demonstrate an inhibitory effect for VSIG10 in an additional reductionist experimental system. Furthermore, it was demonstrated that the VSIG10 mediated T cell inhibition obtained in this experimental set-up can be reversed by an anti-VSIG10 antibody.

In line with the effect observed in human system, over expression of murine VSIG10 in artificial antigen-presenting cells resulted in reduced activity of TCR-transgenic DO11.10 T cells.

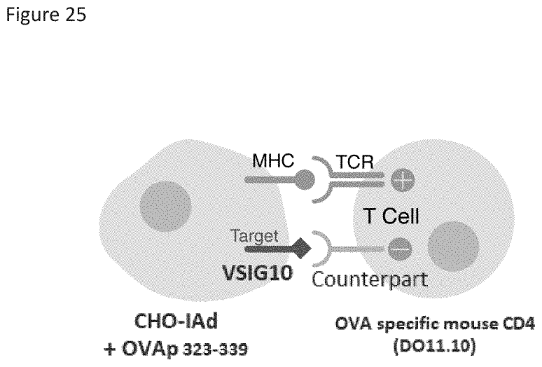

Schematic illustration of the CHO-S-IAd experimental system used for this experiment is described in FIG. 25.

Briefly, VSIG10 was over expressed on CHO-S cells ectopically expressing IAd. The CHO-S-IAd cells (over-expressing VSIG10 or empty vector) were loaded with different concentrations of OVA peptide and co-cultured with CD4⁺ cells isolated from spleens of DO11.10 mice (transgenic mice expressing T cells with a restricted anti-OVA CD4 T cell receptor repertoire). After 5 days of co-culture, CD4 T cells were harvested and T cell proliferation and cytokine secretion were measured.

Table 3 below presents information on the CHO-S-IAd DO11.10 experimental system including cells in the assay, time of incubation and readouts.

TABLE 3

| | |
|---|---|
| Effector cells | CD4+ cells (DO11.10 Tg mice) |
| Target | Mitomycin C-treated CHOS-IAd |
| | (MHC-II) overexpressing |
| | hVSIG10/PDL1/Mock (empty vector) |
| E:T | 5:1; 1X10⁵ T cells and 2X10⁴ CHO-S-IAd+ |
| | cells per well |
| Time ioint | 5 day post co-culture |
| Readouts | FACS: T cells proliferation IFNγ |
| | and TNF and IL-2 (CBA) |

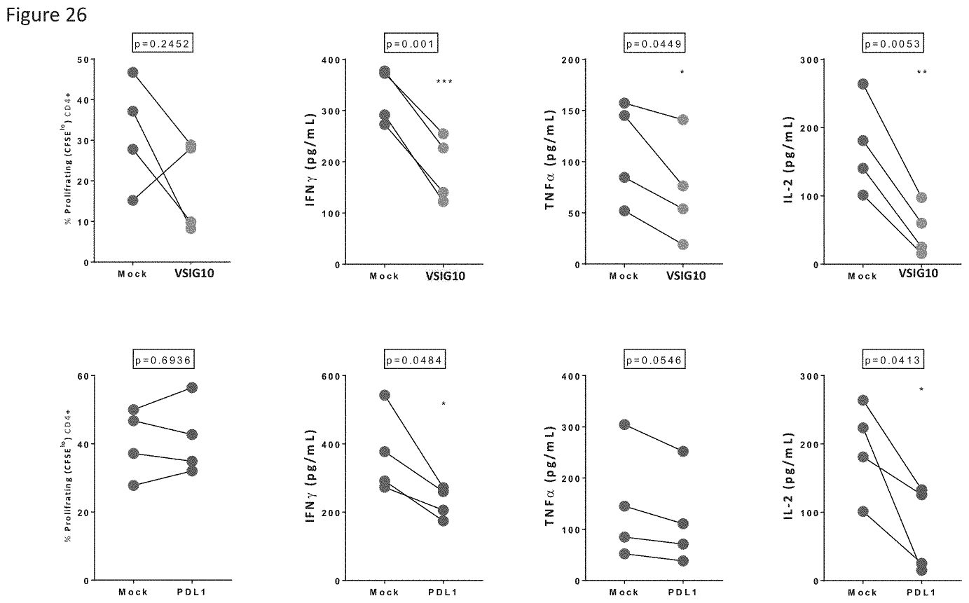

As seen in FIG. 26, VSIG10 over expression on CHO-S-IA^{d} cells mediates

an inhibitory effect on DO11 derived CD4 T cells as manifested by Cytokine secretion and proliferation in a similar magnitude to the effect mediated by PDL1 over expression

### Example 5: In Vivo Study-Effect of VSIG10 Gene Depletion on MC38 Tumor Growth

VSIG10 protein was identified as a novel co-signaling molecule which serves as a coinhibitory ligand for T cell activation. To better understand the role of VSIG10 in immune responses, VSIG10 knockout (KO) mice were generated. In the studies presented in this report, the function of VSIG10 as a novel immune checkpoint was tested by monitoring tumor growth in VSIG10-KO mice relative to wild-type mice.

This Example was done to study the effect of mVSIG10 gene depletion on in vivo growth of MC38 murine colon carcinoma model with and without anti-PDL1 treatment.

Materials and Methods

2.1 Tumor Challenge Experiments:

MC38 colon carcinoma cells were kindly provided by Dr. Charles G. Drake. Cells were cultured in RPMI 1640 (GIBCO) with 10% heat-inactivated FBS (Atlanta Biologicals). For tumor implantation, cells were harvested and washed, counted and suspended to 5×10⁶ cells/ml in cold PBS and placed on ice. VSIG10 KO mice were generated at Ozgene Pty Ltd (Bentley WA, Australia). C57BL/6 Wild-type litter-mate mice from Ozgene served as experimental controls. All mice were 6-8 week old females. The posterior right flank was shaved and disinfected with a 70% Ethanol solution. Tumor cells (0.5×10⁶) were injected subcutaneously into the back-right flank of mice in a volume of 100 ul. Dosing of anti-PDL1 and isotype control mAb was initiated on day 14 post tumor implantations when tumor volumes across groups were in the range of 250-450 mm3; mAbs were administered intra-peritoneally (i.p.) in a final volume/injection of 100 ul every 3 days for 2 weeks for a total of 4 doses. Tumor growth was measured with electronic caliper every 3 days and was reported as 0.5×W2×L mm3. Mice were euthanized with CO2 at either study termination or any of the following clinical endpoints: tumor volume ≥5000 mm3, tumor ulceration, body weight loss ≥20%, or moribund appearance. Mice were maintained in an SPF animal facility for at least 1 week prior to beginning the experiment. All studies were approved by the Institutional animal care and use committee at the Johns Hopkins University (Baltimore, Md., USA).

2.2 Antibodies:

The anti-mouse PDL1 mAb (clone 10F.9G2; Bio X Cell, West Lebanon, N.H., USA) used in this study was described previously [Eppihimer et al, 2002]. The Rat IgG2b (clone LTF-2; Bio X Cell, West Lebanon, N.H., USA) was used as an isotype control Ab. All mAbs were InVivoPlus grade, formulated in sterile PBS and were low in endotoxin (<0.001 EU/g).

Tested mAbs

| | MAb | Clone | Manufacturer | Catalog # |
|---|---|---|---|---|
| 1 | Anti PDL-1 | 10F.9G2 | Bio X Cell | BP0101 |
| 2 | Rat IgG2b, k Isotype | LTF-2 | Bio X Cell | BP0090 |
| | Ctrl. | | | |

Study Design

Six-eight-week-old VSIG10 KO mice or C57BL/6 wild-type female mice were shaved and inoculated subcutaneously with 100 ul of 0.5×10⁶ MC38 tumor cells. At day 14 post-tumor implantations, mice were randomly assigned into treatment groups of n=10-15 (as described below). Mice were treated with mAbs (as detailed below) injected on day 14, 17, 20, and 23, post inoculation. Tumor growth was measured with electronic caliper every 3 days.

| | | | Mice | | | |
|---|---|---|---|---|---|---|
| # | | | per | Dose | # | Vol/Dose |
| Group | Mice | Treatment/mAb | group | (mg/Kg) | Dose | (ul) |
| 1 | Wild Type | Rat IgG2b | 10-15 | 5 | 4 | 100 |
| | | isotype control | | | | |
| 2 | VSIG10 KO | Rat IgG2b | 10-15 | 5 | 4 | 100 |
| | | isotype control | | | | |
| 3 | Wild Type | Anti-PDL1 | 10-15 | 5 | 4 | 100 |
| 4 | VSIG10 KO | Anti-PDL1 | 10-15 | 5 | 4 | 100 |

2.4 Statistical Analysis:

Two-way ANOVA with repeated measures, followed by two-way ANOVA with repeated measures for selected pairs of groups using JUMP (Statistical Discoveries™) software. Analyses of tumor growth measurements were performed by comparing tumor volumes measured on the last day on which 9-10 animals in the relevant control were alive. For each experiment, the number of replicates performed and the number of animals per group are described below for each figure.

Results

In-Vivo Tumor Growth Inhibition in VSIG10 KO Relative to Wild-Type Mice

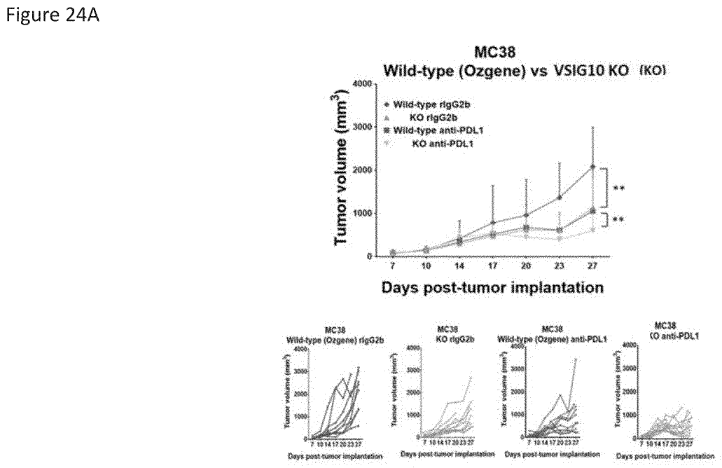

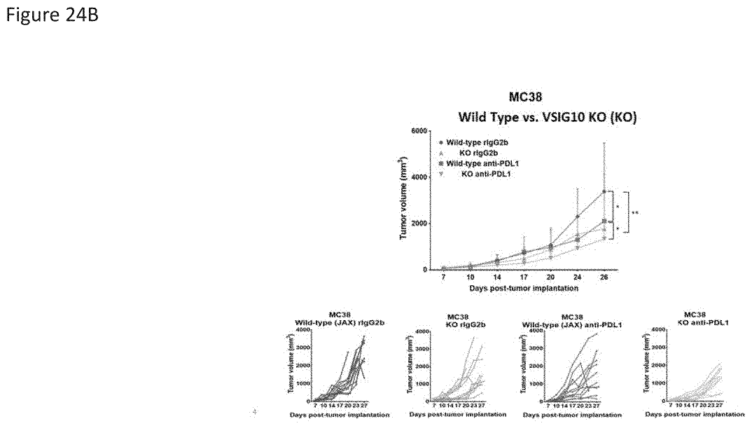

To evaluate the role of mVSIG10 in tumor growth inhibition Wild Type (WT) and VSIG10 KO mice were inoculated with MC38 colon adeno-carcinoma cells. On day 14 post inoculation mice were treated with anti-PDL1 Ab or its isotype control. FIG. 24 shows that reduced tumor growth of the MC38 tumor model inoculated to mVSIG10 KO relative to wild-type mice with and without anti-PDL-1 treatment. A-B. Groups of 10-15 VSIG10 KO or wild type C57BL/6 mice were subcutaneously injected with 5×10⁵ MC38 colon carcinoma cells. On day 14 post tumor inoculation, VSIG10 and wild-type mice were treated with anti-PD-L1 or rIgG2b isotype control Abs. Abs were administered in 5 mg/kg, intra-peritoneally (i.p.) twice per week for 2 weeks. Tumor growth was measured with electronic caliper every 3 days and was reported as 0.5×W2×L mm3 (L is length and W is width of the tumor). A. First experiment. ** indicate p-value<0.01 for WT treated with isotype control versus KO treated with isotype control and WT treated with anti-PDL1 versus KO treated with anti-PDL1 on day 27 B. Repeat study. ** indicates p-value<0.01 for WT treated with isotype versus KO treated with isotype and WT treated with anti-PDL1 versus KO treated with anti-PDL1 on day 27.

As seen in FIG. 24A, Tumor growth inhibition (TGI) was seen in VSIG10 KO mice, compared to WT mice. When we treated mice with anti-PDL1 blocking Ab, significantly reduced tumor growth was evident in both KO and WT mice. However, significant reduced tumor growth was evident for VSIG10 KO mice treated with anti-PD-L1 Ab, compared to WT mice treated with anti-PDL1 Ab. Results were reproduced in a repeat study (FIG. 24B) in which significant TGI was seen in VSIG10 KO mice compared to WT mice as well as in VSIG10 KO mice treated with anti-PDL1 compared to WT mice treated with anti-PDL1. Summary of the TGI values of the 2 experiments is presented in Table 2.

TABLE 2

| | TGI of rIgG2b isotype | TGI of anti-PD-L1 |
|---|---|---|
| | treated VSIG10 | treated VSIG10 |
| | KO vs. wild-type | KO Vs wild-type |
| Study1 (day 24) | 45.25% | 32.94% |
| Study2 | 43.43% | 33.89% |
| (day 27) | | |

Table 2 is a Summary Table of the tumor growth inhibition (TGI) in VSIG10-KO relative to wild-type mice in two experimental repeats with or without Anti-PD-L1 Combination in MC38 Tumor Model.

The above experiments demonstrate that VSIG10 potentially plays a role as a novel B7-like molecule and thus is as a potential target for antibody based cancer immunotherapy. Several mouse and human in vitro experimental systems have demonstrated an immune-modulatory effect for VSIG10. In the studies presented in this report, the in vivo anti-cancer effect of mVSIG10 was evaluated, using mice deficient for the mVSIG10 gene. Significant tumor growth inhibition in VSIG10 KO mice relative to wild type mice was observed in 2 experimental repeats. Furthermore, the combinatorial effect of mVSIG10 depletion and anti-PDL1 treatment were assessed. Treatment with anti-PDL1 begun on day 14 post-implantations and consistently resulted in significant tumor growth inhibition in both VSIG10 KO and wild-type mice. Enhanced anti-PDL1 tumor growth inhibition was evident in VSIG10 KO mice relative to wild type mice suggesting potential translational benefit for a combinatorial regimen of VSIG10 and blocking of PD1-PDL1 pathway.

### Example 6: Evaluation of the Expression of VSIG10 on Myeloid Cells Derived from Human Cancer

Aim: To define the protein expression of VSIG10 on the cell surface of myeloid cells derived from cancer samples.

Method:

Fresh tumor samples (ovarian, endometrial and renal clear cell carcinoma) were processed into single cells on a GentleMACS Tissue Dissociator (Miltenyi Biotec) using the human Tumor Dissociation Kit. Isolated single cells were cryopreserved until use for staining experiments. Endometrial ascites (˜2000 mL) was spun down and erythrocytes lysed using ACK lysis buffer. Peripheral blood mononuclear cells (PBMC) from healthy donors and patients with ovarian cancer were recovered from freshly collected whole blood by layering over Ficoll-Hypaque and density-gradient centrifugation.

Cells from above samples were surface stained with antibodies against the following lineage markers towards analysis by multi-color flow cytometry: CD45, CD303, CD141, CD1 c, CD1c, CD14, CD16, HLA-DR, Lineage cocktail comprising FITC-conjugated antibodies against CD3, CD19 and CD56, as well as live/dead fixable viability dye. PE-labeled anti-VSIG10 antibody and corresponding mIgG1 isotype control was used in a final concentration of 5 μg/ml. The expression of VSIG10 was assessed in the following cell populations: Immune cells (Viability Dye excluded CD45+), Non-immune cells (Viability Dye excluded CD45⁻), cDC (Viability Dye excluded CD45⁺Lin-CD16⁻HLA-DR⁺CD14⁻CD11c⁺), Myeloid DC (Viability Dye excluded CD45⁺Lin⁻CD16⁻HLA-DR⁺CD14⁺CD11c⁺). MFI ratio (MFIr) was calculated by dividing geometric mean fluorescence of anti-VSIG10 by that of the isotype control.

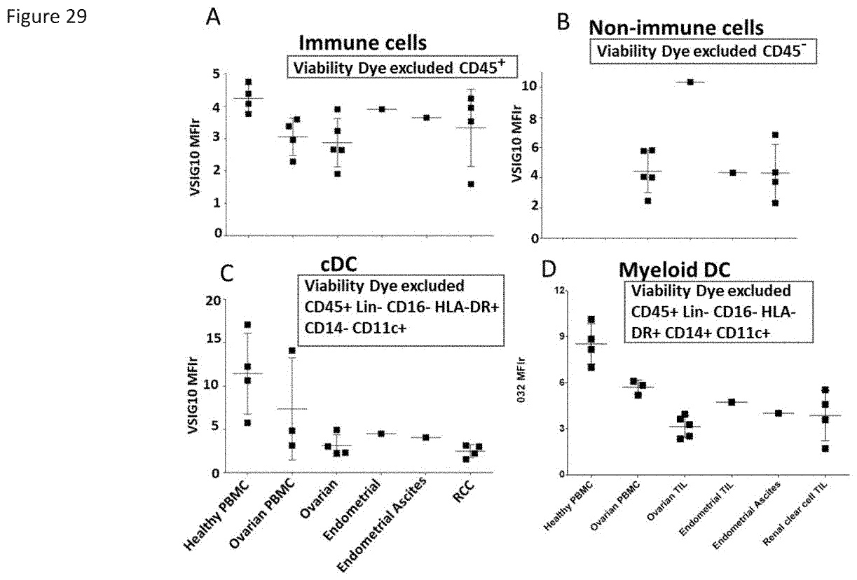

FIG. 29 shows the expression of VSIG10 by FACS on immune cells (FIG. 29A), non-immune cells (FIG. 29B), cDCs (FIG. 29C) and myeloid DCs (FIG. 29D), presented as MFI ratio between anti-VSIG10 stained cells and isotype control.

In this study we have observed cell surface staining of VSIG10 on myeloid cells derived from ovarian, endometrial and renal cancer. Higher surface expression was observed in general on healthy peripheral blood mononuclear cells (PBMC) followed by immune cells from renal clear cell carcinoma. VSIG10 surface expression on cDC and myeloid DC subsets was also higher in PBMCs from healthy donors and patients with ovarian cancer. Additional experimentation with freshly isolated tumor samples (prior to cryopreservation) is needed to define whether the difference between PBMC and tumor myeloid/DC subsets is real or could be attributed to cryopreservation-induced loss of surface VSIG10 antigen.

### Example 7: Effect of VSIG10 on Mouse CD4+ T Cell Response in CHOS-IAd DO11.10 Assay

The aim of the assay described herein is to evaluate the functional capacity of VSIG10 to inhibit the activation of mouse T cells as measured by cytokine secretion and proliferation capability upon co-culture with CHO-S mIAd target cells which overexpress VSIG10 as a ligand.

### Materials

### Reagents and Plastics

CD CHO (Gibco, 10743-11)
RPMI 1640 (Biological Industries, 01-100-1A)
Fetal Bovine Serum-FBS (Biological Industries, 04-127-1A)
Glutamax (Life technologies, 35050-038)
Sodium pyruvate (Biological Industries, 03-042-1B)
2-mercapto-EtOH (Life technologies, 31350-010)
Penicillin-Streptomycin Solution (Biological Industries, 03-031-1B)
Puromycin (invivogen, 58-58-2)
Trypan blue, 0.4% diluted (Biological Industries, 03-102-1B)
Mitomycin C (Sigma, M4287)
OVA peptide (323-339)
PBS (Biological Industries, 02-023-1A)
Ultra pure water (Biological Industries, 01-866-1A)
BSA (Sigma, A7030)
EDTA 2 mM (Sigma, E7889)
5% sodium Azide solution (Teknova, S0208)
Lysis Buffer ×10 (BD, 555899)
EasySep Mouse CD4+ T cell Isolation kit (STEMCELL Technologies, 18000)
Cell proliferation dye eFlour450 (eBiosciences, 65-0842-85)
Viability dye (BD horizon, 562247)
Purified Rat anti mouse CD16/32 (Biolegend, 553142)
AF647-anti mouse IAd (Biolegend, 115010)
APC-Rat IgG2b, k isotype ctrl (Biolegend, 400612)
APC-anti mouse CD4 (Biolegend, 100412)
APC-anti mouse CD274 B7-H1, PDL1 (Biolegend, 124312)
rabbitamVSIG10_488536_9(M:M) GS_pAb
PE-AffiniPure F (ab′)2 Fragment Donkey Anti-Rabbit IgG (Jackson 711-116-152)
CBA Mouse Th1, Th2, Th17 cytokine kit (BD Biosciences, 560485)
125 mL Erlenmeyer (Corning, 431143)
96 well U shape tissue culture plate (Costar, 3799)
15 mL polypropylene tubes (Greiner Bio-one, 188261)
50 mL polypropylene tubes (Greiner Bio-one, 210261)
96 well polypropylene plate (Greiner bio-one, 650201)
5 ml Syringe (BD, 309649)
Cell strainer 40 um (Falcon, Corning, 352340)
5 cm TC plate 60×15 mm multivent dish (Corning, WD-430166)
T75 tissue culture flask (Greiner bio-one, 658175)

### Equipment

Incubator 37° C., 5% CO₂ (Binder)
Centrifuge (Eppendorf, 5810R)
Countess II cell automated counter (life technologies)
Plate shaker DTS4
Macsquant analyzer (Milteneyi)
Cell sorter BD FACSJazz™

### Cells

Purified mouse CD4+ T cells were obtained from spleens of DO. 11 BALB/C mice by using EasySep Mouse CD4+ T cell Isolation kit (STEMCELL Technologies).
CHO-S mIAd cells overexpressing mouse VSIG10 (RC-626) or mouse PDL1 (RC-630) or empty vector transduced cells (EV, RC-323) as control (transduction described in methods section).

### Methods

### Cell Culture

CD4+ T cells were produced and purified from spleens on the co culture day and resuspended in RPMI media which contains 10% FBS, 1% Glutamax, 1% Sodium pyruvate, 1% Penicillin-Streptomycin solution and 0.1% β mercapto ethanol.
CHO-S mIAd transduced cells were cultured in CD-CHO media supplemented with 8 mM Glutamax, 1% Penicillin-Streptomycin Solution and 6 ug/ml Puromycin+50 ug/ml Hygromycin. Day before the co culture cells were diluted to 0.5*10^{\hat{ }6}/ml. Cells were cultured for no longer than 30 days.

### Gene Over-Expression in CHO-S Cells

To stimulate CD4+ T cells, CHO-S cells were transduced with mouse IAd vector (mIAd_Untagged_pDUO_2) to overexpress membrane-bound Mouse IAd fragments.

To asses VSIG10 effects on CD4+ T cells as a ligand on target cells we overexpressed VSIG10 gene in CHO-S mIAd cells.

Briefly, Production of Lentivirus particles (virions) was done at Sirion Biotech by cloning non-optimized mouse VSIG10 sequence (Untagged) into lentiviral expression plasmid pcLV-CMV-VSIG10-IRES-Puro. CHO-S mIAd cells were transduced with Lentivirus particles (4.1E+07 IU/ml) by using 10 MOI.

A no gene construct (pcLV-CMV-MCS-IRES-Puro) was used as negative control (designated CHO-S mIAd EV).

Mouse PDL1 was used as positive control in the assay as an inhibitory ligand (designated CHO-S mIAd mouse PDL1) and was established as follow: Production of Lentivirus particles (virions) was done at Sirion Biotech by cloning optimized mouse PDL-1 sequence (Untagged) into lentiviral expression plasmid pcLV-CMV-mPDL-1-IRES-Puro. CHO-S mIAd cells were transduced with Lentivirus particles (2E+08 IU/ml) by using 20 MOI. Cells were used to generate stable pool, banked, thawed 3-5 days prior to assay and cultured for no more than 30 days.

### Co-culture

CHO-S mIAd overexpressing mouse VSIG10 or mouse PDL1 or EV cells were treated with Mitomycin C at 50 ug/ml, for 1 hr in 37° C. to suppress mitosis, washed twice with DO. 11 media (RPMI, 10% FBS, 1% Glutamax, 1% Sodium pyruvate, 1% PenStrep, 0.1% βME) and seeded in DO. 11 media in co-culture U shape 96w plate at a concentration of 3e4/50 ul/well. OVA peptide was added in a final concentration of 5e-4 or 2.5e-4 ug/50 ul/well (final OVA-p concentration of 0.1 or 0.05 ug/ml) (diluted in DO11 media).

Spleens were harvested from DO.11 mice (6-12 weeks male or female). DO.11 splenocytes were collected and CD4+ T cells were isolated using ‘Mouse CD4+ T Cell isolation kit negative selection’ (EasySep, 18000). The purity of CD4+ T cells was analysed by FACS (>95% purity). Purified CD4+ T cells labeled with CPD (1:1000, eBioscience) to be able to track proliferation in co-culture, washed twice with DO.11 media (RPMI, 10% FBS, 1% Glutamax, 1% Sodium pyruvate, 1% PenStrep, 0.1% (3ME) and resuspend in concentration of 2×10\hat{ }6/ml. CD4+ T cells were seeded on top of the CHO-S mIAd cells in at a concentration of 1e5 cells/50 ul/well. In two repeats, CD4+ T cells were seeded without CPD labeling.

Effector to target ratio—3:1. Final co-culture volume 200 ul. Plate was incubated for four or five days in 37° C., 5% CO2 incubator. Plate's edges (row A/H; column 1/12) were filled with 200 ul/well of media as well to avoid evaporation from the co-culture wells. Each experiment was done in quadruplicates. A total of three experiments were done using CD4+ T cells.

mIAd and Target Validation

On the co-culture day, targets expression of the transfected CHO-S mIAd cells were evaluated by flow cytometry using relevant Abs at conc of 5 ug/ml (APC anti mouse VSIG10 (rabbitamVSIG10_488536_9(M:M)_GS_pAb) or APC anti mouse CD274 B7-H1, PDL1, Biolegend, cat#124312) and surface mIAd levels (AF647 Anti mouse IAd, Biolegend, cat#115010) were evaluated by flow cytometry using for 30 min in 4° C. cells were run In MACSquant and expression was analyzed by FlowJo software. <20% discrepancy in mIAD levels between the tested CHO-S mIAd cells overexpressing mouse VSIG10 or mouse PDL1 and the control EV cells were acceptable (data not shown).

### Assessment of T Cells Functional Capacity

### Proliferation Readout

After four days' co-culture plate were centrifuged. Cell pellets were stained with ZombieNir (for viability), washed and stained with antibody of anti-mouse-CD4-FITC (Biolegend, cat#100406) and mouse Fc block. After 30 min incubation at 4° C., samples were washed and run in MACSquant (Milteney) for CPD low labeling, gating on CD4 sub-populations. Analysis of proliferating cells was made as the total number of CPD low cells in each well [#Proliferating (CPDlo) CD4+/well]. This readout was performed in one experiment only (data not shown).

### Cytometric Bead Array—CBA

Co-culture supernatants were collected after four or five days' co-culture and tested for cytokines by CBA using ¼ volume of each reagent. In brief, capture antibody bead mix was prepared by adding 2 uL/sample per each cytokine capture bead and 12.5 uL mix was added to 96 well polypropylene plate. 50 uL supernatant was then added followed by 12.5 uL detection reagent and spin down. Plates were incubated in plate shaker for 2 hr, 1500 rpm, washed and read in MACSquant.

### Data Analysis

All FACS files were analyzed by FlowJo software.

Two tailed paired parametric T-test was calculated for 4 repeats per CD4+ T cells. P<0.05 was referred to as statistically significant

### Results

### Immune-Modulatory Effect on T Cells by VSIG10

4 co-culture experiments were conducted. Cytokines secretion (IFNγ, TNFa and IL-2) from CD4+ T cells co-cultured with CHO-S mIAd cells overexpressing mouse VSIG10 were decreased in all 4 exp compared to EV cells.

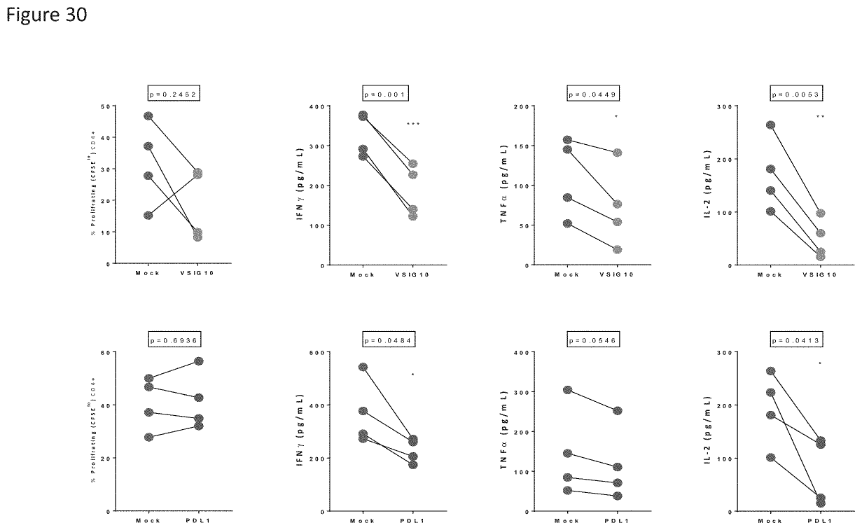

T cells proliferation was tested by CPD and depicted an enhancement in T cells proliferation in ¾ experiments compared to EV cells [% proliferating (CPD low) CD4+/well] (Table 4). The inhibitory effect of human PDL1, as a positive control for the assay was also tested. These inhibitory effects by mouse VSIG10 were statistically significant, as shown in FIG. 30. Mouse PDL1 inhibitory effects were found to be statistically significant for the same readouts tested.

% inhibition of VSIG10 vs. mock

| % CD4+ CFSE low | IFNg | TNFa | IL-2 |
|---|---|---|---|
| — | — | — | — |
| 77% | 58% | 57% | 85% |
| 65% | 49% | 64% | 63% |
| 39% | 41% | 47% | 67% |
| −85% | 32% | 11% | 82% |

Tables 4: CHO-S mIAd over expressing mouse VSIG10 inhibitory effect on mouse CD4+ T cells. The mean of quadruplicates for each overexpressing cell (VSIG10) was compared to the mean of the control EV cells. The percentage of effect is indicated for proliferation and cytokines readouts.

### Summary

Mouse VSIG10 over expression on CHO-S mIAd cells induced a significant inhibitory effect (inhibition >30% vs. EV) on T cells across 4 experiments, as shown by cytokine secretion read out, which was reduced compare to EV transduced cells. The sum of effects seen across 4 repeats meets assay criteria by which a >30% assay window in at least 2 readouts is considered as successes criteria. Assay was validated with a relevant positive control, PDL1. Reduced inhibitory effect was observed in the cytokines readout for mouse PDL1 overexpressing cells.

### Example 8—Additional Experimental Data

FIGS. 31A and 31B show scatter plots, demonstrating the expression of VSIG10 transcripts, that encode the VSIG10 proteins, on a virtual panel of all tissues and conditions using MED discovery engine, demonstrating differential expression of VSIG10 transcripts in several groups of cells from the immune system, mainly in leukocytes, and in various cancer conditions, such as CD10+ leukocytes from ALL and BM-CD34+ cells from AML.

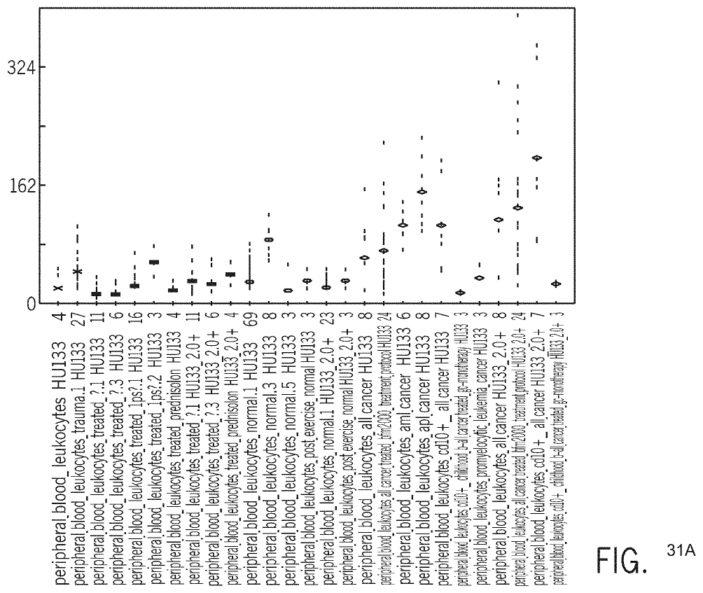

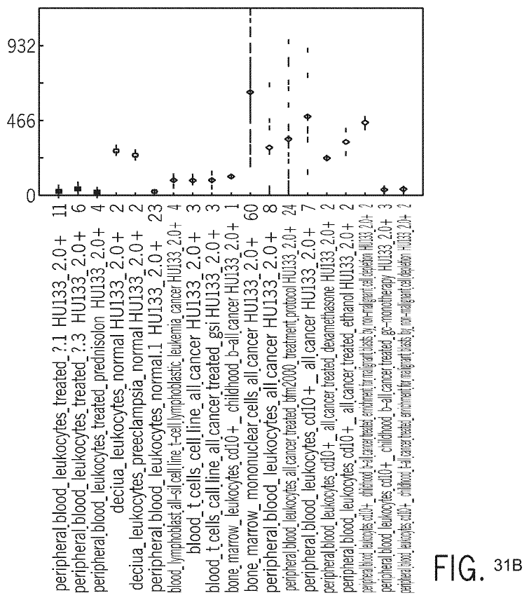

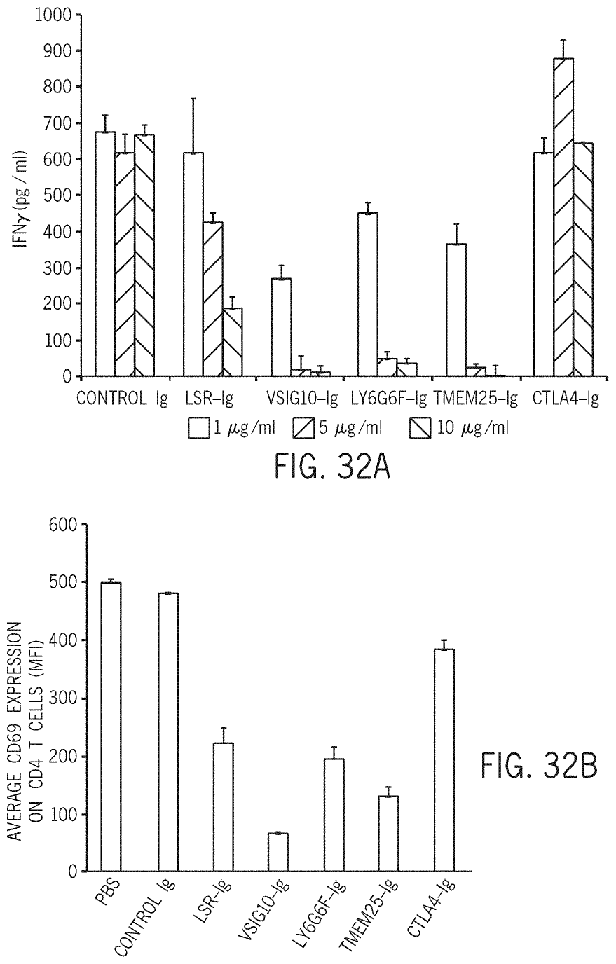

FIGS. 32A and 32B show the effect of VSIG10 fusion protein (SEQ ID NO:24), and other proteins, on CD4 T cell activation, as manifested by reduced IFNγ secretion (A) and reduced expression of the activation marker CD69 (B). Each bar is the mean of duplicate cultures, the error bars indicating the standard deviation (Student t-test,*P<0.05, **p<0.01, compared with control mIgG2a.

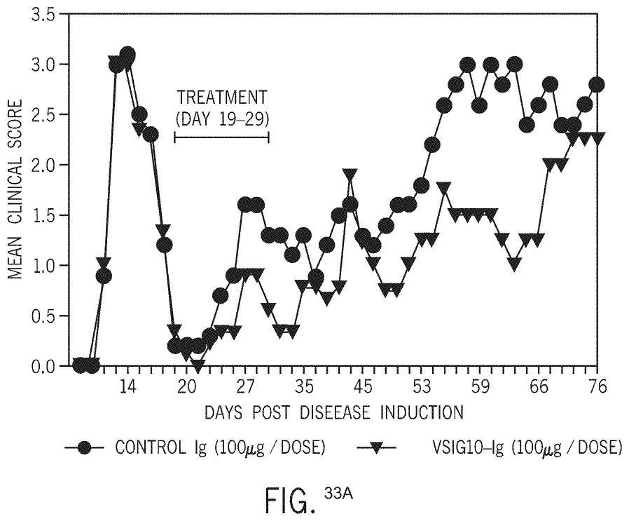

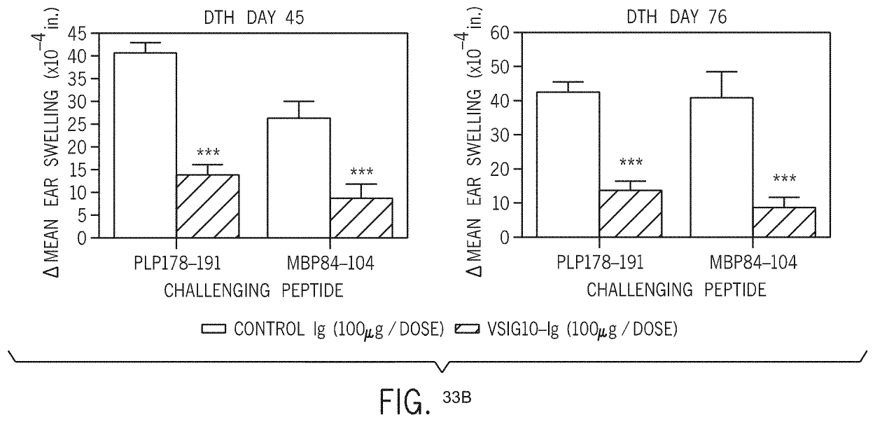

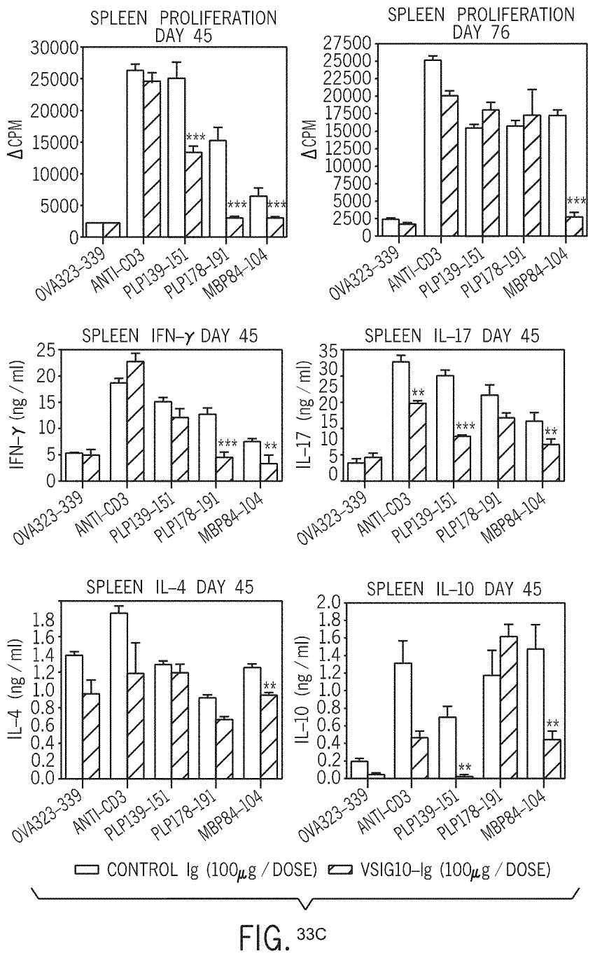

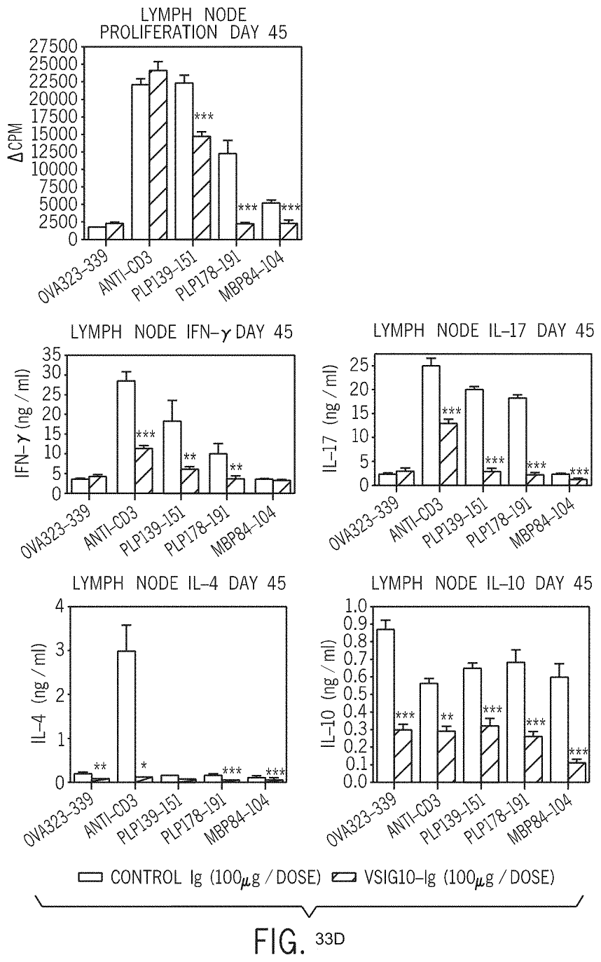

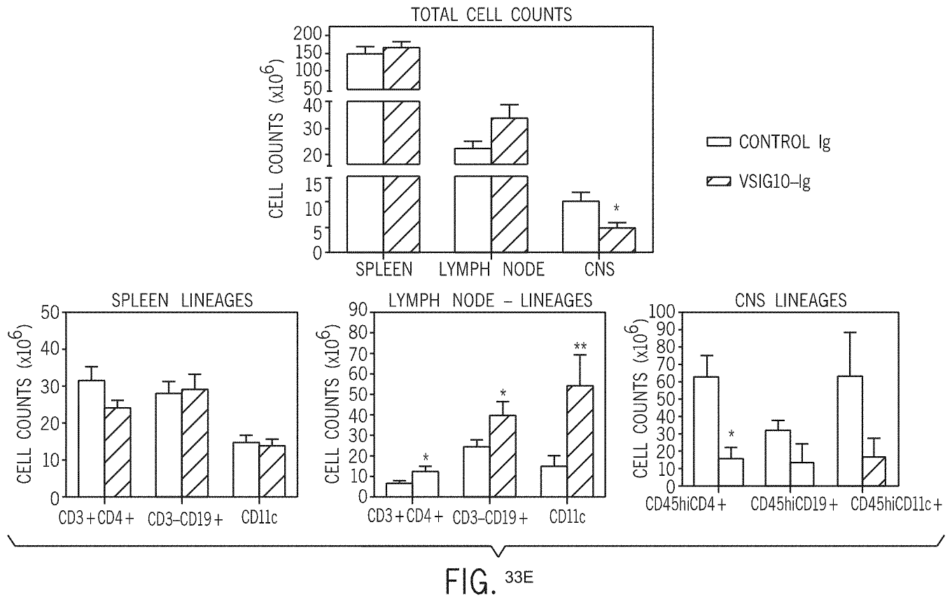

FIGS. 33A-33E show the therapeutic effect of VSIG10-Ig (SEQ ID NO:24) treatment in the PLP139-151-induced R-EAE model in SJL mice. VSIG10-Ig (SEQ ID NO:24) was administered in a therapeutic mode from the onset of disease remission (day 19), at 100 microg/mouse i.p. 3 times per week for two weeks. Therapeutic effects of VSIG10-Ig on clinical symptoms is demonstrated as reduction in Mean Clinical Score (FIG. 33A). In addition, VSIG10-Ig treatment inhibited DTH responses to spread epitopes (PLP178-191 and MBP MBP84-104), on days 45 and 76 after R-EAE induction (FIG. 33B). Also shown is the effect of VSIG10-Ig on ex-vivo recall responses of splenocytes isolated on day 45 and 75 post disease induction (FIG. 33C) and LN cells isolated on day 45 post disease induction (FIG. 33D) as manifested by the effect of VSIG10-Ig treatment on cell proliferation and cytokine secretion (IFNg, IL-17, IL-10 and IL-4). The effect of VSIG10-Ig on cell counts in the spleen, lymph nodes and CNS as well as the different linages present within each of these tissues upon treatment with VSIG10-Ig at 100 ug/dose is shown in FIG. 33E. In this study the effect of VSIG10-Ig was studied in comparison to mIgG2a Ig control that was administered at similar dose and regimen as VSIG10-Ig.

The invention has been described and various embodiments provided relating to manufacture and selection of desired anti-VSIG10 antibodies for use as therapeutics and diagnostic methods wherein the disease or condition is associated with VSIG10 antigen. Different embodiments may optionally be combined herein in any suitable manner, beyond those explicit combinations and subcombinations shown herein. The invention is now further described by the claims which follow.

SEQUENCES:

1, Human Ig kappa SP + VSIG10-ECD + Human IgG1 Fc mutated at C220S of hinge
SEQ ID NO: 1
MEAPAQLLFLLLLWLPDTTGVVIGEVHENVTLHCGNISGLRGQVTWYRNNSEPVFLLSSNSSLRPAEP
RFSLVDATSLHIESLSLGDEGIYTCQEILNVTQWFQVWLQVASGPYQIEVHIVATGTLPNGTLYAARGS
QVDFSCNSSSRPPPVVEWWFQALNSSSESFGHNLTVNFFSLLLISPNLQGNYTCLALNQLSKRHRKV
TTELLVYYPPPSAPQCWAQMASGSFMLQLTCRWDGGYPDPDFLWIEEPGGVIVGKSKLGVEMLSES
QLSDGKKFKCVTSHIVGPESGASCMVQIRGPSLLSEPMKTCFTGGNVTLTCQVSGAYPPAKILWLRN
LTQPEVIIQPSSRHLITQDGQNSTLTIHNCSQDLDEGYYICRADSPVGVREMEIWLSVKEPLNIGGEPK
SSDKTHTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGK
2, 173, VSIG10_Human-ECD_woSP_Human-Fc_C220S SEQ ID NO: 2
>2(173)
VVIGEVHENVTLHCGNISGLRGQVTWYRNNSEPVFLLSSNSSLRPAEPRFSLVDATSLHIESLSLGDEGIY
TCQEILNVTQWFQVWLQVASGPYQIEVHIVATGTLPNGTLYAARGSQVDFSCNSSSRPPPVVEWWFQALNS
SSESFGHNLTVNFFSLLLISPNLQGNYTCLALNQLSKRHRKVTTELLVYYPPPSAPQCWAQMASGSFMLQL
TCRWDGGYPDPDFLWIEEPGGVIVGKSKLGVEMLSESQLSDGKKFKCVTSHIVGPESGASCMVQIRGPSLL
SEPMKTCFTGGNVTLTCQVSGAYPPAKILWLRNLTQPEVIIQPSSRHLITQDGQNSTLTIHNCSQDLDEGY
YICRADSPVGVREMEIWLSVKEPLNIGGEPKSSDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
3, gi|140254891|ref|NP_061959.2_VSIG10 SEQ ID NO: 3
>3
MAAGGSAPEPRVLVCLGALLAGWVAVGLEAVVIGEVHENVTLHCGNISGLRGQVTWYRNNSEPVFLLSSNS
SLRPAEPRFSLVDATSLHIESLSLGDEGIYTCQEILNVTQWFQVWLQVASGPYQIEVHIVATGTLPNGTLY
AARGSQVDFSCNSSSRPPPVVEWWFQALNSSSESFGHNLTVNFFSLLLISPNLQGNYTCLALNQLSKRHRK
VTTELLVYYPPPSAPQCWAQMASGSFMLQLTCRWDGGYPDPDFLWIEEPGGVIVGKSKLGVEMLSESQLSD
GKKFKCVTSHIVGPESGASCMVQIRGPSLLSEPMKTCFTGGNVTLTCQVSGAYPPAKILWLRNLTQPEVII
QPSSRHLITQDGQNSTLTIHNCSQDLDEGYYICRADSPVGVREMEIWLSVKEPLNIGGIVGTIVSLLLLGL
AIISGLLLHYSPVFCWKVGNTSRGQNMDDVMVLVDSEEEEEEEEEEEEDAAVGEQEGAREREELPKEIPKQ
DHIHRVTALVNGNIEQMGNGFQDLQDDSSEE
QSDIVQEEDRPV
4, VSIG10_ECD_31-413 SEQ ID NO: 4
>4
VVIGEVHENVTLHCGNISGLRGQVTWYRNNSEPVFLLSSNSSLRPAEPRFSLVDATSLHIESLSLGDEGIY
TCQEILNVTQWFQVWLQVASGPYQIEVHIVATGTLPNGTLYAARGSQVDFSCNSSSRPPPVVEWWFQALNS
SSESFGHNLTVNFFSLLLISPNLQGNYTCLALNQLSKRHRKVTTELLVYYPPPSAPQCWAQMASGSFMLQL
TCRWDGGYPDPDFLWIEEPGGVIVGKSKLGVEMLSESQLSDGKKFKCVTSHIVGPESGASCMVQIRGPSLL
SEPMKTCFTGGNVTLTCQVSGAYPPAKILWLRNLTQPEVIIQPSSRHLITQDGQNSTLTIHNCSQDLDEGY
YICRADSPVGVRE
MEIWLSVKEPLNIGG
5, VSIG10_Variant_skipping_exon_3_T95617_P6_#ID 6 #LN 439 #EI 97372189
#OG GH #OG MaxORF #OG WT_BASED SEQ ID NO: 5
>5
MAAGGSAPEPRVLVCLGALLAGWVAVGLEAVVIGEVHENVTLHCGNISGLRGQVTWYRNNSEPVFLLSSNS
SLRPAEPRFSLVDATSLHIESLSLGDEGIYTCQEILNVTQWFQVWLQVANPPPSAPQCWAQMASGSFMLQL
TCRWDGGYPDPDFLWIEEPGGVIVGKSKLGVEMLSESQLSDGKKFKCVTSHIVGPESGASCMVQIRGPSLL
SEPMKTCFTGGNVTLTCQVSGAYPPAKILWLRNLTQPEVIIQPSSRHLITQDGQNSTLTIHNCSQDLDEGY
YICRADSPVGVREMEIWLSVKEPLNIGGIVGTIVSLLLLGLAIISGLLLHYSPVFCWKVGNTSRGQNMDDV
MVLVDSEEEEEEEEEEEEDAAVGEQEGAREREELPKEIPKQDHIHRVTALVNGNIEQMGNGFQDLQDDSSE
EQSDIV
QEEDRPV
6, VSIG10_Variant_skipping_exon_3_ECD_31-312 SEQ ID NO: 6
>6
VVIGEVHENVTLHCGNISGLRGQVTWYRNNSEPVFLLSSNSSLRPAEPRFSLVDATSLHIESLSLGDEGIY
TCQEILNVTQWFQVWLQVANPPPSAPQCWAQMASGSFMLQLTCRWDGGYPDPDFLWIEEPGGVIVGKSKLG
VEMLSESQLSDGKKFKCVTSHIVGPESGASCMVQIRGPSLLSEPMKTCFTGGNVTLTCQVSGAYPPAKILW
LRNLTQPEVIIQPSSRHLITQDGQNSTLTIHNCSQDLDEGYYICRADSPVGVREMEIWL
SVKEPLNIGG
19, VSIG10_MOUSE_ECD_with_SP SEQ ID NO: 19
>19
MAGLRVLLCLGALLARQGSAGLQLLLNPSRANLSVRPNSEVLPGIHPDLEAVAIGEVHDNVTLRCGSASGS
RGLVTWYRNDSEPAFLVSFNSSLPPAAPRFSLEDAGALRIEALRLEDDGNYTCQEVLNETHWFPVRLRVAS
GPAYVEVNISATGTLPNGTLYAARGSQVDFNCCSAAQPPPEVEWWIQTHSIPEFLGKNLSANSFTLMLMSQ
NLQGNYTCSATNVLSGRQRKVTTELLVYWPPPSAPQCSVEVSSESTTLELACNWDGGYPDPTFLWTEEPGG
TIMGNSKLQTLSPAQLLEGKKFKCVGNHILGPESGASCVVKLSSPLLPSQPMRTCFVGGNVTLTCEVSGAN
PPARIQWLRNLTQPAIQPSSHYIITQQGQSSSLTIHNCSQDLDEGFYYCQAENLVGVRATN
IWLSVKEPLNIGG
24, CGEN-15031_VSIG10-Mouse-ECD_no_SP_FC_mouse_IgG2a SEQ ID NO: 24
>24
LQLLLNPSRANLSVRPNSEVLPGIHPDLEAVAIGEVHDNVTLRCGSASGSRGLVTWYRNDSEPAFLVSFNS
SLPPAAPRFSLEDAGALRIEALRLEDDGNYTCQEVLNETHWFPVRLRVASGPAYVEVNISATGTLPNGTLY
AARGSQVDFNCCSAAQPPPEVEWWIQTHSIPEFLGKNLSANSFTLMLMSQNLQGNYTCSATNVLSGRQRKV
TTELLVYWPPPSAPQCSVEVSSESTTLELACNWDGGYPDPTFLWTEEPGGTIMGNSKLQTLSPAQLLEGKK
FKCVGNHILGPESGASCVVKLSSPLLPSQPMRTCFVGGNVTLTCEVSGANPPARIQWLRNLTQPAIQPSSH
YIITQQGQSSSLTIHNCSQDLDEGFYYCQAENLVGVRATNIWLSVKEPLNIGGEPRGPTIKPCPPCKCPAP
NLLGGPSVFIFPPKIKDVLMISLSPIVTCVVVDVSEDDPDVQISWFVNNVEVHTAQTQTHREDYNSTLRVV
SALPIQHQDWMSGKEFKCKVNNKDLPAPIERTISKPKGSVRAPQVYVLPPPEEEMTKKQVTLTCMVTDFMP
EDIYVEWTNNGKTELNYKNTEPVLDSDGSYFMYSKLRVEKKNWVERNSYSCSVVHE
GLHNHHTTKSFSRTPG
K
27, Mouse_IgG2a_Fc SEQ ID NO: 27
>27
EPRGPTIKPCPPCKCPAPNLLGGPSVFIFPPKIKDVLMISLSPIVTCVVVDVSEDDPDVQISWFVNNVEVH
TAQTQTHREDYNSTLRVVSALPIQHQDWMSGKEFKCKVNNKDLPAPIERTISKPKGSVRAPQVYVLPPPEE
EMTKKQVTLTCMVTDFMPEDIYVEWTNNGKTELNYKNTEPVLDSDGSYFMYSKLRVEKKNWVERNSYSCSV
VHEGLHNHHTT
KSFSRTPGK
30, gi|298352624|sp|D3YX43.2|VSI10_MOUSE RecName: Full = V-set and
immunoglobulin domain-containing protein 10; Flags: Precursor SEQ ID
NO: 30
>30
MAGLRVLLCLGALLARQGSAGLQLLLNPSRANLSVRPNSEVLPGIHPDLEAVAIGEVHDNVTLRCGSASGS
RGLVTWYRNDSEPAFLVSFNSSLPPAAPRFSLEDAGALRIEALRLEDDGNYTCQEVLNETHWFPVRLRVAS
GPAYVEVNISATGTLPNGTLYAARGSQVDFNCCSAAQPPPEVEWWIQTHSIPEFLGKNLSANSFTLMLMSQ
NLQGNYTCSATNVLSGRQRKVTTELLVYWPPPSAPQCSVEVSSESTTLELACNWDGGYPDPTFLWTEEPGG
TIMGNSKLQTLSPAQLLEGKKFKCVGNHILGPESGASCVVKLSSPLLPSQPMRTCFVGGNVTLTCEVSGAN
PPARIQWLRNLTQPAIQPSSHYIITQQGQSSSLTIHNCSQDLDEGFYYCQAENLVGVRATNIWLSVKEPLN
IGGIVGTVVSLLLLGLAVVSGLTLYYSPAFWWKGGSTFRGQDMGDVMVLVDSEEEEEEEEEEEEKEDVAEE
VEQETNETEELPKGISKHGHIHRVTALVNGNLDRMGNGFQEFQDDSDGQQSGIVQEDGKPV
60, VSIG10_ECD_WITH_SP SEQ ID NO: 60
>60
MAAGGSAPEPRVLVCLGALLAGWVAVGLEAVVIGEVHENVTLHCGNISGLRGQVTWYRNNSEPVFLLSSNS
SLRPAEPRFSLVDATSLHIESLSLGDEGIYTCQEILNVTQWFQVWLQVASGPYQIEVHIVATGTLPNGTLY
AARGSQVDFSCNSSSRPPPVVEWWFQALNSSSESFGHNLTVNFFSLLLISPNLQGNYTCLALNQLSKRHRK
VTTELLVYYPPPSAPQCWAQMASGSFMLQLTCRWDGGYPDPDFLWIEEPGGVIVGKSKLGVEMLSESQLSD
GKKFKCVTSHIVGPESGASCMVQIRGPSLLSEPMKTCFTGGNVTLTCQVSGAYPPAKILWLRNLTQPEVII
QPSSRHLITQDGQNSTLTIHNCSQDLDEGYYICRADSPVGVREME
IWLSVKEPLNIGG
61, VSIG10_Variant_skipping_exon_3_ECD_WITH_SP SEQ ID NO: 61
>61
MAAGGSAPEPRVLVCLGALLAGWVAVGLEAVVIGEVHENVTLHCGNISGLRGQVTWYRNNSEPVFLLSSNS
SLRPAEPRFSLVDATSLHIESLSLGDEGIYTCQEILNVTQWFQVWLQVANPPPSAPQCWAQMASGSFMLQL
TCRWDGGYPDPDFLWIEEPGGVIVGKSKLGVEMLSESQLSDGKKFKCVTSHIVGPESGASCMVQIRGPSLL
SEPMKTCFTGGNVTLTCQVSGAYPPAKILWLRNLTQPEVIIQPSSRHLITQDGQNSTLTIHNCSQDLDEGY
YICRADSPVGVREMEIWLSVKEPLNIGG
70, Human_IgG1_Fc SEQ ID NO: 70
>70
EPKSCDKTHTCPPCPAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRWQQGNVFSCSVM
HEALHNHYTQKSLSLSPGK
156, Human_IgG1_Fc_C220S SEQ ID NO: 156
>156
EPKSSDKTHTCPPCPAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRWQQGNVFSCSVM
HEALHNHYTQK
SLSLSPGK
157, Human_IgG1_Fc_without_hinge CH2 and CH3 regions of a human
immunoglobulin C-Gamma-1 chain SEQ ID NO: 157
>157
APELLGGPSVFLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVFSCSVMHEALHNHYTQKSLSL
SPGK
158, Mouse_IgG2a_Fc_without_hinge CH2 and CH3 regions of a murine
immunoglobulin C-Gamma-2a chain SEQ ID NO: 158
>158
APNLLGGPSVFIFPPKIKDVLMISLSPIVTCVVVDVSEDDPDVQISWFVNNVEVHTAQTQTHREDYNSTLR
VVSALPIQHQDWMSGKEFKCKVNNKDLPAPIERTISKPKGSVRAPQVYVLPPPEEEMTKKQVTLTCMVTDF
MPEDIYVEWTNNGKTELNYKNTEPVLDSDGSYFMYSKLRVEKKNWVERNSYSCSVVHEGLHNHHTT
KSFSRTPGK
174, VSIG10_Skipping-exon-3_Human-ECD_Human-Fc_C220S SEQ ID NO: 174
>174
VVIGEVHENVTLHCGNISGLRGQVTWYRNNSEPVFLLSSNSSLRPAEPRFSLVDATSLHIESLSLGDEGIY
TCQEILNVTQWFQVWLQVANPPPSAPQCWAQMASGSFMLQLTCRWDGGYPDPDFLWIEEPGGVIVGKSKLG
VEMLSESQLSDGKKFKCVTSHIVGPESGASCMVQIRGPSLLSEPMKTCFTGGNVTLTCQVSGAYPPAKILW
LRNLTQPEVIIQPSSRHLITQDGQNSTLTIHNCSQDLDEGYYICRADSPVGVREMEIWLSVKEPLNIGGEP
KSSDKTHTCPPCPAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLYSKLTVDKSRWQQGNVFSCSVMHE
ALHNHYTQKSLSLSP
GK
SEQ ID NO: 200 577Ab Heavy chain: DNA sequence (402 bp)
Leader sequence-FR1-CDR1-FR2-CDR2-FR3-CDR3-FR4
200>
ATGGAATGGAGGATCTTTCTCTTCATCCTGTCAGGAATTGCAGGTGTCCACTCCC
AGGTTCAGCTGCAGCAGTCTGGACCTGAGCTGGTGAGGCCTGGGGCTTCAGTGA
AGATGTCCTGCAAGCCTTCTGGATACAGATTCACTGACTACGCTATAAGTTGGG
TGAAGCAGAGAACTGGACAGGGCCTTGAGTGGGTTGGAGAGATTTATCCTGGA
AATGGTAATACTTACTTCAATGAGAAATTCAAGGACAAGGCCACACTGACTGC
AGACAGATCCTCCAACACAGCCTACATGCAGCTCAGCAGCCTGACATCTGAGGA
CTCTGCGGTCTATTTCTGTGCAAGAGGCTATGCTAACTACCTGCCCTGGGGCCA
AGGGACTCTGGTCACTGTCTCTGCA
SEQ ID NO: 201 577Ab Heavy chain: Amino acids sequence (134 aa)
>201
MEWRIFLFILSGIAGVHSQVQLQQSGPELVRPGASVKMSCKPSGYRFTDYAISWVKQ
RTGQGLEWVGEIYPGNGNTYFNEKFKDKATLTADRSSNTAYMQLSSLTSEDSAVY
FCARGYANYLPWGQGTLVTVSA
SEQ ID NO: 202 577Ab HC-CDR1
>202
DYAIS
SEQ ID NO: 203 577Ab HC-CDR2
>203
EIYPGNGNTYFNEKFKD
SEQ ID NO: 204 577Ab HC-CDR3
>204
GYANYLP
SEQ ID NO: 205 577Ab Light chain: DNA sequence (381 bp)
Leader sequence-FR1-CDR1-FR2-CDR2-FR3-CDR3-FR4
>205
ATGAAGTTTCCTTCTCAACCTCTGCTTTTACTGCTGTTTGGAATCCCAGGCATGAT
ATGTGACATCCAGATGACACAATCTTCATCCTCCTTTTCTGTATCTCTAGGAGACA
GAGTCACCATTACTTGTAAGGCAAGTGAGGACATATATAATCGGTTAGCCTGG
TATCAGCAGAAACCAGGAAATGCTCCTAGGCTCTTAATATCTGGTGCAACCGGT
TTGGAAACTGGGGTTCCTTCAAGAATCAGTGGCAGTGGATCTGGAAGGGATTAC
ACTCTCAGCATTACCAGTCTTCAGACTGAAGATGTTGGTACTTATTACTGTCAAC
AATATTGGAGTACTCCTCGGACGTTCGGTGGAGGCACCAAGTTGGAAATCAAA
SEQ ID NO: 206 577Ab Light chain: Amino acids sequence (127 aa)
Leader sequence-FR1-CDR1-FR2-CDR2-FR3-CDR3-FR4
>206
MKFPSQPLLLLLFGIPGMICDIQMTQSSSSFSVSLGDRVTITCKASEDIYNRLAWYQQ
KPGNAPRLLISGATGLETGVPSRISGSGSGRDYTLSITSLQTEDVGTYYCQQYWSTP
RTFGGGTKLEIK
SEQ ID NO: 207 577Ab LC-CDR1
>207
KASEDIYNRLA
SEQ ID NO: 208 577Ab LC-CDR2
>208
GATGLET
SEQ ID NO: 209 577Ab LC-CDR3
>209
QQYWSTPRT
SEQ ID NO: 210 Human VSIG10 flag sequence-
>210
ATGGCCGCTGGGGGAAGCGCACCTGAACCTCGGGTCCTGGTCTGTCTCGGGGCTCTGCTG
GCTGGATGGGTCGCCGTCGGGCTGGAAGCCGTGGTCATCGGGGAGGTCCACGAAAACGTG
ACCCTCCATTGCGGTAATATTAGTGGGCTGAGGGGTCAGGTCACATGGTACCGGAACAAT
AGCGAACCAGTGTTCCTGCTCAGCTCCAACTCTAGTCTGCGACCTGCAGAGCCAAGGTTT
AGTCTCGTGGACGCCACCTCACTGCACATCGAGTCACTGAGCCTCGGCGATGAAGGAATC
TATACATGTCAGGAGATTCTGAATGTGACTCAGTGGTTCCAGGTCTGGCTGCAGGTGGCT
AGCGGACCCTACCAGATCGAGGTCCATATTGTGGCAACTGGGACCCTGCCTAACGGTACA
CTCTATGCCGCTAGGGGCTCACAGGTGGACTTTAGCTGCAATTCAAGCTCCAGACCCCCT
CCAGTGGTCGAATGGTGGTTCCAGGCCCTGAACTCTAGTTCAGAGTCCTTTGGGCACAAC
CTGACAGTGAATTTCTTTTCCCTGCTCCTGATCTCTCCCAACCTGCAGGGTAATTACACT
TGTCTGGCACTCAATCAGCTGTCTAAGAGGCATAGAAAAGTCACCACAGAGCTCCTGGTG
TACTATCCACCTCCAAGCGCACCTCAGTGCTGGGCTCAGATGGCATCCGGATCTTTCATG
CTGCAGCTCACTTGTAGATGGGACGGCGGATATCCAGACCCCGATTTTCTGTGGATCGAG
GAACCAGGGGGTGTGATTGTCGGCAAGTCTAAACTGGGAGTGGAGATGCTCAGTGAATCA
CAGCTGTCAGATGGAAAGAAATTCAAGTGCGTCACAAGCCACATCGTGGGGCCTGAAAGC
GGTGCTTCCTGTATGGTGCAGATTCGGGGGCCATCTCTCCTGAGTGAGCCCATGAAGACT
TGCTTTACCGGCGGAAACGTCACACTGACTTGTCAAGTGAGCGGCGCCTACCCCCCTGCT
AAAATCCTGTGGCTCCGGAATCTGACTCAGCCTGAGGTGATCATTCAGCCAAGCTCCCGC
CACCTGATCACCCAGGACGGACAGAACTCTACCCTCACAATTCATAATTGCAGTCAGGAC
CTGGATGAGGGCTACTATATCTGTCGCGCTGATTCCCCAGTGGGAGTCCGAGAGATGGAA
ATTTGGCTCTCTGTGAAGGAGCCCCTGAACATCGGGGGTATTGTCGGCACCATCGTGAGC
CTCCTGCTCCTGGGCCTGGCAATCATTAGCGGACTCCTGCTCCACTATTCCCCTGTCTTC
TGCTGGAAAGTGGGGAACACAAGCAGGGGTCAGAATATGGACGATGTGATGGTCCTGGTG
GACTCCGAGGAAGAAGAGGAGGAAGAAGAAGAAGAAGAGGAAGATGCAGCAGTGGGAGAG
CAGGAAGGAGCACGAGAACGCGAGGAACTGCCCAAGGAGATCCCTAAACAGGACCACATT
CATCGCGTCACCGCTCTGGTGAACGGCAATATCGAGCAGATGGGCAACGGATTTCAGGAT
CTGCAGGACGATTCTAGTGAGGAACAGAGTGACATTGTGCAGGAGGAGGACCGACCCGTG
GACTATAAAGACGACGATGATAAGTAA
SEQ ID NO: 211 pMSCV plasmid with Human VSIG10 flag sequence-
>211
TGAAAGACCCCACCTGTAGGTTTGGCAAGCTAGCTTAAGTAACGCCATTTTGCAAGGCAT
GGAAAATACATAACTGAGAATAGAGAAGTTCAGATCAAGGTTAGGAACAGAGAGACAGCA
GAATATGGGCCAAACAGGATATCTGTGGTAAGCAGTTCCTGCCCCGGCTCAGGGCCAAGA
ACAGATGGTCCCCAGATGCGGTCCCGCCCTCAGCAGTTTCTAGAGAACCATCAGATGTTT
CCAGGGTGCCCCAAGGACCTGAAATGACCCTGTGCCTTATTTGAACTAACCAATCAGTTC
GCTTCTCGCTTCTGTTCGCGCGCTTCTGCTCCCCGAGCTCAATAAAAGAGCCCACAACCC
CTCACTCGGCGCGCCAGTCCTCCGATAGACTGCGTCGCCCGGGTACCCGTATTCCCAATA
AAGCCTCTTGCTGTTTGCATCCGAATCGTGGACTCGCTGATCCTTGGGAGGGTCTCCTCA
GATTGATTGACTGCCCACCTCGGGGGTCTTTCATTTGGAGGTTCCACCGAGATTTGGAGA
CCCCTGCCCAGGGACCACCGACCCCCCCGCCGGGAGGTAAGCTGGCCAGCGGTCGTTTCG
TGTCTGTCTCTGTCTTTGTGCGTGTTTGTGCCGGCATCTAATGTTTGCGCCTGCGTCTGT
ACTAGTTAGCTAACTAGCTCTGTATCTGGCGGACCCGTGGTGGAACTGACGAGTTCTGAA
CACCCGGCCGCAACCCTGGGAGACGTCCCAGGGACTTTGGGGGCCGTTTTTGTGGCCCGA
CCTGAGGAAGGGAGTCGATGTGGAATCCGACCCCGTCAGGATATGTGGTTCTGGTAGGAG
ACGAGAACCTAAAACAGTTCCCGCCTCCGTCTGAATTTTTGCTTTCGGTTTGGAACCGAA
GCCGCGCGTCTTGTCTGCTGCAGCGCTGCAGCATCGTTCTGTGTTGTCTCTGTCTGACTG
TGTTTCTGTATTTGTCTGAAAATTAGGGCCAGACTGTTACCACTCCCTTAAGTTTGACCT
TAGGTCACTGGAAAGATGTCGAGCGGATCGCTCACAACCAGTCGGTAGATGTCAAGAAGA
GACGTTGGGTTACCTTCTGCTCTGCAGAATGGCCAACCTTTAACGTCGGATGGCCGCGAG
ACGGCACCTTTAACCGAGACCTCATCACCCAGGTTAAGATCAAGGTCTTTTCACCTGGCC
CGCATGGACACCCAGACCAGGTCCCCTACATCGTGACCTGGGAAGCCTTGGCTTTTGACC
CCCCTCCCTGGGTCAAGCCCTTTGTACACCCTAAGCCTCCGCCTCCTCTTCCTCCATCCG
CCCCGTCTCTCCCCCTTGAACCTCCTCGTTCGACCCCGCCTCGATCCTCCCTTTATCCAG
CCCTCACTCCTTCTCTAGGCGCCGGAATTAGATCTGCCACCATGGCCGCTGGGGGAAGCG
CACCTGAACCTCGGGTCCTGGTCTGTCTCGGGGCTCTGCTGGCTGGATGGGTCGCCGTCG
GGCTGGAAGCCGTGGTCATCGGGGAGGTCCACGAAAACGTGACCCTCCATTGCGGTAATA
TTAGTGGGCTGAGGGGTCAGGTCACATGGTACCGGAACAATAGCGAACCAGTGTTCCTGC
TCAGCTCCAACTCTAGTCTGCGACCTGCAGAGCCAAGGTTTAGTCTCGTGGACGCCACCT
CACTGCACATCGAGTCACTGAGCCTCGGCGATGAAGGAATCTATACATGTCAGGAGATTC
TGAATGTGACTCAGTGGTTCCAGGTCTGGCTGCAGGTGGCTAGCGGACCCTACCAGATCG
AGGTCCATATTGTGGCAACTGGGACCCTGCCTAACGGTACACTCTATGCCGCTAGGGGCT
CACAGGTGGACTTTAGCTGCAATTCAAGCTCCAGACCCCCTCCAGTGGTCGAATGGTGGT
TCCAGGCCCTGAACTCTAGTTCAGAGTCCTTTGGGCACAACCTGACAGTGAATTTCTTTT
CCCTGCTCCTGATCTCTCCCAACCTGCAGGGTAATTACACTTGTCTGGCACTCAATCAGC
TGTCTAAGAGGCATAGAAAAGTCACCACAGAGCTCCTGGTGTACTATCCACCTCCAAGCG
CACCTCAGTGCTGGGCTCAGATGGCATCCGGATCTTTCATGCTGCAGCTCACTTGTAGAT
GGGACGGCGGATATCCAGACCCCGATTTTCTGTGGATCGAGGAACCAGGGGGTGTGATTG
TCGGCAAGTCTAAACTGGGAGTGGAGATGCTCAGTGAATCACAGCTGTCAGATGGAAAGA
AATTCAAGTGCGTCACAAGCCACATCGTGGGGCCTGAAAGCGGTGCTTCCTGTATGGTGC
AGATTCGGGGGCCATCTCTCCTGAGTGAGCCCATGAAGACTTGCTTTACCGGCGGAAACG
TCACACTGACTTGTCAAGTGAGCGGCGCCTACCCCCCTGCTAAAATCCTGTGGCTCCGGA
ATCTGACTCAGCCTGAGGTGATCATTCAGCCAAGCTCCCGCCACCTGATCACCCAGGACG
GACAGAACTCTACCCTCACAATTCATAATTGCAGTCAGGACCTGGATGAGGGCTACTATA
TCTGTCGCGCTGATTCCCCAGTGGGAGTCCGAGAGATGGAAATTTGGCTCTCTGTGAAGG
AGCCCCTGAACATCGGGGGTATTGTCGGCACCATCGTGAGCCTCCTGCTCCTGGGCCTGG
CAATCATTAGCGGACTCCTGCTCCACTATTCCCCTGTCTTCTGCTGGAAAGTGGGGAACA
CAAGCAGGGGTCAGAATATGGACGATGTGATGGTCCTGGTGGACTCCGAGGAAGAAGAGG
AGGAAGAAGAAGAAGAAGAGGAAGATGCAGCAGTGGGAGAGCAGGAAGGAGCACGAGAAC
GCGAGGAACTGCCCAAGGAGATCCCTAAACAGGACCACATTCATCGCGTCACCGCTCTGG
TGAACGGCAATATCGAGCAGATGGGCAACGGATTTCAGGATCTGCAGGACGATTCTAGTG
AGGAACAGAGTGACATTGTGCAGGAGGAGGACCGACCCGTGGACTATAAAGACGACGATG
ATAAGTAAGTTAACGAATTCTACCGGGTAGGGGAGGCGCTTTTCCCAAGGCAGTCTGGAG
CATGCGCTTTAGCAGCCCCGCTGGGCACTTGGCGCTACACAAGTGGCCTCTGGCCTCGCA
CACATTCCACATCCACCGGTAGGCGCCAACCGGCTCCGTTCTTTGGTGGCCCCTTCGCGC
CACCTTCTACTCCTCCCCTAGTCAGGAAGTTCCCCCCCGCCCCGCAGCTCGCGTCGTGCA
GGACGTGACAAATGGAAGTAGCACGTCTCACTAGTCTCGTGCAGATGGACAGCACCGCTG
AGCAATGGAAGCGGGTAGGCCTTTGGGGCAGCGGCCAATAGCAGCTTTGCTCCTTCGCTT
TCTGGGCTCAGAGGCTGGGAAGGGGTGGGTCCGGGGGCGGGCTCAGGGGCGGGCTCAGGG
GCGGGGCGGGCGCCCGAAGGTCCTCCGGAGGCCCGGCATTCTGCACGCTTCAAAAGCGCA
CGTCTGCCGCGCTGTTCTCCTCTTCCTCATCTCCGGGCCTTTCGACCTGCAGCCCAAGCT
TACCATGACCGAGTACAAGCCCACGGTGCGCCTCGCCACCCGCGACGACGTCCCCAGGGC
CGTACGCACCCTCGCCGCCGCGTTCGCCGACTACCCCGCCACGCGCCACACCGTCGATCC
GGACCGCCACATCGAGCGGGTCACCGAGCTGCAAGAACTCTTCCTCACGCGCGTCGGGCT
CGACATCGGCAAGGTGTGGGTCGCGGACGACGGCGCCGCGGTGGCGGTCTGGACCACGCC
GGAGAGCGTCGAAGCGGGGGCGGTGTTCGCCGAGATCGGCCCGCGCATGGCCGAGTTGAG
CGGTTCCCGGCTGGCCGCGCAGCAACAGATGGAAGGCCTCCTGGCGCCGCACCGGCCCAA
GGAGCCCGCGTGGTTCCTGGCCACCGTCGGCGTCTCGCCCGACCACCAGGGCAAGGGTCT
GGGCAGCGCCGTCGTGCTCCCCGGAGTGGAGGCGGCCGAGCGCGCCGGGGTGCCCGCCTT
CCTGGAGACCTCCGCGCCCCGCAACCTCCCCTTCTACGAGCGGCTCGGCTTCACCGTCAC
CGCCGACGTCGAGGTGCCCGAAGGACCGCGCACCTGGTGCATGACCCGCAAGCCCGGTGC
CTGACGCCCGCCCCACGACCCGCAGCGCCCGACCGAAAGGAGCGCACGACCCCATGCATC
GATAAAATAAAAGATTTTATTTAGTCTCCAGAAAAAGGGGGGAATGAAAGACCCCACCTG
TAGGTTTGGCAAGCTAGCTTAAGTAACGCCATTTTGCAAGGCATGGAAAATACATAACTG
AGAATAGAGAAGTTCAGATCAAGGTTAGGAACAGAGAGACAGCAGAATATGGGCCAAACA
GGATATCTGTGGTAAGCAGTTCCTGCCCCGGCTCAGGGCCAAGAACAGATGGTCCCCAGA
TGCGGTCCCGCCCTCAGCAGTTTCTAGAGAACCATCAGATGTTTCCAGGGTGCCCCAAGG
ACCTGAAATGACCCTGTGCCTTATTTGAACTAACCAATCAGTTCGCTTCTCGCTTCTGTT
CGCGCGCTTCTGCTCCCCGAGCTCAATAAAAGAGCCCACAACCCCTCACTCGGCGCGCCA
GTCCTCCGATAGACTGCGTCGCCCGGGTACCCGTGTATCCAATAAACCCTCTTGCAGTTG
CATCCGACTTGTGGTCTCGCTGTTCCTTGGGAGGGTCTCCTCTGAGTGATTGACTACCCG
TCAGCGGGGGTCTTTCATGGGTAACAGTTTCTTGAAGTTGGAGAACAACATTCTGAGGGT
AGGAGTCGAATATTAAGTAATCCTGACTCAATTAGCCACTGTTTTGAATCCACATACTCC
AATACTCCTGAAATAGTTCATTATGGACAGCGCAGAAAGAGCTGGGGAGAATTGTGAAAT
TGTTATCCGCTCACAATTCCACACAACATACGAGCCGGAAGCATAAAGTGTAAAGCCTGG
GGTGCCTAATGAGTGAGCTAACTCACATTAATTGCGTTGCGCTCACTGCCCGCTTTCCAG
TCGGGAAACCTGTCGTGCCAGCTGCATTAATGAATCGGCCAACGCGCGGGGAGAGGCGGT
TTGCGTATTGGGCGCTCTTCCGCTTCCTCGCTCACTGACTCGCTGCGCTCGGTCGTTCGG
CTGCGGCGAGCGGTATCAGCTCACTCAAAGGCGGTAATACGGTTATCCACAGAATCAGGG
GATAACGCAGGAAAGAACATGTGAGCAAAAGGCCAGCAAAAGGCCAGGAACCGTAAAAAG
GCCGCGTTGCTGGCGTTTTTCCATAGGCTCCGCCCCCCTGACGAGCATCACAAAAATCGA
CGCTCAAGTCAGAGGTGGCGAAACCCGACAGGACTATAAAGATACCAGGCGTTTCCCCCT
GGAAGCTCCCTCGTGCGCTCTCCTGTTCCGACCCTGCCGCTTACCGGATACCTGTCCGCC
TTTCTCCCTTCGGGAAGCGTGGCGCTTTCTCATAGCTCACGCTGTAGGTATCTCAGTTCG
GTGTAGGTCGTTCGCTCCAAGCTGGGCTGTGTGCACGAACCCCCCGTTCAGCCCGACCGC
TGCGCCTTATCCGGTAACTATCGTCTTGAGTCCAACCCGGTAAGACACGACTTATCGCCA
CTGGCAGCAGCCACTGGTAACAGGATTAGCAGAGCGAGGTATGTAGGCGGTGCTACAGAG
TTCTTGAAGTGGTGGCCTAACTACGGCTACACTAGAAGAACAGTATTTGGTATCTGCGCT
CTGCTGAAGCCAGTTACCTTCGGAAAAAGAGTTGGTAGCTCTTGATCCGGCAAACAAACC
ACCGCTGGTAGCGGTGGTTTTTTTGTTTGCAAGCAGCAGATTACGCGCAGAAAAAAAGGA
TCTCAAGAAGATCCTTTGATCTTTTCTACGGGGTCTGACGCTCAGTGGAACGAAAACTCA
CGTTAAGGGATTTTGGTCATGAGATTATCAAAAAGGATCTTCACCTAGATCCTTTTAAAT
TAAAAATGAAGTTTTAAATCAATCTAAAGTATATATGAGTAAACTTGGTCTGACAGTTAC
CAATGCTTAATCAGTGAGGCACCTATCTCAGCGATCTGTCTATTTCGTTCATCCATAGTT
GCCTGACTCCCCGTCGTGTAGATAACTACGATACGGGAGGGCTTACCATCTGGCCCCAGT
GCTGCAATGATACCGCGAGACCCACGCTCACCGGCTCCAGATTTATCAGCAATAAACCAG
CCAGCCGGAAGGGCCGAGCGCAGAAGTGGTCCTGCAACTTTATCCGCCTCCATCCAGTCT
ATTAATTGTTGCCGGGAAGCTAGAGTAAGTAGTTCGCCAGTTAATAGTTTGCGCAACGTT
GTTGCCATTGCTACAGGCATCGTGGTGTCACGCTCGTCGTTTGGTATGGCTTCATTCAGC
TCCGGTTCCCAACGATCAAGGCGAGTTACATGATCCCCCATGTTGTGCAAAAAAGCGGTT
AGCTCCTTCGGTCCTCCGATCGTTGTCAGAAGTAAGTTGGCCGCAGTGTTATCACTCATG
GTTATGGCAGCACTGCATAATTCTCTTACTGTCATGCCATCCGTAAGATGCTTTTCTGTG
ACTGGTGAGTACTCAACCAAGTCATTCTGAGAATAGTGTATGCGGCGACCGAGTTGCTCT
TGCCCGGCGTCAATACGGGATAATACCGCGCCACATAGCAGAACTTTAAAAGTGCTCATC
ATTGGAAAACGTTCTTCGGGGCGAAAACTCTCAAGGATCTTACCGCTGTTGAGATCCAGT
TCGATGTAACCCACTCGTGCACCCAACTGATCTTCAGCATCTTTTACTTTCACCAGCGTT
TCTGGGTGAGCAAAAACAGGAAGGCAAAATGCCGCAAAAAAGGGAATAAGGGCGACACGG
AAATGTTGAATACTCATACTCTTCCTTTTTCAATATTATTGAAGCATTTATCAGGGTTAT
TGTCTCATGAGCGGATACATATTTGAATGTATTTAGAAAAATAAACAAATAGGGGTTCCG
CGCACATTTCCCCGAAAAGTGCCACCTGACGTCTAAGAAACCATTATTATCATGACATTA
ACCTATAAAAATAGGCGTATCACGAGGCCCTTTCGTCTCGCGCGTTTCGGTGATGACGGT
GAAAACCTCTGACACATGCAGCTCCCGGAGACGGTCACAGCTTGTCTGTAAGCGGATGCC
GGGAGCAGACAAGCCCGTCAGGGCGCGTCAGCGGGTGTTGGCGGGTGTCGGGGCTGGCTT
AACTATGCGGCATCAGAGCAGATTGTACTGAGAGTGCACCATATGCGGTGTGAAATACCG
CACAGATGCGTAAGGAGAAAATACCGCATCAGGCGCCATTCGCCATTCAGGCTGCGCAAC
TGTTGGGAAGGGCGATCGGTGCGGGCCTCTTCGCTATTACGCCAGCTGGCGAAAGGGGGA
TGTGCTGCAAGGCGATTAAGTTGGGTAACGCCAGGGTTTTCCCAGTCACGACGTTGTAAA
ACGACGGCGCAAGGAATGGTGCATGCAAGGAGATGGCGCCCAACAGTCCCCCGGCCACGG
GGCCTGCCACCATACCCACGCCGAAACAAGCGCTCATGAGCCCGAAGTGGCGAGCCCGAT
CTTCCCCATCGGTGATGTCGGCGATATAGGCGCCAGCAACCGCACCTGTGGCGCCGGTGA
TGCCGGCCACGATGCGTCCGGCGTAGAGGCGATTAGTCCAATTTGTTAAAGACAGGATAT
CAGTGGTCCAGGCTCTAGTTTTGACTCAACAATATCACCAGCTGAAGCCTATAGAGTACG
AGCCATAGATAAAATAAAAGATTTTATTTAGTCTCCAGAAAAAGGGGGGAA
SEQ ID NO: 212 mouse VSIG10 flag sequence
>212
ATGGCAGGGCTCCGCGTGCTCCTCTGTCTCGGTGCTCTCCTCGCTAGGCAGGGTTCCGCA
GGGCTCCAGCTCCTCCTCAATCCCAGCCGCGCCAACCTGAGTGTCCGACCTAATTCAGAG
GTGCTGCCCGGCATCCATCCTGACCTCGAGGCCGTGGCTATTGGAGAAGTCCACGATAAC
GTGACTCTGCGATGCGGATCCGCATCTGGAAGTAGGGGACTGGTGACCTGGTACAGAAAC
GACAGTGAGCCCGCCTTCCTGGTGAGCTTCAACAGCTCCCTCCCACCTGCAGCTCCTCGC
TTCTCTCTGGAGGATGCAGGTGCCCTCCGAATCGAGGCCCTGAGGCTCGAAGACGATGGC
AACTATACTTGTCAGGAGGTGCTGAATGAAACCCATTGGTTTCCTGTCAGGCTGAGAGTG
GCTTCAGGACCAGCATACGTGGAGGTCAACATCAGCGCTACAGGCACTCTGCCCAATGGA
ACCCTCTATGCAGCCAGGGGGTCTCAGGTGGACTTCAACTGCTGTAGTGCTGCACAGCCA
CCCCCTGAGGTGGAATGGTGGATCCAGACCCACTCTATTCCTGAGTTCCTGGGAAAGAAC
CTCTCAGCTAATAGCTTTACACTGATGCTCATGAGCCAGAACCTGCAGGGAAATTACACA
TGCTCAGCAACTAACGTGCTGAGCGGGCGGCAGCGCAAAGTCACCACAGAGCTGCTCGTG
TATTGGCCACCACCTAGCGCACCTCAGTGCTCCGTGGAGGTCTCTAGTGAAAGCACTACC
CTGGAGCTCGCCTGTAATTGGGACGGCGGATACCCTGATCCAACCTTCCTGTGGACAGAG
GAACCAGGGGGTACAATCATGGGCAACTCCAAGCTGCAGACTCTCTCTCCCGCCCAGCTG
CTCGAGGGCAAGAAGTTCAAGTGCGTGGGTAATCATATTCTGGGGCCAGAATCCGGTGCT
TCTTGTGTGGTCAAGCTGTCAAGCCCCCTGCTCCCTAGCCAGCCAATGAGAACCTGCTTC
GTCGGCGGAAACGTGACCCTGACATGTGAGGTGTCCGGGGCCAACCCACCCGCTAGAATC
CAGTGGCTGCGGAATCTCACACAGCCAGCCATTCAGCCCTCCTCTCATTATATCATTACC
CAGCAGGGCCAGAGTTCAAGCCTGACAATCCACAACTGCAGCCAGGACCTGGATGAGGGT
TTTTACTATTGTCAGGCAGAAAACCTGGTGGGCGTCAGAGCCACTAATATTTGGCTGTCC
GTGAAAGAGCCTCTCAATATCGGGGGTATTGTGGGCACAGTGGTCTCTCTGCTCCTGCTC
GGACTGGCTGTGGTCAGCGGACTGACACTCTACTATTCCCCAGCATTCTGGTGGAAGGGC
GGAAGTACTTTTCGGGGCCAGGACATGGGAGATGTGATGGTCCTGGTGGACAGCGAGGAA
GAAGAGGAGGAAGAAGAAGAAGAAGAGGAAAAAGAGGATGTCGCAGAGGAAGTGGAGCAG
GAAACTAACGAAACCGAGGAACTGCCAAAGGGGATCTCCAAACACGGTCATATTCACCGG
GTCACCGCTCTGGTGAACGGCAATCTCGACCGCATGGGGAATGGTTTCCAGGAGTTTCAG
GACGATTCTGACGGGCAGCAGAGTGGTATCGTCCAGGAAGATGGAAAGCCCGTGGACTAC
AAGGACGATGACGATAAATGA
SEQ ID NO: 213 pCDNA3.1 plasmid with mouse VSIG10 flag sequence
>213
GACGGATCGGGAGATCTCCCGATCCCCTATGGTGCACTCTCAGTACAATCTGCTCTGATG
CCGCATAGTTAAGCCAGTATCTGCTCCCTGCTTGTGTGTTGGAGGTCGCTGAGTAGTGCG
CGAGCAAAATTTAAGCTACAACAAGGCAAGGCTTGACCGACAATTGCATGAAGAATCTGC
TTAGGGTTAGGCGTTTTGCGCTGCTTCGCGATGTACGGGCCAGATATACGCGTTGACATT
GATTATTGACTAGTTATTAATAGTAATCAATTACGGGGTCATTAGTTCATAGCCCATATA
TGGAGTTCCGCGTTACATAACTTACGGTAAATGGCCCGCCTGGCTGACCGCCCAACGACC
CCCGCCCATTGACGTCAATAATGACGTATGTTCCCATAGTAACGCCAATAGGGACTTTCC
ATTGACGTCAATGGGTGGAGTATTTACGGTAAACTGCCCACTTGGCAGTACATCAAGTGT
ATCATATGCCAAGTACGCCCCCTATTGACGTCAATGACGGTAAATGGCCCGCCTGGCATT
ATGCCCAGTACATGACCTTATGGGACTTTCCTACTTGGCAGTACATCTACGTATTAGTCA
TCGCTATTACCATGGTGATGCGGTTTTGGCAGTACATCAATGGGCGTGGATAGCGGTTTG
ACTCACGGGGATTTCCAAGTCTCCACCCCATTGACGTCAATGGGAGTTTGTTTTGGCACC
AAAATCAACGGGACTTTCCAAAATGTCGTAACAACTCCGCCCCATTGACGCAAATGGGCG
GTAGGCGTGTACGGTGGGAGGTCTATATAAGCAGAGCTCTCTGGCTAACTAGAGAACCCA
CTGCTTACTGGCTTATCGAAATTAATACGACTCACTATAGGGAGACCCAAGCTGGCTAGC
GTTTAAACTTAAGCTTGGTACCGAGCTCGGATCCACTAGTCCAGTGTGGTGGAATTCAGA
TCTGCCACCATGGCAGGGCTCCGCGTGCTCCTCTGTCTCGGTGCTCTCCTCGCTAGGCAG
GGTTCCGCAGGGCTCCAGCTCCTCCTCAATCCCAGCCGCGCCAACCTGAGTGTCCGACCT
AATTCAGAGGTGCTGCCCGGCATCCATCCTGACCTCGAGGCCGTGGCTATTGGAGAAGTC
CACGATAACGTGACTCTGCGATGCGGATCCGCATCTGGAAGTAGGGGACTGGTGACCTGG
TACAGAAACGACAGTGAGCCCGCCTTCCTGGTGAGCTTCAACAGCTCCCTCCCACCTGCA
GCTCCTCGCTTCTCTCTGGAGGATGCAGGTGCCCTCCGAATCGAGGCCCTGAGGCTCGAA
GACGATGGCAACTATACTTGTCAGGAGGTGCTGAATGAAACCCATTGGTTTCCTGTCAGG
CTGAGAGTGGCTTCAGGACCAGCATACGTGGAGGTCAACATCAGCGCTACAGGCACTCTG
CCCAATGGAACCCTCTATGCAGCCAGGGGGTCTCAGGTGGACTTCAACTGCTGTAGTGCT
GCACAGCCACCCCCTGAGGTGGAATGGTGGATCCAGACCCACTCTATTCCTGAGTTCCTG
GGAAAGAACCTCTCAGCTAATAGCTTTACACTGATGCTCATGAGCCAGAACCTGCAGGGA
AATTACACATGCTCAGCAACTAACGTGCTGAGCGGGCGGCAGCGCAAAGTCACCACAGAG
CTGCTCGTGTATTGGCCACCACCTAGCGCACCTCAGTGCTCCGTGGAGGTCTCTAGTGAA
AGCACTACCCTGGAGCTCGCCTGTAATTGGGACGGCGGATACCCTGATCCAACCTTCCTG
TGGACAGAGGAACCAGGGGGTACAATCATGGGCAACTCCAAGCTGCAGACTCTCTCTCCC
GCCCAGCTGCTCGAGGGCAAGAAGTTCAAGTGCGTGGGTAATCATATTCTGGGGCCAGAA
TCCGGTGCTTCTTGTGTGGTCAAGCTGTCAAGCCCCCTGCTCCCTAGCCAGCCAATGAGA
ACCTGCTTCGTCGGCGGAAACGTGACCCTGACATGTGAGGTGTCCGGGGCCAACCCACCC
GCTAGAATCCAGTGGCTGCGGAATCTCACACAGCCAGCCATTCAGCCCTCCTCTCATTAT
ATCATTACCCAGCAGGGCCAGAGTTCAAGCCTGACAATCCACAACTGCAGCCAGGACCTG
GATGAGGGTTTTTACTATTGTCAGGCAGAAAACCTGGTGGGCGTCAGAGCCACTAATATT
TGGCTGTCCGTGAAAGAGCCTCTCAATATCGGGGGTATTGTGGGCACAGTGGTCTCTCTG
CTCCTGCTCGGACTGGCTGTGGTCAGCGGACTGACACTCTACTATTCCCCAGCATTCTGG
TGGAAGGGCGGAAGTACTTTTCGGGGCCAGGACATGGGAGATGTGATGGTCCTGGTGGAC
AGCGAGGAAGAAGAGGAGGAAGAAGAAGAAGAAGAGGAAAAAGAGGATGTCGCAGAGGAA
GTGGAGCAGGAAACTAACGAAACCGAGGAACTGCCAAAGGGGATCTCCAAACACGGTCAT
ATTCACCGGGTCACCGCTCTGGTGAACGGCAATCTCGACCGCATGGGGAATGGTTTCCAG
GAGTTTCAGGACGATTCTGACGGGCAGCAGAGTGGTATCGTCCAGGAAGATGGAAAGCCC
GTGGACTACAAGGACGATGACGATAAATGAGTTAACGCGGCCGCTCGAGTCTAGAGGGCC
CGTTTAAACCCGCTGATCAGCCTCGACTGTGCCTTCTAGTTGCCAGCCATCTGTTGTTTG
CCCCTCCCCCGTGCCTTCCTTGACCCTGGAAGGTGCCACTCCCACTGTCCTTTCCTAATA
AAATGAGGAAATTGCATCGCATTGTCTGAGTAGGTGTCATTCTATTCTGGGGGGTGGGGT
GGGGCAGGACAGCAAGGGGGAGGATTGGGAAGACAATAGCAGGCATGCTGGGGATGCGGT
GGGCTCTATGGCTTCTGAGGCGGAAAGAACCAGCTGGGGCTCTAGGGGGTATCCCCACGC
GCCCTGTAGCGGCGCATTAAGCGCGGCGGGTGTGGTGGTTACGCGCAGCGTGACCGCTAC
ACTTGCCAGCGCCCTAGCGCCCGCTCCTTTCGCTTTCTTCCCTTCCTTTCTCGCCACGTT
CGCCGGCTTTCCCCGTCAAGCTCTAAATCGGGGGCTCCCTTTAGGGTTCCGATTTAGTGC
TTTACGGCACCTCGACCCCAAAAAACTTGATTAGGGTGATGGTTCACGTAGTGGGCCATC
GCCCTGATAGACGGTTTTTCGCCCTTTGACGTTGGAGTCCACGTTCTTTAATAGTGGACT
CTTGTTCCAAACTGGAACAACACTCAACCCTATCTCGGTCTATTCTTTTGATTTATAAGG
GATTTTGCCGATTTCGGCCTATTGGTTAAAAAATGAGCTGATTTAACAAAAATTTAACGC
GAATTAATTCTGTGGAATGTGTGTCAGTTAGGGTGTGGAAAGTCCCCAGGCTCCCCAGCA
GGCAGAAGTATGCAAAGCATGCATCTCAATTAGTCAGCAACCAGGTGTGGAAAGTCCCCA
GGCTCCCCAGCAGGCAGAAGTATGCAAAGCATGCATCTCAATTAGTCAGCAACCATAGTC
CCGCCCCTAACTCCGCCCATCCCGCCCCTAACTCCGCCCAGTTCCGCCCATTCTCCGCCC
CATGGCTGACTAATTTTTTTTATTTATGCAGAGGCCGAGGCCGCCTCTGCCTCTGAGCTA
TTCCAGAAGTAGTGAGGAGGCTTTTTTGGAGGCCTAGGCTTTTGCAAAAAGCTCCCGGGA
GCTTGTATATCCATTTTCGGATCTGATCAAGAGACAGGATGAGGATCGTTTCGCATGATT
GAACAAGATGGATTGCACGCAGGTTCTCCGGCCGCTTGGGTGGAGAGGCTATTCGGCTAT
GACTGGGCACAACAGACAATCGGCTGCTCTGATGCCGCCGTGTTCCGGCTGTCAGCGCAG
GGGCGCCCGGTTCTTTTTGTCAAGACCGACCTGTCCGGTGCCCTGAATGAACTGCAGGAC
GAGGCAGCGCGGCTATCGTGGCTGGCCACGACGGGCGTTCCTTGCGCAGCTGTGCTCGAC
GTTGTCACTGAAGCGGGAAGGGACTGGCTGCTATTGGGCGAAGTGCCGGGGCAGGATCTC
CTGTCATCTCACCTTGCTCCTGCCGAGAAAGTATCCATCATGGCTGATGCAATGCGGCGG
CTGCATACGCTTGATCCGGCTACCTGCCCATTCGACCACCAAGCGAAACATCGCATCGAG
CGAGCACGTACTCGGATGGAAGCCGGTCTTGTCGATCAGGATGATCTGGACGAAGAGCAT
CAGGGGCTCGCGCCAGCCGAACTGTTCGCCAGGCTCAAGGCGCGCATGCCCGACGGCGAG
GATCTCGTCGTGACCCATGGCGATGCCTGCTTGCCGAATATCATGGTGGAAAATGGCCGC
TTTTCTGGATTCATCGACTGTGGCCGGCTGGGTGTGGCGGACCGCTATCAGGACATAGCG
TTGGCTACCCGTGATATTGCTGAAGAGCTTGGCGGCGAATGGGCTGACCGCTTCCTCGTG
CTTTACGGTATCGCCGCTCCCGATTCGCAGCGCATCGCCTTCTATCGCCTTCTTGACGAG
TTCTTCTGAGCGGGACTCTGGGGTTCGAAATGACCGACCAAGCGACGCCCAACCTGCCAT
CACGAGATTTCGATTCCACCGCCGCCTTCTATGAAAGGTTGGGCTTCGGAATCGTTTTCC
GGGACGCCGGCTGGATGATCCTCCAGCGCGGGGATCTCATGCTGGAGTTCTTCGCCCACC
CCAACTTGTTTATTGCAGCTTATAATGGTTACAAATAAAGCAATAGCATCACAAATTTCA
CAAATAAAGCATTTTTTTCACTGCATTCTAGTTGTGGTTTGTCCAAACTCATCAATGTAT
CTTATCATGTCTGTATACCGTCGACCTCTAGCTAGAGCTTGGCGTAATCATGGTCATAGC
TGTTTCCTGTGTGAAATTGTTATCCGCTCACAATTCCACACAACATACGAGCCGGAAGCA
TAAAGTGTAAAGCCTGGGGTGCCTAATGAGTGAGCTAACTCACATTAATTGCGTTGCGCT
CACTGCCCGCTTTCCAGTCGGGAAACCTGTCGTGCCAGCTGCATTAATGAATCGGCCAAC
GCGCGGGGAGAGGCGGTTTGCGTATTGGGCGCTCTTCCGCTTCCTCGCTCACTGACTCGC
TGCGCTCGGTCGTTCGGCTGCGGCGAGCGGTATCAGCTCACTCAAAGGCGGTAATACGGT
TATCCACAGAATCAGGGGATAACGCAGGAAAGAACATGTGAGCAAAAGGCCAGCAAAAGG
CCAGGAACCGTAAAAAGGCCGCGTTGCTGGCGTTTTTCCATAGGCTCCGCCCCCCTGACG
AGCATCACAAAAATCGACGCTCAAGTCAGAGGTGGCGAAACCCGACAGGACTATAAAGAT
ACCAGGCGTTTCCCCCTGGAAGCTCCCTCGTGCGCTCTCCTGTTCCGACCCTGCCGCTTA
CCGGATACCTGTCCGCCTTTCTCCCTTCGGGAAGCGTGGCGCTTTCTCATAGCTCACGCT
GTAGGTATCTCAGTTCGGTGTAGGTCGTTCGCTCCAAGCTGGGCTGTGTGCACGAACCCC
CCGTTCAGCCCGACCGCTGCGCCTTATCCGGTAACTATCGTCTTGAGTCCAACCCGGTAA
GACACGACTTATCGCCACTGGCAGCAGCCACTGGTAACAGGATTAGCAGAGCGAGGTATG
TAGGCGGTGCTACAGAGTTCTTGAAGTGGTGGCCTAACTACGGCTACACTAGAAGAACAG
TATTTGGTATCTGCGCTCTGCTGAAGCCAGTTACCTTCGGAAAAAGAGTTGGTAGCTCTT
GATCCGGCAAACAAACCACCGCTGGTAGCGGTTTTTTTGTTTGCAAGCAGCAGATTACGC
GCAGAAAAAAAGGATCTCAAGAAGATCCTTTGATCTTTTCTACGGGGTCTGACGCTCAGT
GGAACGAAAACTCACGTTAAGGGATTTTGGTCATGAGATTATCAAAAAGGATCTTCACCT
AGATCCTTTTAAATTAAAAATGAAGTTTTAAATCAATCTAAAGTATATATGAGTAAACTT
GGTCTGACAGTTACCAATGCTTAATCAGTGAGGCACCTATCTCAGCGATCTGTCTATTTC
GTTCATCCATAGTTGCCTGACTCCCCGTCGTGTAGATAACTACGATACGGGAGGGCTTAC
CATCTGGCCCCAGTGCTGCAATGATACCGCGAGACCCACGCTCACCGGCTCCAGATTTAT
CAGCAATAAACCAGCCAGCCGGAAGGGCCGAGCGCAGAAGTGGTCCTGCAACTTTATCCG
CCTCCATCCAGTCTATTAATTGTTGCCGGGAAGCTAGAGTAAGTAGTTCGCCAGTTAATA
GTTTGCGCAACGTTGTTGCCATTGCTACAGGCATCGTGGTGTCACGCTCGTCGTTTGGTA
TGGCTTCATTCAGCTCCGGTTCCCAACGATCAAGGCGAGTTACATGATCCCCCATGTTGT
GCAAAAAAGCGGTTAGCTCCTTCGGTCCTCCGATCGTTGTCAGAAGTAAGTTGGCCGCAG
TGTTATCACTCATGGTTATGGCAGCACTGCATAATTCTCTTACTGTCATGCCATCCGTAA
GATGCTTTTCTGTGACTGGTGAGTACTCAACCAAGTCATTCTGAGAATAGTGTATGCGGC
GACCGAGTTGCTCTTGCCCGGCGTCAATACGGGATAATACCGCGCCACATAGCAGAACTT
TAAAAGTGCTCATCATTGGAAAACGTTCTTCGGGGCGAAAACTCTCAAGGATCTTACCGC
TGTTGAGATCCAGTTCGATGTAACCCACTCGTGCACCCAACTGATCTTCAGCATCTTTTA
CTTTCACCAGCGTTTCTGGGTGAGCAAAAACAGGAAGGCAAAATGCCGCAAAAAAGGGAA
TAAGGGCGACACGGAAATGTTGAATACTCATACTCTTCCTTTTTCAATATTATTGAAGCA
TTTATCAGGGTTATTGTCTCATGAGCGGATACATATTTGAATGTATTTAGAAAAATAAAC
AAATAGGGGTTCCGCGCACATTTCCCCGAAAAGTGCCACCTGACGTC
SEQ ID NO: 214 Human VSIG10 flag sequence amino acid
>214
MAAGGSAPEPRVLVCLGALLAGWVAVGLEAVVIGEVHENVTLHCGNISGLRGQVTWYRNNSEPVFLL
SSNSSLRPAEPRFSLVDATSLHIESLSLGDEGIYTCQEILNVTQWFQVWLQVASGPYQIEVHIVATGTLPN
GTLYAARGSQVDFSCNSSSRPPPVVEWWFQALNSSSESFGHNLTVNFFSLLLISPNLQGNYTCLALNQL
SKRHRKVTIELLVYYPPPSAPQCWAQMASGSFMLQLTCRWDGGYPDPDFLWIEEPGGVIVGKSKLGV
EMLSESQLSDGKKFKCVTSHIVGPESGASCMVQIRGPSLLSEPMKTCFTGGNVTLTCQVSGAYPPAKIL
WLRNLTQPEVIIQPSSRHLITQDGQNSTLTIHNCSQDLDEGYYICRADSPVGVREMEIWLSVKEPLNIGGI
VGTIVSLLLLGLAIISGLLLHYSPVFCWKVGNTSRGQNMDDVMVLVDSEEEEEEEEEEEEDAAVGEQE
GAREREELPKEIPKQDHIHRVTALVNGNIEQMGNGFQDLQDDSSEEQSDIVQEEDRPVDYKDDDDK
SEQ ID NO: 215 mouse VSIG10 flag sequence amino acid
>215
MAGLRVLLCLGALLARQGSAGLQLLLNPSRANLSVRPNSEVLPGIHPDLEAVAIGEVHDNVTLRCGSA
SGSRGLVTWYRNDSEPAFLVSFNSSLPPAAPRFSLEDAGALRIEALRLEDDGNYTCQEVLNETHWFPVR
LRVASGPAYVEVNISATGTLPNGTLYAARGSQVDFNCCSAAQPPPEVEWWIQTHSIPEFLGKNLSANSF
TLMLMSQNLQGNYTCSATNVLSGRQRKVTTELLVYWPPPSAPQCSVEVSSESTTLELACNWDGGYPD
PTFLWIEEPGGTIMGNSKLQTLSPAQLLEGKKFKCVGNHILGPESGASCVVKLSSPLLPSQPMRTCFVG
GNVTLTCEVSGANPPARIQWLRNLTQPAIQPSSHYIITQQGQSSSLTIHNCSQDLDEGFYYCQAENLVGV
RATNIWLSVKEPLNIGGIVGTVVSLLLLGLAVVSGLTLYYSPAFWWKGGSTFRGQDMGDVMVLVDSE
EEEEEEEEEEEKEDVAEEVEQETNEIEELPKGISKHGHIHRVTALVNGNLDRMGNGFQEFQDDSDGQQ
SGIVQEDGKPVDYKDDDDK
SEQ ID NO: 216
AB-576 Heavy chain: DNA sequence (408 bp)
Leader sequence-FR1-CDR1-FR2-CDR2-FR3-CDR3-FR4
ATGGGATGGAGCTGGATCTTTCTCTTCCTCTTGTCAGGAACTGCAGGTGTCCTCTC
TGAGGTCCAGCTGCAACAATCTGGACCTGAGCTGGTGAAGCCTGGGGCTTCAGT
GAAAATGTCCTGTAAGGCTTCTGGATACACATTCACTGACTACTACATGAAGTG
GGTGAAGCAGAGTCATGGAAAGAGCCTTGAGTGGATTGGAGATATTAATCCTA
ACAATGGTGGTACAACCTACAACCAGAAGTTCAAGGGCAAGGCCACATTGA
CTGTGGACAAATCCTCCAACACAGCCTACATGCAGTTCAACAGCCTGACATCTGA
GGACTCTGCAGTCTATTTCTGTGCAAGATTTCGGCTACGAGCTATGGACTACTG
GGGTCAAGGAACCTCAGTCACCGTCTCCTCA
SEQ ID NO: 217
AB-576 Heavy chain: Amino acids sequence (136 aa)
Leader sequence-FR1-CDR1-FR2-CDR2-FR3-CDR3-FR4
MGWSWIFLFLLSGTAGVLSEVQLQQSGPELVKPGASVKMSCKASGYTFTDYYMKW
VKQSHGKSLEWIGDINPNNGGTTYNQKFKGKATLTVDKSSNTAYMQFNSLTSEDS
AVYFCARFRLRAMDYWGQGTSVTVSS
SEQ ID NO: 218 576Ab HC-CDR1
DYYMK
SEQ ID NO: 219 576Ab HC-CDR2
DINPNNGGTTYNQKFKG
SEQ ID NO: 220 576Ab HC-CDR3
FRLRAMDY
SEQ ID NO: 221
AB-576 Light chain: DNA sequence (399 bp)
Leader sequence-FR1-CDR1-FR2-CDR2-FR3-CDR3-FR4
ATGGAATCACAGACTCAGGTCCTCATGTCCCTGCTGTTCTGGGTATCTGGTACCT
GTGGGGACATTGTGATGACACAGTCTCCATCCTCCCTGACTGTGACAGCAGGAGA
GAAGGTCACTATGAGCTGCAAGTCCAGTCAGAGTCTGTTAAACAGTGGAGAT
CGAAAGAACTACTTGACCTGGTACCAGCAGAGACCAGGGCTGCCTCCTAAACT
GTTGATCTACTGGGCATCCACTAGGGAATCTGGGGTCCCTGATCGCTTCACAG
GCAGTGGATCTGGAACAGATTTCACTCTCACCATCAGCAGTGTGCAGTCTGAAGA
CCTGGCAGTTTATTTCTGTCAGAATGATTATATTTATCCGCTCACGTTCGGTGC
TGGGACCAAGCTGGAGCTGAAA
SEQ ID NO: 222
AB-576 Light chain: Amino acids sequence (133 aa)
Leader sequence-FR1-CDR1-FR2-CDR2-FR3-CDR3-FR4
MESQTQVLMSLLFWVSGTCGDIVMTQSPSSLTVTAGEKVTMSCKSSQSLLNSGDRK
NYLTWYQQRPGLPPKLLIYWASTRESGVPDRFTGSGSGTDFTLTISSVQSEDLAVYF
CQNDYIYPLTFGAGTKLELK
SEQ ID NO: 223 576Ab LC-CDR1
KSSQSLLNSGDRKNYLT
SEQ ID NO: 224 576Ab LC-CDR2
WASTRES
SEQ ID NO: 225 576Ab LC-CDR3
QNDYIYPLT

1. A monoclonal or polyclonal antibody or an antigen binding fragment thereof comprising an antigen binding site that binds specifically to an isolated polypeptide comprising a soluble ectodomain of a sequence selected from the group consisting of SEQ ID NOs:3 and 5; for use in treatment of cancer, wherein the cancer cells and/or the immune infiltrating cells in the microenvironment of said cancer express the polypeptide or a transmembrane polypeptide having the sequence selected from the group consisting of SEQ ID NOs:3 and 5.

2. The antibody or the antigen binding fragment of claim 1, wherein the ectodomain is selected from the group consisting of SEQ ID NOs:4 and 6.

3. The antibody or the antigen binding fragment of claim 2, wherein the immune infiltrating cells in the tumor microenvironment are myeloid lineage cells or wherein the cancer cells are epithelial cells, or both.

4. The antibody or the antigen binding fragment of claim 3, wherein the myeloid lineage cells are dendritic cells.

5. The antibody or the antigen binding fragment of claim 4, wherein the dendritic cells are CD1C positive dendritic cells.

6. The antibody or the antigen binding fragment of claim 4, wherein the dendritic cells are CD207 positive dendritic cells.

7. The antibody or the antigen binding fragment of claim 1, comprising a monoclonal antibody selected from the group consisting of 577-Ab and 576-Ab.

8. The antibody or antigen binding fragment of claim 1, comprising a monoclonal antibody binding to the same epitope as the monoclonal antibody selected from the group consisting of 577-Ab and 576-Ab.

9. The antibody or the antigen binding fragment of claim 1, comprising a monoclonal antibody comprising a heavy chain having an amino acid sequence selected from the group consisting of SEQ ID NO:19 and SEQ ID NO:35.

10. The antibody or antigen binding fragment of claim 1, comprising a monoclonal antibody comprising a heavy chain having the same binding specificity as the heavy chain having an amino acid sequence selected from the group consisting of SEQ ID NO:19 and SEQ ID NO:35.

11. The antibody or the antigen binding fragment of claim 1, comprising a monoclonal antibody comprising a light chain having an amino acid sequence selected from the group consisting of SEQ ID NO:24 and SEQ ID NO:40.

12. The antibody or antigen binding fragment of claim 1, comprising a monoclonal antibody comprising a light chain having the same binding specificity as the light chain having an amino acid sequence selected from the group consisting of SEQ ID NO:24 and SEQ ID NO:40.

13. The antibody or the antigen binding fragment of claim 1, comprising any of:
a. a heavy chain having an amino acid sequence of SEQ ID NO: 19 and a light chain having an amino acid sequence of SEQ ID NO: 24; or
b. a heavy chain having an amino acid sequence of SEQ ID NO: 35 and a light chain having an amino acid sequence of SEQ ID NO: 40.

14. The antibody or antigen binding fragment of claim 1, comprising:
a) a heavy chain variable domain comprising a vhCDR1, vhCDR2, and vhCDR3 from an anti-VSIG10 antibody; and
b) a light chain variable domain comprising a vlCDR1, vlCDR2 and vlCDR3 from said anti-VSIG10 antibody;
wherein said anti-VSIG10 antibody is selected from the group consisting of 577-Ab and wherein said SEQ ID Nos are 20, 21, 22 for vhCDR1, vhCDR2, vhCDR3, respectively and 25, 26, 27 for vlCDR1, vlCDR2, vlCDR3 respectively; or
wherein said anti-VSIG10 antibody is selected from the group consisting of 576-Ab and wherein said SEQ ID Nos are 36, 37, 222 for vhCDR1, vhCDR2, vhCDR3, respectively and 41, 42, 43 for vlCDR1, vlCDR2, vlCDR3 respectively.

15. The antibody or antigen binding fragment of claim 14, wherein said antigen binding domain is a scFv single chain Fv (scFv), wherein said heavy chain variable domain and said light chain variable domain are covalently attached via a scFv linker.

16. The antibody or antigen binding fragment of claim 1 that competes for binding with an antibody selected from the group consisting of 577-Ab and 576-Ab.

17. The antibody or the antigen binding fragment of claim 1, wherein the antigen binding site comprises a conformational or linear epitope, and wherein the antigen binding site contains about 3-7 contiguous or non-contiguous amino acids.

18. The antibody or fragment according to claim 1, wherein the antibody is a fully human antibody, chimeric antibody, humanized or primatized antibody.

19. The antibody or the antigen binding fragment according to claim 1, wherein the antibody is selected from the group consisting of Fab, Fab′, F(ab′)2, F(ab′), F(ab), Fv or scFv fragment and minimal recognition unit.

20. The antibody or the antigen binding fragment according to claim 1, wherein the antibody is coupled to a moiety selected from a drug, a radionuclide, a fluorophore, an enzyme, a toxin, a therapeutic agent, or a chemotherapeutic agent; and wherein the detectable marker is a radioisotope, a metal chelator, an enzyme, a fluorescent compound, a bioluminescent compound or a chemiluminescent compound.

21. A pharmaceutical composition comprising an antibody according to claim 1, and further comprising a pharmaceutically acceptable diluent or carrier.

22. A method for treating cancer comprising administering to a subject in need thereof an effective amount of a pharmaceutical composition according to claim 21.

23. A method for treating cancer comprising administering to a subject in need thereof an effective amount of an antibody according to claim 1.

24. The method of claim 23 wherein the treatment is combined with another moiety or therapy useful for treating cancer; wherein the therapy is radiation therapy, antibody therapy, chemotherapy, photodynamic therapy, adoptive T cell therapy, Treg depletion, surgery or in combination therapy with conventional drugs; or wherein the moiety is selected from the group consisting of immunosuppressants, cytotoxic drugs, tumor vaccines, antibodies (e.g. bevacizumab, erbitux), peptides, pepti-bodies, small molecules, chemotherapeutic agents such as cytotoxic and cytostatic agents (e.g. paclitaxel, cisplatin, vinorelbine, docetaxel, gemcitabine, temozolomide, irinotecan, 5FU, carboplatin), immunological modifiers such as interferons and interleukins, immunostimulatory antibodies, growth hormones or other cytokines, folic acid, vitamins, minerals, aromatase inhibitors, RNAi, Histone Deacetylase Inhibitors, and proteasome inhibitors.

25. The method according to claim 24 wherein said second antibody is an anti-checkpoint inhibitor antibody.

26. The method of claim 25, wherein said anti-checkpoint receptor or anti-costimulatory receptor antibody is selected from the group consisting of an anti-PD-1 antibody, an anti-PD-L1 antibody, an anti-PD-L2 antibody, an anti-LAG-3 antibody, an anti-CTLA-4 antibody, an anti-TIM-3 antibody, an anti-BTLA antibody, an anti-VSIG10 antibody, an anti-HVEM antibody, an anti-CEACAM1 antibody, an anti-GITR antibody, an anti-ICOS antibody, an anti-41BB antibody, an anti-OX40 antibody, an anti-KIR antibody, an anti-VISTA antibody, an anti-B7-H3 antibody, an anti-B7-H4 antibody, an anti-CD27 antibody, an anti-CD28 antibody, an anti-CD40 antibody, an anti-CD96 antibody, an anti-SIRPa antibody, an anti-CSF1R antibody, an anti-ILT2 antibody, an anti-ILT3 antibody, an anti-ILT4 antibody and an anti-ILT5 antibody.

27. The method of claim 23, comprising treating a patient for cancer, wherein the cancer is any of melanoma, liver cancer, renal cancer, brain cancer, breast cancer, colon cancer, colorectal cancer, lung cancer, small-cell lung cancer, non-small cell lung cancer, ovarian cancer, pancreatic cancer, prostate cancer, stomach cancer, endometrial cancer, multiple myeloma, Hodgkin's lymphoma, non Hodgkin's lymphoma, acute and chronic lymphoblastic leukemia and acute and chronic myeloid leukemia.

28. The method of claim 23, further comprising obtaining a sample of cancer cells and their microenvironment from the subject; assaying said sample to detect a presence of said isolated polypeptide in an immune cell or in a cancer cell; and if said presence is detected, administering said antibody or fragment thereof, or said pharmaceutical composition, to the subject.
